(19) United States
(12) Patent Application Publication
(10) Pub. No.: US 2025/0170161 A1
(43) Pub. Date: May 29, 2025

(54) INHIBIN SUBUNIT BETA E INHIBITOR AND METHODS OF USE THEREOF

(71) Applicant: RISEN (SHANGHAI) MEDICINAL TECHNOLOGY CO., LTD., Shanghai (CN)

(72) Inventors: Jiasheng LU, Shanghai (CN); Jun PAN, Shanghai (CN); Xinxin MA, Shanghai (CN); Wen LI, Shanghai (CN); Tianlun ZHOU, Shanghai (CN); Dawei CHEN, Shanghai (CN); Xianqi KONG, Dollard-des-Ormeaux (CA)

(21) Appl. No.: 18/917,300

(22) Filed: Oct. 16, 2024

Related U.S. Application Data
(60) Provisional application No. 63/590,562, filed on Oct. 16, 2023.

(30) Foreign Application Priority Data
Aug. 30, 2024 (WO) PCT/CN2024/115763

(51) Int. Cl. A61K 31/713; C12N 15/113

(52) U.S. Cl. CPC A61K 31/713; C12N 15/113; C12N 2310/11; C12N 2310/14; C12N 2310/333; C12N 2310/334; C12N 2310/335; C12N 2310/336

(57) ABSTRACT

There are provided inhibin subunit beta E (INHBE) inhibitors such as RNAi agents and RNAs useful for inhibiting the expression of INHBE, pharmaceutical compositions thereof, and applications thereof in the prevention and/or treatment of INHBE-associated disorders, e.g., metabolic disorders such as metabolic syndrome. In some embodiments the RNAi agents and RNAs comprise at least one non-canonical base pairing nucleotide.

### CROSS-REFERENCE TO RELATED APPLICATIONS

This application claims the benefit of priority from U.S. Provisional Application No. 63/590,562 filed Oct. 16, 2023 and from International (PCT) Application No. PCT/CN2024/115763 filed Aug. 30, 2024, each of which is hereby incorporated by reference in its entirety.

### REFERENCE TO AN ELECTRONIC SEQUENCE LISTING

The content of the electronic sequence listing (40604-135-Sequencelisting-V2.0.xml; Size: 6.09 MB; and Date of Creation: Feb. 8, 2025) is herein incorporated by reference in its entirety.

### FIELD

The present disclosure relates to an inhibin subunit beta E (INHBE) inhibitor such as an RNAi agent or an RNA, as well as pharmaceutical compositions and methods of use thereof for the prevention, treatment, and/or inhibition of diseases and disorders which are associated with the target INHBE gene.

### BACKGROUND

Metabolic syndrome is a pathological condition characterized by abdominal obesity, insulin resistance, hypertension, hyperlipidemia and related disorders. Metabolic syndrome can cause metabolic disorders in the body. It is not only a risk factor for cardiovascular disease, diabetes and kidney disease, but it also increases the risk of cancer and all-cause mortality. Metabolic syndrome affects about 20% to 25% of adults worldwide. The incidence of metabolic syndrome associated with obesity is increasing year by year in children and young adults, due to a combination of high-calorie/low-fiber diet and sedentary lifestyle. Current treatments for metabolic syndrome primarily involve diet and lifestyle changes, but patient compliance is generally poor.

Inhibin subunit beta E gene (INHBE) encodes a secretory protein called activin E. The mRNA of INHBE is mainly expressed in the liver, and it is involved in the regulation of hepatocyte growth and differentiation. It has been reported that insulin stimulates the expression of INHBE in hepatocytes and upregulates INHBE mRNA in the liver of diet-induced obese mice, suggesting that INHBE is involved in glucose metabolism (Hashimoto, O. et al., Life sciences. 2009; 85(13-14):534-40). Further studies showed that activin E was a putative factor for inducing insulin resistance and found that the gene expression of INHBE was positively correlated with insulin resistance and body mass index (BMI) (Sugiyama, M. et al., PLoS One, 2018; 13(3):e0194798). In addition, it has been reported that INHBE is a negative regulator of fat storage in the liver, suggesting that blocking INHBE may be beneficial in the treatment of metabolic diseases related to fat distribution (Akbari, P. et al., Nat Commun, 2022; 13: 4844).

International PCT Publication No. WO2023003922 discloses RNAi agents, such as double-stranded ribonucleic acid (dsRNA) agents, that target genes associated with metabolic disorders (e.g., inhibin subunit beta E (INHBE), activin A receptor type 1C (ACVR1C), perilipin-1 (PLIN1), phosphodiesterase 3B (PDE3B), inhibin subunit beta C (INHBC) gene). International PCT Publication No. WO2023044094 discloses INHBE modulator compositions and methods of use thereof to inhibit expression and/or activity of INHBE to prevent or treat an INHBE-associated disorder. However, development of RNAi drugs with improved efficacy and/or safety against INHBE targets is needed.

### SUMMARY

It is an object of the present disclosure to provide an inhibin subunit beta E (INHBE) inhibitor, such as an RNAi agent or an RNA, as well as pharmaceutical compositions and methods of use thereof for the prevention, treatment, and/or inhibition of diseases and disorders such as metabolic syndrome and related diseases.

The RNAi agents and RNAs of the disclosure have been designed to target the INHBE gene, including portions of the gene that are conserved in orthologs of other mammalian species. RNAi agents typically comprise sense strand and antisense strand which form a duplex, double-stranded RNA (referred to herein as “dsRNA”); RNAi agents comprising dsRNA are also referred to herein as “dsRNAi” agents.

Without intending to be limited by theory, it is believed that the RNAi agents and RNAs of the disclosure and the specific target sites and/or modifications in these RNAi agents and RNAs confer improved efficacy, stability, potency, durability, and/or safety. For example and without limitation, in some embodiments the RNAi agents and/or RNAs of the disclosure demonstrate: (1) improved efficacy and/or potency, e.g., by hybridizing more strongly to the target gene mRNA (as determined, for example, by an increase in Tm of the antisense strand/target mRNA duplex, e.g., an increase in calculated Tm of the antisense strand); and/or (2) improved safety, e.g., by reducing off-target effects, e.g., by reduced or weaker hybridization with off-target RNAs (as determined, for example, by a decrease in Tm for duplexes formed by the antisense strand and off-target RNAs).

The use of RNAi agents of the disclosure enables the targeted degradation of mRNAs of the INHBE target gene in mammals. The present inventors have demonstrated that RNAi agents of the disclosure can effect the RNA-induced silencing complex (RISC)-mediated cleavage of INHBE RNA transcripts, resulting in significant inhibition of expression of the INHBE target gene. In certain embodiments, RNAi agents of the disclosure are more effective (e.g., more potent) and/or more specific (e.g., more safe, less off-target effects) than previous RNAi agents targeting the same gene. In certain embodiments, RNAi agents target specific sites in the INHBE mRNA and/or include modifications of the RNA (e.g., non-canonical base pairing nucleotides, modified nucleotides, chemical modifications) selected to increase efficacy, potency, specificity, and/or safety, as described. In some such embodiments, RNAi agents comprise at least one modified nucleotide, including a non-canonical base pairing nucleotide. Methods and compositions comprising these RNAi agents are useful for treating a subject having an INHBE-associated disease or disorder, e.g., a metabolic disorder. Accordingly, there are provided methods for treating, preventing or inhibiting a metabolic disorder in a subject who would benefit from inhibiting or reducing INHBE expression using RNAi agents and compositions of the disclosure.

In one aspect, the present disclosure provides a double-stranded ribonucleic acid interference (dsRNAi) agent useful for inhibiting expression of inhibin subunit beta E (INHBE) in a cell, wherein the dsRNAi agent comprises a sense strand and an antisense strand forming a double-stranded RNA (dsRNA) region, the antisense strand comprising a region of complementarity to an INHBE mRNA, wherein the region of complementarity comprises at least 15 contiguous nucleotides. In certain embodiments, the dsRNAi agent comprises at least one non-canonical base pairing nucleotide, as described further hereinbelow.

In some embodiments, the dsRNAi agent comprises one, two, three, four, five or more non-canonical base pairing nucleotides. Non-canonical base pairing nucleotides and additional modified nucleotides may be present in the region of complementarity, in the dsRNA region, or at any other position in the dsRNAi agent. Non-canonical base pairing nucleotides and additional modified nucleotides may be included on the antisense strand, the sense strand, or both. In some embodiments, the dsRNAi agent comprises at least one additional modified nucleotide in addition to the non-canonical base pairing nucleotide(s); such additional modified nucleotide(s) may or may not be on the same oligonucleotide strand as the non-canonical base pairing nucleotide(s).

In some embodiments, the dsRNAi agent or RNA has increased efficacy, potency, specificity and/or safety, and/or reduced off-target effects, compared to a dsRNAi agent or RNA having the same nucleotide sequence with no non-canonical base pairing and/or modified nucleotide. In some such embodiments, the melting temperature (Tm) of the dsRNAi agent or RNA is changed by at least 2° C. compared to the Tm of the same nucleotide sequence with no non-canonical base pairing and/or modified nucleotide.

In some embodiments, the dsRNAi agent or RNA of the disclosure comprises a non-canonical base pairing nucleotide and/or modified nucleotide which changes the Tm by at least 2° C., e.g., by 2° C., by over 2° C., by 3° C., by 4° C., by 5° C., or more. In some such embodiments, the Tm is calculated using a formula or algorithm as described herein.

In some embodiments, the dsRNAi agent or RNA of the disclosure comprises at least one non-canonical base pairing nucleotide and/or modified nucleotide at positions 1-11 of the oligonucleotide strand, e.g., the antisense, according to the direction from the 5′ end to the 3′ end.

In some embodiments, the dsRNAi agent or RNA of the disclosure comprises at least one non-canonical base pairing nucleotide and/or modified nucleotide at positions 12-21 of the oligonucleotide strand, e.g., the antisense, according to the direction from the 5′ end to the 3′ end. In some embodiments, a guanine in G₀ is replaced with hypoxanthine (I) in the sequence 5′-N₁N₂G₀N₃N₄-3′ (N₁, N₂, N₃, and N₄ are nucleotides independently containing adenine (A), cytosine (C), guanine (G), thymine (T) or uracil (U) as a base, and G₀ is a nucleotide containing guanine as a base), when at least three (3) bases in N₁, N₂, N₃, and N₄ are adenine (A) or uracil (U). In some such embodiments, at least one of N₁, N₂, N₃, N₄ and G₀ has a modified sugar moiety and/or a modified internucleotide linkage.

In some embodiments, the dsRNAi agent or RNA of the disclosure comprises at least one non-canonical base pairing nucleotide and/or modified nucleotide at position 6, 7 or 8 of the oligonucleotide, according to the direction from the 5′ end to the 3′ end, if at least four (4) of the nucleotides at positions 2-8 are A or U and at least one of the nucleotides at positions 6-8 is G, such that the G at position 6, 7 and/or 8 is replaced with a non-canonical base pairing nucleotide.

In some such embodiments, the dsRNAi agent or RNA of the disclosure comprises at least 15 contiguous nucleotides differing by no more than 3 nucleotides from any one of the nucleotide sequences shown in Table 1 and Table 2. In some such embodiments, the antisense strand comprises at least 15 contiguous nucleotides differing by no more than 3 nucleotides from any one of the nucleotide sequences shown in Table 1 and Table 2, and the sense strand comprises at least 15 nucleotides complementary to the antisense strand. In some such embodiments, the sense strand comprises at least 15 contiguous nucleotides differing by no more than 3 nucleotides from any one of the nucleotide sequences shown in Table 1 and Table 2, and the antisense strand comprises at least 15 nucleotides complementary to the sense strand. In some such embodiments, the antisense strand and/or the sense stand differs by no more than 1, 2 or 3 nucleotides from any one of the nucleotide sequences shown in Table 1 and Table 2. In some embodiments, the antisense strand and/or the sense strand has any one of the nucleotide sequences shown in Table 1 and Table 2. In some embodiments, the dsRNAi agent comprises a dsRNA comprising any one of the duplex sequences shown in Table 1 and Table 2, e.g., any one of IN-001 to IN-488 and INI-001 to INI-308, or any one of IN-489 to IN-546. In some embodiments, the dsRNAi agent comprises a sense strand comprising 15 contiguous nucleotides as set forth in any one of SEQ ID NOs. 1621 to 1689. In some embodiments, the dsRNAi agent comprises a sense strand which is 19 nucleotides in length and which comprises 15 contiguous nucleotides as set forth in any one of SEQ ID NOs. 1621 to 1689. In some embodiments, the dsRNAi agent comprises an antisense strand comprising 15 contiguous nucleotides as set forth in any one of SEQ ID NOs. 1690 to 1758. In some embodiments, the dsRNAi agent comprises an antisense strand which is 21 nucleotides in length and which comprises 15 contiguous nucleotides as set forth in any one of SEQ ID NOs. 1690 to 1758.

It should be understood that, in the present disclosure, reference to a dsRNAi agent, RNA, or nucleotide sequence “differing” from another nucleotide sequence can refer to a difference in nucleotide sequence length, a difference in nucleotides, or a combination of the two.

Examples of non-canonical bases in nucleosides or nucleotides include, without limitation, bases in inosine (I), xanthosine (X), 7-methylguanosine (m7G), N6-methyladenosine (m6A), dihydrouridine, 5-methylcytosine (m5C), pseudouridine (Ψ), and N1-methylpseudouridine (m1Ψ). In certain embodiments, the non-canonical base pairing nucleotide is inosinic acid (I). In certain embodiments, the non-canonical base pairing nucleoside is inosine (I). In some embodiments, at least one guanine (G) in the dsRNAi agent or RNA of the disclosure, e.g., the sense strand and/or the antisense strand, is replaced with hypoxanthine (I). Examples of additional modified nucleosides and nucleotides are described elsewhere herein.

In another aspect, the present disclosure provides a dsRNAi agent comprising a sense strand and an antisense strand, wherein the antisense strand comprises at least 15 contiguous nucleotides differing by no more than 3 (0, 1, 2 or 3) nucleotides from any one of the nucleotide sequences shown in Table 1 or Table 2, and the sense strand has at least 15, 16, 17, 18, 19, 20 or 21 nucleotides complementary to the antisense strand.

In some embodiments, the antisense strand comprises at least 15, 16, 17, 18, 19, 20 or 21 nucleotides differing by 0, 1, 2 or 3 nucleotides from any one of the nucleotide sequences shown in Table 1 or Table 2.

In some embodiments, the sense strand's nucleotide sequence is at least substantially complementary to the antisense strand's nucleotide sequence.

In some embodiments, the sense strand's nucleotide sequence is completely complementary (i.e., 100% complementary) to the antisense strand's nucleotide sequence.

In some embodiments, the lengths of the sense strand and the antisense strand are each independently 17-25 nucleotides. In some embodiments, the lengths of the sense strand and the antisense strand are each independently 19-23 nucleotides. In some embodiments, the lengths of the sense strand and the antisense strand are each independently 19-21 nucleotides.

In some embodiments, the sense strand is 19 nucleotides in length which comprises any one of the following:
15 contiguous nucleotides as set forth in SEQ ID NO 1621: GGGUCAAGCACAGCU;
15 contiguous nucleotides as set forth in SEQ ID NO 1622: AAGCACAGCUAUCCA;
15 contiguous nucleotides as set forth in SEQ ID NO 1623: CAGCUAUCCAUCAGA;
15 contiguous nucleotides as set forth in SEQ ID NO 1624: AUCCAUCAGAUGAUC;
15 contiguous nucleotides as set forth in SEQ ID NO 1625: UCAGAUGAUCUACUU;
15 contiguous nucleotides as set forth in SEQ ID NO 1626: UGAUCUACUUUCAGC;
15 contiguous nucleotides as set forth in SEQ ID NO 1627: UGAGUCCCAGACAAU;
15 contiguous nucleotides as set forth in SEQ ID NO 1628: AGCCAAGCAGCAAAU;
15 contiguous nucleotides as set forth in SEQ ID NO 1629: GUCGUCCCAGAAUAA;
15 contiguous nucleotides as set forth in SEQ ID NO 1630: AUCAGCUUUGCUACU;
15 contiguous nucleotides as set forth in SEQ ID NO 1631: UGCUACUGUCACAGA;
15 contiguous nucleotides as set forth in SEQ ID NO 1632: UUCCUGGCACUCUUU;
15 contiguous nucleotides as set forth in SEQ ID NO 1633: UCUUUGCUUGAGGAU;
15 contiguous nucleotides as set forth in SEQ ID NO 1634: UGGUGUCCUGAAACU;
15 contiguous nucleotides as set forth in SEQ ID NO 1635: CCUGAAACUGCAACU;
15 contiguous nucleotides as set forth in SEQ ID NO 1636: AGGCAACAGCACAGU;
15 contiguous nucleotides as set forth in SEQ ID NO 1637: CCUAGAGCUUAAGAU;
15 contiguous nucleotides as set forth in SEQ ID NO 1638: CUUAAGAUCCGAGCC;
15 contiguous nucleotides as set forth in SEQ ID NO 1639: ACCAUUACGUAGACU;
15 contiguous nucleotides as set forth in SEQ ID NO 1640: CUCAAAGCCAACAAU;
15 contiguous nucleotides as set forth in SEQ ID NO 1641: CUCUACCUGGAUCAU;
15 contiguous nucleotides as set forth in SEQ ID NO 1642: GAUCAUAAUGGCAAU;
15 contiguous nucleotides as set forth in SEQ ID NO 1643: AAGAGACCAAGAUGA;
15 contiguous nucleotides as set forth in SEQ ID NO 1644: CUGGCAAUAUGACUC;
15 contiguous nucleotides as set forth in SEQ ID NO 1645: UGGGCACUUUCUUGU;
15 contiguous nucleotides as set forth in SEQ ID NO 1646: UUCUUGUCUGAGACU;
15 contiguous nucleotides as set forth in SEQ ID NO 1647: UGAGACUCUGGCUUA;
15 contiguous nucleotides as set forth in SEQ ID NO 1648: UUGGGAGAUGGGUAA;
15 contiguous nucleotides as set forth in SEQ ID NO 1649: GAUGGGUAAAGCGUU;
15 contiguous nucleotides as set forth in SEQ ID NO 1650: AAAGCGUUUCUUCUA;
15 contiguous nucleotides as set forth in SEQ ID NO 1651: CUUCUAAAGGGGUCU;
15 contiguous nucleotides as set forth in SEQ ID NO 1652: UACCCAGAAAGCAUG;
15 contiguous nucleotides as set forth in SEQ ID NO 1653: AAGUCCUGUGAGAAG;
15 contiguous nucleotides as set forth in SEQ ID NO 1654: GAAGGCAGAGAAAAA;
15 contiguous nucleotides as set forth in SEQ ID NO 1655: CAGAGAAAAAUUACU;
15 contiguous nucleotides as set forth in SEQ ID NO 1656: UCCCAAGAUGAGAAA;
15 contiguous nucleotides as set forth in SEQ ID NO 1657: GAGGAAGCAGAUAGA;
15 contiguous nucleotides as set forth in SEQ ID NO 1658: CUGUUGAGGUACCUU;
15 contiguous nucleotides as set forth in SEQ ID NO 1659: AGGUACCUUAAGGGA;
15 contiguous nucleotides as set forth in SEQ ID NO 1660: CAGGAGUCAGGAAAA;
15 contiguous nucleotides as set forth in SEQ ID NO 1661: GGGAGACAAGCAUUU;
15 contiguous nucleotides as set forth in SEQ ID NO 1662: ACAAGCAUUUAUACU;
15 contiguous nucleotides as set forth in SEQ ID NO 1663: CAUUUAUACUUUCUU;
15 contiguous nucleotides as set forth in SEQ ID NO 1664: AAAGAAAAUCAACAA;
15 contiguous nucleotides as set forth in SEQ ID NO 1665: CAAAUGUGAGUCAUA;
15 contiguous nucleotides as set forth in SEQ ID NO 1666: GUCAUAAAGAAGGGU;
15 contiguous nucleotides as set forth in SEQ ID NO 1667: AGAGCAACAGUUCUU;
15 contiguous nucleotides as set forth in SEQ ID NO 1668: GGGUGUCCACAAAGU;
15 contiguous nucleotides as set forth in SEQ ID NO 1669: UCCACAAAGUCAAAG;
15 contiguous nucleotides as set forth in SEQ ID NO 1670: AAAGUCAAAGCUAUU;
15 contiguous nucleotides as set forth in SEQ ID NO 1671: AAAGCUAUUUUCAUA;
15 contiguous nucleotides as set forth in SEQ ID NO 1672: AUUUUCAUAAUAAUA;
15 contiguous nucleotides as set forth in SEQ ID NO 1673: AUAAUACUAACAUGU;
15 contiguous nucleotides as set forth in SEQ ID NO 1674: ACUAACAUGUUAUUU;
15 contiguous nucleotides as set forth in SEQ ID NO 1675: AUGUUAUUUGCCUUU;
15 contiguous nucleotides as set forth in SEQ ID NO 1676: AUUUGCCUUUUGAAU;
15 contiguous nucleotides as set forth in SEQ ID NO 1677: UUUUGAAUUCUCAUU;
15 contiguous nucleotides as set forth in SEQ ID NO 1678: UUCUCAUUAUCUUAA;
15 contiguous nucleotides as set forth in SEQ ID NO 1679: UUAUCUUAAAAUUGU;
15 contiguous nucleotides as set forth in SEQ ID NO 1680: GUGUGACAUGUGAUU;
15 contiguous nucleotides as set forth in SEQ ID NO 1681: AUGUGAUUACAUCAU;
15 contiguous nucleotides as set forth in SEQ ID NO 1682: UUACAUCAUCUUUCU;
15 contiguous nucleotides as set forth in SEQ ID NO 1683: AUCUUUCUGACAUCA;
15 contiguous nucleotides as set forth in SEQ ID NO 1684: UCUGACAUCAUUGUU;
15 contiguous nucleotides as set forth in SEQ ID NO 1685: GUUAAUGGAAUGUGU;
15 contiguous nucleotides as set forth in SEQ ID NO 1686: CAGCUUUGCUACUGU;
15 contiguous nucleotides as set forth in SEQ ID NO 1687: AGACUCUGGCUUAUU;
15 contiguous nucleotides as set forth in SEQ ID NO 1688: GCAACAGUUCUUCAA;
and 15 contiguous nucleotides as set forth in SEQ ID NO 1689: AAAGCUAUUUUCAUA.

In some embodiments, the antisense strand is 21 nucleotides in length which comprises any one of the following:
15 contiguous nucleotides as set forth in SEQ ID NO 1690: AGCUGUGCUUGACCC;
15 contiguous nucleotides as set forth in SEQ ID NO 1691: UGGAUAGCUGUGCUU;
15 contiguous nucleotides as set forth in SEQ ID NO 1692: UCUGAUGGAUAGCUG;
15 contiguous nucleotides as set forth in SEQ ID NO 1693: GAUCAUCUGAUGGAU;
15 contiguous nucleotides as set forth in SEQ ID NO 1694: AAGUAGAUCAUCUGA;
15 contiguous nucleotides as set forth in SEQ ID NO 1695: AAGUAGAUCAUCUGA;
15 contiguous nucleotides as set forth in SEQ ID NO 1696: AUUGUCUGGGACUCA;
15 contiguous nucleotides as set forth in SEQ ID NO 1697: AUUUGCUGCUUGGCU;
15 contiguous nucleotides as set forth in SEQ ID NO 1698: UUAUUCUGGGACGAC;
15 contiguous nucleotides as set forth in SEQ ID NO 1699: AGUAGCAAAGCUGAU;
15 contiguous nucleotides as set forth in SEQ ID NO 1700: UCUGUGACAGUAGCA;
15 contiguous nucleotides as set forth in SEQ ID NO 1701: UCUGUGACAGUAGCA;
15 contiguous nucleotides as set forth in SEQ ID NO 1702: AUCCUCAAGCAAAGA;
15 contiguous nucleotides as set forth in SEQ ID NO 1703: AGUUUCAGGACACCA;
15 contiguous nucleotides as set forth in SEQ ID NO 1704: AGUUGCAGUUUCAGG;
15 contiguous nucleotides as set forth in SEQ ID NO 1705: ACUGUGCUGUUGCCU;
15 contiguous nucleotides as set forth in SEQ ID NO 1706: AUCUUAAGCUCUAGG;
15 contiguous nucleotides as set forth in SEQ ID NO 1707: GGCUCGGAUCUUAAG;
15 contiguous nucleotides as set forth in SEQ ID NO 1708: AGUCUACGUAAUGGU;
15 contiguous nucleotides as set forth in SEQ ID NO 1709: AUUGUUGGCUUUGAG;
15 contiguous nucleotides as set forth in SEQ ID NO 1710: AUGAUCCAGGUAGAG;
15 contiguous nucleotides as set forth in SEQ ID NO 1711: AUUGCCAUUAUGAUC;
15 contiguous nucleotides as set forth in SEQ ID NO 1712: UCAUCUUGGUCUCUU;
15 contiguous nucleotides as set forth in SEQ ID NO 1713: AGUCAUAUUGCCAGG;
15 contiguous nucleotides as set forth in SEQ ID NO 1714: ACAAGAAAGUGCCCA;
15 contiguous nucleotides as set forth in SEQ ID NO 1715: AGUCUCAGACAAGAA;
15 contiguous nucleotides as set forth in SEQ ID NO 1716: UAAGCCAGAGUCUCA;
15 contiguous nucleotides as set forth in SEQ ID NO 1717: UUACCCAUCUCCCAA;
15 contiguous nucleotides as set forth in SEQ ID NO 1718: AACGCUUUACCCAUC;
15 contiguous nucleotides as set forth in SEQ ID NO 1719: UAGAAGAAACGCUUU;
15 contiguous nucleotides as set forth in SEQ ID NO 1720: AGACCCCUUUAGAAG;
15 contiguous nucleotides as set forth in SEQ ID NO 1721: CAUGCUUUCUGGGUA;
15 contiguous nucleotides as set forth in SEQ ID NO 1722: CUUCUCACAGGACUU;
15 contiguous nucleotides as set forth in SEQ ID NO 1723: UUUUUCUCUGCCUUC;
15 contiguous nucleotides as set forth in SEQ ID NO 1724: AGUAAUUUUUCUCUG;
15 contiguous nucleotides as set forth in SEQ ID NO 1725: UUUCUCAUCUUGGGA;
15 contiguous nucleotides as set forth in SEQ ID NO 1726: UCUAUCUGCUUCCUC;
15 contiguous nucleotides as set forth in SEQ ID NO 1727: AAGGUACCUCAACAG;
15 contiguous nucleotides as set forth in SEQ ID NO 1728: UCCCUUAAGGUACCU;
15 contiguous nucleotides as set forth in SEQ ID NO 1729: UUUUCCUGACUCCUG;
15 contiguous nucleotides as set forth in SEQ ID NO 1730: AAAUGCUUGUCUCCC;
15 contiguous nucleotides as set forth in SEQ ID NO 1731: AGUAUAAAUGCUUGU;
15 contiguous nucleotides as set forth in SEQ ID NO 1732: AAGAAAGUAUAAAUG;
15 contiguous nucleotides as set forth in SEQ ID NO 1733: UUGUUGAUUUUCUUU;
15 contiguous nucleotides as set forth in SEQ ID NO 1734: UAUGACUCACAUUUG;
15 contiguous nucleotides as set forth in SEQ ID NO 1735: ACCCUUCUUUAUGAC;
15 contiguous nucleotides as set forth in SEQ ID NO 1736: AAGAACUGUUGCUCU;
15 contiguous nucleotides as set forth in SEQ ID NO 1737: ACUUUGUGGACACCC;
15 contiguous nucleotides as set forth in SEQ ID NO 1738: CUUUGACUUUGUGGA;
15 contiguous nucleotides as set forth in SEQ ID NO 1739: AAUAGCUUUGACUUU;
15 contiguous nucleotides as set forth in SEQ ID NO 1740: UAUGAAAAUAGCUUU;
15 contiguous nucleotides as set forth in SEQ ID NO 1741: UAUUAUUAUGAAAAU;
15 contiguous nucleotides as set forth in SEQ ID NO 1742: ACAUGUUAGUAUUAU;
15 contiguous nucleotides as set forth in SEQ ID NO 1743: AAAUAACAUGUUAGU;
15 contiguous nucleotides as set forth in SEQ ID NO 1744: AAAGGCAAAUAACAU;
15 contiguous nucleotides as set forth in SEQ ID NO 1745: AUUCAAAAGGCAAAU;
15 contiguous nucleotides as set forth in SEQ ID NO 1746: AAUGAGAAUUCAAAA;
15 contiguous nucleotides as set forth in SEQ ID NO 1747: UUAAGAUAAUGAGAA;
15 contiguous nucleotides as set forth in SEQ ID NO 1748: ACAAUUUUAAGAUAA;
15 contiguous nucleotides as set forth in SEQ ID NO 1749: AAUCACAUGUCACAC;
15 contiguous nucleotides as set forth in SEQ ID NO 1750: AUGAUGUAAUCACAU;
15 contiguous nucleotides as set forth in SEQ ID NO 1751: AGAAAGAUGAUGUAA;
15 contiguous nucleotides as set forth in SEQ ID NO 1752: UGAUGUCAGAAAGAU;
15 contiguous nucleotides as set forth in SEQ ID NO 1753: AACAAUGAUGUCAGA;
15 contiguous nucleotides as set forth in SEQ ID NO 1754: ACACAUUCCAUUAAC;
15 contiguous nucleotides as set forth in SEQ ID NO 1755: ACAGUAGCAAAGCUG;
15 contiguous nucleotides as set forth in SEQ ID NO 1756: AAUAAGCCAGAGUCU;
15 contiguous nucleotides as set forth in SEQ ID NO 1757: UUGAAGAACUGUUGC;
and 15 contiguous nucleotides as set forth in SEQ ID NO 1758: UAUGAAAAUAGCUUU.

In some embodiments, the sense strand of the disclosure is derived from the mRNA sequence of human INHBE (e.g., Gene ID: 83729, NCBI Reference Sequence: NM_031479.5 (SEQ ID NO: 1619)). Alternatively, the sense strand of the disclosure is derived from a fragment of the mRNA sequence of human INHBE. In some embodiments, the sense strand of the disclosure is derived from the mRNA sequence of cynomolgus monkey INHBE (e.g., Gene ID: 102127493, NCBI Reference Sequence: XM_005571319.3 (SEQ ID NO: 1620)). Alternatively, the sense strand of the disclosure is derived from a fragment of the mRNA sequence of cynomolgus monkey INHBE. The mRNA sequences for human and cynomolgus monkey INHBE are shown in FIGS. 29 and 30.

In some embodiments, the dsRNAi agent or RNA of the disclosure comprises any one of the antisense strand sequences or sense strand sequences shown in Table 1 or Table 2. In some embodiments, the dsRNAi agent or RNA comprises any one of the antisense strand sequences or sense strand sequences set forth in SEQ ID NOs: 1-1590. In some embodiments, the dsRNAi agent or RNA comprises any one of the antisense strand sequences or sense strand sequences set forth in SEQ ID NOs: 1759-1874.

In some embodiments, the dsRNAi agent or RNA comprises an antisense strand sequence and a sense strand sequence shown in any one of the duplex sequences in Table 1 or Table 2. In some embodiments, the dsRNAi agent or RNA comprises any one of the duplex sequences selected from IN-001-IN-488 and INI-001-INI-308. In some embodiments, the dsRNAi agent or RNA comprises any one of the duplex sequences selected from IN-489 to IN-546.

In some embodiments, at least one nucleotide in the sense strand and/or the antisense strand is a modified nucleotide. In some such embodiments, the modified nucleotide is a non-canonical base pairing nucleotide.

In some embodiments, at least one nucleotide in the sense strand and the antisense strand is a modified nucleotide. In some such embodiments, the modified nucleotide is a non-canonical base pairing nucleotide.

In some embodiments, at least one nucleotide in the sense strand or the antisense strand is a modified nucleotide. In some such embodiments, the modified nucleotide is a non-canonical base pairing nucleotide.

In some embodiments, substantially all the nucleotides in the sense strand and/or the antisense strand are modified nucleotides. In some such embodiments, at least one of the modified nucleotides is a non-canonical base pairing nucleotide.

In some embodiments, substantially all the nucleotides in the sense strand and the antisense strand are modified nucleotides. In some such embodiments, at least one of the modified nucleotides is a non-canonical base pairing nucleotide.

In some embodiments, substantially all the nucleotides in the sense strand or the antisense strand are modified nucleotides. In some such embodiments, at least one of the modified nucleotides is a non-canonical base pairing nucleotide.

In some embodiments, all the nucleotides in the sense strand and the antisense strand are modified nucleotides. In some such embodiments, at least one of the modified nucleotides is a non-canonical base pairing nucleotide.

In some embodiments, all the nucleotides in the sense strand are modified nucleotides. In some such embodiments, at least one of the modified nucleotides is a non-canonical base pairing nucleotide.

In some embodiments, all the nucleotides in the antisense strand are modified nucleotides. In some such embodiments, at least one of the modified nucleotides is a non-canonical base pairing nucleotide.

In some embodiments, the modified nucleotide is selected from: 2′-O-methyl-modified nucleotide, 2′-fluoro-modified nucleotide, 2′-deoxy nucleotide, 2′-methoxyethyl-modified nucleotide, 2′-amino-modified nucleotide, 2′-alkyl-modified nucleotide, 2′-alkoxy-modified nucleotide, 2′-F-arabino nucleotide, phosphorothioate-modified nucleotides, abasic nucleotides, morpholino nucleotide, locked nucleotide, inverted nucleotide, and hypoxanthin base-substituted nucleotide (e.g., inosine).

In some embodiments, the modified nucleotide is selected from: 2′-O-methyl-modified nucleotide, 2′-fluoro-modified nucleotide, 2′-deoxy nucleotide, phosphorothioate-modified nucleotide, and inverted base nucleotide (reverse linkage). In some such embodiments, the inverted base nucleotide is selected from: inverted A nucleotide, inverted dA nucleotide, inverted dT nucleotide, inverted C nucleotide and inverted U nucleotide.

In some embodiments, the modified nucleotide includes any one or combination of the following:
(1) According to the direction from the 5′ end to the 3′ end, the nucleotides at positions 2, 4, 12, and 14 of the antisense strand are 2′-fluoro-modified nucleotides, and the nucleotides at the remaining positions are 2′-O-methyl-modified nucleotides;
(2) According to the direction from the 5′ end to the 3′ end, the nucleotides at positions 7, 8, and 9 of the sense strand are 2′-fluoro-modified nucleotides;
(3) According to the direction from the 5′ end to the 3′ end, guanine (the base group of G) at positions 2-8 of the antisense strand is replaced by hypoxanthine (the base group of inosine, I).

In some such embodiments, the modified nucleotide for the base replacement by hypoxanthine at positions 2-8 of the antisense strand further meets any one or combination of the following characteristics:
(i) According to the direction from the 5′ end to the 3′ end, the guanine at positions 6-8 of the antisense strand is replaced by hypoxanthine (I);
(ii) According to the direction from the 5′ end to the 3′ end, the guanine in G₀ in the sequence N₁N₂G₀N₃N₄ of the antisense strand is replaced by hypoxanthine (I) if at least 3 bases of N₁, N₂, N₃ and N₄ are adenine (A) or uracil (U);
(iii) According to the direction from the 5′ end to the 3′ end, the total number of adenine (A) and uracil (U) at positions 2-8 of the antisense strand is no less than 4; and/or
(iv) at least one guanine in G nucleotide in the antisense strand is replaced by hypoxanthine (I), wherein the hypoxanthine replacement causes a difference in melting temperature (ΔTm) of at least 2° C., of 2° C., or of over 2° C., for a dsRNA comprising the antisense strand with hypoxanthine replacement compared to the same dsRNA where the guanine (G) has not been replaced by hypoxanthine (I).

In some embodiments, the antisense strand comprises completely contiguous nucleotide sequence selected from SEQ ID NO: 86, 182, 212, 350, 376, 402, 440, 460, 462, 464, 466, 468, 470, 472, 474, 476, 478, 480, 482, 484, 486, 488, 490, 492, 494, 496, 498, 500, 502, 504, 506, 508, 510, 512, 514, 516, 518, 520, 522, 524, 526, 528, 530, 532, 534, 536, 538, 540, 542, 544, 546, 548, 550, 552, 554, 556, 558, 560, 562, 564, 566, 568, 570, 572, 574, 576, 578, 580, 582, 584, 586, 588, 590, 592, 594, 596, 598, 600, 602, 604, 606, 608, 610, 612, 614, 616, 618, 620, 622, 624, 626, 628, 630, 632, 634, 636, 638, 640, 642, 644, 646, 648, 650, 652, 654, 656, 658, 660, 662, 664, 666, 668, 670, 672, 674, 676, 678, 680, 682, 684, 686, 688, 690, 692, 694, 696, 698, 700, 702, 704, 706, 708, 710, 712, 714, 716, 718, 720, 722, 724, 726, 728, 730, 732, 734, 736, 738, 740, 742, 744, 746, 748, 750, 752, 754, 756, 758, 760, 762, 764, 766, 768, 770, 772, 774, 776, 778, 780, 782, 784, 786, 788, 790, 792, 794, 796, 798, 800, 802, 804, 806, 808, 810, 812, 814, 816, 818, 820, 822, 824, 826, 828, 830, 832, 834, 836, 838, 840, 842, 844, 846, 848, 850, 852, 854, 856, 858, 860, 862, 864, 866, 868, 870, 872, 874, 876, 878, 880, 882, 884, 886, 888, 890, 892, 894, 896, 898, 900, 902, 904, 906, 908, 910, 912, 914, 916, 918, 920, 922, 924, 926, 928, 930, 932, 934, 936, 938, 940, 942, 944, 946, 948, 950, 952, 954, 956, 958, 960, 962, 964, 966, 968, 970, 972, 974, 1760, 1762, 1764, 1766, 1768, 1770, 1772, 1774, 1776, 1778, 1780, 1782, 1784, 1786, 1788, 1790, 1792, 1794, 1796, 1798, 1800, 1802, 1804, 1806, 1808, 1810, 1812, 1814, 1816, 1818, 1820, 1822, 1824, 1826, 1828, 1830, 1832, 1834, 1836, 1838, 1840, 1842, 1844, 1846, 1848, 1850, 1852, 1854, 1856, 1858, 1860, 1862, 1864, 1866, 1868, 1870, 1872 or 1874.

In some embodiments, the sense strand comprises completely contiguous nucleotide sequence selected from SEQ ID NO: 85, 181, 211, 349, 375, 401, 439, 459, 461, 463, 465, 467, 469, 471, 473, 475, 477, 479, 481, 483, 485, 487, 489, 491, 493, 495, 497, 499, 501, 503, 505, 507, 509, 511, 513, 515, 517, 519, 521, 523, 525, 527, 529, 531, 533, 535, 537, 539, 541, 543, 545, 547, 549, 551, 553, 555, 557, 559, 561, 563, 565, 567, 569, 571, 573, 575, 577, 579, 581, 583, 585, 587, 589, 591, 593, 595, 597, 599, 601, 603, 605, 607, 609, 611, 613, 615, 617, 619, 621, 623, 625, 627, 629, 631, 633, 635, 637, 639, 641, 643, 645, 647, 649, 651, 653, 655, 657, 659, 661, 663, 665, 667, 669, 671, 673, 675, 677, 679, 681, 683, 685, 687, 689, 691, 693, 695, 697, 699, 701, 703, 705, 707, 709, 711, 713, 715, 717, 719, 721, 723, 725, 727, 729, 731, 733, 735, 737, 739, 741, 743, 745, 747, 749, 751, 753, 755, 757, 759, 761, 763, 765, 767, 769, 771, 773, 775, 777, 779, 781, 783, 785, 787, 789, 791, 793, 795, 797, 799, 801, 803, 805, 807, 809, 811, 813, 815, 817, 819, 821, 823, 825, 827, 829, 831, 833, 835, 837, 839, 841, 843, 845, 847, 849, 851, 853, 855, 857, 859, 861, 863, 865, 867, 869, 871, 873, 875, 877, 879, 881, 883, 885, 887, 889, 891, 893, 895, 897, 899, 901, 903, 905, 907, 909, 911, 913, 915, 917, 919, 921, 923, 925, 927, 929, 931, 933, 935, 937, 939, 941, 943, 945, 947, 949, 951, 953, 955, 957, 959, 961, 963, 965, 967, 969, 971, 973, 1759, 1761, 1763, 1765, 1767, 1769, 1771, 1773, 1775, 1777, 1779, 1781, 1783, 1785, 1787, 1789, 1791, 1793, 1795, 1797, 1799, 1801, 1803, 1805, 1807, 1809, 1811, 1813, 1815, 1817, 1819, 1821, 1823, 1825, 1827, 1829, 1831, 1833, 1835, 1837, 1839, 1841, 1843, 1845, 1847, 1849, 1851, 1853, 1855, 1857, 1859, 1861, 1863, 1865, 1867, 1869, 1871 or 1873.

In some embodiments, the dsRNAi agent comprises a sense strand sequence and an antisense strand sequence selected from the following pairs: SEQ ID NO: 85 and 86; SEQ ID NO: 181 and 182; SEQ ID NO: 211 and 212; SEQ ID NO: 349 and 350; SEQ ID NO: 375 and 376; SEQ ID NO: 401 and 402; SEQ ID NO: 439 and 440; SEQ ID NO:459 and 460; SEQ ID NO:461 and 462; SEQ ID NO:463 and 464; SEQ ID NO:465 and 466; SEQ ID NO:467 and 468; SEQ ID NO:469 and 470; SEQ ID NO 471 and 472; SEQ ID NO:473 and 474; SEQ ID NO:475 and 476; SEQ ID NO:477 and 478; SEQ ID NO:479 and 480; SEQ ID NO:481 and 482; SEQ ID NO:483 and 484; SEQ ID NO:485 and 486; SEQ ID NO:487 and 488; SEQ ID NO:489 and 490; SEQ ID NO:491 and 492; SEQ ID NO:493 and 494; SEQ ID NO:495 and 496; SEQ ID NO:497 and 498; SEQ ID NO:499 and 500; SEQ ID NO: 501 and 502; SEQ ID NO: 503 and 504; SEQ ID NO: 505 and 506; SEQ ID NO: 507 and 508; SEQ ID NO: 509 and 510; SEQ ID NO: 511 and 512; SEQ ID NO: 513 and 514; SEQ ID NO: 515 and 516; SEQ ID NO: 517 and 518; SEQ ID NO: 519 and 520; SEQ ID NO: 521 and 522; SEQ ID NO: 523 and 524; SEQ ID NO: 525 and 526; SEQ ID NO: 527 and 528; SEQ ID NO: 529 and 530; SEQ ID NO: 531 and 532; SEQ ID NO: 533 and 534; SEQ ID NO: 535 and 536; SEQ ID NO: 537 and 538; SEQ ID NO: 539 and 540; SEQ ID NO: 541 and 542; SEQ ID NO: 543 and 544; SEQ ID NO: 545 and 546; SEQ ID NO: 547 and 548; SEQ ID NO: 549 and 550; SEQ ID NO: 551 and 552; SEQ ID NO: 553 and 554; SEQ ID NO: 555 and 556; SEQ ID NO: 557 and 558; SEQ ID NO: 559 and 560; SEQ ID NO: 561 and 562; SEQ ID NO: 563 and 564; SEQ ID NO: 565 and 566; SEQ ID NO: 567 and 568; SEQ ID NO: 569 and 570; SEQ ID NO: 571 and 572; SEQ ID NO: 573 and 574; SEQ ID NO: 575 and 576; SEQ ID NO: 577 and 578; SEQ ID NO: 579 and 580; SEQ ID NO: 581 and 582; SEQ ID NO: 583 and 584; SEQ ID NO: 585 and 586; SEQ ID NO: 587 and 588; SEQ ID NO: 589 and 590; SEQ ID NO: 591 and 592; SEQ ID NO: 593 and 594; SEQ ID NO: 595 and 596; SEQ ID NO: 597 and 598; SEQ ID NO: 599 and 600; SEQ ID NO: 601 and 602; SEQ ID NO: 603 and 604; SEQ ID NO: 605 and 606; SEQ ID NO: 607 and 608; SEQ ID NO: 609 and 610; SEQ ID NO: 611 and 612; SEQ ID NO: 613 and 614; SEQ ID NO: 615 and 616; SEQ ID NO: 617 and 618; SEQ ID NO: 619 and 620; SEQ ID NO: 621 and 622; SEQ ID NO: 623 and 624; SEQ ID NO: 625 and 626; SEQ ID NO: 627 and 628; SEQ ID NO: 629 and 630; SEQ ID NO: 631 and 632; SEQ ID NO: 633 and 634; SEQ ID NO: 635 and 636; SEQ ID NO: 637 and 638; SEQ ID NO: 639 and 640; SEQ ID NO: 641 and 642; SEQ ID NO: 643 and 644; SEQ ID NO: 645 and 646; SEQ ID NO: 647 and 648; SEQ ID NO: 649 and 650; SEQ ID NO: 651 and 652; SEQ ID NO: 653 and 654; SEQ ID NO: 655 and 656; SEQ ID NO: 657 and 658; SEQ ID NO: 659 and 660; SEQ ID NO: 661 and 662; SEQ ID NO: 663 and 664; SEQ ID NO: 665 and 666; SEQ ID NO: 667 and 668; SEQ ID NO: 669 and 670; SEQ ID NO: 671 and 672; SEQ ID NO: 673 and 674; SEQ ID NO: 675 and 676; SEQ ID NO: 677 and 678; SEQ ID NO: 679 and 680; SEQ ID NO: 681 and 682; SEQ ID NO: 683 and 684; SEQ ID NO: 685 and 686; SEQ ID NO: 687 and 688; SEQ ID NO: 689 and 690; SEQ ID NO: 691 and 692; SEQ ID NO: 693 and 694; SEQ ID NO: 695 and 696; SEQ ID NO: 697 and 698; SEQ ID NO: 699 and 700; SEQ ID NO: 701 and 702; SEQ ID NO: 703 and 704; SEQ ID NO: 705 and 706; SEQ ID NO: 707 and 708; SEQ ID NO: 709 and 710; SEQ ID NO: 711 and 712; SEQ ID NO: 713 and 714; SEQ ID NO: 715 and 716; SEQ ID NO: 717 and 718; SEQ ID NO: 719 and 720; SEQ ID NO: 721 and 722; SEQ ID NO: 723 and 724; SEQ ID NO: 725 and 726; SEQ ID NO: 727 and 728; SEQ ID NO: 729 and 730; SEQ ID NO: 731 and 732; SEQ ID NO: 733 and 734; SEQ ID NO: 735 and 736; SEQ ID NO: 737 and 738; SEQ ID NO: 739 and 740; SEQ ID NO: 741 and 742; SEQ ID NO: 743 and 744; SEQ ID NO: 745 and 746; SEQ ID NO: 747 and 748; SEQ ID NO: 749 and 750; SEQ ID NO: 751 and 752; SEQ ID NO: 753 and 754; SEQ ID NO: 755 and 756; SEQ ID NO: 757 and 758; SEQ ID NO: 759 and 760; SEQ ID NO: 761 and 762; SEQ ID NO: 763 and 764; SEQ ID NO: 765 and 766; SEQ ID NO: 767 and 768; SEQ ID NO: 769 and 770; SEQ ID NO: 771 and 772; SEQ ID NO: 773 and 774; SEQ ID NO: 775 and 776; SEQ ID NO: 777 and 778; SEQ ID NO: 779 and 780; SEQ ID NO: 781 and 782; SEQ ID NO: 783 and 784; SEQ ID NO: 785 and 786; SEQ ID NO: 787 and 788; SEQ ID NO: 789 and 790; SEQ ID NO: 791 and 792; SEQ ID NO: 793 and 794; SEQ ID NO: 795 and 796; SEQ ID NO: 797 and 798; SEQ ID NO: 799 and 800; SEQ ID NO: 801 and 802; SEQ ID NO: 803 and 804; SEQ ID NO: 805 and 806; SEQ ID NO: 807 and 808; SEQ ID NO: 809 and 810; SEQ ID NO: 811 and 812; SEQ ID NO: 813 and 814; SEQ ID NO: 815 and 816; SEQ ID NO: 817 and 818; SEQ ID NO: 819 and 820; SEQ ID NO: 821 and 822; SEQ ID NO: 823 and 824; SEQ ID NO: 825 and 826; SEQ ID NO: 827 and 828; SEQ ID NO: 829 and 830; SEQ ID NO: 831 and 832; SEQ ID NO: 833 and 834; SEQ ID NO: 835 and 836; SEQ ID NO: 837 and 838; SEQ ID NO: 839 and 840; SEQ ID NO: 841 and 842; SEQ ID NO: 843 and 844; SEQ ID NO: 845 and 846; SEQ ID NO: 847 and 848; SEQ ID NO: 849 and 850; SEQ ID NO: 851 and 852; SEQ ID NO: 853 and 854; SEQ ID NO: 855 and 856; SEQ ID NO: 857 and 858; SEQ ID NO:859 and 860; SEQ ID NO:861 and 862; SEQ ID NO: 863 and 864; SEQ ID NO:865 and 866; SEQ ID NO:867 and 868; SEQ ID NO:869 and 870; SEQ ID NO:871 and 872; SEQ ID NO:873 and 874; SEQ ID NO:875 and 876; SEQ ID NO:877 and 878; SEQ ID NO:879 and 880; SEQ ID NO:881 and 882; SEQ ID NO:883 and 884; SEQ ID NO:885 and 886; SEQ ID NO:887 and 888; SEQ ID NO:889 and 890; SEQ ID NO:891 and 892; SEQ ID NO:893 and 894; SEQ ID NO:895 and 896; SEQ ID NO:897 and 898; SEQ ID NO:899 and 900; SEQ ID NO:901 and 902; SEQ ID NO:903 and 904; SEQ ID NO:905 and 906; SEQ ID NO:907 and 908; SEQ ID NO:909 and 910; SEQ ID NO: 911 and 912; SEQ ID NO:913 and 914; SEQ ID NO:915 and 916; SEQ ID NO:917 and 918; SEQ ID NO:919 and 920; SEQ ID NO:921 and 922; SEQ ID NO:923 and 924; SEQ ID NO:925 and 926; SEQ ID NO:927 and 928; SEQ ID NO:929 and 930; SEQ ID NO:931 and 932; SEQ ID NO:933 and 934; SEQ ID NO:935 and 936; SEQ ID NO:937 and 938; SEQ ID NO:939 and 940; SEQ ID NO:941 and 942; SEQ ID NO:943 and 944; SEQ ID NO:945 and 946; SEQ ID NO:947 and 948; SEQ ID NO:949 and 950; SEQ ID NO:951 and 952; SEQ ID NO:953 and 954; SEQ ID NO:955 and 956; SEQ ID NO:957 and 958; SEQ ID NO:959 and 960; SEQ ID NO:961 and 962; SEQ ID NO:963 and 964; SEQ ID NO:965 and 966; SEQ ID NO:967 and 968; SEQ ID NO:969 and 970; SEQ ID NO:971 and 972; SEQ ID NO:973 and 974; SEQ ID NO: 1759 and 1760; SEQ ID NO:1761 and 1762; SEQ ID NO:1763 and 1764; SEQ ID NO:1765 and 1766; SEQ ID NO:1767 and 1768; SEQ ID NO:1769 and 1770; SEQ ID NO:1771 and 1772; SEQ ID NO:1773 and 1774; SEQ ID NO:1775 and 1776; SEQ ID NO:1777 and 1778; SEQ ID NO:1779 and 1780; SEQ ID NO:1781 and 1782; SEQ ID NO:1783 and 1784; SEQ ID NO:1785 and 1786; SEQ ID NO:1787 and 1788; SEQ ID NO:1789 and 1790; SEQ ID NO:1791 and 1792; SEQ ID NO:1793 and 1794; SEQ ID NO:1795 and 1796; SEQ ID NO:1797 and 1798; SEQ ID NO:1799 and 1800; SEQ ID NO:1801 and 1802; SEQ ID NO:1803 and 1804; SEQ ID NO:1805 and 1806; SEQ ID NO:1807 and 1808; SEQ ID NO:1809 and 1810; SEQ ID NO:1811 and 1812; SEQ ID NO:1813 and 1814; SEQ ID NO:1815 and 1816; SEQ ID NO:1817 and 1818; SEQ ID NO:1819 and 1820; SEQ ID NO:1821 and 1822; SEQ ID NO:1823 and 1824; SEQ ID NO:1825 and 1826; SEQ ID NO:1827 and 1828; SEQ ID NO:1829 and 1830; SEQ ID NO:1831 and 1832; SEQ ID NO:1833 and 1834; SEQ ID NO:1835 and 1836; SEQ ID NO:1837 and 1838; SEQ ID NO:1839 and 1840; SEQ ID NO:1841 and 1842; SEQ ID NO:1843 and 1844; SEQ ID NO:1845 and 1846; SEQ ID NO:1847 and 1848; SEQ ID NO:1849 and 1850; SEQ ID NO:1851 and 1852; SEQ ID NO:1853 and 1854; SEQ ID NO:1855 and 1856; SEQ ID NO:1857 and 1858; SEQ ID NO:1859 and 1860; SEQ ID NO:1861 and 1862; SEQ ID NO:1863 and 1864; SEQ ID NO:1865 and 1866; SEQ ID NO:1867 and 1868; SEQ ID NO:1869 and 1870; SEQ ID NO:1871 and 1872; or, SEQ ID NO:1873 and 1874.

In some embodiments, the dsRNAi agent comprises a sense strand sequence and an antisense strand sequence selected from any one of duplexes IN-001-IN-488 and INI-001-INI-308. In some embodiments, the dsRNAi agent comprises a sense strand sequence and an antisense strand sequence selected from any one of duplexes IN-489 to IN-546.

In some embodiments, the antisense oligonucleotides of the disclosure are substantially complementary to the target mRNA, e.g., INHBE mRNA, and comprise a contiguous nucleotide sequence which is at least about 85% complementary over its entire length to any one of the sense strand oligonucleotides provided herein or a portion thereof, e.g., about 85%, 86%, 87%, 88%, 89%, 90%, 91%, 92%, 93%, 94%, 95%, 96%, 97%, 98%, or about 99% complementary.

In some embodiments, the antisense oligonucleotides of the disclosure are substantially complementary to any one of the sense strand oligonucleotides disclosed herein, and comprise a contiguous nucleotide sequence which is at least about 85% complementary over its entire length to any one of the sense strand oligonucleotides provided herein or a portion thereof, e.g., about 85%, 86%, 87%, 88%, 89%, 90%, 91%, 92%, 93%, 94%, 95%, 96%, 97%, 98%, or about 99% complementary.

In some embodiments, dsRNAi agent of the disclosure includes a sense strand that is substantially complementary to an antisense oligonucleotide which is, in turn, complementary to the target mRNA, e.g, INHBR mRNA, wherein the sense strand is at least about 85% complementary over its entire length to any one of the antisense strand oligonucleotides provided herein or a portion thereof, e.g., about 85%, 86%, 87%, 88%, 89%, 90%, 91%, 92%, 93%, 94%, 95%, 96%, 97%, 98%, or about 99% complementary.

In some embodiments, the double-stranded region of a dsRNAi agent is equal to or at least, 10, 11, 12, 13, 14, 15, 16, 17, 18, 19, 20, 21, 22, 23, 23, 24, 25, 26, 27, 28, 29, 30 or more nucleotide pairs in length.

In some embodiments, the antisense strand of a dsRNAi agent is equal to or at least 14, 15, 16, 17, 18, 19, 20, 21, 22, 23, 23, 24, 25, 26, 27, 28, 29, or 30 nucleotides in length.

In some embodiments, the sense strand of a dsRNAi agent is equal to or at least 10, 11, 12, 13, 14, 15, 16, 17, 18, 19, 20, 21, 22, 23, 23, 24, 25, 26, 27, 28, 29, or 30 nucleotides in length.

In some embodiments, the sense and antisense strands of the dsRNAi agent are each independently 15 to 30 nucleotides in length.

In some embodiments, the sense and antisense strands of the dsRNAi agent are each independently 19 to 25 nucleotides in length.

In some embodiments, the sense and antisense strands of the dsRNAi agent are each independently 21 to 23 nucleotides in length.

In some embodiments, the sense strand of the dsRNAi agent is 21 nucleotides in length, and the antisense strand is 23 nucleotides in length, wherein the strands form a double-stranded region of 21 consecutive base pairs having a 2-nucleotide long single stranded overhang at the 3′-end.

In another aspect, the present disclosure provides an RNAi agent comprising a dsRNA provided herein, and a targeting ligand. The targeting ligand is typically conjugated to the dsRNA and acts to target the RNAi agent to a cell.

In some embodiments, the dsRNAi agent comprises the dsRNA, and at least one targeting ligand conjugated thereto, e.g., conjugated to the sense strand of the dsRNA. In some such embodiments, the 3′ end of the sense strand is conjugated to the targeting ligand(s).

In some embodiments, the targeting ligand of the present disclosure specifically targets asialoglycoprotein receptors (ASGPR), e.g., on the surface of liver cells. In some such embodiments, the targeting ligand comprises N-acetyl-galactosamine (GalNAc), or, the targeting ligand is a GalNAc derivative. In some embodiments, the targeting ligand is any targeting moiety disclosed in WO2022266753A1. Unless obviously contradicted, the entire contents of WO2022266753A1 are hereby incorporated by reference.

In some embodiments, the dsRNAi agent has the structure selected from any one of formula 1 to formula 33, wherein R² is the dsRNA. According to common knowledge in the art, R² forms a dsRNAi agent by conjugating the 3′ end or the 5′ end of the sense strand to a targeting ligand. In some embodiments, the 3′ end of the sense strand is conjugated to the targeting ligand. In other embodiments, the 5′ end of the sense strand is conjugated to the targeting ligand.

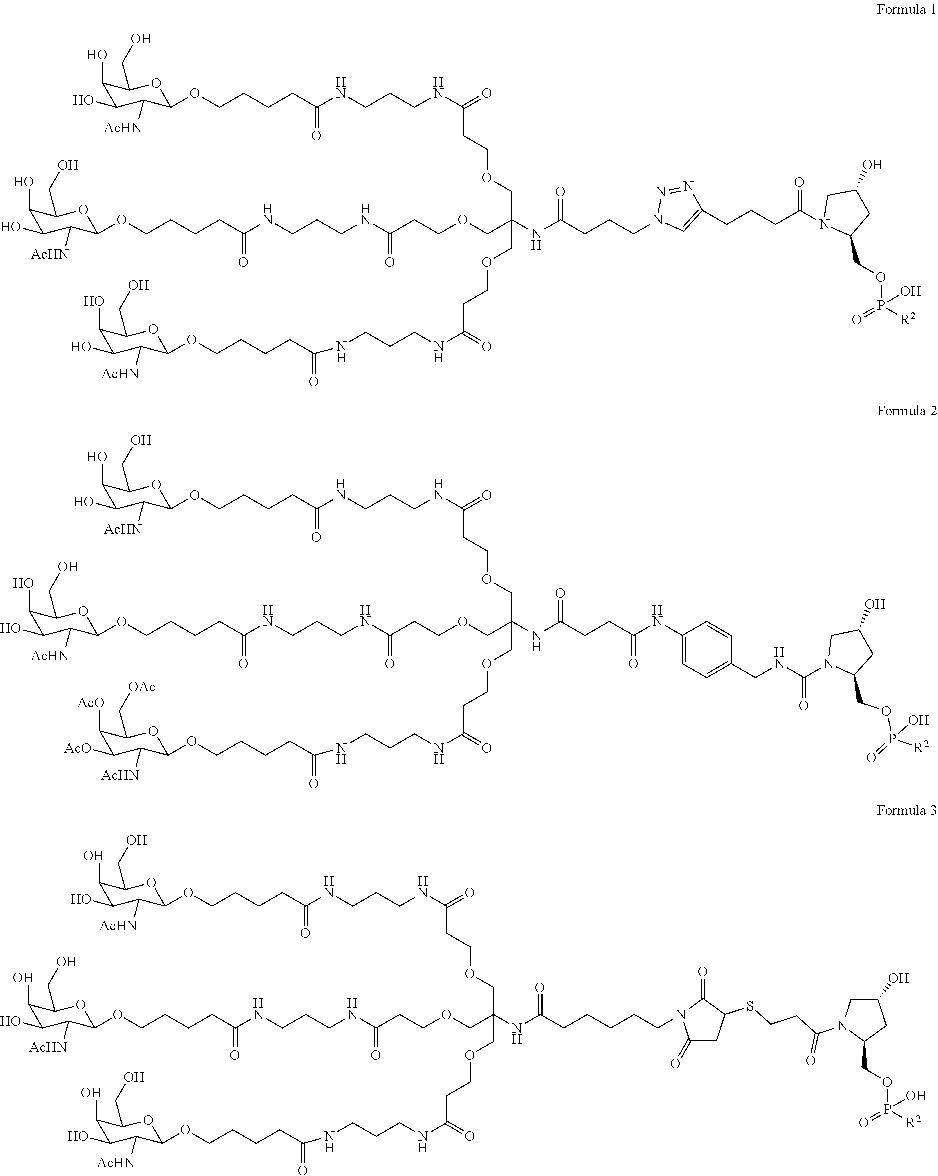

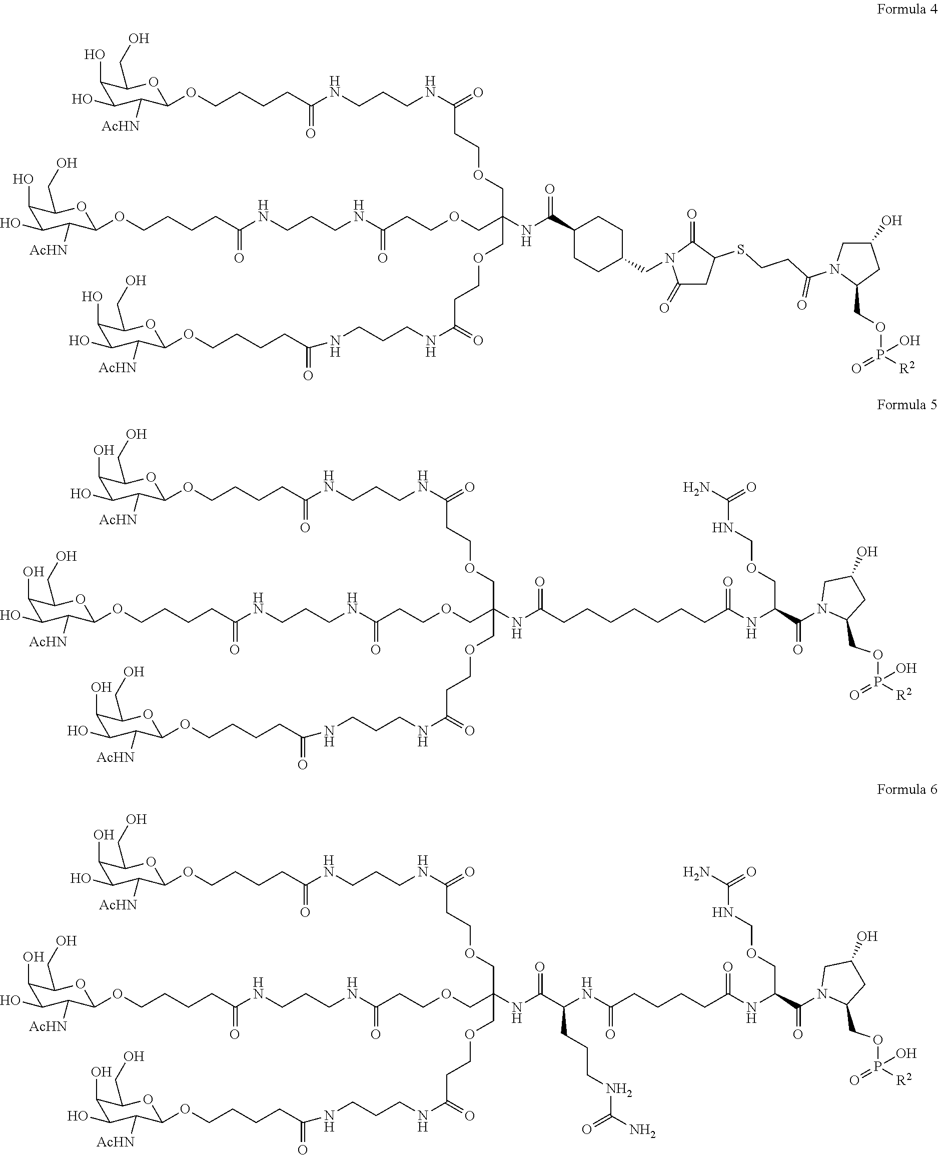

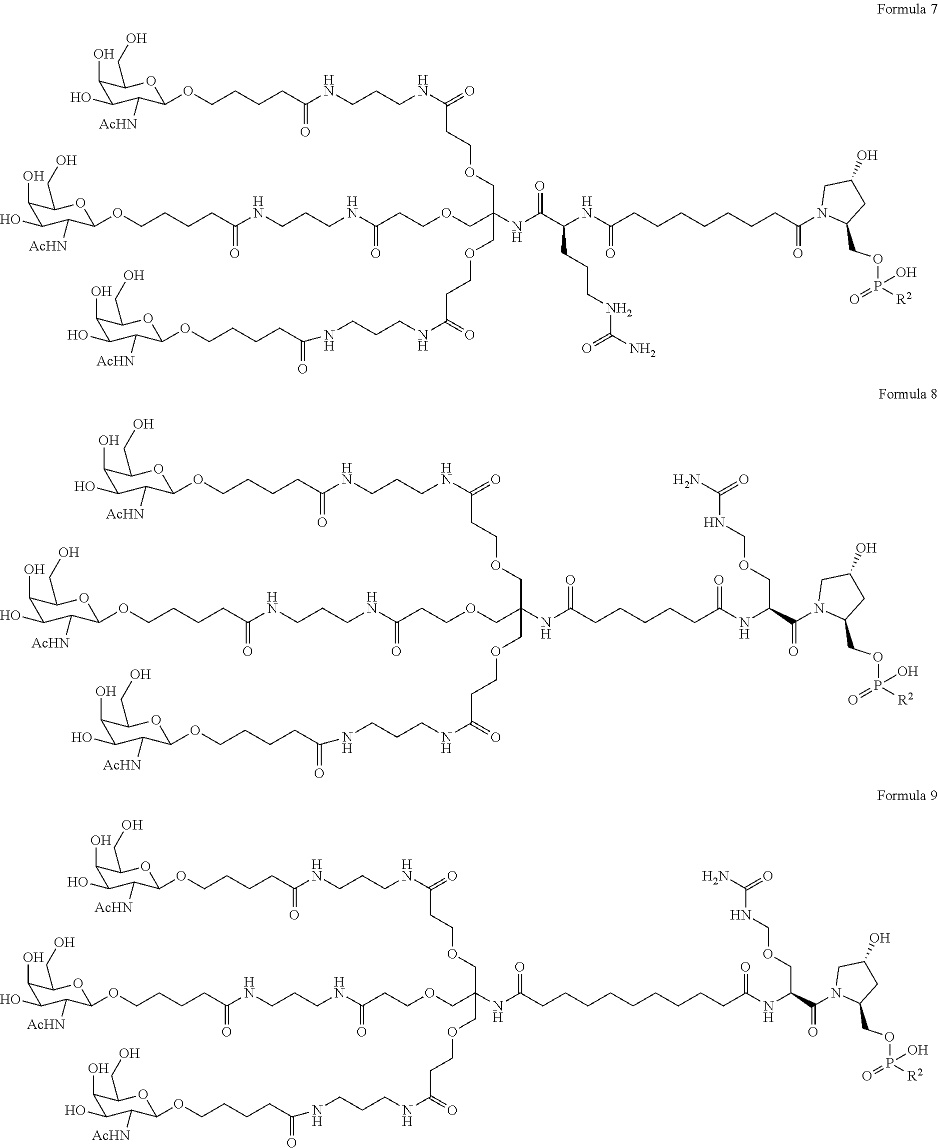

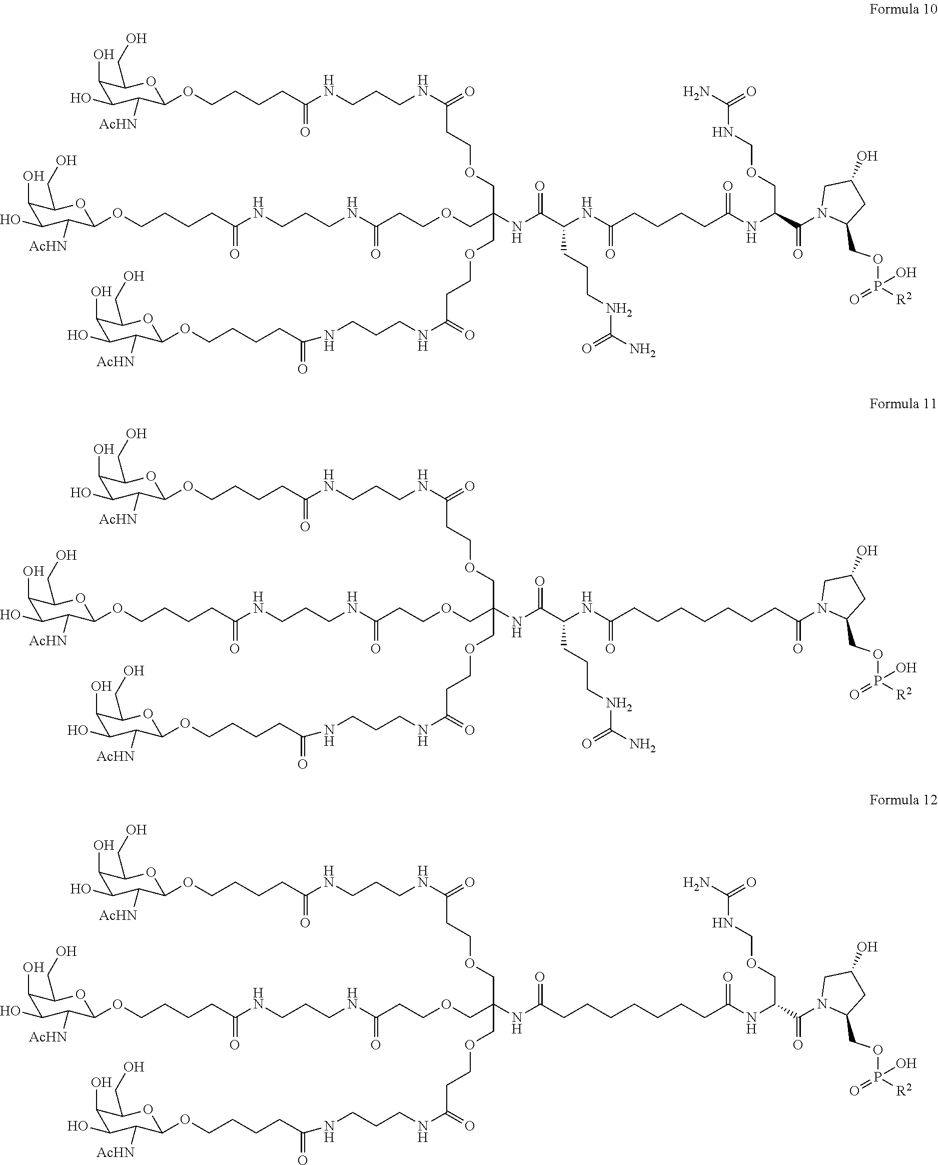

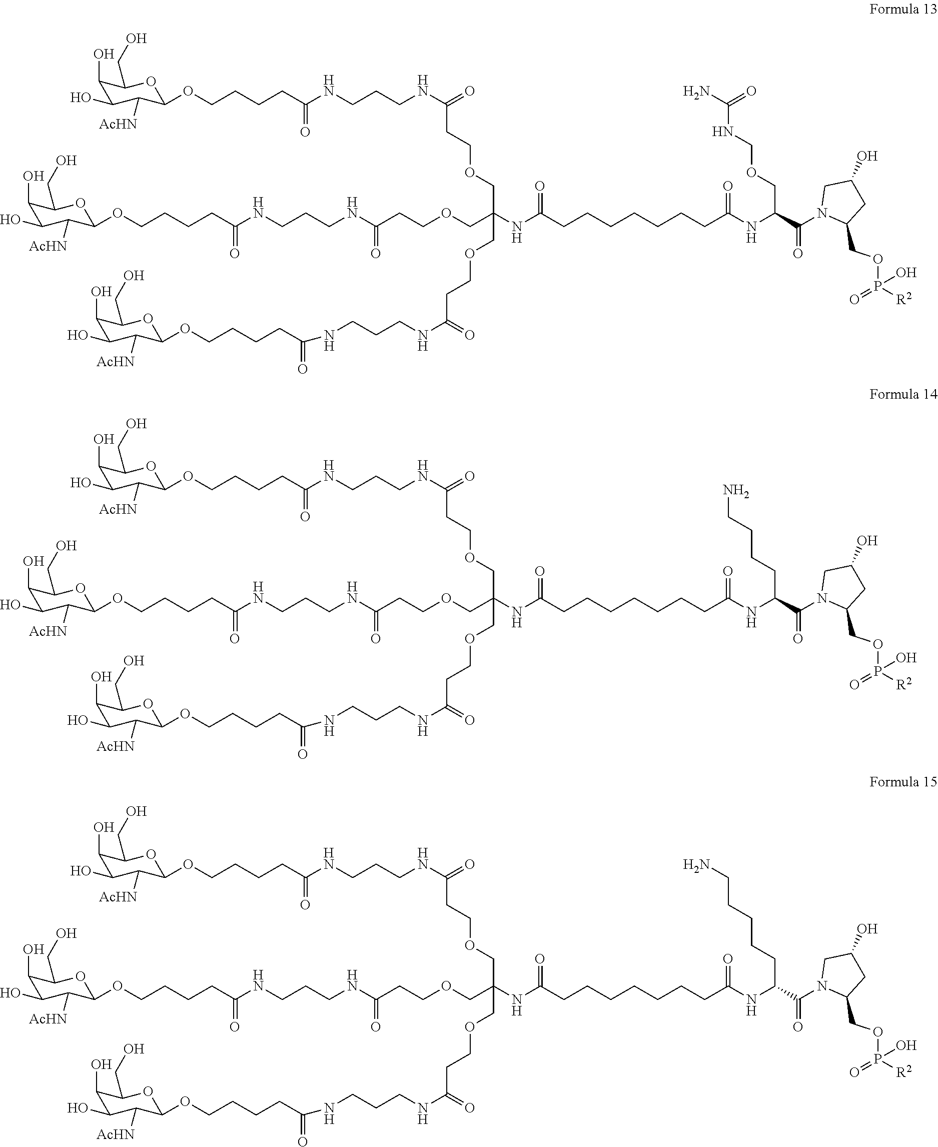

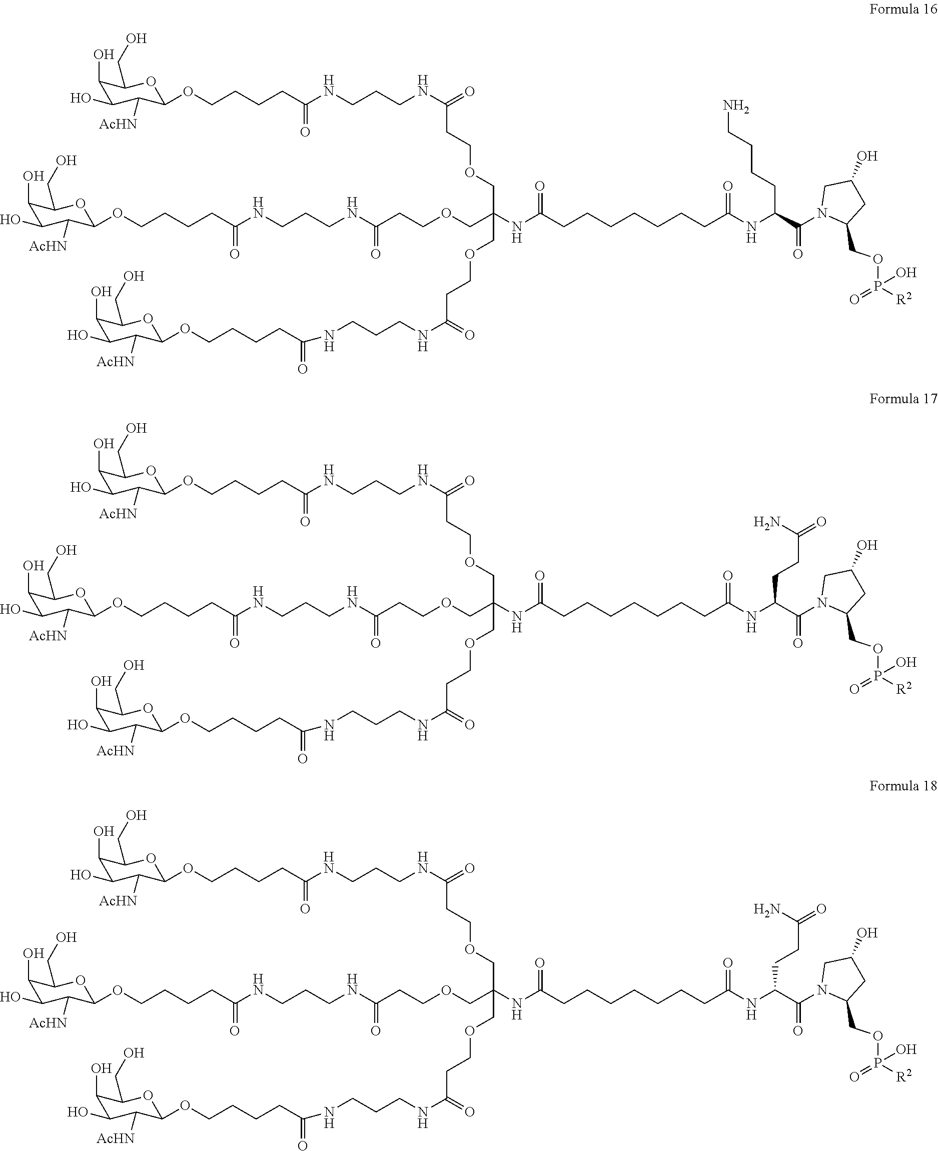

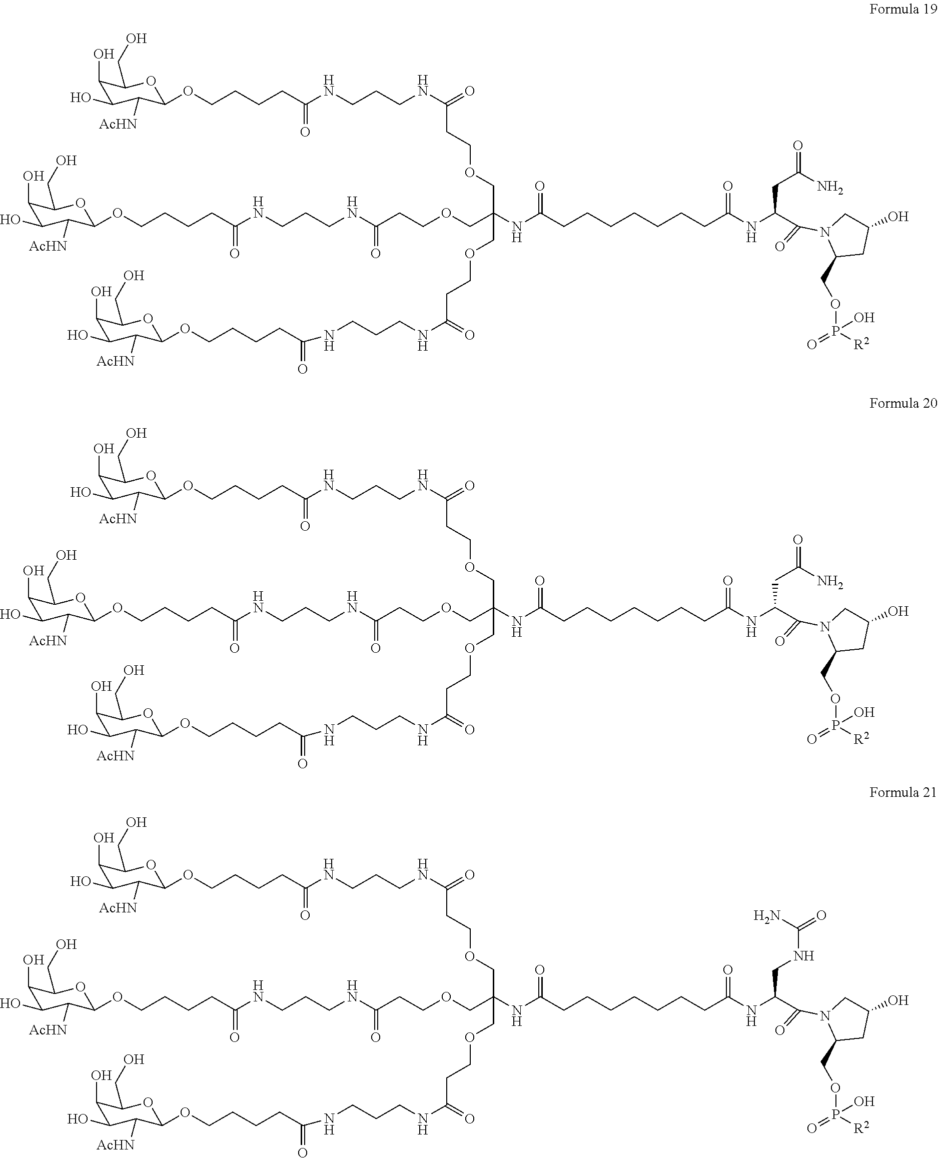

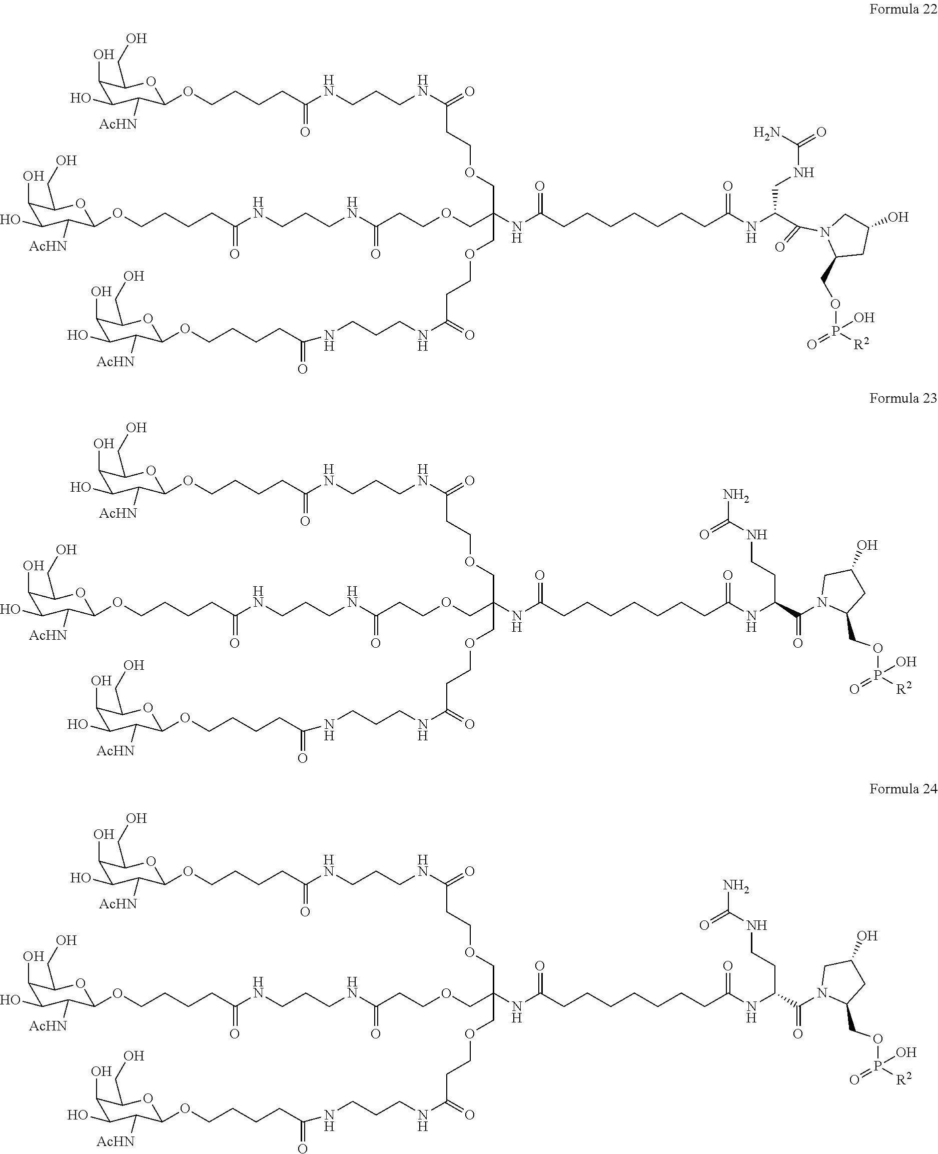

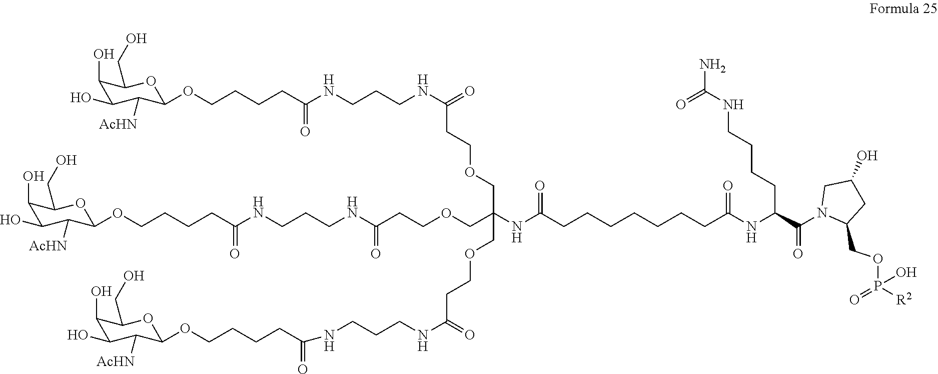

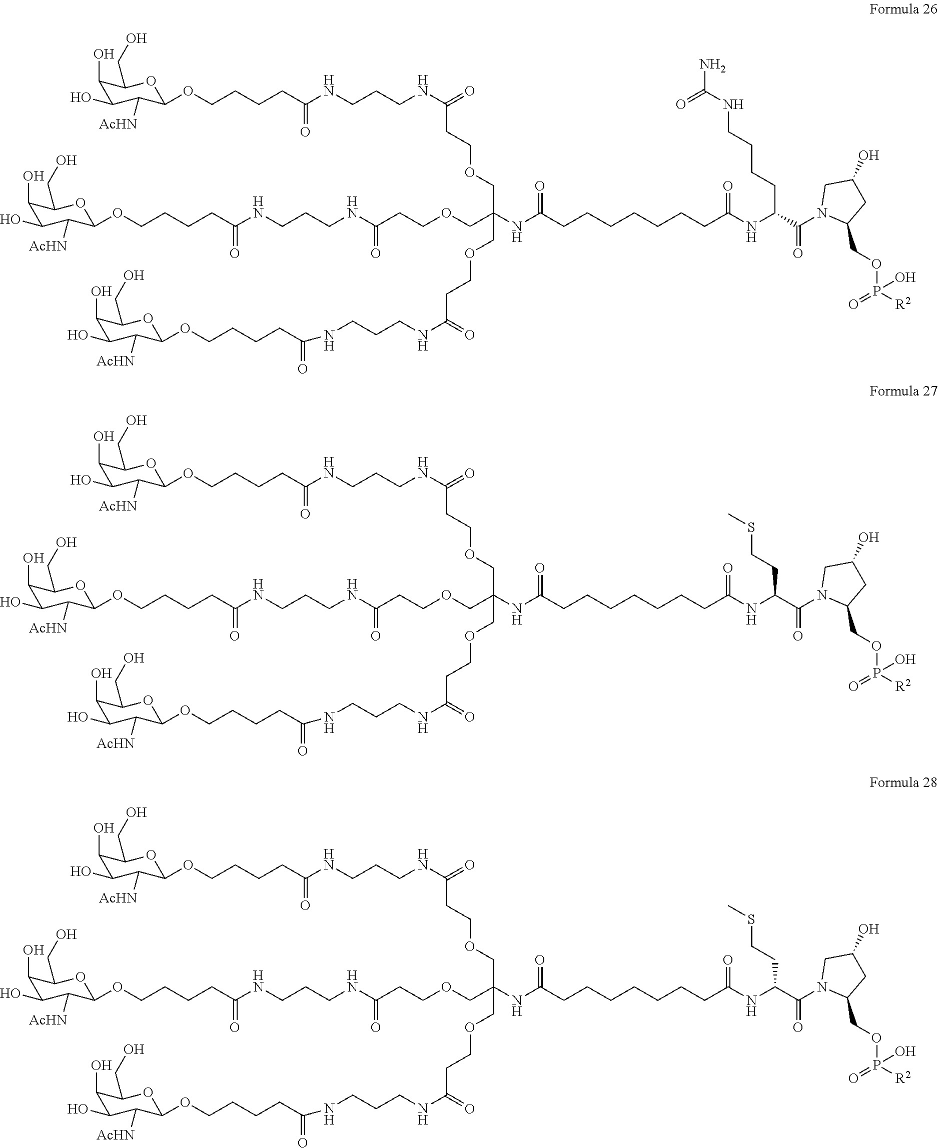

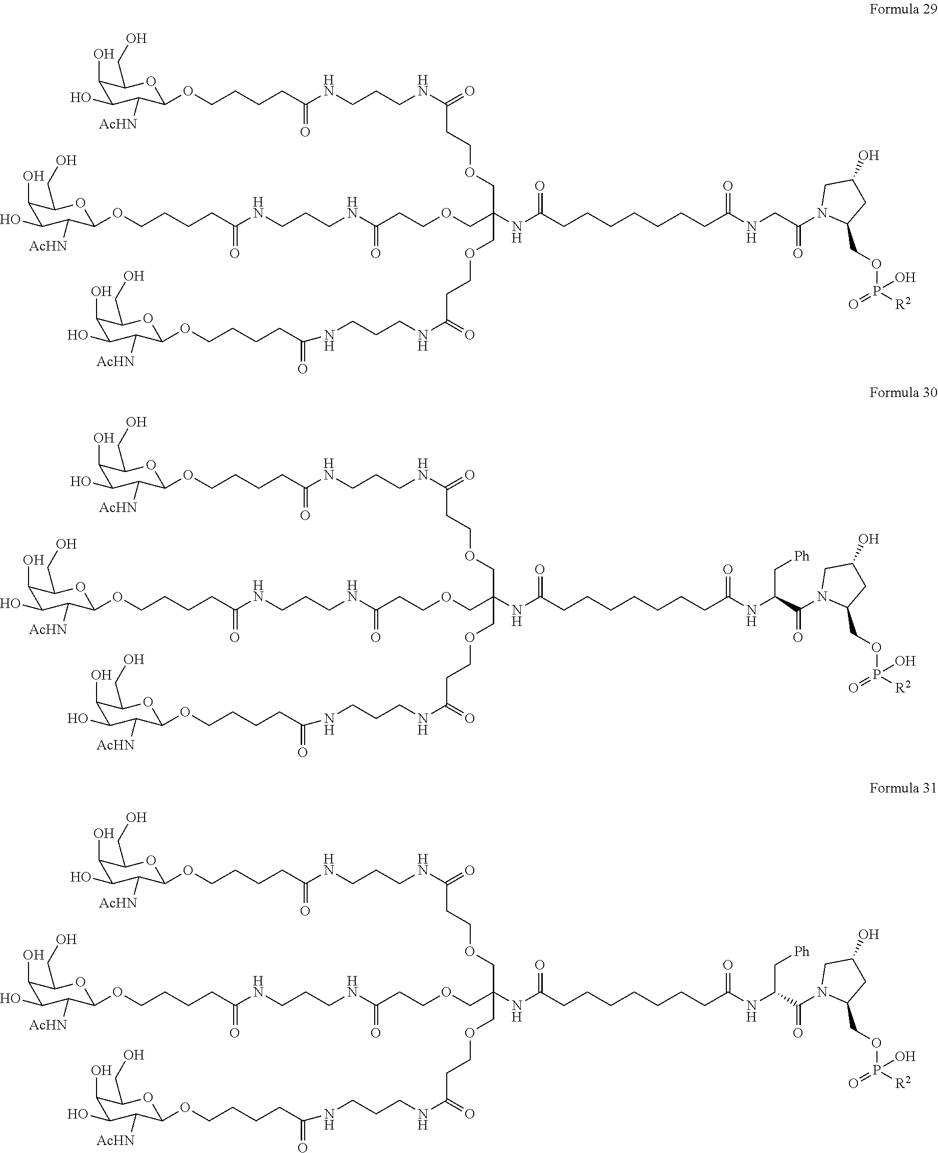

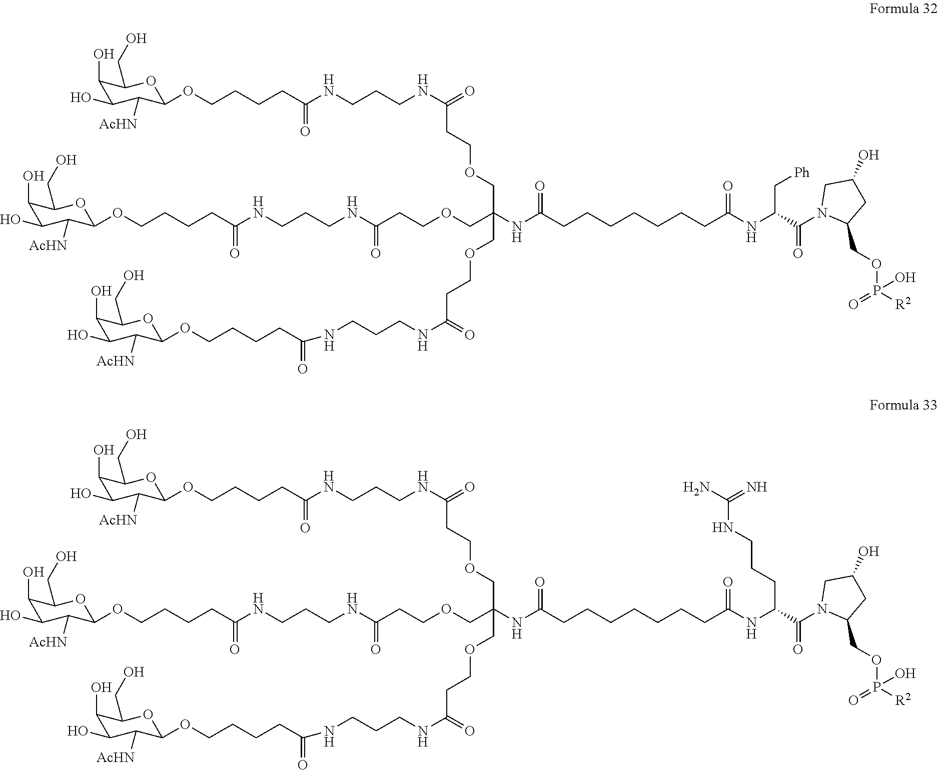

In another aspect, the present disclosure provides a cell, a vector, a host cell, and/or a pharmaceutical composition comprising a dsRNAi agent or RNA described herein.

In some embodiments, the pharmaceutical composition comprises the dsRNAi agent or RNA, or a pharmaceutically acceptable salt thereof, and a pharmaceutically acceptable carrier. The pharmaceutical composition of the present disclosure can be practically used for the prevention and/or treatment of INHBE-associated diseases and disorders, as discussed further hereinbelow.

In some embodiments, the pharmaceutical composition is formulated for administration by injection or infusion, e.g., for intravenous, subcutaneous, intraperitoneal, or intramuscular administration. In some embodiments, the pharmaceutical composition is formulated for subcutaneous administration. In some embodiments, the pharmaceutical composition is formulated for intravenous administration.

In some embodiments, the carrier of the pharmaceutical composition is an unbuffered solution or a buffered solution. Typical unbuffered solutions include without limitation saline or water. Typical buffered solutions include without limitation one or more of acetate, citrate, prolamine, carbonate, phosphate, and any combination thereof. In some embodiments, a buffer solution is phosphate buffered saline (PBS).

In another aspect of the present disclosure, a method for inhibiting the expression of INHBE in a cell is provided. The method includes contacting the cell with a dsRNAi agent or RNA of the disclosure such that the mRNA transcript of the INHBE gene is degraded, thereby inhibiting expression of the INHBE gene in the cell.

In some embodiments, the cells are in a subject. In some embodiments, the cells are hepatocytes. In some embodiments, the cells are adipocytes. In some embodiments, the subject is a human.

In some embodiments, INHBE expression is inhibited by at least about 30%, at least about 35%, at least about 40%, at least about 45%, at least about 50%, at least about 55%, at least about 60%, at least about 65%, at least about 70%, at least about 75%, at least about 80%, at least about 85%, at least about 90% or at least about 95%.

In another aspect, the present disclosure provides methods of treating a subject having a disorder mediated by INHBE expression, e.g., an INHBE-associated disease or disorder. The methods comprise administering to the subject a therapeutically effective amount of the dsRNAi agent of the present disclosure, such that expression of the INHBE gene is inhibited in the subject.

In some embodiments, the subject is a human.

In some embodiments, the subject has a metabolic disorder.

In some embodiments, the metabolic disorder is one or more of metabolic syndrome, type 2 diabetes, obesity, pre-diabetes, elevated triglyceride levels, lipodystrophy, liver inflammation, fatty liver, hypercholesterolemia, disorders associated with elevated liver enzymes, nonalcoholic steatohepatitis, cardiovascular disease and kidney disease. The metabolic syndrome includes, but is not limited to, one or more of abdominal obesity, insulin resistance, hypertension, hyperlipidemia and dyslipidemia).

In some embodiments of methods of the disclosure, the inhibition of expression of the INHBE gene in the cell or in the subject reduces the protein level of the INHBE gene in the subject's serum by at least about 50%, at least about 60%, at least about 70%, at least about 80%, at least about 90%, or at least about 95%, as compared to the level before or without administration of the dsRNAi agent.

In some embodiments of methods of the disclosure, the dsRNAi agent is administered to the subject at a dose of about 0.01 mg/kg to about 50 mg/kg, or at a dose of about 0.10 mg/kg to about 50 mg/kg, for example, at a dose of about 0.01 mg/kg to about 10 mg/kg (e.g., about 3 mg/kg, about 4 mg/kg, about 5 mg/kg, about 6 mg/kg, about 7 mg/kg, about 8 mg/kg, or about 9 mg/kg), about 0.5 mg/kg to about 50 mg/kg, about 10 mg/kg to about 30 mg/kg, about 10 mg/kg to about 20 mg/kg, about 15 mg/kg to about 20 mg/kg, about 15 mg/kg to about 25 mg/kg, about 15 mg/kg to about 30 mg/kg, or about 20 mg/kg to about 30 mg/kg.

In some embodiments of methods of the disclosure, the method further comprises determining the level of INHBE in a sample from the subject. In some embodiments, the level of INHBE in a sample from the subject is determined before, during and/or after administering the dsRNAi agent to the subject. Any suitable sample may be used such as, for example and without limitation, a blood sample, a serum sample, or a sample of liver tissue.

In some embodiments of methods of the disclosure, the method further comprises administering to the subject an additional therapeutic agent to treat an INHBE-associated disorder or a metabolic disorder. Examples of such additional therapeutic agents include, without limitation: insulin, a glucagon-like peptide 1 (GLP-1) agonist, a glucose-dependent insulinotropic polypeptide (GIP) receptor agonist, a glucagon receptor agonist, a sulfonylurea, a seglitinide, a biguanide, a thiazolidinedione, an alpha-glucosidase inhibitor, an SGLT2 inhibitor, a DPP-4 inhibitor, an HMG-CoA reductase inhibitor, a statin, and any combination of the foregoing.

In some embodiments, the dsRNAi agent of the disclosure may be administered simultaneously or sequentially with an additional therapeutic agent. In some embodiments, the dsRNAi agent is administered before or after an additional therapeutic agent, such as a standard therapeutic agent for an INHBE-associated disorder or a metabolic disorder.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

For a better understanding of the invention and to show more clearly how it may be carried into effect, reference will now be made by way of example to the accompanying drawings, which illustrate aspects and features according to embodiments of the present invention, and in which:

FIGS. 1-4, 5A and 5B are graphs of relative INHBE mRNA level vs. siRNA concentration and show the results of detecting the knockdown activity of the indicated siRNA on human INHBE in HEP3B cells using rt-PCR (full curve).

FIG. 6 shows the results of using the reporter gene method to detect the knockdown activity (single dose) of siRNA on human (top) and mouse (bottom) INHBE.

FIGS. 7 and 8 show the results of using the reporter gene method to detect the knockdown activity of siRNA on human INHBE (3 doses).

FIG. 9 shows the results of detecting the knockdown activity (3 doses) of siRNA on mouse INHBE using the reporter gene method.

FIGS. 10-15 show the results of using the reporter gene method to detect the knockdown activity of siRNA on human INHBE (full curve).

FIG. 16 shows the results of detecting siRNA knockdown activity (full curve) on murine INHBE using the reporter gene method.

FIGS. 17-19 and FIG. 21 show the results of using the reporter gene method to detect the knockdown activity of siRNA on human INHBE (3 doses).

FIG. 20 and FIG. 22 show the result of using the reporter gene method to detect the knockdown activity of siRNA on human INHBE (2 doses).

FIGS. 23 and 24 show the result of using the reporter gene method to detect the knockdown activity of siRNA on human INHBE (full curve).

FIG. 25 shows the results of siRNA stability test in serum.

FIG. 26 shows the results of siRNA stability test in human liver S9 fraction.

FIG. 27 shows the changes in siRNA content in the liver of each group (3 mice per group) at different time points after administration.

FIG. 28 shows the percentage of different siRNA content in the mouse liver normalized to each siRNA content in the liver tissue of mice (3 mice per group) 7 days after administration.

FIG. 29 shows the mRNA sequence for human INHBE.

FIG. 30 shows the mRNA sequence for cynomolgus monkey INHBE.

### DETAILED DESCRIPTION

The present disclosure provides RNAi agents and compositions which effect the RNA-induced silencing complex (RISC)-mediated cleavage of RNA transcripts of the target gene inhibin subunit beta E (INHBE). The gene may be within a cell, such as an adipocyte and/or a hepatocyte or other liver cell, e.g., a cell within a subject, such as a human. The use of these RNAi agents and compositions enables the targeted degradation of INHBE mRNAs in mammals. As inhibitors of INHBE expression, RNAi agents and compositions of the disclosure are useful for the prevention, treatment, and/or inhibition of INHBE-associated diseases or disorders, such as metabolic syndrome and related conditions.

Accordingly, the present disclosure provides methods for treating, preventing or inhibiting an INHBE-associated disease or disorder, such as without limitation a metabolic disorder, e.g., metabolic syndrome; a disorder of carbohydrates, e.g., type II diabetes, pre-diabetes; a lipid metabolism disorder, e.g., a hyperlipidemia, hypertension, lipodystrophy; a kidney disease; a cardiovascular disease; or a disorder of body weight, e.g., obesity, overweight; using RNAi compositions which effect the RNA-induced silencing complex (RISC)-mediated cleavage of RNA transcripts of the inhibin subunit beta E (INHBE) target gene.

The RNAi agents of the disclosure comprise an antisense RNA strand having a region which is up to about 30 nucleotides or less in length, e.g., 19-30, 19-29, 19-28, 19-27, 19-26, 19-25, 19-24, 19-23, 19-22, 19-21, 19-20, 20-30, 20-29, 20-28, 20-27, 20-26, 20-25, 20-24,20-23, 20-22, 20-21, 21-30, 21-29, 21-28, 21-27, 21-26, 21-25, 21-24, 21-23, 21-22, 15, 15-16, 15-17, 15-20, 15-21, 15-22, 15-23, 15-17, or at least 15 nucleotides in length, which region is substantially complementary to at least part of an mRNA transcript of the INHBE target gene.

In certain embodiments, one or both of the strands of the dsRNAi agents of the disclosure is up to 66 nucleotides in length, e.g., 36-66, 26-36, 25-36, 31-60, 22-43, or 27-53 nucleotides in length, with a region of at least 15 contiguous nucleotides that is substantially complementary to at least a part of an mRNA transcript of the INHBE target gene. In some embodiments, such RNAi agents having longer length antisense strands may, for example, include a second RNA strand (the sense strand) of 20-60 nucleotides in length wherein the sense and antisense strands form a double-stranded region (duplex) of 15-30 contiguous nucleotides.

The use of RNAi agents of the disclosure enables the targeted degradation of mRNAs of the INHBE target gene in mammals. The present inventors have demonstrated that RNAi agents of the disclosure can effect the RNA-induced silencing complex (RISC)-mediated cleavage of INHBE RNA transcripts, resulting in significant inhibition of expression of the INHBE target gene. In certain embodiments, RNAi agents of the disclosure are more effective (e.g., more potent) and/or more specific (e.g., more safe, less off-target effects) than previous RNAi agents targeting the same gene. In certain embodiments, RNAi agents target specific sites in the INHBE mRNA and/or include modifications of the RNA (e.g., modified nucleotides, chemical modifications) selected to increase efficacy, potency, specificity, and/or safety, as described. In some such embodiments, RNAi agents comprise at least one modified nucleotide, such as a non-canonical base pairing nucleotide. Methods and compositions comprising these RNAi agents are useful for treating a subject having an INHBE-associated disease or disorder, e.g., a metabolic disorder. Accordingly, there are provided methods for treating, preventing or inhibiting a metabolic disorder in a subject who would benefit from inhibiting or reducing INHBE expression using RNAi agents and compositions of the disclosure.

The present disclosure also provides methods for preventing at least one symptom in a subject having a disorder that would benefit from inhibiting or reducing INHBE expression. The following detailed description discloses how to make and use RNAi agents and compositions thereof to inhibit the expression of the INHBE target gene, as well as compositions, uses, and methods for treating subjects that would benefit from inhibition and/or reduction of the expression of the INHBE target gene, e.g., subjects susceptible to or diagnosed with a metabolic disorder.

### Definitions

In order to provide a clear and consistent understanding of the terms used in the present specification, a number of definitions are provided below. Moreover, unless defined otherwise, all technical and scientific terms as used herein have the same meaning as commonly understood to one of ordinary skill in the art to which this invention pertains.

The use of the word “a” or “an” when used in conjunction with the term “comprising” in the claims and/or the specification may mean “one”, but it is also consistent with the meaning of “one or more”, “at least one”, and “one or more than one”. Similarly, the word “another” may mean at least a second or more.

The term “or” is used herein to mean, and is used interchangeably with, the term “and/or”, unless context clearly indicates otherwise. For example, “sense strand or antisense strand” is understood as “sense strand or antisense strand or sense strand and antisense strand.”

As used in this specification and claim(s), the words “comprising” (and any form of comprising, such as “comprise” and “comprises”), “having” (and any form of having, such as “have” and “has”), “including” (and any form of including, such as “include” and “includes”) and “containing” (and any form of containing, such as “contain” and “contains”), are inclusive or open-ended and do not exclude additional, unrecited elements or process steps.

The term “about” is used herein to indicate that a value includes an inherent variation of error for the device or the method being employed to determine the value. The term “about” when used in conjunction with a numerical value is meant to encompass a numerical value within a range having a lower limit of 5% less and an upper limit of 5% greater than the stated numerical value, including but not limited to ±5%, ±2%, ±1%, and +0.1%, as these variations are suitable for performing the disclosed methods. When about is present before a series of numbers or a range, it is understood that “about” can modify each of the numbers in the series or range.

The term “at least”, “no less than”, or “or more” prior to a number or series of numbers is understood to include the number adjacent to the term “at least”, and all subsequent numbers or integers that could logically be included, as clear from context. For example, the number of nucleotides in a nucleic acid molecule must be an integer. For example, “at least 15 nucleotides of a 17 nucleotide nucleic acid molecule” means that 15, 16, or 17 nucleotides have the indicated property. When “at least” is present before a series of numbers or a range, it is understood that “at least” can modify each of the numbers in the series or range.

As used herein, “no more than” or “or less” is understood as the value adjacent to the phrase and logical lower values or integers, as logical from context, to zero. For example, a duplex with an overhang of “no more than 2 nucleotides” has a 2, 1, or 0 nucleotide overhang. When “no more than” is present before a series of numbers or a range, it is understood that “no more than” can modify each of the numbers in the series or range. As used herein, ranges include both the upper and lower limit.

The term “inhibin subunit beta E” is used interchangeably with the term “INHBE”, and is also known as inhibin beta E chain, inhibin beta E, inhibin R E, activin E, activin R E, activin beta E and MGC4638. The sequence of a human INHBE mRNA transcript can be found at, for example, GenBank Accession No. NM_031479.5. The sequence of mouse INHBE mRNA can be found at, for example, GenBank Accession No. NM_008382.3 (SEQ ID NO: 1875). The sequence of cynomolgus monkey INHBE mRNA can be found at, for example, GenBank Accession No. XM_005571319.3. Additional examples of INHBE mRNA sequences are readily available through publicly available databases, e.g., GenBank, UniProt, OMIM, and the Macaca genome project web site. Further information on INHBE can be found, for example, at www.ncbi.nlm.nih.gov/gene/?term=INHBE. The entire contents of each of the foregoing GenBank Accession numbers and the Gene database numbers are incorporated herein by reference as of the date of filing this application.

The term “target sequence” or “target nucleic acid” or “target mRNA” refers to a contiguous portion of the nucleotide sequence of an mRNA molecule formed during the transcription of a target gene, including mRNA that is a product of RNA processing of a primary transcription product. In one embodiment, the target portion of the sequence is at least long enough to serve as a substrate for RNAi-directed cleavage at or near that portion of the nucleotide sequence of an mRNA molecule formed during the transcription of a target gene. In one embodiment, the target sequence is within the protein coding region of the target gene. In another embodiment, the target sequence is within the 3′ UTR of the target gene. The target nucleic acid can be a cellular gene (or mRNA transcribed from the gene) whose expression is associated with a particular disorder or disease state. In some embodiments, the target sequence is from about 19-36 nucleotides in length, e.g., about 19-30 nucleotides in length, e.g., 19-30, 19-29, 19-28, 19-27, 19-26, 19-25, 19-24, 19-23, 19-22, 19-21, 19-20, 20-30, 20-29, 20-28, 20-27, 20-26, 20-25, 20-24, 20-23, 20-22, 20-21, 21-30, 21-29, 21-28, 21-27, 21-26, 21-25, 21-24, 21-23, or 21-22 nucleotides in length. In some embodiments, the target sequence is from about 15-30 nucleotides in length, e.g., about 15-23 nucleotides in length, e.g., 15-30, 15-29, 15-28, 15-27, 15-26, 15-25, 15-24, 15-23, 15-22, 15-21, 15-20, 17-30, 17-29, 18-28, 17-27, 16-26, 15-25, 15-24, 16-23, 16-22, 16-21, 16-30, 16-29, 16-28, 16-27, 16-26, 16-25, 16-24, 17-25, 17-23, or 15-17 nucleotides in length. In certain embodiments, the target sequence is 17-25 nucleotides in length, 19-21 nucleotides in length, 19-23 nucleotides in length, or 21-23 nucleotides in length. Ranges and lengths intermediate to the above recited ranges and lengths are also contemplated to be part of the disclosure.

As used herein, the term “strand comprising a sequence” refers to an oligonucleotide comprising a chain of nucleotides that is described by the sequence referred to using the standard nucleotide nomenclature.

The terms “siRNA”, “RNAi agent,” “siRNA agent,” and “RNA interference agent” are used interchangeably herein to refer to a bioactive agent that contains RNA and which mediates the targeted cleavage of an RNA transcript via an RNA-induced silencing complex (RISC) pathway. RNAi agents directs the sequence-specific degradation of mRNA through a process known as RNA interference. The RNAi agent modulates, e.g., inhibits, the expression of a gene in a cell, e.g., a cell within a subject, such as a mammalian subject, such as a human. In some embodiments, the RNAi agent used in the compositions, uses and methods of the disclosure comprises a double-stranded RNA (dsRNA) or duplex of the disclosure and may be referred to herein as a “double-stranded RNAi agent”, a “dsRNAi agent” or a “dsRNA agent”.

In certain embodiments, a dsRNAi agent of the disclosure includes a double-stranded RNA agent which, when introduced into cells, is processed by the endonuclease known as Dicer into short interfering RNAs. The short interfering RNAs are incorporated into an RISC where one or more helicases unwind the RNA duplex, enabling the complementary antisense strand to guide target recognition. Upon binding to the target mRNA, one or more endonucleases within the RISC cleave the target mRNA to induce silencing. Thus, in other embodiments an siRNA agent relates to a single stranded RNA generated within a cell and which promotes formation of a RISC complex to effect silencing of the target gene. In some such embodiments, the RNAi agent is a single-stranded siRNA (ssRNAi) that can be introduced into a cell or organism to inhibit a target mRNA. Single-stranded RNAi agents bind to the RISC endonuclease, Argonaute 2, which then cleaves the target mRNA. ssRNAi agents are generally 15-30 nucleotides long and may be chemically modified. Any of the antisense oligonucleotides described herein may be used as a ssRNAi agent as described herein. In some embodiments, an ssRNAi agent comprises at least one non-canonical base pairing nucleotide. In some embodiments an ssRNAi agent comprises at least one modified nucleotide.

The term “double-stranded RNA” or “dsRNA” refers to a complex of ribonucleic acid molecules, having a duplex structure comprising two anti-parallel and substantially complementary nucleic acid strands, referred to as having “sense” and “antisense” orientations with respect to a target RNA. In some embodiments of the disclosure, a double-stranded RNA (dsRNA) triggers the degradation of a target RNA, e.g., an mRNA, e.g., an mRNA for a target gene (e.g., INHBE), through a post-transcriptional gene-silencing mechanism referred to herein as RNA interference or RNAi. In general, the majority of nucleotides of each strand of a dsRNA molecule are ribonucleotides, but as described in detail herein, each or both strands can also include one or more non-ribonucleotides, e.g., a deoxyribonucleotide or a modified nucleotide or a non-canonical nucleotide. Each strand of a dsRNA molecule can range in length from 12-40 nucleotides. For example, each strand can be from 14-40 nucleotides in length, 17-37 nucleotides in length, 25-37 nucleotides in length, 17-25 nucleotides in length, 17-22 in length, 19-25 nucleotides in length, 19-23 nucleotides in length, 19-21 nucleotides in length, 15-23 nucleotides in length, 15-17 nucleotides in length, or 21-23 nucleotides in length, and the length of the sense and antisense strands can be equal or unequal without limitation.

The term “antisense strand” refers to the strand of an RNAi agent (e.g., a dsRNA, dsRNAi) that includes a region that is substantially complementary to a target sequence (e.g., an INHBE mRNA). As used herein, the term “region of complementarity” refers to a region of the antisense strand that is substantially complementary to a target sequence. Where the region of complementarity is not fully complementary to the target sequence, there may be mismatches in internal or terminal regions of the molecule. Typically, the most tolerated mismatches are found within the terminal region, e.g., within 5, 4, 3 or 2 nucleotides of the 5′- and/or 3′-end of the dsRNA. The length of antisense and sense strands of a dsRNA can be the same or different, as described herein and as known in the art.

Where a first sequence is referred to as “substantially complementary” with respect to a second sequence, the two sequences can be fully complementary (i.e., complementary over the entire length of one or both nucleotide sequences), or they can form one or more, but generally not more than 5, 4, 3, 2, or 1 mismatched base pairs upon hybridization for a duplex of up to 30 base pairs, while retaining the ability to hybridize under appropriate conditions (e.g., under conditions relevant to their application, e.g., inhibition of gene expression, such as physiological conditions). It should be noted that where two oligonucleotides are designed to form, upon hybridization, one or more single stranded overhangs, such overhangs shall not be regarded as mismatches with regard to the determination of complementarity. For example, a dsRNA comprising one oligonucleotide 21 nucleotides in length and another oligonucleotide 23 nucleotides in length, wherein the longer oligonucleotide comprises a sequence of 21 nucleotides that is fully complementary to the shorter oligonucleotide, can yet be referred to as “fully complementary” for the purposes described herein.

The term “sense strand” as used herein, refers to the strand of a dsRNA that includes a region that is substantially complementary to a region of the antisense strand.

As used herein, and unless otherwise indicated, the term “complementary,” when used to describe a first nucleotide sequence in relation to a second nucleotide sequence, refers to the ability of an oligonucleotide or polynucleotide comprising the first nucleotide sequence to hybridize and form a duplex structure under certain conditions with an oligonucleotide or polynucleotide comprising the second nucleotide sequence, as will be understood by the skilled person. Such conditions can, for example, be stringent conditions, where stringent conditions may include: 400 mM NaCl, 40 mM PIPES pH 6.4, 1 mM EDTA, 50° C. or 70° C. for 12-16 hours followed by washing. Other conditions, such as physiologically relevant conditions as may be encountered inside an organism, can apply. For example, a complementary sequence is sufficient to allow the relevant function of the nucleic acid to proceed, e.g., RNAi. The skilled person will be able to determine the set of conditions most appropriate for a test of complementarity of two sequences in accordance with the ultimate application of the hybridized nucleotides.

The terms “complementary,” “fully complementary” and “substantially complementary” herein can be used with respect to the base pairing between the sense strand and the antisense strand of a dsRNA, or between two oligonucleotides or polynucleotides, such as the antisense strand of a dsRNAi agent and a target sequence, as will be understood from the context of their use.

The term “melting temperature” or “Tm” is used herein to refer to the temperature at which 50% of the double-stranded RNA (dsRNA) molecules are opened or denatured (i.e., 50% of the double-stranded RNA molecules are separated into single strands, 50% of complementary oligonucleotide strands are not hybridized to each other). For a given oligonucleotide, its corresponding Tm value can be obtained by calculation using any accepted formula or software known in the art. For example and without limitation, Tm can be calculated using the OligoAnalyzer™ tool from Integrated DNA Technologies (IDT) (Coralville, Iowa, USA); using the Tm calculation tool at the website http://insilico.ehu.es/tm.php?formula=basic; and the like. The term “ΔTm” refers to the difference in Tm (e.g., calculated Tm) between two different oligonucleotides (e.g., an unmodified oligonucleotide and an oligonucleotide of the same sequence comprising one or more modified nucleotides). In certain embodiments, ΔTm is used to refer to the difference in melting temperature between two dsRNA regions or duplexes of the disclosure, wherein one of the dsRNA regions or duplexes comprises at least one nucleotide replacement with a modified nucleotide, e.g., a non-canonical base pairing nucleotide. In some embodiments, ΔTm is used to refer to the difference in melting temperature between two oligonucleotides (e.g., two antisense strands, two sense strands), wherein one of the oligonucleotides comprises at least one nucleotide replacement with a non-canonical base pairing nucleotide.

“G”, “C”, “A”, “T”, and “U” each generally stand for a nucleotide that contains guanine (G), cytosine (C), adenine (A), thymine (also referred to as 5-methyluracil) (T), and uracil (U) as a base, respectively. However, it will be understood that the term “ribonucleotide” or “nucleotide” can also refer to a modified nucleotide, as further detailed below. G, C, A, T and U are referred to herein as “canonical” nucleotides. Canonical nucleotides are defined as shown in Table A. Accordingly, guanine, cytosine, adenine, thymine, and uracil are referred to herein as “canonical” bases. Canonical bases A, U, G, C, and T are the common bases used for RNA or DNA construction. G, C, A, T and U can also be referred to herein as corresponding “canonical” bases when only base groups or base portions are described without causing ambiguity. For canonical bases, in most cases A pairs with U (T in DNA) and G pairs with C, following Watson-Crick base pair rules (referred to herein as “canonical base pairing”).

TABLE A
Definitions of canonical nucleotides.

| Abbreviation | Name |
|---|---|
| A | adenosine-3′-phosphate |
| C | cytidine-3′-phosphate |
| G | guanosine-3′-phosphate |
| U | uridine-3′-phosphate |
| T | 5-methyluridine-3′-phosphate |

“Non-canonical” base pairing that does not follow Watson-Crick base pair rules is also possible. For example, G-U wobble base pairing occurs commonly and has pairing strength (e.g., thermodynamic stability) comparable to that of a Watson-Crick base pair. Hence, adenine and cytosine anywhere in the nucleotide sequence of a dsRNAi agent of the disclosure can be replaced with guanine and uracil respectively, to form G-U Wobble base pairing with the target sequence.

Certain modified nucleotides also demonstrate non-canonical base pairing. Such modified nucleotides have different base pairing characteristics than the canonical nucleotides from which they are derived. For example, a modified nucleotide with hypoxanthine as its base (such as inosinic acid) can base pair with nucleotides containing adenine, cytosine, or uracil. Hence, nucleotides containing uracil, guanine, or adenine can be replaced in the nucleotide sequence of a dsRNAi agent of the disclosure by a modified nucleotide containing the base hypoxanthine (e.g., inosinic acid). In certain embodiments, one or more canonical nucleotide in the dsRNAi agent of the disclosure is replaced with a modified nucleotide having different base pairing characteristics; such replacement moieties are referred to herein as “non-canonical base pairing nucleotides”. Non-canonical base pairing nucleotides of the disclosure include both nucleotides capable of non-Watson-Crick or wobble base pairing and/or modified nucleotides with different base pairing characteristics compared to the canonical nucleotides they replace. Sequences containing such non-canonical base pairing nucleotides are suitable for the RNAi agents, compositions and methods of the disclosure.

It should be understood that non-canonical base pairing nucleotides (e.g., modified nucleotides having different base pairing characteristics than the canonical nucleotides which they replace) may differ not only in the base pairs which they form, but also in the strength or stability of those pairings. Non-canonical base pairing may be stronger or weaker than canonical base pairing. For example, the pairing of m1Ψ (which is modified from U) with A is stronger than the pairing of U with A, and m1Ψ-G pairing is even stronger than m1Ψ-A pairing. Hence by replacing a canonical nucleotide with a non-canonical base pairing nucleotide, it is possible to change the base pairing strength and hence the Tm of an antisense-sense strand duplex and/or of hybridization between an antisense strand and a target RNA (e.g., INHBE mRNA). Thus, in some embodiments, a canonical nucleotide is replaced with a non-canonical base pairing nucleotide, thereby altering the Tm of the oligonucleotide (e.g., altering the calculated Tm of the oligonucleotide, altering the Tm of resulting double-stranded RNA molecules, e.g., duplex of the antisense strand hybridized with the target mRNA and/or the sense strand). Without wishing to be limited by theory, by altering the Tm of an oligonucleotide through replacement of at least one canonical nucleotide with a non-canonical base pairing nucleotide, the efficacy, potency, specificity, safety and/or off-target effects of the dsRNAi agent can be modulated. For example and without limitation, the efficacy or potency of the dsRNAi agent may be increased by increasing pairing strength to the desired target mRNA and/or decreasing pairing to off-target mRNAs. Similarly, undesirable off-target effects may be reduced by increasing pairing strength to the desired target mRNA and/or decreasing pairing strength for off-target mRNAs. Hence in some embodiments, the RNAi agent of the disclosure has improved efficacy, potency, specificity and/or safety compared to similar RNAi agents which do not include at least one non-canonical base pairing nucleotide.

In certain embodiments of dsRNAi agents, antisense strands, and sense strands of the disclosure, at least one canonical nucleotide is replaced with a non-canonical base pairing nucleotide. In some such embodiments, one nucleotide is replaced with a non-canonical base pairing nucleotide, i.e., the dsRNAi agent, antisense strand, or sense strand comprises one non-canonical base pairing nucleotide. In some embodiments, two, three, four five or more nucleotides are replaced with non-canonical base pairing nucleotides, i.e., the dsRNAi agent, antisense strand, or sense strand comprises two, three, four five or more non-canonical base pairing nucleotides. In some embodiments, 5%, 10%, 15%, 20%, 25%, 30%, 35%, 40%, 45%, 50%, 55%, 60%, 65%, 70%, 75%, 80%, 85%, 90%, 95% or more of the nucleotides in an oligonucleotide or dsRNAi agent are modified nucleotides, e.g., non-canonical base pairing nucleotides. In some embodiments, all of the nucleotides in the dsRNAi agent, e.g., the sense strand and/or the antisense strand, are modified nucleotides, e.g., non-canonical base pairing nucleotides. In some embodiments, at least one nucleotide in the dsRNAi agent, e.g., the sense strand and/or the antisense, is a modified nucleotide, e.g., a non-canonical base pairing nucleotide.

In certain embodiments, therefore, the RNAi agent of the disclosure comprises at least one non-canonical base pairing nucleotide, i.e., at least one nucleotide in the antisense strand and/or the sense strand is replaced by a non-canonical base pairing nucleotide. In some such embodiments, the at least one non-canonical base pairing nucleotide is present on the antisense strand. In some such embodiments, the at least one non-canonical base pairing nucleotide is present on the sense strand. In some such embodiments, at least one non-canonical base pairing nucleotide is present on both the antisense strand and the sense stand. In some embodiments, the at least one non-canonical base pairing nucleotide is present in the region of complementarity, i.e., the region in an oligonucleotide that is substantially complementary to a target sequence, e.g., the region in an antisense strand of the disclosure complementary to the target mRNA (e.g., INHBE mRNA).

In certain embodiments, replacement of at least one canonical nucleotide with a non-canonical base pairing nucleotide changes the melting temperature (Tm) of the oligonucleotide or dsRNA duplex. In some embodiments, the Tm is changed by at least 2° C. (i.e., ΔTm is at least about 2° C.). In some embodiments, ΔTm is about 2° C. In some embodiments, ΔTm is over 2° C. In some embodiments, ΔTm is about 2.5° C., 3° C., 3.5° C., 4° C., 4.5° C., or 5° C.

In certain embodiments, the at least one non-canonical base pairing nucleotide is present at positions 1-11 of the antisense strand, e.g., at position 1, 2, 3, 4, 5, 6, 7, 8, 9, 10, and/or 11, according to the direction from the 5′ end to the 3′ end.

In certain embodiments, the at least one non-canonical base pairing nucleotide is present at positions 12-21 of the antisense strand, e.g., at position 12, 13, 14, 15, 16, 17, 18, 19, 20, and/or 21, according to the direction from the 5′ end to the 3′ end.

In certain embodiments of oligonucleotides and dsRNAi agents of the disclosure, according to the direction from the 5′ end to the 3′ end, if at least four (4) of the nucleotides at positions 2-8 of the antisense strand are A or U and at least one (1) of the nucleotides at positions 6-8 is G, then a G at position 6, 7 and/or 8 is replaced with a non-canonical base pairing nucleotide.

In certain embodiments of oligonucleotides and dsRNAi agents of the disclosure, guanine (G) in the following sequence is replaced with a non-canonical base, e.g., hypoxanthine (I), if at least three (3) bases of N₁, N₂, N₃, and N₄ are adenine (A) or uracil (U), wherein N₁, N₂, N₃, and N₄ are nucleotides independently containing adenine (A), cytosine (C), guanine (G), thymine (T) or uracil (U) as a base, and G₀ is a nucleotide containing guanine as a base: 5′-N₁N₂G₀N₃N₄-3′.

It should be understood that all non-canonical base pairing nucleosides (or nucleotides) and non-canonical bases disclosed herein or known in the art are suitable for use in RNAi agents, compositions and methods of the disclosure. Non-limiting examples of non-canonical base pairing nucleosides (or nucleotides) or non-canonical bases are defined as shown in Table B. In some embodiments, a non-canonical base pairing nucleoside (or nucleotide) or a non-canonical base is a modified group shown in Table B. In some embodiments, a non-canonical base pairing nucleoside is inosine (I). In some embodiments, a non-canonical base pairing nucleotide is a canonical nucleotide capable of wobble pairing. Combinations of the foregoing are also included.

TABLE B
Examples of non-canonical base pairing
nucleosides (or nucleotides) and non-
canonical bases in accordance with certain embodiments.

| Abbreviation | Name |
|---|---|
| I | hypoxanthine; inosine; inosinic acid |
| X | xanthosine |
| m7G | 7-methylguanosine |
| m6A | N⁶-methyladenosine |
| DHU | dihydrouridine |
| m5C | 5-methylcytidine |
| Ψ | pseudouridine (also known as 5-(β-D- |
| | Ribofuranosyl)pyrimidine-2,4(1H,3H)- |
| | dione or 5-ribosyluracil) |
| m1Ψ | N¹-methylpseudouridine |

The term “modified nucleotide” is used herein to refer to any nucleotide that independently has a modified sugar moiety, a modified internucleotide linkage and/or a modified nucleobase. Thus, the term “modified nucleotide” encompasses substitution, addition, or removal of, for example, a functional group or atom of an internucleoside linkage, sugar moiety or nucleobase. Modifications suitable for use in RNAi agents of the present disclosure include all types of modifications disclosed herein or known in the art. In some embodiments, a modified nucleotide is a non-canonical base pairing nucleotide as defined herein, i.e., a nucleotide having different pairing characteristics than the canonical nucleotide it replaces. In other embodiments, a modified nucleotide may not have different pairing characteristics but may nevertheless possess characteristics such as stability, resistance to degradation (e.g., resistance to nucleases), manufacturability, and the like, which are desirable or advantageous for the RNAi agents, compositions and methods of the disclosure. In certain embodiments the RNAi agent of the disclosure comprises at least one modified nucleotide in addition to at least one non-canonical base pairing nucleotide, i.e., in addition to including at least one non-canonical base pairing nucleotide, at least one additional nucleotide in the antisense strand and/or the sense strand is replaced by an additional modified nucleotide (which may or may not also be a non-canonical base pairing nucleotide). In some such embodiments, the at least one additional modified nucleotide is present on the antisense strand. In some such embodiments, the at least one additional modified nucleotide is present on the sense strand. In some such embodiments, at least one additional modified nucleotide is present on both the antisense strand and the sense stand. In some such embodiments, the at least one additional modified nucleotide is present on the same strand as the at least one non-canonical base pairing nucleotide. In other embodiments, the at least one additional modified nucleotide is not present on the same strand as the at least one non-canonical base pairing nucleotide, i.e., is present on the other strand. In some embodiments, the at least one additional modified nucleotide is present in the region of complementarity, i.e., the region in an oligonucleotide that is substantially complementary to a target sequence, e.g., the region in an antisense strand of the disclosure complementary to the target mRNA (e.g., INHBE mRNA).

In the present disclosure, non-limiting examples of common modified nucleotides and related moieties are defined as shown in Table C. In some embodiments, a modified nucleotide is a modified nucleotide shown in Table C. In some embodiments, the at least one additional modified nucleotide in a RNAi agent of the disclosure is a modified nucleotide shown in Table C.

TABLE C
Definitions of modified nucleotides in accordance
with certain embodiments.

| Abbreviation | Name |
|---|---|
| N | any nucleotide |
| N* | thiophosphate-modified nucleotide |
| mN | 2′-O-methyl-modified nucleotide |
| mA* | 2′-O-methyl-adenosine-3′-thiophosphate-phosphate |
| fN | 2′-fluoro-modified nucleotide |
| fA* | 2′-fluoro-adenosine-3′-thiophosphate-phosphate |
| dN | 2′-deoxyribonucleotide |
| VP | 5′-Vinylphosphonate |
| irN | inverted nucleotide |

As used herein, the modification pattern of inverted nucleotides (also called inverted bases) refers to those bases with linkages reversed from normal 5′ to 3′ linkages (i.e., 5′ to 5′ linkages or 3′ to 3′ linkages).

In some embodiments, inclusion of a deoxy-nucleotide can be considered to constitute a modified nucleotide.

It should be understood that all types of modifications disclosed herein or known in the art are suitable for use in RNAi agents, compositions and methods of the disclosure.

The term “derivative”, as used herein, is understood as being a substance similar in structure to another compound but differing in some slight structural detail.

The term “inhibiting”, as used herein, is used interchangeably with “reducing,” “silencing”, “downregulating”, “suppressing” and other similar terms, and includes any level of inhibition. As a non-limiting example, “inhibiting” refers to reducing or effectively reducing the onset or progression of a metabolic disorder or related disease in a subject, including a reduction in one or more aspects of the disease (such as symptoms, tissue characteristics, cell activity, inflammatory activity, or immune activity, etc.), or with no detectable deterioration in symptoms in the subject.

As used herein, the phrase “inhibiting expression of a gene” (e.g., “inhibiting INHBE”) refers to inhibiting the expression of RNA transcripts (e.g., INHBE mRNA) encoded by said gene compared to an appropriate reference (e.g., reference cell, cell population, sample or subject) and/or to reducing the level of RNA transcripts (e.g., INHBE mRNA)(e.g., by degradation) and/or to reducing the amount, level and/or activity of the gene products (e.g., RNAs, proteins) in a cell, cell population, sample or subject. The phrase “inhibiting INHBE expression” thus refers to a reduction of the amount or level or activity of INHBE mRNA and/or INHBE protein in a cell, cell population, sample or subject compared to an appropriate reference (e.g. a reference cell, cell population, sample or subject), such as an inhibition by at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 91%, at least About 92%, at least about 93%, at least about 94%, at least about 95%, at least about 96%, at least about 97%, at least about 98%, or at least about 99%, compared to the appropriate reference.

As used herein, the phrase “contacting a cell with an RNAi agent,” such as a dsRNAi agent, includes contacting a cell by any possible means. Contacting a cell with an RNAi agent includes contacting a cell in vitro with the or contacting a cell in vivo with the RNAi agent. The contacting may be done directly or indirectly. Thus, for example, the RNAi agent may be put into physical contact with the cell, or alternatively, the RNAi agent may be put into a situation that will permit or cause it to subsequently come into contact with the cell. Contacting a cell in vitro may be done, for example, by incubating the cell with the RNAi agent. Contacting a cell in vivo may be done, for example, by injecting the RNAi agent into or near the tissue where the cell is located, or by injecting the RNAi agent into another area, e.g., the bloodstream or the subcutaneous space, such that the agent will subsequently reach the tissue where the cell to be contacted is located. For example, the RNAi agent may contain or be coupled to a targeting ligand, e.g., GalNAc, that directs the RNAi agent to a site of interest, e.g., the liver. In other embodiments, the RNAi agent may contain or be coupled to one or more C22 hydrocarbon chains and one or more GalNAc derivatives. In other embodiments, the RNAi agent contains or is coupled to one or more C22 hydrocarbon chains and does not contain or is not coupled to one or more GalNAc derivatives. Combinations of in vitro and in vivo methods of contacting are also possible. For example, a cell may also be contacted in vitro with an RNAi agent of the disclosure and subsequently transplanted into a subject. In certain embodiments, contacting a cell with an RNAi agent includes facilitating or effecting uptake or absorption into the cell. Absorption or uptake of an RNAi agent can occur through unaided diffusion or active cellular processes, or by auxiliary agents or devices. Introducing an RNAi agent into a cell may be in vitro or in vivo. For example, for in vivo introduction, an RNAi agent can be injected into a tissue site or administered systemically. In vitro introduction into a cell includes methods known in the art such as electroporation and lipofection.

The term “subject” includes animals, including mammals and humans, particularly humans. Non-limiting examples of subjects include humans, monkeys, cows, rabbits, sheep, goats, pigs, dogs, cats, rats, mice, and transgenic species thereof. As used herein, the term “cyno” or “cynomolgus monkey” refers to cynomolgus monkeys. In certain embodiments, the subject is a human.

“Treating” or “treatment” of any disease or disorder refers, in some embodiments, to ameliorating at least one disease or disorder. In certain embodiments “treating” or “treatment” refers to ameliorating at least one physical parameter, which may or may not be discernible by the patient. In certain embodiments, “treating” or “treatment” refers to inhibiting the disease or disorder, either physically (e.g., stabilization of a discernible symptom), physiologically (e.g., stabilization of a physical parameter), or both. In some embodiments, “treating” or “treatment” refers to improving the quality of life or reducing the symptoms or side effects of a disease in a subject in need thereof “Therapeutically effective amount” means the amount of a dsRNA or a dsRNAi agent that, when administered to a subject for treating or preventing a disease, is sufficient to effect such treatment or prevention of the disease. The “therapeutically effective amount” will vary depending on the compound or RNAi agent, the disease and its severity, and the age, weight, etc., of the subject having the disease to be treated or prevented. As used herein, the term “therapeutically effective amount” refers to an amount of a compound or composition sufficient to prevent, treat, inhibit, reduce, ameliorate or eliminate one or more causes, symptoms, or complications of a disease or disorder such as, for example, metabolic syndrome. The terms “effective amount” and “therapeutically effective amount” are used interchangeably herein.

“Preventing” or “prevention” or “prophylaxis” of any disease or disorder refers, in some embodiments, to at least the reduction of likelihood of the risk of (or susceptibility to) acquiring a disease or disorder (i.e., causing at least one of the clinical symptoms of the disease not to develop in a patient that may be exposed to or predisposed to the disease but does not yet experience or display symptoms of the disease).

The phrase “pharmaceutically acceptable” as used herein refers to those compounds, materials, compositions, or dosage forms which are, within the scope of sound medical judgment, suitable for use in contact with the tissues of human subjects and animal subjects without excessive toxicity, irritation, allergic response, or other problem or complication, commensurate with a reasonable benefit/risk ratio.

The phrase “pharmaceutically acceptable carrier” as used herein refers to a pharmaceutically acceptable material, composition or vehicle, such as a liquid or solid filler, diluent, excipient, manufacturing auxiliary (such as a lubricating talc, magnesium stearate, calcium or zinc stearate, or stearic acid), or solvent-encapsulating materials (involved in carrying or transporting the compound or dsRNAi agent from one organ or part of the body to another).

### RNA Interference

The present disclosure provides a small interfering RNA (siRNA or RNAi) agent that inhibits the expression of the target gene INHBE in a cell via an RNA interference (RNAi) process. In some embodiments, the RNAi agent comprises a double-stranded ribonucleic acid (dsRNA) molecule for inhibiting expression of the INHBE gene in a cell (e.g., an adipocyte and/or a liver cell, e.g., a hepatocyte). An siRNA or RNAi agent comprising a dsRNA molecule is also referred to herein as a “dsRNAi” agent. In certain embodiments the cell is found within a subject. In some embodiments the subject is a mammal, e.g., a human. In some embodiments the subject has, suffers from, or is predisposed to a metabolic disorder (e.g., metabolic syndrome), a carbohydrate disorder (e.g., type 2 diabetes, pre-diabetes), a lipid metabolism disorder (e.g., hyperlipidemia, hypertension, lipodystrophy), kidney disease, cardiovascular disease, and/or a weight disorder (e.g., obesity, overweight). The dsRNAi agent of the disclosure includes an antisense strand having a region of complementarity to at least a portion of an mRNA formed in the expression of INHBE. In some embodiments the complementary region is from about 19-30 nucleotides in length (e.g., about 30, 29, 28, 27, 26, 25, 24, 23, 22, 21, 20, or 19 nucleotides in length). In some embodiments the complementary region is from about 15-30 nucleotides in length (e.g., about 30, 29, 28, 27, 26, 25, 24, 23, 22, 21, 20, 19, 18, 17, 16, or 15 nucleotides in length). In some embodiments the complementary region comprises at least 15 contiguous nucleotides. In certain embodiments, the dsRNAi agent comprises at least one modified nucleotide, as described herein. In certain embodiments, the dsRNAi agent comprises at least on non-canonical base pairing nucleotide, as described herein.

In accordance with methods of the disclosure, cells expressing the target gene are contacted with the siRNA agent which inhibits expression of the target gene (e.g., human, primate, non-primate, or mouse INHBE gene). In some embodiments, expression of the target gene is inhibited by at least about 50%. Inhibition of expression of the target gene may be determined using any suitable method, for example and without limitation, by PCR or branched DNA (bDNA) methods, or by protein methods, e.g., by immunofluorescence analysis, using e.g., Western blot or flow cytometry techniques. In some embodiments, inhibition of expression is determined by the rt-PCR method (e.g., as described in the Examples hereinbelow, using, for example, siRNA at a concentration of 10 nM in a cell line of a suitable organism). In some embodiments, inhibition of expression in vivo is determined using an animal model, e.g., by knockdown of the human gene in a rodent expressing the human gene (e.g., a mouse expressing the human INHBE gene). In some such embodiments the siRNA is administered to the subject (e.g., the animal model) as a single dose, e.g., in a single dose at 3 mg/kg, 6 mg/kg, or 9 mg/kg).

A dsRNA includes two RNA strands that are complementary and hybridize to form a duplex structure under conditions in which the dsRNA will be used (e.g., under physiological conditions). One strand of the dsRNA (the antisense strand) includes a region of complementarity that is substantially, and in some embodiments fully, complementary to a target sequence. Target sequences can be derived from the sequence of an mRNA formed during the expression of an INHBE. The other strand (the sense strand) includes a region that is complementary to the antisense strand such that when the two strands are combined under appropriate conditions, they will hybridize and form a duplex structure. As described elsewhere herein and known in the art, the complementary sequence of the dsRNA can also be included as a self-complementary region of a single nucleic acid molecule rather than on separate oligonucleotides. Generally, the duplex structure is from 15 to 30 base pairs in length, such as without limitation 15-29, 15-28, 15-27, 15-26, 15-25, 15-24, 15-23, 15-22, 15-21, 15-20, 15-19, 15-18, 15-17, 17-25, 18-30, 18-29, 18-28, 18-27, 18-26, 18-25, 18-24, 18-23, 18-22, 18-21, 18-20, 19-30, 19-29, 19-28, 19-27, 19-26, 19-25, 19-24, 19-23, 19-22, 19-21, 19-20, 20-30, 20-29, 20-28, 20-27, 20-26, 20-25, 20-24, 20-23, 20-22, 20-21, 21-30, 21-29, 21-28, 21-27, 21-26, 21-25, 21-24, 21-23, or 21-22 base pairs in length. In certain embodiments, the length of the duplex structure is 17 to 25 base pairs, such as without limitation 17-23, 17-25, 18-25, 18-24, 18-23, 18-22, 18-21, 18-20, 19-25, 19-24, 19-23, 19-22, 19-21, 19-20, 20-25, 20-24, 20-23, 20-22, 20-21, 21-25, 21-24, 21-23, 21-22, 22-25, 22-24, 22-23, 23-25, 23-24 or 24-25 base pairs in length, e.g., 19-21 base pairs in length. Ranges and lengths intermediate to the above ranges and lengths are also considered to be part of the disclosure.

Similarly, the region of complementarity to the target sequence is 15 to 30 nucleotides in length, e.g., 15-29, 15-28, 15-27, 15-26, 15-25, 15-24, 15-23, 15-22, 15-21, 15-20, 15-19, 15-18, 15-17, 18-30, 18-29, 18-28, 18-27, 18-26, 18-25, 18-24, 18-23, 18-22, 18-21, 18-20, 19-30, 19-29, 19-28, 19-27, 19-26, 19-25, 19-24, 19-23, 19-22, 19-21, 19-20, 20-30, 20-29, 20-28, 20-27, 20-26, 20-25, 20-24,20-23, 20-22, 20-21, 21-30, 21-29, 21-28, 21-27, 21-26, 21-25, 21-24, 21-23, or 21-22 nucleotides in length, for example 19-23 nucleotides in length or 21-23 nucleotides in length. Ranges and lengths intermediate to the above recited ranges and lengths are also contemplated to be part of the disclosure.

In some embodiments, the duplex structure is 19 to 30 base pairs in length. Similarly, the region of complementarity to the target sequence is 19 to 30 nucleotides in length.

In some embodiments, the duplex structure is 15 to 23 base pairs in length. Similarly, the region of complementarity to the target sequence is 15 to 23 nucleotides in length.

In some embodiments, the dsRNA is about 19 to about 23 nucleotides in length, or about 25 to about 30 nucleotides in length. In some embodiments, the dsRNA is about 15 to about 23 nucleotides in length, or about 17 to about 23 nucleotides in length, or about 17 to about 25 nucleotides in length, or about 19 to about 21 nucleotides in length.

The duplex region is a primary functional portion of a dsRNA, e.g., a duplex region of about 15 to about 30 base pairs, or about 17 to about 30 base pairs, or about 19 to about 30 base pairs, e.g., about 15-23, 15-25, 17-25, 17-23, 19-30, 19-29, 19-28, 19-27, 19-26, 19-25, 19-24, 19-23, 19-22, 19-21, 19-20, 20-30, 20-29, 20-28, 20-27, 20-26, 20-25, 20-24,20-23, 20-22, 20-21, 21-30, 21-29, 21-28, 21-27, 21-26, 21-25, 21-24, 21-23, or 21-22 base pairs. Thus, in one embodiment, to the extent that it becomes processed to a functional duplex, of e.g., 15-30 base pairs or at least 15 base pairs, that targets a desired RNA for cleavage, an RNA molecule or complex of RNA molecules having a duplex region greater than 30 base pairs is a dsRNA. In another embodiment, a dsRNA is not a naturally occurring miRNA. In another embodiment, an siRNA agent useful to target INHBE gene expression is not generated in the target cell by cleavage of a larger dsRNA.

A dsRNA as described herein can further include one or more single-stranded nucleotide overhangs, e.g., 1-4, 2-4, 1-3, 2-3, 1, 2, 3, or 4 nucleotides. dsRNAs having at least one nucleotide overhang can have superior inhibitory properties relative to their blunt-ended counterparts. A nucleotide overhang can comprise or consist of a nucleotide/nucleoside analog, including a deoxynucleotide/nucleoside. The overhang(s) can be on the sense strand, the antisense strand, or any combination thereof. Furthermore, the nucleotide(s) of an overhang can be present on the 5′-end, the 3′-end, or both ends of an antisense or sense strand of a dsRNA.

“Blunt” or “blunt end” means that there are no unpaired nucleotides at the end of the dsRNA, i.e., no nucleotide overhang. A “blunt ended” dsRNA is double-stranded over its entire length, i.e., no nucleotide overhang at either end of the molecule. The dsRNAi agents of the disclosure include dsRNAs with no nucleotide overhang at one end (i.e., agents with one overhang and one blunt end) or with no nucleotide overhangs at either end. In some embodiments such oligonucleotides are double-stranded over their entire length.

A dsRNA can be synthesized by standard methods known in the art. Double-stranded RNAi compounds of the invention can be prepared using a two-step procedure. First, each strand of a double-stranded RNA molecule is prepared separately and then annealed. Individual strands of siRNA compounds can be prepared using solution phase or solid phase organic synthesis or both. An advantage of organic synthesis is that oligonucleotide chains comprising non-natural or modified nucleotides can be readily prepared. Similarly, single-stranded oligonucleotides of the technology can be prepared using solution-phase or solid-phase organic synthesis, or both.

In an aspect, a dsRNA of the disclosure includes at least two nucleotide sequences, a sense sequence and an antisense sequence. In some embodiments, the sense strand is selected from the group of sequences provided in any one of Tables 1 and 2, and the corresponding antisense strand of the sense strand is selected from the group of sequences in any one of Tables 1 and 2. In this aspect, one of the two sequences is complementary to the other of the two sequences, with one of the sequences being substantially complementary to a sequence of an mRNA generated in the expression of a target gene. As such, in this aspect, a dsRNA will include two oligonucleotides, where one oligonucleotide is described as the sense strand in any one of Tables 1 and 2, and the second oligonucleotide is described as the corresponding antisense strand of the sense strand in any one of Tables 1 and 2.

In some embodiments, the sense and/or antisense strand is selected from the sense and/or antisense strand of any one of duplexes IN-043, IN-091, IN-106, IN-176, IN-189, IN-202, or IN-221.

It can be understood that although the sequences in Table 1 are un-modified or un-conjugated sequences, the RNA of the siRNA of the disclosure, e.g., a dsRNA of the disclosure, may comprise any one of the sequences set forth in any one of Tables 1 and 2 that is un-modified, un-conjugated, or modified or conjugated differently than described therein. In other words, the disclosure encompasses dsRNA of Tables 1 and 2 which are un-modified, un-conjugated, modified, or conjugated, as described herein.

The skilled person understands dsRNAs having a duplex structure of about 20 to 23 base pairs, e.g., 21, base pairs have been hailed as particularly effective in inducing RNA interference (Elbashir et al., EMBO 2001, 20:6877-6888). However, others have found that shorter or longer RNA duplex structures can also be effective (Chu and Rana (2007) RNA 14:1714-1719; Kim et al. (2005) Nat Biotech 23:222-226). In some embodiments, the dsRNA described herein can include at least one strand of a length of minimally 21 nucleotides. It can be reasonably expected that shorter duplexes having any one of the sequences in any one of Tables 1 and 2 minus only a few nucleotides on one or both ends can be similarly effective as compared to the dsRNAs described above. Hence, dsRNAs having a sequence of at least 12, 13, 14, 15, 19, 20, or more contiguous nucleotides derived from any one of the sequences of any one of Tables 1 and 2, and differing in their ability to inhibit the expression of an INHBE gene by not more than about 5, 10, 15, 20, 25, or 30% inhibition from a dsRNA comprising the full sequence, are contemplated to be within the scope of the present disclosure.

### Modifications for the RNAi Agent

In some embodiments, the RNA of the dsRNAi agent of the disclosure is un-modified, and does not comprise, e.g., chemical modifications or conjugations as known in the art and described herein. In other embodiments, the RNA of an dsRNAi agent of the disclosure, e.g., a dsRNA, is chemically modified to enhance stability or to provide other beneficial characteristics. In some embodiments, substantially all the nucleotides of an RNA of the invention are modified. In other embodiments, all the nucleotides of an RNA or substantially all the nucleotides of an RNA are modified. In some embodiments, not more than 5, 4, 3, 2, or 1 unmodified nucleotides are present in a strand of RNA of the disclosure, e.g., oligonucleotide, antisense strand, sense strand, or dsRNA.

In some embodiments, the dsRNAi agents comprise at least one nucleic acid modification described herein. For example, the dsRNAi agents may comprise at least one modification selected from the group consisting of modified internucleoside linkage, modified nucleobase, modified sugar, and any combinations thereof. Without limitations, such a modification can be present anywhere in the dsRNAi agent of the disclosure. In certain embodiments, the dsRNAi agents comprise at least one non-canonical base pairing nucleotide. Without limitations, such nucleotides can be present anywhere in the dsRNAi agent of the disclosure. In some embodiments, the at least one modified nucleotide and/or non-canonical base pairing nucleotide changes the melting temperature of the oligonucleotide, e.g., ΔTm is at least 2° C., e.g., about 2° C., over 2° C., about 2-5° C., about 3° C., about 4° C., or about 5° C.

In one embodiment, the dsRNAi agents of the disclosure comprise one or more targeting ligands, e.g., one or more GalNAc derivatives, and comprise at least one additional nucleic acid modification described herein. For example, the dsRNAi agents may comprise at least one modification selected from the group consisting of modified internucleoside linkage, modified nucleobase, modified sugar, and any combinations thereof. Without limitations, such a modification can be present anywhere in the dsRNAi agent of the disclosure. For example, the modification can be present in one of the RNA molecules. Modifications include, for example and without limitation, end modifications, e.g., 5′-end modifications (phosphorylation, conjugation, inverted linkages) or 3′-end modifications (conjugation, DNA nucleotides, inverted linkages, etc.); base modifications, e.g., replacement with stabilizing bases, destabilizing bases, or bases that base pair with an expanded repertoire of partners, removal of bases (abasic nucleotides), or conjugated bases; sugar modifications (e.g., at the 2′-position or 4′-position) or replacement of the sugar; or backbone modifications, including modification or replacement of the phosphodiester linkages. Specific examples of RNAi agents useful in the embodiments described herein include, but are not limited to, RNAs containing modified backbones or no natural internucleoside linkages. RNAs having modified backbones include, among others, those that do not have a phosphorus atom in the backbone. In some embodiments, a modified RNAi agent will have a phosphorus atom in its internucleoside backbone.

In some embodiments, backbone modifications refer to internucleoside linkages or backbones including, but not limited to, phosphorothioate moiety, chiral phosphorothioate, phosphorothioate, phosphorodithioate, phosphotriester, aminoalkyl-phosphotriester, chiral phosphonate, phosphinate, phosphoramidate, thioalkylphosphonate, thioalkylphosphotriester, and morpholino link, wherein the adjacent nucleoside unit pairs are linked 3′-5′ to 5′-3′ or 2′-5′ to 5′-2′.

In some embodiments, the sense strand of a dsRNA may contain 1, 2, 3, 4, 5, or 6 phosphorothioate linkages (phosphorothioate-modified nucleotides), and the antisense strand of a dsRNA may contain 1, 2, 3, 4, 5 or 6 phosphorothioate linkages (phosphorothioate modified nucleotides). In some embodiments, the sense strand of a dsRNA can contain 1 or 2 phosphorothioate linkages, and the antisense strand of an siRNA can contain 1, 2, 3, or 4 phosphorothioate linkages.

In some embodiments, the sense strand of the dsRNA contains 2 phosphorothioate internucleoside linkages. In some embodiments, the phosphorothioate internucleoside linkage is located between nucleotides at position 1-3 from the 5′ end of the sense strand. In some embodiments, the phosphorothioate internucleoside linkage is located between nucleotides at position 1-3 from the 3′ end of the sense strand. In some embodiments, one phosphorothioate internucleoside linkage is at the 5′ end of the sense strand, and another phosphorothioate linkage is at the 3′ end of the sense strand. In some embodiments, the sense strand of the dsRNAi contains 1 phosphorothioate internucleoside linkage. In some embodiments, the phosphorothioate internucleoside linkage is located between nucleotides at position 1-2 from the 5′ end of the sense strand. In some embodiments, the phosphorothioate internucleoside linkage is located between nucleotides at position 2-3 from the 5′ end of the sense strand. In some embodiments, the targeting ligand is attached to the sense strand via a phosphorothioate linkage.

In some embodiments, the antisense strand of the dsRNA contains 4 phosphorothioate internucleoside linkages. In some embodiments, the four phosphorothioate internucleoside linkages are located between the nucleotides at position 1-3 from the 5′ end of the antisense strand and at position 1-3 from the 3′ end of the antisense strand. In some embodiments, the antisense strand of the dsRNA contains 3 phosphorothioate internucleoside linkages. In some embodiments, the three phosphorothioate internucleoside linkages are located between nucleotides at position 1-2 from the 5′ end of the antisense strand and at position 1-3 from the 3′ end of the antisense strand. In some embodiments, the three phosphorothioate internucleoside linkages are located between nucleotides at position 1-3 from the 5′ end of the antisense strand and at position 1-2 positions from the 3′ end of the antisense strand. In some embodiments, the antisense strand of the dsRNA contains 2 phosphorothioate internucleoside linkages. In some embodiments, the two phosphorothioate internucleoside linkages are located between the nucleotides at position 1-2 from the 5′ end of the antisense strand and at position 1-2 from the 3′ end of the antisense strand.

In some embodiments, the antisense strand of the dsRNA comprises the nucleotide (from 5′ end→3′ end) sequence of any one of the antisense strand sequences in Table 1 or 2. In some embodiments, the sense strand of the dsRNA comprises the nucleotide (from 5′ end→3′ end) sequence of any one of the sense strand sequences in Table 1 or 2. In some embodiments, the antisense strand of the dsRNA comprises the nucleotide (from 5′ end→3′ end) sequence of any one of the antisense strands in Tables 1 or 2, and the sense strand comprises the nucleotide (from 5′ end→3′ end) sequence of any one of the sense strands in Tables 1 or 2.

In some embodiments, the modified nucleotide is selected from: 2′-O-methyl-modified nucleotide, 2′-fluoro-modified nucleotide, 2′-deoxy nucleotide, 2′-methoxyethyl-modified nucleotide, 2′-amino-modified nucleotide, 2′-alkyl-modified nucleotide, 2′-alkoxy-modified nucleotide, 2′-F-arabino nucleotide, phosphorothioate-modified nucleotides, abasic nucleotides, morpholino nucleotide, locked nucleotide, inverted nucleotide, and hypoxanthin base-substituted nucleotide (i.e., inosine).

In some embodiments, the modified nucleotide is selected from: 2′-O-methyl-modified nucleotide, 2′-fluoro-modified nucleotide, 2′-deoxy nucleotide, phosphorothioate-modified nucleotide, and inverted base nucleotide (reverse linkage). In some embodiments, the inverted base nucleotide is selected from: inverted A nucleotide, inverted dA nucleotide, inverted dT nucleotide, inverted C nucleotide and inverted U nucleotide.

Exemplary modified nucleotides or nucleobases include, but are not limited to: synthetic and natural nucleosides or nucleobases such as inosine, xanthine, hypoxanthine, nebularine, isoguanisine, tubercidine, 2-(halo)adenine, 2-(alkyl)adenine, 2-(propyl)adenine, 2-(amino)adenine, 2-(aminoalkyll)adenine, 2-(aminopropyl)adenine, 2-(methylthio)-N6-(isopentenyl)adenine, 6-(alkyl)adenine, 6-(methyl)adenine, 7-(deaza)adenine, 8-(alkenyl)adenine, 8-(alkyl)adenine, 8-(alkynyl)adenine, 8-(amino)adenine, 8-(halo)adenine, 8-(hydroxyl)adenine, 8-(thioalkyl)adenine, 8-(thiol)adenine, N6-(isopentyl)adenine, N6-(methyl)adenine, N6, N6-(dimethyl)adenine, 2-(alkyl)guanine,2-(propyl)guanine, 6-(alkyl)guanine, 6-(methyl)guanine, 7-(alkyl)guanine,7-(methyl)guanine, 7-(deaza)guanine, 8-(alkyl)guanine, 8-(alkenyl)guanine, 8-(alkynyl)guanine, 8-(amino)guanine, 8-(halo)guanine, 8-(hydroxyl)guanine, 8-(thioalkyl)guanine, 8-(thiol)guanine, N-(methyl)guanine, 2-(thio)cytosine, 3-(deaza)-5-(aza)cytosine, 3-(alkyl)cytosine, 3-(methyl)cytosine, 5-(alkyl)cytosine, 5-(alkynyl)cytosine, 5-(halo)cytosine, 5-(methyl)cytosine, 5-(propenyl)cytosine, 5-(propynyl)cytosine, 5-(trifluoromethyl)cytosine, 6-(azo)cytosine, N4-(acetyl)cytosine, 3-(3-amino-3-carboxypropyl)uracil, 2-(thio)uracil, 5-(methyl)-2-(thio)uracil, 5-(methylaminomethyl)-2-(thio)uracil, 4-(thio)uracil, 5-(methyl)-4-(thio)uracil, 5-(methylaminomethyl)-4-(thio)uracil, 5-(methyl)-2,4-(dithio)uracil, 5-(methylaminomethyl)-2,4-(dithio)uracil, 5-(2-aminopropyl)uracil, 5-(alkyl)uracil, 5-(alkynyl)uracil, 5-(allylamino)uracil, 5-(aminoallyl)uracil, 5-(aminoalkyl)uracil, 5-(guanidiniumalkyl)uracil, 5-(1,3-diazole-1-alkyl)uracil, 5-(cyanoalkyl)uracil, 5-(dialkylaminoalkyl)uracil, 5-(dimethylaminoalkyl)uracil, 5-(halo)uracil, 5-(methoxy)uracil, uracil-5-oxyacetic acid, 5-(methoxycarbonylmethyl)-2-(thio)uracil, 5-(methoxycarbonyl-methyl)uracil, 5-(propenyl))uracil, 5-(propynyl)uracil, 5-(trifluoromethyl)uracil, 6-(azo)uracil, dihydrouracil, N3-(methyl)uracil, 5-uracil (i.e., pseudo uracil), 2-(thio)pseudouracil,4-(thio)pseudouracil,2,4-(dithio)psuedouracil,5-(alkyl)pseudouracil, 5-(methyl)pseudouracil, 5-(alkyl)-2-(thio)pseudouracil, 5-(methyl)-2-(thio)pseudouracil, 5-(alkyl)-4-(thio)pseudouracil, 5-(methyl)-4-(thio)pseudouracil, 5-(alkyl)-2,4-(dithio)pseudouracil, 5-(methyl)-2,4-(dithio)pseudouracil, I-substituted pseudouracil, I-substituted 2(thio)-pseudo uracil, I-substituted 4-(thio)pseudo uracil, I-substituted 2,4-(dithio)pseudouracil, 1-(aminocarbonylethylenyl)-pseudouracil, 1-(aminocarbonylethylenyl)-2(thio)pseudouracil, 1-(aminocarbonylethylenyl)-4-(thio)pseudo uracil, 1-(aminocarbonylethylenyl)-2,4-(dithio)pseudouracil, 1-(aminoalkylaminocarbonylethylenyl)-pseudouracil, 1-(aminoalkylaminocarbonylethylenyl)-2(thio)-pseudouracil, 1-(aminoalkylaminocarbonylethylenyl)-4-(thio)pseudo uracil, 1-(aminoalkylaminocarbonylethylenyl)-2,4-(dithio)pseudouracil, 1,3-(diaza)-2-(oxo)-phenoxazin-1-yl, 1-(aza)-2-(thio)-3-(aza)-phenoxazin-1-yl, 1,3-(diaza)-2-(oxo)-phenthiazin-1-yl, 1-(aza)-2-(thio)-3-(aza)-phenthiazin-1-yl, 7-substituted 1,3-(diaza)-2-(oxo)-phenoxazin-1-yl, 7-substituted 1-(aza)-2-(thio)-3-(aza)-phenoxazin-1-yl, 7-substituted 1,3-(diaza)-2-(oxo)-phenthiazin-1-yl, 7-substituted 1(aza)-2-(thio)-3-(aza)-phenthiazin-1-yl, 7-(aminoalkylhydroxy)-1,3-(diaza)-2-(oxo)-phenoxazin-1-yl, 7-(aminoalkylhydroxy)-1-(aza)-2-(thio)-3-(aza)-phenoxazin-1-yl, 7-(aminoalkylhydroxy)-1,3-(diaza)-2-(oxo)-phenthiazin-1-yl, 7-(aminoalkylhydroxy)-1-(aza)-2-(thio)-3-(aza)-phenthiazin-1-yl, 7-(guanidiniumalkylhydroxy)-1,3-(diaza)-2-(oxo)-phenoxazin-1-yl, 7-(guanidiniumalkylhydroxy)-1-(aza)-2-(thio)-3-(aza)-phenoxazin-1-yl, 7-(guanidiniumalkyl-hydroxy)-1,3-(diaza)-2-(oxo)phenthiazin-1-yl, 7-(guanidiniumalkylhydroxy)-1-(aza)-2-(thio)-3-(aza)-phenthiazin-1-yl, 1,3,5-(triaza)-2,6-(dioxa)-naphthalene, inosinyl, 2-aza-inosinyl, 7-deaza-inosinyl, nitroimidazolyl, nitropyrazolyl, nitrobenzimidazolyl, nitroindazolyl, aminoindolyl, pyrrolopyrimidinyl, 3-(methyl)isocarbostyrilyl, 5-(methyl)isocarbostyrilyl, 3-(methyl)-7-(propynyl)isocarbostyrilyl, 7-(aza)indolyl, 6-(methyl)-7-(aza)indolyl, imidizopyridinyl, 9-(methyl)-imidizopyridinyl, pyrrolopyrizinyl, isocarbostyrilyl, 7-(propynyl)isocarbostyrilyl, propynyl-7-(aza)indolyl, 2,4,5-(trimethyl)phenyl, 4-(methyl)indolyl, 4,6-(dimethyl)indolyl, phenyl, napthalenyl, anthracenyl, phenanthracenyl, pyrenyl, stilbenyl, tetracenyl, pentacenyl, difluorotolyl, 4-(fluoro)-6-(methyl)benzimidazole, 4-(methyl)benzimidazole, 6-(azo)thymine, 2-pyridinone, 5-nitroindole, 3-nitropyrrole, 6-(aza)pyrimidine, 2-(amino)purine, 2,6-(diamino)purine, 5-substituted pyrimidines, N2-substituted purines, N6-substituted purines, 06-substituted purines, substituted 1,2,4-triazoles, pyrrolo-pyrimidin-2-on-3-yl, 6-phenyl-pyrrolopyrimidin-2-on-3-yl, para-substituted-6-phenyl-pyrrolo-pyrimidin-2-on-3-yl, ortho-substituted-6-phenyl-pyrrolo-pyrimidin-2-on-3-yl, bis-ortho-substituted-6-phenyl-pyrrolo-pyrimidin-2-on-3-yl, para-(aminoalkylhydroxy)-6-phenyl-pyrrolo-pyrimidin-2-on-3-yl, ortho-(aminoalkylhydroxy)-6-phenyl-pyrrolo-pyrimidin-2-on-3-yl, bis-ortho-(aminoalkylhydroxy)-6-phenyl-pyrrolo-pyrimidin-2-on-3-yl, pyridopyrimidin-3-yl, 2-oxo-7-amino-pyridopyrimidin-3-yl, 2-oxo-pyridopyrimidine-3-yl, or any O-alkylated or N-alkylated derivatives thereof. Alternatively, substituted or modified analogs of any of the above nucleotides or nucleobases can be used in RNAi agents, compositions, and methods of the disclosure.

In some embodiments, the modified nucleotide and/or non-canonical base pairing nucleotide includes any one or combination of the following:
(1) According to the direction from the 5′ end to the 3′ end, the nucleotides at positions 2, 4, 12, and 14 of the antisense strand are 2′-fluoro-modified nucleotides, and the nucleotides at the remaining positions are 2′-O-methyl-modified nucleotides;
(2) According to the direction from the 5′ end to the 3′ end, the nucleotides at positions 7, 8, and 9 of the sense strand are 2′-fluoro-modified nucleotides;
(3) According to the direction from the 5′ end to the 3′ end, G at positions 2-8 of the antisense strand is replaced by inosine (I).

In some such embodiments, the modified nucleotide for the base replacement of hypoxanthine at positions 2-8 of the antisense strand further meets any one or combination of the following characteristics:
(i) According to the direction from the 5′ end to the 3′ end, guanine at positions 6-8 of the antisense strand is replaced by hypoxanthine (I);
(ii) According to the direction from the 5′ end to the 3′ end, guanine in G₀ in the sequence N₁N₂G₀N₃N₄ of the antisense strand is replaced by hypoxanthine (I) if at least 3 bases of N₁, N₂, N₃ and N₄ are A or U, where N₁, N₂, N₃, and N₄ are nucleotides independently containing adenine (A), cytosine (C), guanine (G), thymine (T) or uracil (U) as a base, and G₀ is a nucleotide containing guanine as a base;
(iii) According to the direction from the 5′ end to the 3′ end, the total number of adenine (A) and uracil (U) at positions 2-8 of the antisense strand is no less than 4; and/or
(iv) at least one guanine in G nucleotide in the antisense strand is replaced by hypoxanthine (I), wherein the hypoxanthine replacement causes a difference in melting temperature (ΔTm) of at least 2° C., of 2° C., or of over 2° C., for a dsRNA comprising the antisense strand with hypoxanthine replacement compared to the same dsRNA where the guanine (G) has not been replaced by hypoxanthine (I).

In some embodiments, the dsRNAi agents further comprise a phosphate or phosphate mimic at the 5′-end of the antisense strand. In some embodiments, the phosphate mimic is a 5′-vinyl phosphonate (VP). In some embodiments, the 5′-end of the antisense strand of the dsRNAi agent does not contain a 5′-vinyl phosphonate (VP).

Ends of the RNAi agents of the disclosure can be modified. Such modifications can be at one end or both ends. For example, the 3′ and/or 5′ ends of an RNA can be conjugated to other functional molecular entities such as labeling moieties, e.g., fluorophores (e.g., pyrene, TAMRA, fluorescein, Cy3 or Cy5 dyes) or protecting groups (based, e.g., on sulfur, silicon, boron or ester). The functional molecular entities can be attached to the sugar through a phosphate group and/or a linker. The terminal atom of the linker can connect to or replace the linking atom of the phosphate group or the C-3′ or C-5′ O, N, S or C group of the sugar. Alternatively, the linker can connect to or replace the terminal atom of a nucleotide surrogate (e.g., PNAs). When a linker/phosphate-functional molecular entity-linker/phosphate array is interposed between two strands of a double-stranded oligomeric compound, this array can substitute for a hairpin loop in a hairpin-type oligomeric compound. Terminal modifications can also be useful for monitoring distribution, and in such cases the preferred groups to be added include fluorophores, e.g., fluorescein or an Alexa dye, e.g., Alexa 488. Terminal modifications can also be useful for enhancing uptake; non-limiting useful modifications for this include targeting ligands.

The present disclosure also includes various salts, mixed salts, and free acid forms of the dsRNAi agents. In some embodiments, the dsRNAi agent is in a free acid form. In other embodiments, the dsRNAi agent is in a salt form. In one embodiment, the dsRNAi agent is in a sodium salt form. As commonly known in the field, when a dsRNAi agent is in the sodium salt form, sodium ions are present in the agent as counterions for the phosphodiester or phosphorothiotate groups.

In certain embodiments, the dsRNAi agent of the disclosure is further modified by covalent attachment of one or more conjugate groups. In general, conjugate groups modify one or more properties of the attached dsRNAi agent of the invention including but not limited to pharmacodynamic, pharmacokinetic, binding, absorption, cellular distribution, cellular uptake, charge and clearance. Conjugate groups are routinely used in the chemical arts and are linked directly or via an optional linking moiety or linking group to a parent compound such as an oligomeric compound. A preferred list of conjugate groups includes without limitation, intercalators, reporter molecules, polyamines, polyamides, polyethylene glycols, thioethers, polyethers, cholesterols, thiocholesterols, cholic acid moieties, folate, lipids, phospholipids, biotin, phenazine, phenanthridine, anthraquinone, adamantane, acridine, fluoresceins, rhodamines, coumarins and dyes.

In some embodiments, the targeting ligand of the disclosure comprises N-acetyl-galactosamine (GalNAc), or a GalNAc derivative, such as L96. In some embodiments, the targeting ligand is any targeting moiety disclosed in International PCT Application Publication No. WO2022266753A1. Unless obviously contradicted, the entire contents of WO2022266753A1 are hereby incorporated by reference.

In some embodiments, the structure of dsRNAi agent is selected from formula 1 to formula 33, wherein R² is the dsRNA. According to common knowledge in the art, R² generally forms a dsRNAi agent by conjugating the 3′ end or 5′ end of the sense strand to a targeting ligand. In some embodiments, the 3′ end of the sense strand is conjugated to the targeting ligand.

### Delivery and Use of an RNAi Agent

The delivery of an RNAi agent of the disclosure to a cell e.g., a cell within a subject, such as a human subject (e.g., a subject in need thereof, such as a subject having a metabolic disorder), can be achieved in several different ways. For example, delivery may be performed by contacting a cell with an RNAi agent of the disclosure either in vitro or in vivo. In vivo delivery may also be performed directly by administering a composition (or pharmaceutical composition) comprising an RNAi agent, e.g., a dsRNA, to a subject. Alternatively, in vivo delivery may be performed indirectly by administering one or more vectors that encode and direct the expression of the RNAi agent.

In one embodiment, the cells are liver cells, e.g., hepatocytes. In one embodiment, the cells are adipocytes. In certain embodiments, the RNAi agent is taken up by one or more tissues or cell types present in an organ, e.g., liver, adipose tissue.

Another aspect of the disclosure relates to a method of inhibiting or reducing the expression and/or activity of INHBE gene in a subject, comprising administering to the subject the dsRNAi agent of the disclosure. In some embodiments, the method comprises administering to a subject a therapeutically effective amount of a dsRNAi agent of the disclosure, such that expression of the INHBE gene is inhibited or reduced in the subject, e.g., in a cell in the subject. In some embodiments, the method comprises contacting a cell with a dsRNAi agent of the disclosure such that expression of the INHBE gene is inhibited or reduced in the cell. In some such embodiments, mRNA transcripts of a target gene, e.g., INHBE, are degraded in the subject or the cell, thereby inhibiting or reducing expression of INHBE gene in the subject or the cell.

Another aspect of the disclosure relates to a method of treating a subject having a metabolic disorder or at risk of having or developing a metabolic disorder, comprising administering to the subject a therapeutically effective amount of the dsRNAi agent of the disclosure, such that the subject is treated.

Another aspect of the disclosure relates to a method of treating or preventing a metabolic disorder in a subject in need thereof, comprising administering to the subject a therapeutically effective amount of the dsRNAi agent of the disclosure, such that the metabolic disorder is treated or prevented.

Another aspect of the disclosure relates to a method of treating or preventing an INHBE-associated disease or disorder in a subject in need thereof, comprising administering to the subject a therapeutically effective amount of the dsRNAi agent of the disclosure, such that the INHBE-associated disease or disorder is treated or prevented. The term “INHBE-associated disease or disorder” includes any disease or disorder that is caused by, mediated by, or associated with INHBE gene expression or protein production, and includes any disease or disorder that would benefit or be ameliorated by a decrease in INHBE gene expression or protein activity. Examples of INHBE-associated diseases or disorders include, without limitation, metabolic disorders, metabolic syndrome, type 2 diabetes, obesity, pre-diabetes, elevated triglyceride levels, lipodystrophy, liver inflammation, fatty liver, hypercholesterolemia, disorders associated with elevated liver enzymes, nonalcoholic steatohepatitis, cardiovascular disease, kidney disease, abdominal obesity, insulin resistance, hypertension, hyperlipidemia, a cardiometabolic disorder, and cancers associated with INHBE expression.

In some embodiments of methods of the disclosure, the subject is a human.

In some embodiments of methods of the disclosure, the subject has a metabolic disorder.

In some embodiments of methods of the disclosure, the metabolic disorder is one or more of metabolic syndrome, type 2 diabetes, obesity, pre-diabetes, elevated triglyceride levels, lipodystrophy, liver inflammation, fatty liver, hypercholesterolemia, disorders associated with elevated liver enzymes, nonalcoholic steatohepatitis, cardiovascular disease and kidney disease. In some embodiments metabolic syndrome includes, but is not limited to, one or more of abdominal obesity, insulin resistance, hypertension, and hyperlipidemia.

In some embodiments of methods of the disclosure, the INHBE-associated disease or disorder is one or more of a metabolic disorder, metabolic syndrome, type 2 diabetes, obesity, pre-diabetes, elevated triglyceride levels, lipodystrophy, liver inflammation, fatty liver, hypercholesterolemia, disorders associated with elevated liver enzymes, nonalcoholic steatohepatitis, cardiovascular disease, kidney disease, abdominal obesity, insulin resistance, hypertension, hyperlipidemia, a cardiometabolic disorder, and a cancer associated with INHBE expression.

Non-limiting examples of metabolic disorders include disorders of carbohydrates, e.g., diabetes, type I diabetes, type II diabetes, galactosemia, hereditary fructose intolerance, fructose 1,6-diphosphatase deficiency, glycogen storage disorders, congenital disorders of glycosylation, insulin resistance, insulin insufficiency, hyperinsulinemia, impaired glucose tolerance (IGT), abnormal glycogen metabolism; disorders of amino acid metabolism, e.g., maple syrup urine disease (MSUD), homocystinuria; disorders of organic acid metabolism, e.g., methylmalonic aciduria, 3-methylglutaconic aciduria-Barth syndrome, glutaric aciduria, 2-hydroxyglutaric aciduria—D and L forms; disorders of fatty acid beta-oxidation, e.g., medium-chain acyl-CoA dehydrogenase deficiency (MCAD), long-chain 3-hydroxyacyl-CoA dehydrogenase deficiency (LCHAD), very-long-chain acyl-CoA dehydrogenase deficiency (VLCAD); disorders of lipid metabolism, e.g., GMI Gangliosidosis, Tay-Sachs Disease, Sandhoff Disease, Fabry Disease, Gaucher Disease, Niemann-Pick Disease, Krabbe Disease, Mucolipidoses, Mucopolysaccharidoses; disorders of lipid distribution and/or storage, e.g., lipodystrophy, mitochondrial disorders, e.g., mitochondrial cardiomyopathies, Leigh disease, mitochondrial encephalopathy, lactic acidosis, stroke-like episodes (MELAS); myoclonic epilepsy with ragged-red fibers (MERRF); neuropathy; ataxia; retinitis pigmentosa (NARP); Barth syndrome; and peroxisomal disorders, e.g., Zellweger Syndrome (cerebrohepatorenal syndrome), XLinked Adrenoleukodystrophy, Refsum Disease.

In some embodiments, metabolic disorders are associated with body fat distribution and include, but are not limited to metabolic syndrome, type 2 diabetes, hyperlipidemia or dyslipidemia (high or altered circulating levels of low-density lipoprotein cholesterol (LDL-C), triglycerides, very low-density lipoprotein cholesterol (VLDL-C), apolipoprotein B or other lipid fractions), obesity (particularly abdominal obesity), lipodystrophy (such as an inability to deposit fat in adipose depots regionally (partial lipodystrophy) or in the whole body (lipoatrophy)), insulin resistance or higher or altered insulin levels at fasting or during a metabolic challenge, liver fat deposition or fatty liver body fat distribution and their complications (such as, for example, cirrhosis, fibrosis, or inflammation of the liver), nonalcoholic steatohepatitis, other types of liver inflammation, higher or elevated or altered liver enzyme levels or other markers of liver damage, inflammation or fat deposition in the liver, higher blood pressure and/or hypertension, higher blood sugar or glucose or hyperglycemia, metabolic syndrome, coronary artery disease, and other atherosclerotic conditions, and complications thereof. In some embodiments, metabolic disorders are associated with a body fat distribution characterized by higher accumulation of fat around the waist (such as greater abdominal fat or larger waist circumference) and/or lower accumulation of fat around the hips (such as lower gluteofemoral fat or smaller hip circumference), resulting in a greater waist-to-hip ratio (WHR), and higher cardio-metabolic risk independent of body mass index (BMI).

In one embodiment, a metabolic disorder is metabolic syndrome. The term “metabolic syndrome,” as used herein, refers to a disorder that includes a clustering of components that reflect overnutrition, sedentary lifestyles, genetic factors, increasing age, and resultant excess adiposity. Metabolic syndrome includes the clustering of abdominal obesity, insulin resistance, dyslipidemia, and elevated blood pressure and is associated with other comorbidities including the prothrombotic state, proinflammatory state, nonalcoholic fatty liver disease, and reproductive disorders. Metabolic syndrome is associated with an approximate doubling of cardiovascular disease risk and a 5-fold increased risk for incident type 2 diabetes mellitus. Abdominal adiposity (e.g., a large waist circumference (high waist-to-hip ratio)), high blood pressure, insulin resistance and dislipidemia are central to metabolic syndrome and its individual components (e.g., central obesity, fasting blood glucose (FBG)/pre-diabetes/diabetes, hypercholesterolemia, hypertriglyceridemia, and hypertension).

In one embodiment, a metabolic disorder is a disorder of carbohydrates. In one embodiment, the disorder of carbohydrates is diabetes. As used herein, the term “diabetes” refers to a group of metabolic disorders characterized by high blood sugar (glucose) levels which result from defects in insulin secretion or action, or both. The two most common types of diabetes, namely type 1 diabetes (also referred to as “type I diabetes”) and type 2 diabetes (also referred to as “type II diabetes”) both result from the body's inability to regulate insulin. Insulin is a hormone released by the pancreas in response to increased levels of blood sugar (glucose) in the blood.

The term “type I diabetes,” as used herein, refers to a chronic disease that occurs when the pancreas produces too little insulin to regulate blood sugar levels appropriately. Type I diabetes is also referred to as insulin-dependent diabetes mellitus, IDDM, and juvenile onset diabetes. People with type I diabetes (insulin-dependent diabetes) generally produce little or no insulin at all. Type I diabetes may result from progressive autoimmune destruction of the pancreatic beta-cells with subsequent insulin deficiency. People with type I diabetes must regularly inject insulin.

In type II diabetes (also referred to as noninsulin-dependent diabetes mellitus, NDDM), the pancreas continues to manufacture insulin, sometimes even at higher than normal levels, however the body develops resistance to its effects, resulting in a relative insulin deficiency. Obesity is a risk factor for type II diabetes, and the majority of people with this disorder are obese.

In some embodiments, diabetes includes pre-diabetes. “Pre-diabetes” refers to one or more early diabetic conditions including impaired glucose utilization, abnormal or impaired fasting glucose levels, impaired glucose tolerance, impaired insulin sensitivity and insulin resistance. Pre-diabetes is a major risk factor for the development of type 2 diabetes mellitus, cardiovascular disease and mortality. Much focus has been given to developing therapeutic interventions that prevent the development of type 2 diabetes by effectively treating pre-diabetes.

Diabetes can be diagnosed by the administration of a glucose tolerance test. Clinically, diabetes is often divided into several basic categories. Primary examples of these categories include, autoimmune diabetes mellitus, non-insulin-dependent diabetes mellitus (type 2 NDDM or NIDDM), insulin-dependent diabetes mellitus (type 1 IDDM), non-autoimmune diabetes mellitus, and maturity-onset diabetes of the young (MODY). A further category, often referred to as secondary, refers to diabetes brought about by some identifiable condition which causes or allows a diabetic syndrome to develop. Examples of secondary categories include without limitation, diabetes caused by pancreatic disease, hormonal abnormalities, drug- or chemical-induced diabetes, diabetes caused by insulin receptor abnormalities, diabetes associated with genetic syndromes, and diabetes of other causes.

In one embodiment, a metabolic disorder is a disorder of lipid metabolism. As used herein, a “lipid metabolism disorder” or “disorder of lipid metabolism” refers to any disorder associated with or caused by a disturbance in lipid metabolism. This term also includes any disorder, disease or condition that can lead to hyperlipidemia, or condition characterized by abnormal elevation of levels of any or all lipids and/or lipoproteins in the blood. This term refers to an inherited disorder, such as familial hypertriglyceridemia, familial partial lipodystrophy type 1 (FPLDI), or an induced or acquired disorder, such as a disorder induced or acquired as a result of a disease, disorder or condition (e.g., renal failure), a diet, or intake of certain drugs (e.g., as a result of highly active antiretroviral therapy (HAART) used for treating, e.g., AIDS or HIV). This term also refers to a disorder of fat distribution and/or storage, e.g., lipodystrophy.

Additional examples of disorders of lipid metabolism include, but are not limited to, atherosclerosis, dyslipidemia, hypertriglyceridemia (including drug-induced hypertriglyceridemia, diuretic-induced hypertriglyceridemia, alcohol-induced hypertriglyceridemia, beta-adrenergic blocking agent-induced hypertriglyceridemia, estrogen-induced hypertriglyceridemia, glucocorticoid-induced hypertriglyceridemia, retinoid-induced hypertriglyceridemia, cimetidine-induced hypertriglyceridemia, and familial hypertriglyceridemia), acute pancreatitis associated with hypertriglyceridemia, chylomicron syndrome, familial chylomicronemia, Apo-E deficiency or resistance, LPL deficiency or hypoactivity, hyperlipidemia (including familial combined hyperlipidemia), hypercholesterolemia, lipodystrophy, gout associated with hypercholesterolemia, xanthomatosis (subcutaneous cholesterol deposits), hyperlipidemia with heterogeneous LPL deficiency, hyperlipidemia with high LDL and heterogeneous LPL deficiency, fatty liver disease, or non-alcoholic stetohepatitis (NASH).

In one embodiment, a metabolic disorder is a cardiovascular disease. Cardiovascular diseases may include without limitation coronary artery disease (also called ischemic heart disease), hypertension, inflammation associated with coronary artery disease, restenosis, peripheral vascular diseases, or stroke.

In one embodiment, a metabolic disorder is a kidney disease. Kidney diseases may include without limitation chronic kidney disease, diabetic nephrophathy, diabetic kidney disease, or gout.

In one embodiment, a metabolic disorder is a disorder related to body weight. Body weight disorders may include without limitation obesity, hypo-metabolic states, hypothyroidism, uremia, and other conditions associated with weight gain (including rapid weight gain), weight loss, maintenance of weight loss, or risk of weight regain following weight loss.

In one embodiment, a metabolic disorder is a blood sugar disorder. Blood sugar disorders may include without limitation diabetes, hypertension, and polycystic ovarian syndrome (PCOS) related to insulin resistance.

Other exemplary metabolic disorders include without limitation renal transplantation, nephrotic syndrome, Cushing's syndrome, acromegaly, systemic lupus erythematosus, dysglobulinemia, lipodystrophy, glycogenosis type I, and Addison's disease.

In one embodiment, a metabolic disorder is primary hypertension. “Primary hypertension” may be a result of environmental or genetic causes (e.g., a result of no obvious underlying medical cause). In another embodiment, a metabolic disorder is secondary hypertension. “Secondary hypertension” has an identifiable underlying disorder which can be of multiple etiologies, including renal, vascular, and endocrine causes, e.g., renal parenchymal disease (e.g., polycystic kidneys, glomerular or interstitial disease), renal vascular disease (e.g., renal artery stenosis, fibromuscular dysplasia), endocrine disorders (e.g., adrenocorticosteroid or mineralocorticoid excess, pheochromocytoma, hyperthyroidism or hypothyroidism, growth hormone excess, hyperparathyroidism), coarctation of the aorta, or oral contraceptive use.

In one embodiment, a metabolic disorder is resistant hypertension. “Resistant hypertension” refers to blood pressure that remains above goal (e.g., above 130 mm Hg systolic or above 90 diastolic) in spite of concurrent use of three antihypertensive agents of different classes, one of which is a thiazide diuretic. Subjects whose blood pressure is controlled with four or more medications are also considered to have resistant hypertension.

In some embodiments of methods of the disclosure, the expression of the INHBE gene in the subject or the cell reduces the INHBE protein level in the subject's serum by at least 50%, at least 60%, at least 70%, at least 80%, at least 90%, or at least 95%.

In some embodiments of methods of the disclosure, the dsRNAi agent is administered to the subject at a dose of from about 0.01 mg/kg to about 50 mg/kg, or at a dose of about 0.10 mg/kg to about 50 mg/kg, for example and without limitation, at a dose of about 0.01 mg/kg to about 10 mg/kg (e.g., about 2 mg/kg, about 3 mg/kg, about 4 mg/kg, about 5 mg/kg, about 6 mg/kg, about 7 mg/kg, about 8 mg/kg, or about 9 mg/kg), about 0.5 mg/kg to about 50 mg/kg, about 10 mg/kg to about 30 mg/kg, about 10 mg/kg to about 20 mg/kg, about 15 mg/kg to about 20 mg/kg, about 15 mg/kg to about 25 mg/kg, about 15 mg/kg to 30 mg/kg, or about 20 mg/kg to about 30 mg/kg.

In some embodiments of methods of the disclosure, the method further comprises determining the level of INHBE in a sample from the subject, e.g., in a blood, serum, liver tissue or adipose tissue sample. The level of INHBE may be determined in a sample obtained from the subject before administration of the dsRNAi agent, after administration of the dsRNAi agent, or during administration of the dsRNAi agent (e.g., to monitor efficacy or efficiency of treatments, to monitor INHBE mRNA and/or protein levels before, during or after treatment, etc.).

In some embodiments of methods of the disclosure, the method further comprises administering to the subject an additional therapeutic agent for treating a metabolic disorder. Examples of additional therapeutic agent(s) include, but are not limited to, insulin, glucagon-like peptide 1 agonist, glucose-dependent insulinotropic polypeptide (GIP) receptor agonist, glucagon receptor agonist, sulfonylurea, seglitinide, biguanide, thiazolidinedione, alpha-glucosidase inhibitor, SGLT2 inhibitor, DPP-4 inhibitor, HMG-CoA reductase inhibitor, statin, and any combination of the foregoing. Additional therapeutic agents may be administered simultaneously or sequentially with the dsRNAi agent or composition of the disclosure. In some embodiments, the dsRNAi agent is administered before an additional therapeutic agent. In other embodiments, the dsRNAi agent is administered after an additional therapeutic agent. In certain embodiments, the dsRNAi agent and the additional therapeutic agent are administered at the same time.

In some embodiments, a dsRNAi agent of the disclosure is administered by injection or by infusion. In one embodiment, a dsRNAi agent is administered subcutaneously. In one embodiment, a dsRNAi agent is administered intramuscularly. In one embodiment, a dsRNAi agent is administered intravenously. In some embodiments, a dsRNAi agent is administered by pulmonary systemic administration, such as intranasal administration or oral inhalation administration.

In some embodiments, a pharmaceutical composition of the disclosure comprises a dsRNAi agent of the disclosure or a pharmaceutically acceptable salt thereof, and a pharmaceutically acceptable carrier. The pharmaceutical composition can be practically used for the prevention and/or treatment of various INHBE-associated diseases or disorders as described herein. Acceptable carriers (or excipients) are substances other than active pharmaceutical ingredients (APIs, therapeutic products, such as the dsRNAi agents of the disclosure) that are intentionally included in the drug delivery system. The carrier or excipient is not or is not intended to be therapeutically effective in the intended dosage. For example and without limitation, carriers or excipients can play the following roles:
a) facilitate the handling of the drug delivery system during manufacture;
b) protect, support or enhance the stability, bioavailability or tolerability of the API;
c) facilitate in product identification; and/or
d) enhance any other property such as the overall safety, efficacy or delivery of the API during storage or use.

Carriers or excipients include, but are not limited to, the following components: absorption enhancers, anti-adherents, antifoaming agents, antioxidants, binders, buffers, carriers, coatings, colorants, delivery enhancers, delivery polymers substances, detergents, dextran, dextrose, diluents, disintegrants, emulsifiers, bulking agents, fillers, flavoring agents, glidants, wetting agents, lubricants, oils, polymers, preservatives, saline, salts, solvents, sugars, surfactants, suspending agents, sustained release matrices, sweeteners, thickeners, tonicity agents, vehicles, water repellents and wetting agents.

In some embodiments, the carrier of the pharmaceutical composition is an unbuffered solution or a buffered solution. Typical unbuffered solutions are saline or water, and buffered solutions include one or more of acetate, citrate, prolamine, carbonate and phosphate. In some embodiments, the buffer solution is phosphate buffered saline (PBS).

### Examples

The present invention will be more readily understood by referring to the following examples, which are provided to illustrate the invention and are not to be construed as limiting the scope thereof in any manner. The entire contents of all references, patents and published patent applications cited throughout this application, as well as the Figures, are hereby incorporated herein by reference.

Unless defined otherwise or the context clearly dictates otherwise, all technical and scientific terms used herein have the same meaning as commonly understood by one of ordinary skill in the art to which this invention belongs. It should be understood that any methods and materials similar or equivalent to those described herein can be used in the practice or testing of the invention. Unless otherwise specified, the materials and instruments used in the present disclosure are generally commercially available.

### Example 1. Synthesis of siRNA

### 1.1 Target Sequence Screening

siRNAs were designed according to the full-length INHBE mRNA sequence, and all sequences were obtained from the NCBI gene database (https://www.ncbi.nlm.nih.gov/gene/). All siRNAs were designed to ensure complete sequence identity with humans (Gene ID: 83729; SEQ ID NO: 1619) and cynomolgus monkeys (Gene ID: 102127493; SEQ ID NO: 1620).

After scanning the full-length sequence, all potential siRNA sequences with a length of 19 nucleotides and matching with the sequences of cynomolgus monkeys were selected. All the potential sequences were compared to human whole transcriptome mRNA sequences by BLAST and siRNAs with potential off-target effects were removed. The activity of all siRNAs was evaluated by the principle of rational design of siRNAs, and siRNAs with low theoretical activity were removed.

1.2 siRNA Synthesis

The siRNAs were designed to target INHBE in different regions and two additional nucleotides complementary to the mRNA or dTdT were added to the antisense strand for siRNA sequences with blunt ends (for example, IN-151 to IN-230), and then synthesized and annealed by Suzhou Biosyntech Co., Ltd. The siRNA sequences are shown in Table 1 and Table 2.

TABLE 1
Sense and antisense strand sequences of unmodified INHBE dsRNAi agents

| | Sense Strand Sequences | SEQ | Antisense Strand Sequences | SEQ |
|---|---|---|---|---|
| Duplex | 5′ to 3′ | ID NO: | 5′ to 3′ | ID NO: |
| IN-001 | GUGAGGGUCAAGCACAGCU | 1 | AGCUGUGCUUGACCCUCACAG | 2 |
| IN-002 | GAGGGUCAAGCACAGCUAU | 3 | AUAGCUGUGCUUGACCCUCAC | 4 |
| IN-003 | GGGUCAAGCACAGCUAUCC | 5 | GGAUAGCUGUGCUUGACCCUC | 6 |
| IN-004 | GGUCAAGCACAGCUAUCCA | 7 | UGGAUAGCUGUGCUUGACCCU | 8 |
| IN-005 | GUCAAGCACAGCUAUCCAU | 9 | AUGGAUAGCUGUGCUUGACCC | 10 |
| IN-006 | UCAAGCACAGCUAUCCAUC | 11 | GAUGGAUAGCUGUGCUUGACC | 12 |
| IN-007 | CAAGCACAGCUAUCCAUCA | 13 | UGAUGGAUAGCUGUGCUUGAC | 14 |
| IN-008 | AAGCACAGCUAUCCAUCAG | 15 | CUGAUGGAUAGCUGUGCUUGA | 16 |
| IN-009 | AGCACAGCUAUCCAUCAGA | 17 | UCUGAUGGAUAGCUGUGCUUG | 18 |
| IN-010 | GCACAGCUAUCCAUCAGAU | 19 | AUCUGAUGGAUAGCUGUGCUU | 20 |
| IN-011 | CACAGCUAUCCAUCAGAUG | 21 | CAUCUGAUGGAUAGCUGUGCU | 22 |
| IN-012 | ACAGCUAUCCAUCAGAUGA | 23 | UCAUCUGAUGGAUAGCUGUGC | 24 |
| IN-013 | CAGCUAUCCAUCAGAUGAU | 25 | AUCAUCUGAUGGAUAGCUGUG | 26 |
| IN-014 | AGCUAUCCAUCAGAUGAUC | 27 | GAUCAUCUGAUGGAUAGCUGU | 28 |
| IN-015 | GCUAUCCAUCAGAUGAUCU | 29 | AGAUCAUCUGAUGGAUAGCUG | 30 |
| IN-016 | CUAUCCAUCAGAUGAUCUA | 31 | UAGAUCAUCUGAUGGAUAGCU | 32 |
| IN-017 | AUCCAUCAGAUGAUCUACU | 33 | AGUAGAUCAUCUGAUGGAUAG | 34 |
| IN-018 | UCCAUCAGAUGAUCUACUU | 35 | AAGUAGAUCAUCUGAUGGAUA | 36 |
| IN-019 | CCAUCAGAUGAUCUACUUU | 37 | AAAGUAGAUCAUCUGAUGGAU | 38 |
| IN-020 | CAUCAGAUGAUCUACUUUC | 39 | GAAAGUAGAUCAUCUGAUGGA | 40 |
| IN-021 | AUCAGAUGAUCUACUUUCA | 41 | UGAAAGUAGAUCAUCUGAUGG | 42 |
| IN-022 | CAGAUGAUCUACUUUCAGC | 43 | GCUGAAAGUAGAUCAUCUGAU | 44 |
| IN-023 | GAUGAUCUACUUUCAGCCU | 45 | AGGCUGAAAGUAGAUCAUCUG | 46 |
| IN-024 | AUGAUCUACUUUCAGCCUU | 47 | AAGGCUGAAAGUAGAUCAUCU | 48 |
| IN-025 | UUCCUGAGUCCCAGACAAU | 49 | AUUGUCUGGGACUCAGGAAGG | 50 |
| IN-026 | UCCUGAGUCCCAGACAAUA | 51 | UAUUGUCUGGGACUCAGGAAG | 52 |
| IN-027 | UGAGUCCCAGACAAUAGAA | 53 | UUCUAUUGUCUGGGACUCAGG | 54 |
| IN-028 | GUCCCAGACAAUAGAAGAC | 55 | GUCUUCUAUUGUCUGGGACUC | 56 |
| IN-029 | CAAUAGAAGACAGGUGGCU | 57 | AGCCACCUGUCUUCUAUUGUC | 58 |
| IN-030 | AUAGAAGACAGGUGGCUGU | 59 | ACAGCCACCUGUCUUCUAUUG | 60 |
| IN-031 | AGGUGUGGCAGUGGUGUCU | 61 | AGACACCACUGCCACACCUAC | 62 |
| IN-032 | CAUUGGCCCCCAGCAAUCA | 63 | UGAUUGCUGGGGGCCAAUGAG | 64 |
| IN-033 | AGCUAGCCAAGCAGCAAAU | 65 | AUUUGCUGCUUGGCUAGCUCC | 66 |
| IN-034 | UAGCCAAGCAGCAAAUCCU | 67 | AGGAUUUGCUGCUUGGCUAGC | 68 |
| IN-035 | ACCAGUCGUCCCAGAAUAA | 69 | UUAUUCUGGGACGACUGGUCA | 70 |
| IN-036 | CAGUCGUCCCAGAAUAACU | 71 | AGUUAUUCUGGGACGACUGGU | 72 |
| IN-037 | AGUCGUCCCAGAAUAACUC | 73 | GAGUUAUUCUGGGACGACUGG | 74 |
| IN-038 | UCGUCCCAGAAUAACUCAU | 75 | AUGAGUUAUUCUGGGACGACU | 76 |
| IN-039 | GGAAUGGGGAGGAGGUCAU | 77 | AUGACCUCCUCCCCAUUCCCU | 78 |
| IN-040 | GAGGUCAUCAGCUUUGCUA | 79 | UAGCAAAGCUGAUGACCUCCU | 80 |
| IN-041 | GGUCAUCAGCUUUGCUACU | 81 | AGUAGCAAAGCUGAUGACCUC | 82 |
| IN-042 | GUCAUCAGCUUUGCUACUG | 83 | CAGUAGCAAAGCUGAUGACCU | 84 |
| IN-043 | UCAUCAGCUUUGCUACUGU | 85 | ACAGUAGCAAAGCUGAUGACC | 86 |
| IN-044 | CAUCAGCUUUGCUACUGUC | 87 | GACAGUAGCAAAGCUGAUGAC | 88 |
| IN-045 | GCUUUGCUACUGUCACAGA | 89 | UCUGUGACAGUAGCAAAGCUG | 90 |
| IN-046 | UUUGCUACUGUCACAGACU | 91 | AGUCUGUGACAGUAGCAAAGC | 92 |
| IN-047 | ACUGUCACAGACUCCACUU | 93 | AAGUGGAGUCUGUGACAGUAG | 94 |
| IN-048 | ACCCUUCCUGGCACUCUUU | 95 | AAAGAGUGCCAGGAAGGGUGG | 96 |
| IN-049 | CUUCCUGGCACUCUUUGCU | 97 | AGCAAAGAGUGCCAGGAAGGG | 98 |
| IN-050 | GCACUCUUUGCUUGAGGAU | 99 | AUCCUCAAGCAAAGAGUGCCA | 100 |
| IN-051 | ACUCUUUGCUUGAGGAUCU | 101 | AGAUCCUCAAGCAAAGAGUGC | 102 |
| IN-052 | CUCUUUGCUUGAGGAUCUU | 103 | AAGAUCCUCAAGCAAAGAGUG | 104 |
| IN-053 | GCUGGCAUACCUUAACUCU | 105 | AGAGUUAAGGUAUGCCAGCCC | 106 |
| IN-054 | AGUGGCUUGAGGGGUGAGA | 107 | UCUCACCCCUCAAGCCACUAG | 108 |
| IN-055 | CUUGAGGGGUGAGAAGUCU | 109 | AGACUUCUCACCCCUCAAGCC | 110 |
| IN-056 | AGUCUGGUGUCCUGAAACU | 111 | AGUUUCAGGACACCAGACUUC | 112 |
| IN-057 | CUGGUGUCCUGAAACUGCA | 113 | UGCAGUUUCAGGACACCAGAC | 114 |
| IN-058 | GUGUCCUGAAACUGCAACU | 115 | AGUUGCAGUUUCAGGACACCA | 116 |
| IN-059 | UGUCCUGAAACUGCAACUA | 117 | UAGUUGCAGUUUCAGGACACC | 118 |
| IN-060 | GUCCUGAAACUGCAACUAG | 119 | CUAGUUGCAGUUUCAGGACAC | 120 |
| IN-061 | AGAAGGCAACAGCACAGUU | 121 | AACUGUGCUGUUGCCUUCUAG | 122 |
| IN-062 | AGGCAACAGCACAGUUACU | 123 | AGUAACUGUGCUGUUGCCUUC | 124 |
| IN-063 | CCUUCCUAGAGCUUAAGAU | 125 | AUCUUAAGCUCUAGGAAGGGC | 126 |
| IN-064 | CUUCCUAGAGCUUAAGAUC | 127 | GAUCUUAAGCUCUAGGAAGGG | 128 |
| IN-065 | AGAGCUUAAGAUCCGAGCC | 129 | GGCUCGGAUCUUAAGCUCUAG | 130 |
| IN-066 | GAGCUUAAGAUCCGAGCCA | 131 | UGGCUCGGAUCUUAAGCUCUA | 132 |
| IN-067 | GCUUAAGAUCCGAGCCAAU | 133 | AUUGGCUCGGAUCUUAAGCUC | 134 |
| IN-068 | CCUGCGACCCCCUUAUGUU | 135 | AACAUAAGGGGGUCGCAGGCU | 136 |
| IN-069 | UGUUGCAGGCGAGACCAUU | 137 | AAUGGUCUCGCCUGCAACAUA | 138 |
| IN-070 | CGAGACCAUUACGUAGACU | 139 | AGUCUACGUAAUGGUCUCGCC | 140 |
| IN-071 | GAGACCAUUACGUAGACUU | 141 | AAGUCUACGUAAUGGUCUCGC | 142 |
| IN-072 | AUUACGUAGACUUCCAGGA | 143 | UCCUGGAAGUCUACGUAAUGG | 144 |
| IN-073 | AGGGGUACCAGCUGAAUUA | 145 | UAAUUCAGCUGGUACCCCUCG | 146 |
| IN-074 | AUUGCUGCCUCUUUCCAUU | 147 | AAUGGAAAGAGGCAGCAAUGC | 148 |
| IN-075 | CUUUCCAUUCUGCCGUCUU | 149 | AAGACGGCAGAAUGGAAAGAG | 150 |
| IN-076 | CCUCCUCAAAGCCAACAAU | 151 | AUUGUUGGCUUUGAGGAGGCU | 152 |
| IN-077 | CCUCAAAGCCAACAAUCCU | 153 | AGGAUUGUUGGCUUUGAGGAG | 154 |
| IN-078 | CUCAAAGCCAACAAUCCUU | 155 | AAGGAUUGUUGGCUUUGAGGA | 156 |
| IN-079 | CCUGCCAGUACCUCCUGUU | 157 | AACAGGAGGUACUGGCAGGCC | 158 |
| IN-080 | UCUCCUCUACCUGGAUCAU | 159 | AUGAUCCAGGUAGAGGAGAGA | 160 |
| IN-081 | CUCCUCUACCUGGAUCAUA | 161 | UAUGAUCCAGGUAGAGGAGAG | 162 |
| IN-082 | UCCUCUACCUGGAUCAUAA | 163 | UUAUGAUCCAGGUAGAGGAGA | 164 |
| IN-083 | CCUCUACCUGGAUCAUAAU | 165 | AUUAUGAUCCAGGUAGAGGAG | 166 |
| IN-084 | CCUGGAUCAUAAUGGCAAU | 167 | AUUGCCAUUAUGAUCCAGGUA | 168 |
| IN-085 | UGGAUCAUAAUGGCAAUGU | 169 | ACAUUGCCAUUAUGAUCCAGG | 170 |
| IN-086 | AUAAUGGCAAUGUGGUCAA | 171 | UUGACCACAUUGCCAUUAUGA | 172 |
| IN-087 | AUGUGGUCAAGACGGAUGU | 173 | ACAUCCGUCUUGACCACAUUG | 174 |
| IN-088 | AGACGGAUGUGCCAGAUAU | 175 | AUAUCUGGCACAUCCGUCUUG | 176 |
| IN-089 | UUUGGAGUGAAGAGACCAA | 177 | UUGGUCUCUUCACUCCAAAGC | 178 |
| IN-090 | AGUGAAGAGACCAAGAUGA | 179 | UCAUCUUGGUCUCUUCACUCC | 180 |
| IN-091 | GUGAAGAGACCAAGAUGAA | 181 | UUCAUCUUGGUCUCUUCACUC | 182 |
| IN-092 | GAAGAGACCAAGAUGAAGU | 183 | ACUUCAUCUUGGUCUCUUCAC | 184 |
| IN-093 | AAGAGACCAAGAUGAAGUU | 185 | AACUUCAUCUUGGUCUCUUCA | 186 |
| IN-094 | AGAGACCAAGAUGAAGUUU | 187 | AAACUUCAUCUUGGUCUCUUC | 188 |
| IN-095 | UGACUGGAGGCAUCAGAUU | 189 | AAUCUGAUGCCUCCAGUCACA | 190 |
| IN-096 | AACAACCACCUGGCAAUAU | 191 | AUAUUGCCAGGUGGUUGUUGG | 192 |
| IN-097 | CCACCUGGCAAUAUGACUC | 193 | GAGUCAUAUUGCCAGGUGGUU | 194 |
| IN-098 | CCUGGCAAUAUGACUCACU | 195 | AGUGAGUCAUAUUGCCAGGUG | 196 |
| IN-099 | CUGGCAAUAUGACUCACUU | 197 | AAGUGAGUCAUAUUGCCAGGU | 198 |
| IN-100 | CAAAUGGGCACUUUCUUGU | 199 | ACAAGAAAGUGCCCAUUUGGG | 200 |
| IN-101 | AAUGGGCACUUUCUUGUCU | 201 | AGACAAGAAAGUGCCCAUUUG | 202 |
| IN-102 | CACUUUCUUGUCUGAGACU | 203 | AGUCUCAGACAAGAAAGUGCC | 204 |
| IN-103 | ACUUUCUUGUCUGAGACUC | 205 | GAGUCUCAGACAAGAAAGUGC | 206 |
| IN-104 | CUUUCUUGUCUGAGACUCU | 207 | AGAGUCUCAGACAAGAAAGUG | 208 |
| IN-105 | UGUCUGAGACUCUGGCUUA | 209 | UAAGCCAGAGUCUCAGACAAG | 210 |
| IN-106 | UCUGAGACUCUGGCUUAUU | 211 | AAUAAGCCAGAGUCUCAGACA | 212 |
| IN-107 | UUAUUCCAGGUUGGCUGAU | 213 | AUCAGCCAACCUGGAAUAAGC | 214 |
| IN-108 | UGUGUUGGGAGAUGGGUAA | 215 | UUACCCAUCUCCCAACACAUC | 216 |
| IN-109 | GUGUUGGGAGAUGGGUAAA | 217 | UUUACCCAUCUCCCAACACAU | 218 |
| IN-110 | GGGAGAUGGGUAAAGCGUU | 219 | AACGCUUUACCCAUCUCCCAA | 220 |
| IN-111 | GGAGAUGGGUAAAGCGUUU | 221 | AAACGCUUUACCCAUCUCCCA | 222 |
| IN-112 | GGGUAAAGCGUUUCUUCUA | 223 | UAGAAGAAACGCUUUACCCAU | 224 |
| IN-113 | GGUAAAGCGUUUCUUCUAA | 225 | UUAGAAGAAACGCUUUACCCA | 226 |
| IN-114 | GUAAAGCGUUUCUUCUAAA | 227 | UUUAGAAGAAACGCUUUACCC | 228 |
| IN-115 | UAAAGCGUUUCUUCUAAAG | 229 | CUUUAGAAGAAACGCUUUACC | 230 |
| IN-116 | GUUUCUUCUAAAGGGGUCU | 231 | AGACCCCUUUAGAAGAAACGC | 232 |
| IN-117 | UUUCUUCUAAAGGGGUCUA | 233 | UAGACCCCUUUAGAAGAAACG | 234 |
| IN-118 | GGUCUACCCAGAAAGCAUG | 235 | CAUGCUUUCUGGGUAGACCCC | 236 |
| IN-119 | UCUACCCAGAAAGCAUGAU | 237 | AUCAUGCUUUCUGGGUAGACC | 238 |
| IN-120 | CUACCCAGAAAGCAUGAUU | 239 | AAUCAUGCUUUCUGGGUAGAC | 240 |
| IN-121 | UACCCAGAAAGCAUGAUUU | 241 | AAAUCAUGCUUUCUGGGUAGA | 242 |
| IN-122 | CCAGAAAGCAUGAUUUCCU | 243 | AGGAAAUCAUGCUUUCUGGGU | 244 |
| IN-123 | AAGCAUGAUUUCCUGCCCU | 245 | AGGGCAGGAAAUCAUGCUUUC | 246 |
| IN-124 | AUGAUUUCCUGCCCUAAGU | 247 | ACUUAGGGCAGGAAAUCAUGC | 248 |
| IN-125 | CCCUAAGUCCUGUGAGAAG | 249 | CUUCUCACAGGACUUAGGGCA | 250 |
| IN-126 | CCUAAGUCCUGUGAGAAGA | 251 | UCUUCUCACAGGACUUAGGGC | 252 |
| IN-127 | CUAAGUCCUGUGAGAAGAU | 253 | AUCUUCUCACAGGACUUAGGG | 254 |
| IN-128 | AAGUCCUGUGAGAAGAUGU | 255 | ACAUCUUCUCACAGGACUUAG | 256 |
| IN-129 | GAGGGAAGGCAGAGAAAAA | 257 | UUUUUCUCUGCCUUCCCUCCC | 258 |
| IN-130 | AGGGAAGGCAGAGAAAAAU | 259 | AUUUUUCUCUGCCUUCCCUCC | 260 |
| IN-131 | GGGAAGGCAGAGAAAAAUU | 261 | AAUUUUUCUCUGCCUUCCCUC | 262 |
| IN-132 | GGAAGGCAGAGAAAAAUUA | 263 | UAAUUUUUCUCUGCCUUCCCU | 264 |
| IN-133 | GAAGGCAGAGAAAAAUUAC | 265 | GUAAUUUUUCUCUGCCUUCCC | 266 |
| IN-134 | AAGGCAGAGAAAAAUUACU | 267 | AGUAAUUUUUCUCUGCCUUCC | 268 |
| IN-135 | AGGCAGAGAAAAAUUACUU | 269 | AAGUAAUUUUUCUCUGCCUUC | 270 |
| IN-136 | CCUCUCCCAAGAUGAGAAA | 271 | UUUCUCAUCUUGGGAGAGGCU | 272 |
| IN-137 | UCUCCCAAGAUGAGAAAGU | 273 | ACUUUCUCAUCUUGGGAGAGG | 274 |
| IN-138 | GGAGGAGGAAGCAGAUAGA | 275 | UCUAUCUGCUUCCUCCUCCCC | 276 |
| IN-139 | GAGGAGGAAGCAGAUAGAU | 277 | AUCUAUCUGCUUCCUCCUCCC | 278 |
| IN-140 | AGGGCUGUUGAGGUACCUU | 279 | AAGGUACCUCAACAGCCCUUA | 280 |
| IN-141 | GGCUGUUGAGGUACCUUAA | 281 | UUAAGGUACCUCAACAGCCCU | 282 |
| IN-142 | GUUGAGGUACCUUAAGGGA | 283 | UCCCUUAAGGUACCUCAACAG | 284 |
| IN-143 | UUGAGGUACCUUAAGGGAA | 285 | UUCCCUUAAGGUACCUCAACA | 286 |
| IN-144 | GAGGUACCUUAAGGGAAGG | 287 | CCUUCCCUUAAGGUACCUCAA | 288 |
| IN-145 | GGAAGGUCAAGAGGGAGAU | 289 | AUCUCCCUCUUGACCUUCCCU | 290 |
| IN-146 | GAAACAGGAGUCAGGAAAA | 291 | UUUUCCUGACUCCUGUUUCUG | 292 |
| IN-147 | AAACAGGAGUCAGGAAAAU | 293 | AUUUUCCUGACUCCUGUUUCU | 294 |
| IN-148 | AACAGGAGUCAGGAAAAUG | 295 | CAUUUUCCUGACUCCUGUUUC | 296 |
| IN-149 | GUCAGGAAAAUGAGGCACU | 297 | AGUGCCUCAUUUUCCUGACUC | 298 |
| IN-151 | GCACUAAGCCUAAGAAGUU | 299 | AACUUCUUAGGCUUAGUGC | 300 |
| IN-152 | UAAGAAGUUCCCUGGUUUU | 301 | AAAACCAGGGAACUUCUUA | 302 |
| IN-153 | CACUGGGAGACAAGCAUUU | 303 | AAAUGCUUGUCUCCCAGUG | 304 |
| IN-154 | ACUGGGAGACAAGCAUUUA | 305 | UAAAUGCUUGUCUCCCAGU | 306 |
| IN-155 | CUGGGAGACAAGCAUUUAU | 307 | AUAAAUGCUUGUCUCCCAG | 308 |
| IN-156 | UGGGAGACAAGCAUUUAUA | 309 | UAUAAAUGCUUGUCUCCCA | 310 |
| IN-157 | GGGAGACAAGCAUUUAUAC | 311 | GUAUAAAUGCUUGUCUCCC | 312 |
| IN-158 | GGAGACAAGCAUUUAUACU | 313 | AGUAUAAAUGCUUGUCUCC | 314 |
| IN-159 | GAGACAAGCAUUUAUACUU | 315 | AAGUAUAAAUGCUUGUCUC | 316 |
| IN-160 | AGACAAGCAUUUAUACUUU | 317 | AAAGUAUAAAUGCUUGUCU | 318 |
| IN-161 | ACAAGCAUUUAUACUUUCU | 319 | AGAAAGUAUAAAUGCUUGU | 320 |
| IN-162 | CAAGCAUUUAUACUUUCUU | 321 | AAGAAAGUAUAAAUGCUUG | 322 |
| IN-163 | GCAUUUAUACUUUCUUUCU | 323 | AGAAAGAAAGUAUAAAUGC | 324 |
| IN-164 | CAUUUAUACUUUCUUUCUU | 325 | AAGAAAGAAAGUAUAAAUG | 326 |
| IN-165 | GGAGAAAGAAAAUCAACAA | 327 | UUGUUGAUUUUCUUUCUCC | 328 |
| IN-166 | AGAAAGAAAAUCAACAAAU | 329 | AUUUGUUGAUUUUCUUUCU | 330 |
| IN-167 | UCAACAAAUGUGAGUCAUA | 331 | UAUGACUCACAUUUGUUGA | 332 |
| IN-168 | CAACAAAUGUGAGUCAUAA | 333 | UUAUGACUCACAUUUGUUG | 334 |
| IN-169 | CAAAUGUGAGUCAUAAAGA | 335 | UCUUUAUGACUCACAUUUG | 336 |
| IN-170 | GUGAGUCAUAAAGAAGGGU | 337 | ACCCUUCUUUAUGACUCAC | 338 |
| IN-171 | UGAGUCAUAAAGAAGGGUU | 339 | AACCCUUCUUUAUGACUCA | 340 |
| IN-172 | GAGUCAUAAAGAAGGGUUA | 341 | UAACCCUUCUUUAUGACUC | 342 |
| IN-173 | CAUAAAGAAGGGUUAGGGU | 343 | ACCCUAACCCUUCUUUAUG | 344 |
| IN-174 | AUGGUCCAGAGCAACAGUU | 345 | AACUGUUGCUCUGGACCAU | 346 |
| IN-175 | GUCCAGAGCAACAGUUCUU | 347 | AAGAACUGUUGCUCUGGAC | 348 |
| IN-176 | CAGAGCAACAGUUCUUCAA | 349 | UUGAAGAACUGUUGCUCUG | 350 |
| IN-177 | GAGCAACAGUUCUUCAAGU | 351 | ACUUGAAGAACUGUUGCUC | 352 |
| IN-178 | ACAGUUCUUCAAGUGUACU | 353 | AGUACACUUGAAGAACUGU | 354 |
| IN-179 | GUGUACUCUGUAGGCUUCU | 355 | AGAAGCCUACAGAGUACAC | 356 |
| IN-180 | GCUUCUGGGAGGUCCCUUU | 357 | AAAGGGACCUCCCAGAAGC | 358 |
| IN-181 | UCAGGGGUGUCCACAAAGU | 359 | ACUUUGUGGACACCCCUGA | 360 |
| IN-182 | GGGUGUCCACAAAGUCAAA | 361 | UUUGACUUUGUGGACACCC | 362 |
| IN-183 | GGUGUCCACAAAGUCAAAG | 363 | CUUUGACUUUGUGGACACC | 364 |
| IN-184 | UGUCCACAAAGUCAAAGCU | 365 | AGCUUUGACUUUGUGGACA | 366 |
| IN-185 | UCCACAAAGUCAAAGCUAU | 367 | AUAGCUUUGACUUUGUGGA | 368 |
| IN-186 | CCACAAAGUCAAAGCUAUU | 369 | AAUAGCUUUGACUUUGUGG | 370 |
| IN-187 | CACAAAGUCAAAGCUAUUU | 371 | AAAUAGCUUUGACUUUGUG | 372 |
| IN-188 | ACAAAGUCAAAGCUAUUUU | 373 | AAAAUAGCUUUGACUUUGU | 374 |
| IN-189 | AGUCAAAGCUAUUUUCAUA | 375 | UAUGAAAAUAGCUUUGACU | 376 |
| IN-190 | GUCAAAGCUAUUUUCAUAA | 377 | UUAUGAAAAUAGCUUUGAC | 378 |
| IN-191 | UCAAAGCUAUUUUCAUAAU | 379 | AUUAUGAAAAUAGCUUUGA | 380 |
| IN-192 | AGCUAUUUUCAUAAUAAUA | 381 | UAUUAUUAUGAAAAUAGCU | 382 |
| IN-193 | GCUAUUUUCAUAAUAAUAC | 383 | GUAUUAUUAUGAAAAUAGC | 384 |
| IN-194 | CUAUUUUCAUAAUAAUACU | 385 | AGUAUUAUUAUGAAAAUAG | 386 |
| IN-195 | AUUUUCAUAAUAAUACUAA | 387 | UUAGUAUUAUUAUGAAAAU | 388 |
| IN-196 | CAUAAUAAUACUAACAUGU | 389 | ACAUGUUAGUAUUAUUAUG | 390 |
| IN-197 | AUAAUAAUACUAACAUGUU | 391 | AACAUGUUAGUAUUAUUAU | 392 |
| IN-198 | AUAAUACUAACAUGUUAUU | 393 | AAUAACAUGUUAGUAUUAU | 394 |
| IN-199 | UAAUACUAACAUGUUAUUU | 395 | AAAUAACAUGUUAGUAUUA | 396 |
| IN-200 | ACUAACAUGUUAUUUGCCU | 397 | AGGCAAAUAACAUGUUAGU | 398 |
| IN-201 | UAACAUGUUAUUUGCCUUU | 399 | AAAGGCAAAUAACAUGUUA | 400 |
| IN-202 | CAUGUUAUUUGCCUUUUGA | 401 | UCAAAAGGCAAAUAACAUG | 402 |
| IN-203 | UGUUAUUUGCCUUUUGAAU | 403 | AUUCAAAAGGCAAAUAACA | 404 |
| IN-204 | GUUAUUUGCCUUUUGAAUU | 405 | AAUUCAAAAGGCAAAUAAC | 406 |
| IN-205 | UGCCUUUUGAAUUCUCAUU | 407 | AAUGAGAAUUCAAAAGGCA | 408 |
| IN-206 | GCCUUUUGAAUUCUCAUUA | 409 | UAAUGAGAAUUCAAAAGGC | 410 |
| IN-207 | CCUUUUGAAUUCUCAUUAU | 411 | AUAAUGAGAAUUCAAAAGG | 412 |
| IN-208 | UGAAUUCUCAUUAUCUUAA | 413 | UUAAGAUAAUGAGAAUUCA | 414 |
| IN-209 | GAAUUCUCAUUAUCUUAAA | 415 | UUUAAGAUAAUGAGAAUUC | 416 |
| IN-210 | AUUCUCAUUAUCUUAAAAU | 417 | AUUUUAAGAUAAUGAGAAU | 418 |
| IN-211 | CUCAUUAUCUUAAAAUUGU | 419 | ACAAUUUUAAGAUAAUGAG | 420 |
| IN-212 | CAUUAUCUUAAAAUUGUAU | 421 | AUACAAUUUUAAGAUAAUG | 422 |
| IN-213 | AUUAUCUUAAAAUUGUAUU | 423 | AAUACAAUUUUAAGAUAAU | 424 |
| IN-214 | GGCCGUGUGACAUGUGAUU | 425 | AAUCACAUGUCACACGGCC | 426 |
| IN-215 | GCCGUGUGACAUGUGAUUA | 427 | UAAUCACAUGUCACACGGC | 428 |
| IN-216 | CCGUGUGACAUGUGAUUAC | 429 | GUAAUCACAUGUCACACGG | 430 |
| IN-217 | CGUGUGACAUGUGAUUACA | 431 | UGUAAUCACAUGUCACACG | 432 |
| IN-218 | GUGUGACAUGUGAUUACAU | 433 | AUGUAAUCACAUGUCACAC | 434 |
| IN-219 | UGACAUGUGAUUACAUCAU | 435 | AUGAUGUAAUCACAUGUCA | 436 |
| IN-220 | ACAUGUGAUUACAUCAUCU | 437 | AGAUGAUGUAAUCACAUGU | 438 |
| IN-221 | CAUGUGAUUACAUCAUCUU | 439 | AAGAUGAUGUAAUCACAUG | 440 |
| IN-222 | GUGAUUACAUCAUCUUUCU | 441 | AGAAAGAUGAUGUAAUCAC | 442 |
| IN-223 | GAUUACAUCAUCUUUCUGA | 443 | UCAGAAAGAUGAUGUAAUC | 444 |
| IN-224 | CAUCAUCUUUCUGACAUCA | 445 | UGAUGUCAGAAAGAUGAUG | 446 |
| IN-225 | UCAUCUUUCUGACAUCAUU | 447 | AAUGAUGUCAGAAAGAUGA | 448 |
| IN-226 | UCUUUCUGACAUCAUUGUU | 449 | AACAAUGAUGUCAGAAAGA | 450 |
| IN-227 | CUUUCUGACAUCAUUGUUA | 451 | UAACAAUGAUGUCAGAAAG | 452 |
| IN-228 | CAUUGUUAAUGGAAUGUGU | 453 | ACACAUUCCAUUAACAAUG | 454 |
| IN-229 | UGUUAAUGGAAUGUGUGCU | 455 | AGCACACAUUCCAUUAACA | 456 |
| IN-230 | GUUAAUGGAAUGUGUGCUU | 457 | AAGCACACAUUCCAUUAAC | 458 |
| IN-231 | UCAUCAGCUUUGCUACUGU | 459 | ACAGUAGCAAAGCUGAUGAUC | 460 |
| IN-232 | UCAUCAGCUUUGCUACUGU | 461 | ACAGUAGCAAAGCUGAUGAUU | 462 |
| IN-233 | UCAUCAGCUUUGCUACUGU | 463 | ACAGUAGCAAAGCUGAUGACU | 464 |
| IN-234 | CCAUCAGCUUUGCUACUGU | 465 | ACAGUAGCAAAGCUGAUGGUU | 466 |
| IN-235 | GCAUCAGCUUUGCUACUGU | 467 | ACAGUAGCAAAGCUGAUGCUU | 468 |
| IN-236 | CAUCAGCUUUGCUACUGUC | 469 | GACAGUAGCAAAGCUGAUGAC | 470 |
| IN-237 | CAUCAGCUUUGCUACUGUA | 471 | UACAGUAGCAAAGCUGAUGAC | 472 |
| IN-238 | CAUCAGCUUUGCUACUGUC | 473 | GACAGUAGCAAAGCUGAUGCC | 474 |
| IN-239 | GCUGAGACUCUGGCUUAUU | 475 | AAUAAGCCAGAGUCUCAGCCA | 476 |
| IN-240 | CCUGAGACUCUGGCUUAUU | 477 | AAUAAGCCAGAGUCUCAGGCA | 478 |
| IN-241 | UCUGAGACUCUGGCUUAUU | 479 | AAUAAGCCAGAGUCUCAGAUA | 480 |
| IN-242 | UCUGAGACUCUGGCUUAUU | 481 | AAUAAGCCAGAGUCUCAGAUU | 482 |
| IN-243 | UCUGAGACUCUGGCUUAUU | 483 | AAUAAGCCAGAGUCUCAGACU | 484 |
| IN-244 | GUGAAGAGACCAAGAUGAA | 485 | UUCAUCUUGGUCUCUUCACUU | 486 |
| IN-245 | CAGAGCAACAGUUCUUCAA | 487 | UUGAAGAACUGUUGCUCUGGU | 488 |
| IN-246 | CAGAGCAACAGUUCUUCAA | 489 | UUGAAGAACUGUUGCUCUGUU | 490 |
| IN-247 | CAGAGCAACAGUUCUUCAA | 491 | UUGAAGAACUGUUGCUCUGUA | 492 |
| IN-248 | GGUCAAAGCUAUUUUCAUA | 493 | UAUGAAAAUAGCUUUGACCUU | 494 |
| IN-249 | CGUCAAAGCUAUUUUCAUA | 495 | UAUGAAAAUAGCUUUGACGUU | 496 |
| IN-250 | CAUGUGAUUACAUCAUCUU | 497 | AAGAUGAUGUAAUCACAUGUU | 498 |
| IN-478 | CAUGUUAUUUGCCUUUUGA | 953 | UCAAAAGGCAAAUAACAUGUU | 954 |

TABLE 2
Sense and antisense strand sequences of modified INHBE dsRNAi agents

| | Sense Strand Sequences | SEQ | Antisense Strand Sequences | SEQ |
|---|---|---|---|---|
| Duplex | 5′ to 3′ | ID NO: | 5′ to 3′ | ID NO: |
| IN-251 | mC*mA*mGmUmCmGfUfCfCmC | 499 | mA*dG*mUmUdAmUdTmCmUmG | 500 |
| | mAmGmAmAmUmAmAmCmU | | mGdGmAfCmGmAmCmUmG*mG* | |
| | | | mU | |
| IN-252 | UCAUCAGCUUUGCUACUGU | 501 | mAfCmAfGmUfAmGfCmAmAmAf | 502 |
| | | | GmCfUmGfAmUfGmAmCmC | |
| IN-253 | UCAUCAGCUUUGCUACUGU | 503 | mAfCmAfGmUfAmGfCfAmAmAf | 504 |
| | | | GmCfUmGfAmUfGmAmCmC | |
| IN-254 | UCAUCAGCUUUGCUACUGU | 505 | mAfCmAfGmUfAmGfCmAfAmAf | 506 |
| | | | GmCfUmGfAmUfGmAmCmC | |
| IN-255 | UCAUCAGCUUUGCUACUGU | 507 | mAfCmAfGmUfAmGfCmAmAfAf | 508 |
| | | | GmCfUmGfAmUfGmAmCmC | |
| IN-256 | UCAUCAGCUUUGCUACUGU | 509 | mAmCmAfGmUfAmGfCmAmAmA | 510 |
| | | | fGmCfUmGfAmUfGmAmCmC | |
| IN-257 | UCAUCAGCUUUGCUACUGU | 511 | mAfCmAmGmUfAmGfCmAmAmA | 512 |
| | | | fGmCfUmGfAmUfGmAmCmC | |
| IN-258 | UCAUCAGCUUUGCUACUGU | 513 | mAfCmAfGmUmAmGfCmAmAmA | 514 |
| | | | fGmCfUmGfAmUfGmAmCmC | |
| IN-259 | UCAUCAGCUUUGCUACUGU | 515 | mAfCmAfGmUfAmGmCmAmAmA | 516 |
| | | | fGmCfUmGfAmUfGmAmCmC | |
| IN-260 | UCAUCAGCUUUGCUACUGU | 517 | mAfCmAfGmUfAmGfCmAmAmA | 518 |
| | | | mGmCfUmGfAmUfGmAmCmC | |
| IN-261 | UCAUCAGCUUUGCUACUGU | 519 | mAfCmAfGmUfAmGfCmAmAmAf | 520 |
| | | | GmCmUmGfAmUfGmAmCmC | |
| IN-262 | UCAUCAGCUUUGCUACUGU | 521 | mAfCmAfGmUfAmGfCmAmAmAf | 522 |
| | | | GmCfUmGmAmUfGmAmCmC | |
| IN-263 | UCAUCAGCUUUGCUACUGU | 523 | mAfCmAfGmUfAmGfCmAmAmAf | 524 |
| | | | GmCfUmGfAmUmGmAmCmC | |
| IN-264 | UCAUCAGCUUUGCUACUGU | 525 | mA*fCmAfGmUfAmGfCmAmAmA | 526 |
| | | | fGmCfUmGfAmUfGmAmC*mC | |
| IN-265 | UCAUCAGCUUUGCUACUGU | 527 | mA*fC*mAfGmUfAmGfCmAmAm | 528 |
| | | | AfGmCfUmGfAmUfGmA*mC*mC | |
| IN-266 | UCAUCAGCUUUGCUACUGU | 529 | mAdCmAfGdUfAdGfCmAmAmAd | 530 |
| | | | GmCfUmGfAmUfGmAmCmC | |
| IN-267 | UCAUCAGCUUUGCUACUGU | 531 | mAfCmAfImUfAmGfCmAmAmAf | 532 |
| | | | GmCfUmGfAmUfGmAmCmC | |
| IN-268 | UCAUCAGCUUUGCUACUGU | 533 | mAfCmAfGmUfAmIfCmAmAmAf | 534 |
| | | | GmCfUmGfAmUfGmAmCmC | |
| IN-269 | mUmCmAmUfCfAfGfCfUfUmUm | 535 | ACAGUAGCAAAGCUGAUGACC | 536 |
| | GmCmUmAmCmUmGmU | | | |
| IN-270 | mUmCmAmUmCfAfGfCfUfUmU | 537 | ACAGUAGCAAAGCUGAUGACC | 538 |
| | mGmCmUmAmCmUmGmU | | | |
| IN-271 | mUmCmAmUmCmAfGfCfUfUm | 539 | ACAGUAGCAAAGCUGAUGACC | 540 |
| | UmGmCmUmAmCmUmGmU | | | |
| IN-272 | mUmCmAmUmCmAmGfCfUfUm | 541 | ACAGUAGCAAAGCUGAUGACC | 542 |
| | UmGmCmUmAmCmUmGmU | | | |
| IN-273 | mUmCmAmUfCmAmGmCfUfUm | 543 | ACAGUAGCAAAGCUGAUGACC | 544 |
| | UmGmCmUmAmCmUmGmU | | | |
| IN-274 | mUmCmAmUfCfAmGmCmUfUm | 545 | ACAGUAGCAAAGCUGAUGACC | 546 |
| | UmGmCmUmAmCmUmGmU | | | |
| IN-275 | mUmCmAmUfCfAfGmCmUmUm | 547 | ACAGUAGCAAAGCUGAUGACC | 548 |
| | UmGmCmUmAmCmUmGmU | | | |
| IN-276 | mUmCmAmUfCfAfGfCmUmUm | 549 | ACAGUAGCAAAGCUGAUGACC | 550 |
| | UmGmCmUmAmCmUmGmU | | | |
| IN-277 | mUmCmAmUfCfAfGfCfUmUmU | 551 | ACAGUAGCAAAGCUGAUGACC | 552 |
| | mGmCmUmAmCmUmGmU | | | |
| IN-278 | mU*mCmAmUfCfAfGfCfUfUmU | 553 | ACAGUAGCAAAGCUGAUGACC | 554 |
| | mGmCmUmAmCmUmGmU | | | |
| IN-279 | mU*mC*mAmUfCfAfGfCfUfUm | 555 | ACAGUAGCAAAGCUGAUGACC | 556 |
| | UmGmCmUmAmCmUmGmU | | | |
| IN-280 | CAUCAGCUUUGCUACUGUC | 557 | mGfAmCfAmGfUmAfGmCmAmAf | 558 |
| | | | AmGfCmUfGmAfUmGmAmC | |
| IN-281 | mCmAmUmCfAfGfCfUfUfUmGm | 559 | GACAGUAGCAAAGCUGAUGAC | 560 |
| | CmUmAmCmUmGmUmC | | | |
| IN-282 | mUmCmAmUfCfAfGfCfUfUmUm | 561 | ACAGUAGCAAAGCUGAUGACC | 562 |
| | GmCmUmAmCmUmGirU | | | |
| IN-283 | mCmAmUmCfAfGfCfUfUfUmGm | 563 | GACAGUAGCAAAGCUGAUGAC | 564 |
| | CmUmAmCmUmGmUirC | | | |
| IN-284 | mU*mC*mAmUfCfAfGfCfUfUm | 565 | mA*fC*mAfGmUfAmGfCmAmAm | 566 |
| | UmGmCmUmAmCmUmGmU | | AfGmCfUmGfAmUfGmA*mC*mC | |
| IN-285 | irdTmCmAmUfCfAfGfCfUfUmU | 567 | ACAGUAGCAAAGCUGAUGACC | 568 |
| | mGmCmUmAmCmUmGmU | | | |
| IN-286 | UCUGAGACUCUGGCUUAUU | 569 | mAfAmUfAmAfGmCfCmAmGmAf | 570 |
| | | | GmUfCmUfCmAfGmAmCmA | |
| IN-287 | UCUGAGACUCUGGCUUAUU | 571 | mAfAmUfAmAfGmCfCfAmGmAf | 572 |
| | | | GmUfCmUfCmAfGmAmCmA | |
| IN-288 | UCUGAGACUCUGGCUUAUU | 573 | mAfAmUfAmAfGmCfCmAfGmAf | 574 |
| | | | GmUfCmUfCmAfGmAmCmA | |
| IN-289 | UCUGAGACUCUGGCUUAUU | 575 | mAfAmUfAmAfGmCfCmAmGfAf | 576 |
| | | | GmUfCmUfCmAfGmAmCmA | |
| IN-290 | UCUGAGACUCUGGCUUAUU | 577 | mAmAmUfAmAfGmCfCmAmGmA | 578 |
| | | | fGmUfCmUfCmAfGmAmCmA | |
| IN-291 | UCUGAGACUCUGGCUUAUU | 579 | mAfAmUmAmAfGmCfCmAmGmA | 580 |
| | | | fGmUfCmUfCmAfGmAmCmA | |
| IN-292 | UCUGAGACUCUGGCUUAUU | 581 | mAfAmUfAmAmGmCfCmAmGmA | 582 |
| | | | fGmUfCmUfCmAfGmAmCmA | |
| IN-293 | UCUGAGACUCUGGCUUAUU | 583 | mAfAmUfAmAfGmCmCmAmGmA | 584 |
| | | | fGmUfCmUfCmAfGmAmCmA | |
| IN-294 | UCUGAGACUCUGGCUUAUU | 585 | mAfAmUfAmAfGmCfCmAmGmA | 586 |
| | | | mGmUfCmUfCmAfGmAmCmA | |
| IN-295 | UCUGAGACUCUGGCUUAUU | 587 | mAfAmUfAmAfGmCfCmAmGmAf | 588 |
| | | | GmUmCmUfCmAfGmAmCmA | |
| IN-296 | UCUGAGACUCUGGCUUAUU | 589 | mAfAmUfAmAfGmCfCmAmGmAf | 590 |
| | | | GmUfCmUmCmAfGmAmCmA | |
| IN-297 | UCUGAGACUCUGGCUUAUU | 591 | mAfAmUfAmAfGmCfCmAmGmAf | 592 |
| | | | GmUfCmUfCmAmGmAmCmA | |
| IN-298 | UCUGAGACUCUGGCUUAUU | 593 | mA*fAmUfAmAfGmCfCmAmGmA | 594 |
| | | | fGmUfCmUfCmAfGmAmC*mA | |
| IN-299 | UCUGAGACUCUGGCUUAUU | 595 | mA*fA*mUfAmAfGmCfCmAmGm | 596 |
| | | | AfGmUfCmUfCmAfGmA*mC*mA | |
| IN-300 | UCUGAGACUCUGGCUUAUU | 597 | mAdAmUfAdAfGdCfCmAmGmAd | 598 |
| | | | GmUfCmUfCmAfGmAmCmA | |
| IN-301 | UCUGAGACUCUGGCUUAUU | 599 | mAfAmUfAmAfImCfCmAmGmAf | 600 |
| | | | GmUfCmUfCmAfGmAmCmA | |
| IN-302 | mUmCmUmGfAfGfAfCfUfCmUm | 601 | AAUAAGCCAGAGUCUCAGACA | 602 |
| | GmGmCmUmUmAmUmU | | | |
| IN-303 | mUmCmUmGmAfGfAfCfUfCmU | 603 | AAUAAGCCAGAGUCUCAGACA | 604 |
| | mGmGmCmUmUmAmUmU | | | |
| IN-304 | mUmCmUmGmAmGfAfCfUfCm | 605 | AAUAAGCCAGAGUCUCAGACA | 606 |
| | UmGmGmCmUmUmAmUmU | | | |
| IN-305 | mUmCmUmGmAmGmAfCfUfCm | 607 | AAUAAGCCAGAGUCUCAGACA | 608 |
| | UmGmGmCmUmUmAmUmU | | | |
| IN-306 | mUmCmUmGfAmGmAmCfUfCm | 609 | AAUAAGCCAGAGUCUCAGACA | 610 |
| | UmGmGmCmUmUmAmUmU | | | |
| IN-307 | mUmCmUmGfAfGmAmCmUfCm | 611 | AAUAAGCCAGAGUCUCAGACA | 612 |
| | UmGmGmCmUmUmAmUmU | | | |
| IN-308 | mUmCmUmGfAfGfAmCmUmCm | 613 | AAUAAGCCAGAGUCUCAGACA | 614 |
| | UmGmGmCmUmUmAmUmU | | | |
| IN-309 | mUmCmUmGfAfGfAfCmUmCm | 615 | AAUAAGCCAGAGUCUCAGACA | 616 |
| | UmGmGmCmUmUmAmUmU | | | |
| IN-310 | mUmCmUmGfAfGfAfCfUmCmU | 617 | AAUAAGCCAGAGUCUCAGACA | 618 |
| | mGmGmCmUmUmAmUmU | | | |
| IN-311 | mU*mCmUmGfAfGfAfCfUfCmU | 619 | AAUAAGCCAGAGUCUCAGACA | 620 |
| | mGmGmCmUmUmAmUmU | | | |
| IN-312 | mU*mC*mUmGfAfGfAfCfUfCm | 621 | AAUAAGCCAGAGUCUCAGACA | 622 |
| | UmGmGmCmUmUmAmUmU | | | |
| IN-313 | mUmCmUmGfAfGfAfCfUfCmUm | 623 | AAUAAGCCAGAGUCUCAGACA | 624 |
| | GmGmCmUmUmAmUirdT | | | |
| IN-314 | mU*mC*mUmGfAfGfAfCfUfCm | 625 | mA*fA*mUfAmAfGmCfCmAmGm | 626 |
| | UmGmGmCmUmUmAmUmU | | AfGmUfCmUfCmAfGmA*mC*mA | |
| IN-315 | irdTmCmUmGfAfGfAfCfUfCmU | 627 | AAUAAGCCAGAGUCUCAGACA | 628 |
| | mGmGmCmUmUmAmUmU | | | |
| IN-316 | GUGAAGAGACCAAGAUGAA | 629 | mUfUmCfAmUfCmUfUmGmGmUf | 630 |
| | | | CmUfCmUfUmCfAmCmUmC | |
| IN-317 | GUGAAGAGACCAAGAUGAA | 631 | mUfUmCfAmUfCmUfUfGmGmUfC | 632 |
| | | | mUfCmUfUmCfAmCmUmC | |
| IN-318 | GUGAAGAGACCAAGAUGAA | 633 | mUfUmCfAmUfCmUfUmGfGmUfC | 634 |
| | | | mUfCmUfUmCfAmCmUmC | |
| IN-319 | GUGAAGAGACCAAGAUGAA | 635 | mUfUmCfAmUfCmUfUmGmGfUfC | 636 |
| | | | mUfCmUfUmCfAmCmUmC | |
| IN-320 | GUGAAGAGACCAAGAUGAA | 637 | mUmUmCfAmUfCmUfUmGmGmU | 638 |
| | | | fCmUfCmUfUmCfAmCmUmC | |
| IN-321 | GUGAAGAGACCAAGAUGAA | 639 | mUfUmCmAmUfCmUfUmGmGmU | 640 |
| | | | fCmUfCmUfUmCfAmCmUmC | |
| IN-322 | GUGAAGAGACCAAGAUGAA | 641 | mUfUmCfAmUmCmUfUmGmGmU | 642 |
| | | | fCmUfCmUfUmCfAmCmUmC | |
| IN-323 | GUGAAGAGACCAAGAUGAA | 643 | mUfUmCfAmUfCmUmUmGmGmU | 644 |
| | | | fCmUfCmUfUmCfAmCmUmC | |
| IN-324 | GUGAAGAGACCAAGAUGAA | 645 | mUfUmCfAmUfCmUfUmGmGmU | 646 |
| | | | mCmUfCmUfUmCfAmCmUmC | |
| IN-325 | GUGAAGAGACCAAGAUGAA | 647 | mUfUmCfAmUfCmUfUmGmGmUf | 648 |
| | | | CmUmCmUfUmCfAmCmUmC | |
| IN-326 | GUGAAGAGACCAAGAUGAA | 649 | mUfUmCfAmUfCmUfUmGmGmUf | 650 |
| | | | CmUfCmUmUmCfAmCmUmC | |
| IN-327 | GUGAAGAGACCAAGAUGAA | 651 | mUfUmCfAmUfCmUfUmGmGmUf | 652 |
| | | | CmUfCmUfUmCmAmCmUmC | |
| IN-328 | GUGAAGAGACCAAGAUGAA | 653 | mU*fUmCfAmUfCmUfUmGmGmU | 654 |
| | | | fCmUfCmUfUmCfAmCmU*mC | |
| IN-329 | GUGAAGAGACCAAGAUGAA | 655 | mU*fU*mCfAmUfCmUfUmGmGm | 656 |
| | | | UfCmUfCmUfUmCfAmC*mU*mC | |
| IN-330 | GUGAAGAGACCAAGAUGAA | 657 | mUdUmCfAdUfCdUfUmGmGmUd | 658 |
| | | | CmUfCmUfUmCfAmCmUmC | |
| IN-331 | mGmUmGmAfAfGfAfGfAfCmC | 659 | UUCAUCUUGGUCUCUUCACUU | 660 |
| | mAmAmGmAmUmGmAmA | | | |
| IN-332 | mGmUmGmAmAfGfAfGfAfCmC | 661 | UUCAUCUUGGUCUCUUCACUU | 662 |
| | mAmAmGmAmUmGmAmA | | | |
| IN-333 | mGmUmGmAmAmGfAfGfAfCm | 663 | UUCAUCUUGGUCUCUUCACUU | 664 |
| | CmAmAmGmAmUmGmAmA | | | |
| IN-334 | mGmUmGmAmAmGmAfGfAfCm | 665 | UUCAUCUUGGUCUCUUCACUU | 666 |
| | CmAmAmGmAmUmGmAmA | | | |
| IN-335 | mGmUmGmAfAmGmAmGfAfCm | 667 | UUCAUCUUGGUCUCUUCACUU | 668 |
| | CmAmAmGmAmUmGmAmA | | | |
| IN-336 | mGmUmGmAfAfGmAmGmAfCm | 669 | UUCAUCUUGGUCUCUUCACUU | 670 |
| | CmAmAmGmAmUmGmAmA | | | |
| IN-337 | mGmUmGmAfAfGfAmGmAmCm | 671 | UUCAUCUUGGUCUCUUCACUU | 672 |
| | CmAmAmGmAmUmGmAmA | | | |
| IN-338 | mGmUmGmAfAfGfAfGmAmCm | 673 | UUCAUCUUGGUCUCUUCACUU | 674 |
| | CmAmAmGmAmUmGmAmA | | | |
| IN-339 | mGmUmGmAfAfGfAfGfAmCmC | 675 | UUCAUCUUGGUCUCUUCACUU | 676 |
| | mAmAmGmAmUmGmAmA | | | |
| IN-340 | mG*mUmGmAfAfGfAfGfAfCmC | 677 | UUCAUCUUGGUCUCUUCACUU | 678 |
| | mAmAmGmAmUmGmAmA | | | |
| IN-341 | mG*mU*mGmAfAfGfAfGfAfCm | 679 | UUCAUCUUGGUCUCUUCACUU | 680 |
| | CmAmAmGmAmUmGmAmA | | | |
| IN-342 | mG*mU*mGmAfAfGfAfGfAfCm | 681 | mU*fU*mCfAmUfCmUfUmGmGm | 682 |
| | CmAmAmGmAmUmGmAmA | | UfCmUfCmUfUmCfAmC*mU*mC | |
| IN-343 | irdAmGmUmGmAfAfGfAfGfAfC | 683 | UUCAUCUUGGUCUCUUCACUU | 684 |
| | mCmAmAmGmAmUmGmAmA | | | |
| IN-344 | irdAmUmGmAfAfGfAfGfAfCmC | 685 | UUCAUCUUGGUCUCUUCAUUU | 686 |
| | mAmAmGmAmUmGmAmA | | | |
| IN-345 | CAGAGCAACAGUUCUUCAA | 687 | mUfUmGfAmAfGmAfAmCmUmGf | 688 |
| | | | UmUfGmCfUmCfUmGmGmA | |
| IN-346 | CAGAGCAACAGUUCUUCAA | 689 | mUfUmGfAmAfGmAfAfCmUmGf | 690 |
| | | | UmUfGmCfUmCfUmGmGmA | |
| IN-347 | CAGAGCAACAGUUCUUCAA | 691 | mUfUmGfAmAfGmAfAmCfUmGf | 692 |
| | | | UmUfGmCfUmCfUmGmGmA | |
| IN-348 | CAGAGCAACAGUUCUUCAA | 693 | mUfUmGfAmAfGmAfAmCmUfGf | 694 |
| | | | UmUfGmCfUmCfUmGmGmA | |
| IN-349 | CAGAGCAACAGUUCUUCAA | 695 | mUmUmGfAmAfGmAfAmCmUmG | 696 |
| | | | fUmUfGmCfUmCfUmGmGmA | |
| IN-350 | CAGAGCAACAGUUCUUCAA | 697 | mUfUmGmAmAfGmAfAmCmUmG | 698 |
| | | | fUmUfGmCfUmCfUmGmGmA | |
| IN-351 | CAGAGCAACAGUUCUUCAA | 699 | mUfUmGfAmAmGmAfAmCmUmG | 700 |
| | | | fUmUfGmCfUmCfUmGmGmA | |
| IN-352 | CAGAGCAACAGUUCUUCAA | 701 | mUfUmGfAmAfGmAmAmCmUmG | 702 |
| | | | fUmUfGmCfUmCfUmGmGmA | |
| IN-353 | CAGAGCAACAGUUCUUCAA | 703 | mUfUmGfAmAfGmAfAmCmUmG | 704 |
| | | | mUmUfGmCfUmCfUmGmGmA | |
| IN-354 | CAGAGCAACAGUUCUUCAA | 705 | mUfUmGfAmAfGmAfAmCmUmGf | 706 |
| | | | UmUmGmCfUmCfUmGmGmA | |
| IN-355 | CAGAGCAACAGUUCUUCAA | 707 | mUfUmGfAmAfGmAfAmCmUmGf | 708 |
| | | | UmUfGmCmUmCfUmGmGmA | |
| IN-356 | CAGAGCAACAGUUCUUCAA | 709 | mUfUmGfAmAfGmAfAmCmUmGf | 710 |
| | | | UmUfGmCfUmCmUmGmGmA | |
| IN-357 | CAGAGCAACAGUUCUUCAA | 711 | mU*fUmGfAmAfGmAfAmCmUmG | 712 |
| | | | fUmUfGmCfUmCfUmGmG*mA | |
| IN-358 | CAGAGCAACAGUUCUUCAA | 713 | mU*fU*mGfAmAfGmAfAmCmUm | 714 |
| | | | GfUmUfGmCfUmCfUmG*mG*mA | |
| IN-359 | CAGAGCAACAGUUCUUCAA | 715 | mUdTmGfAdAfGdAfAmCmUmGd | 716 |
| | | | TmUfGmCfUmCfUmGmGmA | |
| IN-360 | CAGAGCAACAGUUCUUCAA | 717 | mUfUmIfAmAfGmAfAmCmUmGf | 718 |
| | | | UmUfGmCfUmCfUmGmGmA | |
| IN-361 | CAGAGCAACAGUUCUUCAA | 719 | mUfUmGfAmAfImAfAmCmUmGf | 720 |
| | | | UmUfGmCfUmCfUmGmGmA | |
| IN-362 | mCmAmGmAfGfCfAfAfCfAmGm | 721 | UUGAAGAACUGUUGCUCUGGA | 722 |
| | UmUmCmUmUmCmAmA | | | |
| IN-363 | mCmAmGmAmGfCfAfAfCfAmG | 723 | UUGAAGAACUGUUGCUCUGGA | 724 |
| | mUmUmCmUmUmCmAmA | | | |
| IN-364 | mCmAmGmAmGmCfAfAfCfAm | 725 | UUGAAGAACUGUUGCUCUGGA | 726 |
| | GmUmUmCmUmUmCmAmA | | | |
| IN-365 | mCmAmGmAmGmCmAfAfCfAm | 727 | UUGAAGAACUGUUGCUCUGGA | 728 |
| | GmUmUmCmUmUmCmAmA | | | |
| IN-366 | mCmAmGmAfGmCmAmAfCfAm | 729 | UUGAAGAACUGUUGCUCUGGA | 730 |
| | GmUmUmCmUmUmCmAmA | | | |
| IN-367 | mCmAmGmAfGfCmAmAmCfAm | 731 | UUGAAGAACUGUUGCUCUGGA | 732 |
| | GmUmUmCmUmUmCmAmA | | | |
| IN-368 | mCmAmGmAfGfCfAmAmCmAm | 733 | UUGAAGAACUGUUGCUCUGGA | 734 |
| | GmUmUmCmUmUmCmAmA | | | |
| IN-369 | mCmAmGmAfGfCfAfAmCmAm | 735 | UUGAAGAACUGUUGCUCUGGA | 736 |
| | GmUmUmCmUmUmCmAmA | | | |
| IN-370 | mCmAmGmAfGfCfAfAfCmAmG | 737 | UUGAAGAACUGUUGCUCUGGA | 738 |
| | mUmUmCmUmUmCmAmA | | | |
| IN-371 | mC*mAmGmAfGfCfAfAfCfAmG | 739 | UUGAAGAACUGUUGCUCUGGA | 740 |
| | mUmUmCmUmUmCmAmA | | | |
| IN-372 | mC*mA*mGmAfGfCfAfAfCfAm | 741 | UUGAAGAACUGUUGCUCUGGA | 742 |
| | GmUmUmCmUmUmCmAmA | | | |
| IN-373 | mC*mA*mGmAfGfCfAfAfCfAm | 743 | mU*fU*mGfAmAfGmAfAmCmUm | 744 |
| | GmUmUmCmUmUmCmAmA | | GfUmUfGmCfUmCfUmG*mG*mA | |
| IN-374 | AGUCAAAGCUAUUUUCAUA | 745 | mUfAmUfGmAfAmAfAmUmAmGf | 746 |
| | | | CmUfUmUfGmAfCmGmUmU | |
| IN-375 | AGUCAAAGCUAUUUUCAUA | 747 | mUfAmUfGmAfAmAfAfUmAmGf | 748 |
| | | | CmUfUmUfGmAfCmGmUmU | |
| IN-376 | AGUCAAAGCUAUUUUCAUA | 749 | mUfAmUfGmAfAmAfAmUfAmGf | 750 |
| | | | CmUfUmUfGmAfCmGmUmU | |
| IN-377 | AGUCAAAGCUAUUUUCAUA | 751 | mUfAmUfGmAfAmAfAmUmAfGf | 752 |
| | | | CmUfUmUfGmAfCmGmUmU | |
| IN-378 | AGUCAAAGCUAUUUUCAUA | 753 | mUmAmUfGmAfAmAfAmUmAmG | 754 |
| | | | fCmUfUmUfGmAfCmGmUmU | |
| IN-379 | AGUCAAAGCUAUUUUCAUA | 755 | mUfAmUmGmAfAmAfAmUmAmG | 756 |
| | | | fCmUfUmUfGmAfCmGmUmU | |
| IN-380 | AGUCAAAGCUAUUUUCAUA | 757 | mUfAmUfGmAmAmAfAmUmAmG | 758 |
| | | | fCmUfUmUfGmAfCmGmUmU | |
| IN-381 | AGUCAAAGCUAUUUUCAUA | 759 | mUfAmUfGmAfAmAmAmUmAmG | 760 |
| | | | fCmUfUmUfGmAfCmGmUmU | |
| IN-382 | AGUCAAAGCUAUUUUCAUA | 761 | mUfAmUfGmAfAmAfAmUmAmG | 762 |
| | | | mCmUfUmUfGmAfCmGmUmU | |
| IN-383 | AGUCAAAGCUAUUUUCAUA | 763 | mUfAmUfGmAfAmAfAmUmAmGf | 764 |
| | | | CmUmUmUfGmAfCmGmUmU | |
| IN-384 | AGUCAAAGCUAUUUUCAUA | 765 | mUfAmUfGmAfAmAfAmUmAmGf | 766 |
| | | | CmUfUmUmGmAfCmGmUmU | |
| IN-385 | AGUCAAAGCUAUUUUCAUA | 767 | mUfAmUfGmAfAmAfAmUmAmGf | 768 |
| | | | CmUfUmUfGmAmCmGmUmU | |
| IN-386 | AGUCAAAGCUAUUUUCAUA | 769 | mU*fAmUfGmAfAmAfAmUmAm | 770 |
| | | | GfCmUfUmUfGmAfCmGmU*mU | |
| IN-387 | AGUCAAAGCUAUUUUCAUA | 771 | mU*fA*mUfGmAfAmAfAmUmAm | 772 |
| | | | GfCmUfUmUfGmAfCmG*mU*mU | |
| IN-388 | AGUCAAAGCUAUUUUCAUA | 773 | mUdAmUfGdAfAdAfAmUmAmGd | 774 |
| | | | CmUfUmUfGmAfCmGmUmU | |
| IN-389 | AGUCAAAGCUAUUUUCAUA | 775 | mUfAmUfImAfAmAfAmUmAmGf | 776 |
| | | | CmUfUmUfGmAfCmGmUmU | |
| IN-390 | mAmGmUmCfAfAfAfGfCfUmA | 777 | UAUGAAAAUAGCUUUGACUUU | 778 |
| | mUmUmUmUmCmAmUmA | | | |
| IN-391 | mAmGmUmCmAfAfAfGfCfUmA | 779 | UAUGAAAAUAGCUUUGACUUU | 780 |
| | mUmUmUmUmCmAmUmA | | | |
| IN-392 | mAmGmUmCmAmAfAfGfCfUm | 781 | UAUGAAAAUAGCUUUGACUUU | 782 |
| | AmUmUmUmUmCmAmUmA | | | |
| IN-393 | mAmGmUmCmAmAmAfGfCfUm | 783 | UAUGAAAAUAGCUUUGACUUU | 784 |
| | AmUmUmUmUmCmAmUmA | | | |
| IN-394 | mAmGmUmCfAmAmAmGfCfUm | 785 | UAUGAAAAUAGCUUUGACUUU | 786 |
| | AmUmUmUmUmCmAmUmA | | | |
| IN-395 | mAmGmUmCfAfAmAmGmCfUm | 787 | UAUGAAAAUAGCUUUGACUUU | 788 |
| | AmUmUmUmUmCmAmUmA | | | |
| IN-396 | mAmGmUmCfAfAfAmGmCmUm | 789 | UAUGAAAAUAGCUUUGACUUU | 790 |
| | AmUmUmUmUmCmAmUmA | | | |
| IN-397 | mAmGmUmCfAfAfAfGmCmUm | 791 | UAUGAAAAUAGCUUUGACUUU | 792 |
| | AmUmUmUmUmCmAmUmA | | | |
| IN-398 | mAmGmUmCfAfAfAfGfCmUmA | 793 | UAUGAAAAUAGCUUUGACUUU | 794 |
| | mUmUmUmUmCmAmUmA | | | |
| IN-399 | mA*mGmUmCfAfAfAfGfCfUmA | 795 | UAUGAAAAUAGCUUUGACUUU | 796 |
| | mUmUmUmUmCmAmUmA | | | |
| IN-400 | mA*mG*mUmCfAfAfAfGfCfUm | 797 | UAUGAAAAUAGCUUUGACUUU | 798 |
| | AmUmUmUmUmCmAmUmA | | | |
| IN-401 | mA*mG*mUmCfAfAfAfGfCfUm | 799 | mU*fA*mUfGmAfAmAfAmUmAm | 800 |
| | AmUmUmUmUmCmAmUmA | | GfCmUfUmUfGmAfCmG*mU*mU | |
| IN-402 | CAUGUGAUUACAUCAUCUU | 801 | mAfAmGfAmUfGmAfUmGmUmAf | 802 |
| | | | AmUfCmAfCmAfUmGmUmC | |
| IN-403 | CAUGUGAUUACAUCAUCUU | 803 | mAfAmGfAmUfGmAfUfGmUmAf | 804 |
| | | | AmUfCmAfCmAfUmGmUmC | |
| IN-404 | CAUGUGAUUACAUCAUCUU | 805 | mAfAmGfAmUfGmAfUmGfUmAf | 806 |
| | | | AmUfCmAfCmAfUmGmUmC | |
| IN-405 | CAUGUGAUUACAUCAUCUU | 807 | mAfAmGfAmUfGmAfUmGmUfAf | 808 |
| | | | AmUfCmAfCmAfUmGmUmC | |
| IN-406 | CAUGUGAUUACAUCAUCUU | 809 | mAmAmGfAmUfGmAfUmGmUmA | 810 |
| | | | fAmUfCmAfCmAfUmGmUmC | |
| IN-407 | CAUGUGAUUACAUCAUCUU | 811 | mAfAmGmAmUfGmAfUmGmUmA | 812 |
| | | | fAmUfCmAfCmAfUmGmUmC | |
| IN-408 | CAUGUGAUUACAUCAUCUU | 813 | mAfAmGfAmUmGmAfUmGmUmA | 814 |
| | | | fAmUfCmAfCmAfUmGmUmC | |
| IN-409 | CAUGUGAUUACAUCAUCUU | 815 | mAfAmGfAmUfGmAmUmGmUmA | 816 |
| | | | fAmUfCmAfCmAfUmGmUmC | |
| IN-410 | CAUGUGAUUACAUCAUCUU | 817 | mAfAmGfAmUfGmAfUmGmUmA | 818 |
| | | | mAmUfCmAfCmAfUmGmUmC | |
| IN-411 | CAUGUGAUUACAUCAUCUU | 819 | mAfAmGfAmUfGmAfUmGmUmAf | 820 |
| | | | AmUmCmAfCmAfUmGmUmC | |
| IN-412 | CAUGUGAUUACAUCAUCUU | 821 | mAfAmGfAmUfGmAfUmGmUmAf | 822 |
| | | | AmUfCmAmCmAfUmGmUmC | |
| IN-413 | CAUGUGAUUACAUCAUCUU | 823 | mAfAmGfAmUfGmAfUmGmUmAf | 824 |
| | | | AmUfCmAfCmAmUmGmUmC | |
| IN-414 | CAUGUGAUUACAUCAUCUU | 825 | mA*fAmGfAmUfGmAfUmGmUm | 826 |
| | | | AfAmUfCmAfCmAfUmGmU*mC | |
| IN-415 | CAUGUGAUUACAUCAUCUU | 827 | mA*fA*mGfAmUfGmAfUmGmUm | 828 |
| | | | AfAmUfCmAfCmAfUmG*mU*mC | |
| IN-416 | CAUGUGAUUACAUCAUCUU | 829 | mAdAmGfAdUfGdAfUmGmUmAd | 830 |
| | | | AmUfCmAfCmAfUmGmUmC | |
| IN-417 | CAUGUGAUUACAUCAUCUU | 831 | mAfAmIfAmUfGmAfUmGmUmAf | 832 |
| | | | AmUfCmAfCmAfUmGmUmC | |
| IN-418 | CAUGUGAUUACAUCAUCUU | 833 | mAfAmGfAmUfImAfUmGmUmAf | 834 |
| | | | AmUfCmAfCmAfUmGmUmC | |
| IN-419 | mCmAmUmGfUfGfAfUfUfAmC | 835 | AAGAUGAUGUAAUCACAUGUC | 836 |
| | mAmUmCmAmUmCmUmU | | | |
| IN-420 | mCmAmUmGmUfGfAfUfUfAmC | 837 | AAGAUGAUGUAAUCACAUGUC | 838 |
| | mAmUmCmAmUmCmUmU | | | |
| IN-421 | mCmAmUmGmUmGfAfUfUfAm | 839 | AAGAUGAUGUAAUCACAUGUC | 840 |
| | CmAmUmCmAmUmCmUmU | | | |
| IN-422 | mCmAmUmGmUmGmAfUfUfAm | 841 | AAGAUGAUGUAAUCACAUGUC | 842 |
| | CmAmUmCmAmUmCmUmU | | | |
| IN-423 | mCmAmUmGfUmGmAmUfUfAm | 843 | AAGAUGAUGUAAUCACAUGUC | 844 |
| | CmAmUmCmAmUmCmUmU | | | |
| IN-424 | mCmAmUmGfUfGmAmUmUfAm | 845 | AAGAUGAUGUAAUCACAUGUC | 846 |
| | CmAmUmCmAmUmCmUmU | | | |
| IN-425 | mCmAmUmGfUfGfAmUmUmAm | 847 | AAGAUGAUGUAAUCACAUGUC | 848 |
| | CmAmUmCmAmUmCmUmU | | | |
| IN-426 | mCmAmUmGfUfGfAfUmUmAm | 849 | AAGAUGAUGUAAUCACAUGUC | 850 |
| | CmAmUmCmAmUmCmUmU | | | |
| IN-427 | mCmAmUmGfUfGfAfUfUmAmC | 851 | AAGAUGAUGUAAUCACAUGUC | 852 |
| | mAmUmCmAmUmCmUmU | | | |
| IN-428 | mC*mAmUmGfUfGfAfUfUfAmC | 853 | AAGAUGAUGUAAUCACAUGUC | 854 |
| | mAmUmCmAmUmCmUmU | | | |
| IN-429 | mC*mA*mUmGfUfGfAfUfUfAm | 855 | AAGAUGAUGUAAUCACAUGUC | 856 |
| | CmAmUmCmAmUmCmUmU | | | |
| IN-430 | mC*mA*mUmGfUfGfAfUfUfAm | 857 | mA*fA*mGfAmUfGmAfUmGmUm | 858 |
| | CmAmUmCmAmUmCmUmU | | AfAmUfCmAfCmAfUmG*mU*mC | |
| IN-431 | mG*mC*mUmGmAmGfAfCfUm | 859 | mA*fA*mUmAmAmGmCmCmAm | 860 |
| | CmUmGmGmCmUmUmAmUmU | | GmAmGmUfCmUmCmAmGmC*m | |
| | | | C*mA | |
| IN-432 | mG*mC*mUmGmAmGfAfCfUm | 861 | mA*fA*mUmAmAmGmCmCmAfG | 862 |
| | CmUmGmGmCmUmUmAmUmU | | mAmGmUfCmUmCmAmGmC*mC | |
| | | | *mA | |
| IN-433 | mG*mC*mUmGmAmGfAfCfUm | 863 | mA*fA*mUmAmAmGmCmCmAm | 864 |
| | CmUmGmGmCmUmUmAmUmU | | GmAmGmUfCmUmCmAmGmG*m | |
| | | | C*mA | |
| IN-434 | irmG*mC*mUmGmAmGfAfCfUm | 865 | mA*fA*mUmAmAmGmCmCmAm | 866 |
| | CmUmGmGmCmUmUmAmUmU | | GmAmGmUfCmUmCmAmGmC*m | |
| | | | C*mA | |
| IN-435 | mC*mC*mAmUmCmAfGfCfUmU | 867 | mA*fC*mAmGmUmAmGmCfAmA | 868 |
| | mUmGmCmUmAmCmUmGmU | | mAfGmCfUmGmAmUmGmG*mU* | |
| | | | mU | |
| IN-436 | mC*mC*mAmUmCmAfGfCfUmU | 869 | mA*fC*mAmGmUmAmGmCfAmA | 870 |
| | mUmGmCmUmAmCmUmGmU | | mAfGmCmUmGmAmUmGmG*mU | |
| | | | *mU | |
| IN-437 | mC*mC*mAmUmCmAfGfCfUmU | 871 | mA*fC*mAmGmUmAmGfCfAmA | 872 |
| | mUmGmCmUmAmCmUmGmU | | mAfGmCfUmGmAmUmGmG*mU* | |
| | | | mU | |
| IN-438 | mC*mC*mAmUmCmAfGfCfUmU | 873 | mA*fC*mAmGmUmAmGfCfAmA | 874 |
| | mUmGmCmUmAmCmUmGmU | | mAfGmCmUmGmAmUmGmG*mU | |
| | | | *mU | |
| IN-439 | mC*mA*mUmGfUfGfAmUmUm | 875 | mA*fA*mGmAmUmImAmUmGmU | 876 |
| | AmCmAmUmCmAmUmCmUmU | | fAmAmUmCmAmCmAmUmG*mU | |
| | | | *mC | |
| IN-440 | mC*mA*mUmGfUfGfAmUmUm | 877 | mA*fA*mGmAfUmImAmUmGmUf | 878 |
| | AmCmAmUmCmAmUmCmUmU | | AmAmUmCmAmCmAmUmG*mU* | |
| | | | mC | |
| IN-441 | mC*mA*mUmGfUfGfAmUmUm | 879 | mA*fA*mGmAfUmIfAmUmGmUf | 880 |
| | AmCmAmUmCmAmUmCmUmU | | AmAmUmCmAmCmAmUmG*mU* | |
| | | | mC | |
| IN-442 | mC*mA*mUmGfUfGfAmUmUm | 881 | mA*dA*mGmAdUmIdAmUmGmUf | 882 |
| | AmCmAmUmCmAmUmCmUmU | | AdAmUmCmAmCmAmUmG*mU* | |
| | | | mC | |
| IN-443 | mC*mA*mUmGmUmGmAmUmU | 883 | mA*fA*mGmAmUmImAmUmGmU | 884 |
| | mAmCmAmUmCmAmUmCmUm | | fAmAmUmCmAmCmAmUmG*mU | |
| | U | | *mC | |
| IN-444 | mC*mA*mUmGfUfGfAmUmUm | 885 | mA*fA*mGmAmUmGmAmUmGm | 886 |
| | AmCmAmUmCmAmUmCmUmU | | UfAmAmUmCmAmCmAmUmG*m | |
| | | | U*mC | |
| IN-445 | mC*mA*mUmGfUfGfAmUmUm | 887 | mA*fA*mGmAfUmGmAmUmGmU | 888 |
| | AmCmAmUmCmAmUmCmUmU | | fAmAmUmCmAmCmAmUmG*mU | |
| | | | *mC | |
| IN-446 | mC*mA*mUmGfUfGfAmUmUm | 889 | mA*fA*mGmAfUmGfAmUmGmUf | 89 |
| | AmCmAmUmCmAmUmCmUmU | | AmAmUmCmAmCmAmUmG*mU* | |
| | | | mC | |
| IN-447 | mC*mA*mUmGfUfGfAmUmUm | 891 | mA*dA*mGmAdUmGdAmUmGmU | 892 |
| | AmCmAmUmCmAmUmCmUmU | | fAdAmUmCmAmCmAmUmG*mU* | |
| | | | mC | |
| IN-448 | mC*mA*mUmGmUmGmAmUmU | 893 | mA*fA*mGmAmUmGmAmUmGm | 894 |
| | mAmCmAmUmCmAmUmCmUm | | UfAmAmUmCmAmCmAmUmG*m | |
| | U | | U*mC | |
| IN-449 | CAUGUUAUUUGCCUUUUGA | 895 | mUfCmAfAmAfAmGfGmCmAmAf | 896 |
| | | | AmUfAmAfCmAfUmGmUmU | |
| IN-450 | CAUGUUAUUUGCCUUUUGA | 897 | mUfCmAfAmAfAmGfGfCmAmAf | 898 |
| | | | AmUfAmAfCmAfUmGmUmU | |
| IN-451 | CAUGUUAUUUGCCUUUUGA | 899 | mUfCmAfAmAfAmGfGmCfAmAf | 900 |
| | | | AmUfAmAfCmAfUmGmUmU | |
| IN-452 | CAUGUUAUUUGCCUUUUGA | 901 | mUfCmAfAmAfAmGfGmCmAfAf | 902 |
| | | | AmUfAmAfCmAfUmGmUmU | |
| IN-453 | CAUGUUAUUUGCCUUUUGA | 903 | mUmCmAfAmAfAmGfGmCmAmA | 904 |
| | | | fAmUfAmAfCmAfUmGmUmU | |
| IN-454 | CAUGUUAUUUGCCUUUUGA | 905 | mUfCmAmAmAfAmGfGmCmAmA | 906 |
| | | | fAmUfAmAfCmAfUmGmUmU | |
| IN-455 | CAUGUUAUUUGCCUUUUGA | 907 | mUfCmAfAmAmAmGfGmCmAmA | 908 |
| | | | fAmUfAmAfCmAfUmGmUmU | |
| IN-456 | CAUGUUAUUUGCCUUUUGA | 909 | mUfCmAfAmAfAmGmGmCmAmA | 910 |
| | | | fAmUfAmAfCmAfUmGmUmU | |
| IN-457 | CAUGUUAUUUGCCUUUUGA | 911 | mUfCmAfAmAfAmGfGmCmAmA | 912 |
| | | | mAmUfAmAfCmAfUmGmUmU | |
| IN-458 | CAUGUUAUUUGCCUUUUGA | 913 | mUfCmAfAmAfAmGfGmCmAmAf | 914 |
| | | | AmUmAmAfCmAfUmGmUmU | |
| IN-459 | CAUGUUAUUUGCCUUUUGA | 915 | mUfCmAfAmAfAmGfGmCmAmAf | 916 |
| | | | AmUfAmAmCmAfUmGmUmU | |
| IN-460 | CAUGUUAUUUGCCUUUUGA | 917 | mUfCmAfAmAfAmGfGmCmAmAf | 918 |
| | | | AmUfAmAfCmAmUmGmUmU | |
| IN-461 | CAUGUUAUUUGCCUUUUGA | 919 | mU*fCmAfAmAfAmGfGmCmAmA | 920 |
| | | | fAmUfAmAfCmAfUmGmU*mU | |
| IN-462 | CAUGUUAUUUGCCUUUUGA | 921 | mU*fC*mAfAmAfAmGfGmCmAm | 922 |
| | | | AfAmUfAmAfCmAfUmG*mU*mU | |
| IN-463 | CAUGUUAUUUGCCUUUUGA | 923 | mUdCmAfAdAfAdGfGmCmAmAd | 924 |
| | | | AmUfAmAfCmAfUmGmUmU | |
| IN-464 | CAUGUUAUUUGCCUUUUGA | 925 | mUfCmAfAmAfAmIfGmCmAmAf | 926 |
| | | | AmUfAmAfCmAfUmmGmmUmmU | |
| IN-465 | CAUGUUAUUUGCCUUUUGA | 927 | mUfCmAfAmAfAmGfImCmAmAf | 928 |
| | | | AmUfAmAfCmAfUmmmGmmmU | |
| | | | mmmU | |
| IN-466 | mCmAmUmGfUfUfAfUfUfUmG | 929 | UCAAAAGGCAAAUAACAUGUU | 930 |
| | mCmCmUmUmUmUmGmA | | | |
| IN-467 | mCmAmUmGmUfUfAfUfUfUmG | 931 | UCAAAAGGCAAAUAACAUGUU | 932 |
| | mCmCmUmUmUmUmGmA | | | |
| IN-468 | mCmAmUmGmUmUfAfUfUfUm | 933 | UCAAAAGGCAAAUAACAUGUU | 934 |
| | GmCmCmUmUmUmUmGmA | | | |
| IN-469 | mCmAmUmGmUmUmAfUfUfUm | 935 | UCAAAAGGCAAAUAACAUGUU | 936 |
| | GmCmCmUmUmUmUmGmA | | | |
| IN-470 | mCmAmUmGfUmUmAmUfUfUm | 937 | UCAAAAGGCAAAUAACAUGUU | 938 |
| | GmCmCmUmUmUmUmGmA | | | |
| IN-471 | mCmAmUmGfUfUmAmUmUfUm | 939 | UCAAAAGGCAAAUAACAUGUU | 940 |
| | GmCmCmUmUmUmUmGmA | | | |
| IN-472 | mCmAmUmGfUfUfAmUmUmUm | 941 | UCAAAAGGCAAAUAACAUGUU | 942 |
| | GmCmCmUmUmUmUmGmA | | | |
| IN-473 | mCmAmUmGfUfUfAfUmUmUm | 943 | UCAAAAGGCAAAUAACAUGUU | 944 |
| | GmCmCmUmUmUmUmGmA | | | |
| IN-474 | mCmAmUmGfUfUfAfUfUmUmG | 945 | UCAAAAGGCAAAUAACAUGUU | 946 |
| | mCmCmUmUmUmUmGmA | | | |
| IN-475 | mC*mAmUmGfUfUfAfUfUfUmG | 947 | UCAAAAGGCAAAUAACAUGUU | 948 |
| | mCmCmUmUmUmUmGmA | | | |
| IN-476 | mC*mA*mUmGfUfUfAfUfUfUm | 949 | UCAAAAGGCAAAUAACAUGUU | 950 |
| | GmCmCmUmUmUmUmGmA | | | |
| IN-477 | mC*mA*mUmGfUfUfAfUfUfUm | 951 | mU*fC*mAfAmAfAmGfGmCmAm | 952 |
| | GmCmCmUmUmUmUmGmA | | AfAmUfAmAfCmAfUmG*mU*mU | |
| IN-479 | mA*mG*mUmCmAmAmAmGmC | 955 | mU*fA*mUmGmAfAmAfAmUmA | 956 |
| | mUmAmUmUmUmUmCmA*mU* | | mGmCmUfUmUmGmAmCmU*mU | |
| | mA | | *mU | |
| IN-480 | mA*mG*mUmCmAmAmAmGmC | 957 | mU*fA*mUmGfAfAmAfAmUmAm | 958 |
| | mUmAmUmUmUmUmCmA*mU* | | GmCmUfUmUmGmAmCmU*mU* | |
| | mA | | mU | |
| IN-481 | mA*mG*mUmCmAmAmAmGmC | 959 | mU*fA*mUmGmAfAfAfAmUmAm | 960 |
| | mUmAmUmUmUmUmCmA*mU* | | GmCmUfUmUmGmAmCmU*mU* | |
| | mA | | mU | |
| IN-482 | mA*mG*mUmCmAmAmAmGmC | 961 | mU*fA*mUmGfAfAfAfAmUmAm | 962 |
| | mUmAmUmUmUmUmCmA*mU* | | GmCmUfUmUmGmAmCmU*mU* | |
| | mA | | mU | |
| IN-483 | mA*mG*mUmCmAmAmAmGmC | 963 | mU*dA*mUfGdAfAdAfAmUmAm | 964 |
| | mUmAmUmUmUmUmCmA*mU* | | GdCmUfUmUfGmAfCmU*mU*mU | |
| | mA | | | |
| IN-484 | mC*mG*mUmCmAmAmAmGmC | 965 | mU*dA*mUfGdAfAdAfAmUmAm | 966 |
| | mUmAmUmUmUmUmCmA*mU* | | GdCmUfUmUfGmAfCmG*mU*mU | |
| | mA | | | |
| IN-485 | mA*mG*mUmCmAmAfAfGfCm | 967 | mU*fA*mUmGmAfAmAfAmUmA | 968 |
| | UmAmUmUmUmUmCmA*mU*m | | mGmCmUfUmUmGmAmCmU*mU | |
| | A | | *mU | |
| IN-486 | mC*mA*mUmGmUmUfAfUfUm | 969 | mU*fC*mAmAmAmAmGmGmCm | 970 |
| | UmGmCmCmUmUmUmU*mG*m | | AmAmAmUfAmAmCmAmUmG*m | |
| | A | | U*mU | |
| IN-487 | mC*mA*mUmGmUmUfAfUfUm | 971 | mU*dC*mAfAdAfAdGfGmCmAm | 972 |
| | UmGmCmCmUmUmUmU*mG*m | | AdAmUfAmAfCmAfUmG*mU*mU | |
| | A | | | |
| IN-488 | mC*mA*mUmGmUmUfAfUfUm | 973 | mU*fC*mAmAmAmAmGmImCmA | 974 |
| | UmGmCmCmUmUmUmU*mG*m | | mAmAmUfAmAmCmAmUmG*mU | |
| | A | | *mU | |
| INI-001 | GUGAGGGUCAAGCACAGCU | 975 | AICUGUGCUUGACCCUCACAG | 976 |
| INI-002 | GAGGGUCAAGCACAGCUAU | 977 | AUAICUGUGCUUGACCCUCAC | 978 |
| INI-003 | GGGUCAAGCACAGCUAUCC | 979 | GIAUAGCUGUGCUUGACCCUC | 980 |
| INI-004 | GGUCAAGCACAGCUAUCCA | 981 | UIGAUAGCUGUGCUUGACCCU | 982 |
| INI-005 | GUCAAGCACAGCUAUCCAU | 983 | AUIGAUAGCUGUGCUUGACCC | 984 |
| INI-006 | UCAAGCACAGCUAUCCAUC | 985 | GAUIGAUAGCUGUGCUUGACC | 986 |
| INI-007 | CAAGCACAGCUAUCCAUCA | 987 | UIAUGGAUAGCUGUGCUUGAC | 988 |
| INI-008 | AAGCACAGCUAUCCAUCAG | 989 | CUIAUGGAUAGCUGUGCUUGA | 990 |
| INI-009 | AGCACAGCUAUCCAUCAGA | 991 | UCUIAUGGAUAGCUGUGCUUG | 992 |
| INI-010 | GCACAGCUAUCCAUCAGAU | 993 | AUCUIAUGGAUAGCUGUGCUU | 994 |
| INI-011 | CACAGCUAUCCAUCAGAUG | 995 | CAUCUIAUGGAUAGCUGUGCU | 996 |
| INI-012 | ACAGCUAUCCAUCAGAUGA | 997 | UCAUCUIAUGGAUAGCUGUGC | 998 |
| INI-013 | CAGCUAUCCAUCAGAUGAU | 999 | AUCAUCUIAUGGAUAGCUGUG | 1000 |
| INI-014 | GCUAUCCAUCAGAUGAUCU | 1001 | AIAUCAUCUGAUGGAUAGCUG | 1002 |
| INI-015 | CUAUCCAUCAGAUGAUCUA | 1003 | UAIAUCAUCUGAUGGAUAGCU | 1004 |
| INI-016 | AUCCAUCAGAUGAUCUACU | 1005 | AIUAGAUCAUCUGAUGGAUAG | 1006 |
| INI-017 | UCCAUCAGAUGAUCUACUU | 1007 | AAIUAGAUCAUCUGAUGGAUA | 1008 |
| INI-018 | CCAUCAGAUGAUCUACUUU | 1009 | AAAIUAGAUCAUCUGAUGGAU | 1010 |
| INI-019 | CAUCAGAUGAUCUACUUUC | 1011 | GAAAIUAGAUCAUCUGAUGGA | 1012 |
| INI-020 | AUCAGAUGAUCUACUUUCA | 1013 | UIAAAGUAGAUCAUCUGAUGG | 1014 |
| INI-021 | CAGAUGAUCUACUUUCAGC | 1015 | GCUIAAAGUAGAUCAUCUGAU | 1016 |
| INI-022 | GAUGAUCUACUUUCAGCCU | 1017 | AIGCUGAAAGUAGAUCAUCUG | 1018 |
| INI-023 | AUGAUCUACUUUCAGCCUU | 1019 | AAIGCUGAAAGUAGAUCAUCU | 1020 |
| INI-024 | UUCCUGAGUCCCAGACAAU | 1021 | AUUIUCUGGGACUCAGGAAGG | 1022 |
| INI-025 | UCCUGAGUCCCAGACAAUA | 1023 | UAUUIUCUGGGACUCAGGAAG | 1024 |
| INI-026 | UGAGUCCCAGACAAUAGAA | 1025 | UUCUAUUIUCUGGGACUCAGG | 1026 |
| INI-027 | CAAUAGAAGACAGGUGGCU | 1027 | AICCACCUGUCUUCUAUUGUC | 1028 |
| INI-028 | AUAGAAGACAGGUGGCUGU | 1029 | ACAICCACCUGUCUUCUAUUG | 1030 |
| INI-029 | AGGUGUGGCAGUGGUGUCU | 1031 | AIACACCACUGCCACACCUAC | 1032 |
| INI-030 | CAUUGGCCCCCAGCAAUCA | 1033 | UIAUUGCUGGGGGCCAAUGAG | 1034 |
| INI-031 | AGCUAGCCAAGCAGCAAAU | 1035 | AUUUICUGCUUGGCUAGCUCC | 1036 |
| INI-032 | UAGCCAAGCAGCAAAUCCU | 1037 | AIGAUUUGCUGCUUGGCUAGC | 1038 |
| INI-033 | ACCAGUCGUCCCAGAAUAA | 1039 | UUAUUCUIGGACGACUGGUCA | 1040 |
| INI-034 | CAGUCGUCCCAGAAUAACU | 1041 | AIUUAUUCUGGGACGACUGGU | 1042 |
| INI-035 | AGUCGUCCCAGAAUAACUC | 1043 | GAIUUAUUCUGGGACGACUGG | 1044 |
| INI-036 | UCGUCCCAGAAUAACUCAU | 1045 | AUIAGUUAUUCUGGGACGACU | 1046 |
| INI-037 | GGAAUGGGGAGGAGGUCAU | 1047 | AUIACCUCCUCCCCAUUCCCU | 1048 |
| INI-038 | GAGGUCAUCAGCUUUGCUA | 1049 | UAICAAAGCUGAUGACCUCCU | 1050 |
| INI-039 | GGUCAUCAGCUUUGCUACU | 1051 | AIUAGCAAAGCUGAUGACCUC | 1052 |
| INI-040 | GUCAUCAGCUUUGCUACUG | 1053 | CAIUAGCAAAGCUGAUGACCU | 1054 |
| INI-041 | UCAUCAGCUUUGCUACUGU | 1055 | ACAIUAGCAAAGCUGAUGACC | 1056 |
| INI-042 | CAUCAGCUUUGCUACUGUC | 1057 | GACAIUAGCAAAGCUGAUGAC | 1058 |
| INI-043 | GCUUUGCUACUGUCACAGA | 1059 | UCUIUGACAGUAGCAAAGCUG | 1060 |
| INI-044 | UUUGCUACUGUCACAGACU | 1061 | AIUCUGUGACAGUAGCAAAGC | 1062 |
| INI-045 | ACUGUCACAGACUCCACUU | 1063 | AAIUGGAGUCUGUGACAGUAG | 1064 |
| INI-046 | ACCCUUCCUGGCACUCUUU | 1065 | AAAIAGUGCCAGGAAGGGUGG | 1066 |
| INI-047 | CUUCCUGGCACUCUUUGCU | 1067 | AICAAAGAGUGCCAGGAAGGG | 1068 |
| INI-048 | ACUCUUUGCUUGAGGAUCU | 1069 | AIAUCCUCAAGCAAAGAGUGC | 1070 |
| INI-049 | CUCUUUGCUUGAGGAUCUU | 1071 | AAIAUCCUCAAGCAAAGAGUG | 1072 |
| INI-050 | GCUGGCAUACCUUAACUCU | 1073 | AIAGUUAAGGUAUGCCAGCCC | 1074 |
| INI-051 | CUUGAGGGGUGAGAAGUCU | 1075 | AIACUUCUCACCCCUCAAGCC | 1076 |
| INI-052 | AGUCUGGUGUCCUGAAACU | 1077 | AIUUUCAGGACACCAGACUUC | 1078 |
| INI-053 | CUGGUGUCCUGAAACUGCA | 1079 | UICAGUUUCAGGACACCAGAC | 1080 |
| INI-054 | GUGUCCUGAAACUGCAACU | 1081 | AIUUGCAGUUUCAGGACACCA | 1082 |
| INI-055 | UGUCCUGAAACUGCAACUA | 1083 | UAIUUGCAGUUUCAGGACACC | 1084 |
| INI-056 | GUCCUGAAACUGCAACUAG | 1085 | CUAIUUGCAGUUUCAGGACAC | 1086 |
| INI-057 | AGAAGGCAACAGCACAGUU | 1087 | AACUIUGCUGUUGCCUUCUAG | 1088 |
| INI-058 | AGGCAACAGCACAGUUACU | 1089 | AIUAACUGUGCUGUUGCCUUC | 1090 |
| INI-059 | CCUUCCUAGAGCUUAAGAU | 1091 | AUCUUAAICUCUAGGAAGGGC | 1092 |
| INI-060 | AGAGCUUAAGAUCCGAGCC | 1093 | GICUCGGAUCUUAAGCUCUAG | 1094 |
| INI-061 | GAGCUUAAGAUCCGAGCCA | 1095 | UIGCUCGGAUCUUAAGCUCUA | 1096 |
| INI-062 | GCUUAAGAUCCGAGCCAAU | 1097 | AUUIGCUCGGAUCUUAAGCUC | 1098 |
| INI-063 | CCUGCGACCCCCUUAUGUU | 1099 | AACAUAAIGGGGUCGCAGGCU | 1100 |
| INI-064 | UGUUGCAGGCGAGACCAUU | 1101 | AAUIGUCUCGCCUGCAACAUA | 1102 |
| INI-065 | CGAGACCAUUACGUAGACU | 1103 | AIUCUACGUAAUGGUCUCGCC | 1104 |
| INI-066 | GAGACCAUUACGUAGACUU | 1105 | AAIUCUACGUAAUGGUCUCGC | 1106 |
| INI-067 | AUUACGUAGACUUCCAGGA | 1107 | UCCUIGAAGUCUACGUAAUGG | 1108 |
| INI-068 | AGGGGUACCAGCUGAAUUA | 1109 | UAAUUCAICUGGUACCCCUCG | 1110 |
| INI-069 | AUUGCUGCCUCUUUCCAUU | 1111 | AAUIGAAAGAGGCAGCAAUGC | 1112 |
| INI-070 | CUUUCCAUUCUGCCGUCUU | 1113 | AAIACGGCAGAAUGGAAAGAG | 1114 |
| INI-071 | CCUCCUCAAAGCCAACAAU | 1115 | AUUIUUGGCUUUGAGGAGGCU | 1116 |
| INI-072 | CCUCAAAGCCAACAAUCCU | 1117 | AIGAUUGUUGGCUUUGAGGAG | 1118 |
| INI-073 | CUCAAAGCCAACAAUCCUU | 1119 | AAIGAUUGUUGGCUUUGAGGA | 1120 |
| INI-074 | CCUGCCAGUACCUCCUGUU | 1121 | AACAIGAGGUACUGGCAGGCC | 1122 |
| INI-075 | UCUCCUCUACCUGGAUCAU | 1123 | AUIAUCCAGGUAGAGGAGAGA | 1124 |
| INI-076 | CUCCUCUACCUGGAUCAUA | 1125 | UAUIAUCCAGGUAGAGGAGAG | 1126 |
| INI-077 | UCCUCUACCUGGAUCAUAA | 1127 | UUAUIAUCCAGGUAGAGGAGA | 1128 |
| INI-078 | CCUCUACCUGGAUCAUAAU | 1129 | AUUAUIAUCCAGGUAGAGGAG | 1130 |
| INI-079 | CCUGGAUCAUAAUGGCAAU | 1131 | AUUICCAUUAUGAUCCAGGUA | 1132 |
| INI-080 | UGGAUCAUAAUGGCAAUGU | 1133 | ACAUUICCAUUAUGAUCCAGG | 1134 |
| INI-081 | AUAAUGGCAAUGUGGUCAA | 1135 | UUIACCACAUUGCCAUUAUGA | 1136 |
| INI-082 | AUGUGGUCAAGACGGAUGU | 1137 | ACAUCCIUCUUGACCACAUUG | 1138 |
| INI-083 | AGACGGAUGUGCCAGAUAU | 1139 | AUAUCUIGCACAUCCGUCUUG | 1140 |
| INI-084 | UUUGGAGUGAAGAGACCAA | 1141 | UUIGUCUCUUCACUCCAAAGC | 1142 |
| INI-085 | AGUGAAGAGACCAAGAUGA | 1143 | UCAUCUUIGUCUCUUCACUCC | 1144 |
| INI-086 | UGACUGGAGGCAUCAGAUU | 1145 | AAUCUIAUGCCUCCAGUCACA | 1146 |
| INI-087 | AACAACCACCUGGCAAUAU | 1147 | AUAUUICCAGGUGGUUGUUGG | 1148 |
| INI-088 | CCACCUGGCAAUAUGACUC | 1149 | GAIUCAUAUUGCCAGGUGGUU | 1150 |
| INI-089 | CCUGGCAAUAUGACUCACU | 1151 | AIUGAGUCAUAUUGCCAGGUG | 1152 |
| INI-090 | CUGGCAAUAUGACUCACUU | 1153 | AAIUGAGUCAUAUUGCCAGGU | 1154 |
| INI-091 | CAAAUGGGCACUUUCUUGU | 1155 | ACAAIAAAGUGCCCAUUUGGG | 1156 |
| INI-092 | AAUGGGCACUUUCUUGUCU | 1157 | AIACAAGAAAGUGCCCAUUUG | 1158 |
| INI-093 | CACUUUCUUGUCUGAGACU | 1159 | AIUCUCAGACAAGAAAGUGCC | 1160 |
| INI-094 | ACUUUCUUGUCUGAGACUC | 1161 | GAIUCUCAGACAAGAAAGUGC | 1162 |
| INI-095 | CUUUCUUGUCUGAGACUCU | 1163 | AIAGUCUCAGACAAGAAAGUG | 1164 |
| INI-096 | UGUCUGAGACUCUGGCUUA | 1165 | UAAICCAGAGUCUCAGACAAG | 1166 |
| INI-097 | UCUGAGACUCUGGCUUAUU | 1167 | AAUAAICCAGAGUCUCAGACA | 1168 |
| INI-098 | UUAUUCCAGGUUGGCUGAU | 1169 | AUCAICCAACCUGGAAUAAGC | 1170 |
| INI-099 | GGGAGAUGGGUAAAGCGUU | 1171 | AACICUUUACCCAUCUCCCAA | 1172 |
| INI-100 | GGAGAUGGGUAAAGCGUUU | 1173 | AAACICUUUACCCAUCUCCCA | 1174 |
| INI-101 | GGGUAAAGCGUUUCUUCUA | 1175 | UAIAAGAAACGCUUUACCCAU | 1176 |
| INI-102 | GGUAAAGCGUUUCUUCUAA | 1177 | UUAIAAGAAACGCUUUACCCA | 1178 |
| INI-103 | GUAAAGCGUUUCUUCUAAA | 1179 | UUUAIAAGAAACGCUUUACCC | 1180 |
| INI-104 | UAAAGCGUUUCUUCUAAAG | 1181 | CUUUAIAAGAAACGCUUUACC | 1182 |
| INI-105 | GUUUCUUCUAAAGGGGUCU | 1183 | AIACCCCUUUAGAAGAAACGC | 1184 |
| INI-106 | UUUCUUCUAAAGGGGUCUA | 1185 | UAIACCCCUUUAGAAGAAACG | 1186 |
| INI-107 | GGUCUACCCAGAAAGCAUG | 1187 | CAUICUUUCUGGGUAGACCCC | 1188 |
| INI-108 | UCUACCCAGAAAGCAUGAU | 1189 | AUCAUICUUUCUGGGUAGACC | 1190 |
| INI-109 | CUACCCAGAAAGCAUGAUU | 1191 | AAUCAUICUUUCUGGGUAGAC | 1192 |
| INI-110 | UACCCAGAAAGCAUGAUUU | 1193 | AAAUCAUICUUUCUGGGUAGA | 1194 |
| INI-111 | CCAGAAAGCAUGAUUUCCU | 1195 | AIGAAAUCAUGCUUUCUGGGU | 1196 |
| INI-112 | AAGCAUGAUUUCCUGCCCU | 1197 | AIGGCAGGAAAUCAUGCUUUC | 1198 |
| INI-113 | AUGAUUUCCUGCCCUAAGU | 1199 | ACUUAIGGCAGGAAAUCAUGC | 1200 |
| INI-114 | AAGGCAGAGAAAAAUUACU | 1201 | AIUAAUUUUUCUCUGCCUUCC | 1202 |
| INI-115 | AGGCAGAGAAAAAUUACUU | 1203 | AAIUAAUUUUUCUCUGCCUUC | 1204 |
| INI-116 | GGAGGAGGAAGCAGAUAGA | 1205 | UCUAUCUICUUCCUCCUCCCC | 1206 |
| INI-117 | AGGGCUGUUGAGGUACCUU | 1207 | AAIGUACCUCAACAGCCCUUA | 1208 |
| INI-118 | GGCUGUUGAGGUACCUUAA | 1209 | UUAAIGUACCUCAACAGCCCU | 1210 |
| INI-119 | GAAACAGGAGUCAGGAAAA | 1211 | UUUUCCUIACUCCUGUUUCUG | 1212 |
| INI-120 | GUCAGGAAAAUGAGGCACU | 1213 | AIUGCCUCAUUUUCCUGACUC | 1214 |
| INI-121 | UAAGAAGUUCCCUGGUUUU | 1215 | AAAACCAIGGAACUUCUUA | 1216 |
| INI-122 | CACUGGGAGACAAGCAUUU | 1217 | AAAUICUUGUCUCCCAGUG | 1218 |
| INI-123 | ACUGGGAGACAAGCAUUUA | 1219 | UAAAUICUUGUCUCCCAGU | 1220 |
| INI-124 | CUGGGAGACAAGCAUUUAU | 1221 | AUAAAUICUUGUCUCCCAG | 1222 |
| INI-125 | UGGGAGACAAGCAUUUAUA | 1223 | UAUAAAUICUUGUCUCCCA | 1224 |
| INI-126 | GGAGACAAGCAUUUAUACU | 1225 | AIUAUAAAUGCUUGUCUCC | 1226 |
| INI-127 | GAGACAAGCAUUUAUACUU | 1227 | AAIUAUAAAUGCUUGUCUC | 1228 |
| INI-128 | AGACAAGCAUUUAUACUUU | 1229 | AAAIUAUAAAUGCUUGUCU | 1230 |
| INI-129 | ACAAGCAUUUAUACUUUCU | 1231 | AIAAAGUAUAAAUGCUUGU | 1232 |
| INI-130 | CAAGCAUUUAUACUUUCUU | 1233 | AAIAAAGUAUAAAUGCUUG | 1234 |
| INI-131 | GCAUUUAUACUUUCUUUCU | 1235 | AIAAAGAAAGUAUAAAUGC | 1236 |
| INI-132 | CAUUUAUACUUUCUUUCUU | 1237 | AAIAAAGAAAGUAUAAAUG | 1238 |
| INI-133 | GGAGAAAGAAAAUCAACAA | 1239 | UUIUUGAUUUUCUUUCUCC | 1240 |
| INI-134 | AGAAAGAAAAUCAACAAAU | 1241 | AUUUIUUGAUUUUCUUUCU | 1242 |
| INI-135 | UCAACAAAUGUGAGUCAUA | 1243 | UAUIACUCACAUUUGUUGA | 1244 |
| INI-136 | CAACAAAUGUGAGUCAUAA | 1245 | UUAUIACUCACAUUUGUUG | 1246 |
| INI-137 | CAAAUGUGAGUCAUAAAGA | 1247 | UCUUUAUIACUCACAUUUG | 1248 |
| INI-138 | AUGGUCCAGAGCAACAGUU | 1249 | AACUIUUGCUCUGGACCAU | 1250 |
| INI-139 | GUCCAGAGCAACAGUUCUU | 1251 | AAIAACUGUUGCUCUGGAC | 1252 |
| INI-140 | CAGAGCAACAGUUCUUCAA | 1253 | UUIAAGAACUGUUGCUCUG | 1254 |
| INI-141 | GAGCAACAGUUCUUCAAGU | 1255 | ACUUIAAGAACUGUUGCUC | 1256 |
| INI-142 | ACAGUUCUUCAAGUGUACU | 1257 | AIUACACUUGAAGAACUGU | 1258 |
| INI-143 | GUGUACUCUGUAGGCUUCU | 1259 | AIAAGCCUACAGAGUACAC | 1260 |
| INI-144 | GCUUCUGGGAGGUCCCUUU | 1261 | AAAIGGACCUCCCAGAAGC | 1262 |
| INI-145 | UCAGGGGUGUCCACAAAGU | 1263 | ACUUUIUGGACACCCCUGA | 1264 |
| INI-146 | GGGUGUCCACAAAGUCAAA | 1265 | UUUIACUUUGUGGACACCC | 1266 |
| INI-147 | GGUGUCCACAAAGUCAAAG | 1267 | CUUUIACUUUGUGGACACC | 1268 |
| INI-148 | UGUCCACAAAGUCAAAGCU | 1269 | AICUUUGACUUUGUGGACA | 1270 |
| INI-149 | UCCACAAAGUCAAAGCUAU | 1271 | AUAICUUUGACUUUGUGGA | 1272 |
| INI-150 | CCACAAAGUCAAAGCUAUU | 1273 | AAUAICUUUGACUUUGUGG | 1274 |
| INI-151 | CACAAAGUCAAAGCUAUUU | 1275 | AAAUAICUUUGACUUUGUG | 1276 |
| INI-152 | ACAAAGUCAAAGCUAUUUU | 1277 | AAAAUAICUUUGACUUUGU | 1278 |
| INI-153 | AGUCAAAGCUAUUUUCAUA | 1279 | UAUIAAAAUAGCUUUGACU | 1280 |
| INI-154 | GUCAAAGCUAUUUUCAUAA | 1281 | UUAUIAAAAUAGCUUUGAC | 1282 |
| INI-155 | UCAAAGCUAUUUUCAUAAU | 1283 | AUUAUIAAAAUAGCUUUGA | 1284 |
| INI-156 | CUAUUUUCAUAAUAAUACU | 1285 | AIUAUUAUUAUGAAAAUAG | 1286 |
| INI-157 | AUUUUCAUAAUAAUACUAA | 1287 | UUAIUAUUAUUAUGAAAAU | 1288 |
| INI-158 | CAUAAUAAUACUAACAUGU | 1289 | ACAUIUUAGUAUUAUUAUG | 1290 |
| INI-159 | AUAAUAAUACUAACAUGUU | 1291 | AACAUIUUAGUAUUAUUAU | 1292 |
| INI-160 | ACUAACAUGUUAUUUGCCU | 1293 | AIGCAAAUAACAUGUUAGU | 1294 |
| INI-161 | UAACAUGUUAUUUGCCUUU | 1295 | AAAIGCAAAUAACAUGUUA | 1296 |
| INI-162 | CAUGUUAUUUGCCUUUUGA | 1297 | UCAAAAIGCAAAUAACAUG | 1298 |
| INI-163 | UGCCUUUUGAAUUCUCAUU | 1299 | AAUIAGAAUUCAAAAGGCA | 1300 |
| INI-164 | GCCUUUUGAAUUCUCAUUA | 1301 | UAAUIAGAAUUCAAAAGGC | 1302 |
| INI-165 | CCUUUUGAAUUCUCAUUAU | 1303 | AUAAUIAGAAUUCAAAAGG | 1304 |
| INI-166 | UGAAUUCUCAUUAUCUUAA | 1305 | UUAAIAUAAUGAGAAUUCA | 1306 |
| INI-167 | GAAUUCUCAUUAUCUUAAA | 1307 | UUUAAIAUAAUGAGAAUUC | 1308 |
| INI-168 | AUUCUCAUUAUCUUAAAAU | 1309 | AUUUUAAIAUAAUGAGAAU | 1310 |
| INI-169 | CGUGUGACAUGUGAUUACA | 1311 | UIUAAUCACAUGUCACACG | 1312 |
| INI-170 | GUGUGACAUGUGAUUACAU | 1313 | AUIUAAUCACAUGUCACAC | 1314 |
| INI-171 | UGACAUGUGAUUACAUCAU | 1315 | AUIAUGUAAUCACAUGUCA | 1316 |
| INI-172 | ACAUGUGAUUACAUCAUCU | 1317 | AIAUGAUGUAAUCACAUGU | 1318 |
| INI-173 | CAUGUGAUUACAUCAUCUU | 1319 | AAIAUGAUGUAAUCACAUG | 1320 |
| INI-174 | GUGAUUACAUCAUCUUUCU | 1321 | AIAAAGAUGAUGUAAUCAC | 1322 |
| INI-175 | GAUUACAUCAUCUUUCUGA | 1323 | UCAIAAAGAUGAUGUAAUC | 1324 |
| INI-176 | CAUCAUCUUUCUGACAUCA | 1325 | UIAUGUCAGAAAGAUGAUG | 1326 |
| INI-177 | UCAUCUUUCUGACAUCAUU | 1327 | AAUIAUGUCAGAAAGAUGA | 1328 |
| INI-178 | UCUUUCUGACAUCAUUGUU | 1329 | AACAAUIAUGUCAGAAAGA | 1330 |
| INI-179 | CUUUCUGACAUCAUUGUUA | 1331 | UAACAAUIAUGUCAGAAAG | 1332 |
| INI-180 | UGUUAAUGGAAUGUGUGCU | 1333 | AICACACAUUCCAUUAACA | 1334 |
| INI-181 | GUUAAUGGAAUGUGUGCUU | 1335 | AAICACACAUUCCAUUAAC | 1336 |
| INI-182 | GUGAGGGUCAAGCACAGCU | 1337 | AGCUIUGCUUGACCCUCACAG | 1338 |
| INI-183 | GAGGGUCAAGCACAGCUAU | 1339 | AUAGCUIUGCUUGACCCUCAC | 1340 |
| INI-184 | GGGUCAAGCACAGCUAUCC | 1341 | GGAUAICUGUGCUUGACCCUC | 1342 |
| INI-185 | GGUCAAGCACAGCUAUCCA | 1343 | UGIAUAGCUGUGCUUGACCCU | 1344 |
| INI-186 | GUCAAGCACAGCUAUCCAU | 1345 | AUGIAUAGCUGUGCUUGACCC | 1346 |
| INI-187 | UCAAGCACAGCUAUCCAUC | 1347 | GAUGIAUAGCUGUGCUUGACC | 1348 |
| INI-188 | CAAGCACAGCUAUCCAUCA | 1349 | UGAUIGAUAGCUGUGCUUGAC | 1350 |
| INI-189 | AAGCACAGCUAUCCAUCAG | 1351 | CUGAUIGAUAGCUGUGCUUGA | 1352 |
| INI-190 | AGCACAGCUAUCCAUCAGA | 1353 | UCUGAUIGAUAGCUGUGCUUG | 1354 |
| INI-191 | GCACAGCUAUCCAUCAGAU | 1355 | AUCUGAUIGAUAGCUGUGCUU | 1356 |
| INI-192 | AUCCAUCAGAUGAUCUACU | 1357 | AGUAIAUCAUCUGAUGGAUAG | 1358 |
| INI-193 | UCCAUCAGAUGAUCUACUU | 1359 | AAGUAIAUCAUCUGAUGGAUA | 1360 |
| INI-194 | CCAUCAGAUGAUCUACUUU | 1361 | AAAGUAIAUCAUCUGAUGGAU | 1362 |
| INI-195 | CAUCAGAUGAUCUACUUUC | 1363 | GAAAGUAIAUCAUCUGAUGGA | 1364 |
| INI-196 | AUCAGAUGAUCUACUUUCA | 1365 | UGAAAIUAGAUCAUCUGAUGG | 1366 |
| INI-197 | CAGAUGAUCUACUUUCAGC | 1367 | GCUGAAAIUAGAUCAUCUGAU | 1368 |
| INI-198 | GAUGAUCUACUUUCAGCCU | 1369 | AGICUGAAAGUAGAUCAUCUG | 1370 |
| INI-199 | AUGAUCUACUUUCAGCCUU | 1371 | AAGICUGAAAGUAGAUCAUCU | 1372 |
| INI-200 | UUCCUGAGUCCCAGACAAU | 1373 | AUUGUCUIGGACUCAGGAAGG | 1374 |
| INI-201 | CAUUGGCCCCCAGCAAUCA | 1375 | UGAUUICUGGGGGCCAAUGAG | 1376 |
| INI-202 | AGCUAGCCAAGCAGCAAAU | 1377 | AUUUGCUICUUGGCUAGCUCC | 1378 |
| INI-203 | UAGCCAAGCAGCAAAUCCU | 1379 | AGIAUUUGCUGCUUGGCUAGC | 1380 |
| INI-204 | UCGUCCCAGAAUAACUCAU | 1381 | AUGAIUUAUUCUGGGACGACU | 1382 |
| INI-205 | GAGGUCAUCAGCUUUGCUA | 1383 | UAGCAAAICUGAUGACCUCCU | 1384 |
| INI-206 | GGUCAUCAGCUUUGCUACU | 1385 | AGUAICAAAGCUGAUGACCUC | 1386 |
| INI-207 | GUCAUCAGCUUUGCUACUG | 1387 | CAGUAICAAAGCUGAUGACCU | 1388 |
| INI-208 | UCAUCAGCUUUGCUACUGU | 1389 | ACAGUAICAAAGCUGAUGACC | 1390 |
| INI-209 | CAUCAGCUUUGCUACUGUC | 1391 | GACAGUAICAAAGCUGAUGAC | 1392 |
| INI-210 | GCUUUGCUACUGUCACAGA | 1393 | UCUGUIACAGUAGCAAAGCUG | 1394 |
| INI-211 | UUUGCUACUGUCACAGACU | 1395 | AGUCUIUGACAGUAGCAAAGC | 1396 |
| INI-212 | ACUGUCACAGACUCCACUU | 1397 | AAGUIGAGUCUGUGACAGUAG | 1398 |
| INI-213 | ACCCUUCCUGGCACUCUUU | 1399 | AAAGAIUGCCAGGAAGGGUGG | 1400 |
| INI-214 | CUUCCUGGCACUCUUUGCU | 1401 | AGCAAAIAGUGCCAGGAAGGG | 1402 |
| INI-215 | GCUGGCAUACCUUAACUCU | 1403 | AGAIUUAAGGUAUGCCAGCCC | 1404 |
| INI-216 | AGUCUGGUGUCCUGAAACU | 1405 | AGUUUCAIGACACCAGACUUC | 1406 |
| INI-217 | CUGGUGUCCUGAAACUGCA | 1407 | UGCAIUUUCAGGACACCAGAC | 1408 |
| INI-218 | GUGUCCUGAAACUGCAACU | 1409 | AGUUICAGUUUCAGGACACCA | 1410 |
| INI-219 | UGUCCUGAAACUGCAACUA | 1411 | UAGUUICAGUUUCAGGACACC | 1412 |
| INI-220 | GUCCUGAAACUGCAACUAG | 1413 | CUAGUUICAGUUUCAGGACAC | 1414 |
| INI-221 | AGAAGGCAACAGCACAGUU | 1415 | AACUGUICUGUUGCCUUCUAG | 1416 |
| INI-222 | AGGCAACAGCACAGUUACU | 1417 | AGUAACUIUGCUGUUGCCUUC | 1418 |
| INI-223 | AGAGCUUAAGAUCCGAGCC | 1419 | GGCUCIGAUCUUAAGCUCUAG | 1420 |
| INI-224 | GAGCUUAAGAUCCGAGCCA | 1421 | UGICUCGGAUCUUAAGCUCUA | 1422 |
| INI-225 | GCUUAAGAUCCGAGCCAAU | 1423 | AUUGICUCGGAUCUUAAGCUC | 1424 |
| INI-226 | UGUUGCAGGCGAGACCAUU | 1425 | AAUGIUCUCGCCUGCAACAUA | 1426 |
| INI-227 | CGAGACCAUUACGUAGACU | 1427 | AGUCUACIUAAUGGUCUCGCC | 1428 |
| INI-228 | AUUACGUAGACUUCCAGGA | 1429 | UCCUGIAAGUCUACGUAAUGG | 1430 |
| INI-229 | AUUGCUGCCUCUUUCCAUU | 1431 | AAUGIAAAGAGGCAGCAAUGC | 1432 |
| INI-230 | CUUUCCAUUCUGCCGUCUU | 1433 | AAGACIGCAGAAUGGAAAGAG | 1434 |
| INI-231 | CCUCCUCAAAGCCAACAAU | 1435 | AUUGUUIGCUUUGAGGAGGCU | 1436 |
| INI-232 | CCUCAAAGCCAACAAUCCU | 1437 | AGIAUUGUUGGCUUUGAGGAG | 1438 |
| INI-233 | CUCAAAGCCAACAAUCCUU | 1439 | AAGIAUUGUUGGCUUUGAGGA | 1440 |
| INI-234 | CCUGCCAGUACCUCCUGUU | 1441 | AACAGIAGGUACUGGCAGGCC | 1442 |
| INI-235 | AGACGGAUGUGCCAGAUAU | 1443 | AUAUCUGICACAUCCGUCUUG | 1444 |
| INI-236 | UUUGGAGUGAAGAGACCAA | 1445 | UUGIUCUCUUCACUCCAAAGC | 1446 |
| INI-237 | CCUGGCAAUAUGACUCACU | 1447 | AGUIAGUCAUAUUGCCAGGUG | 1448 |
| INI-238 | CUGGCAAUAUGACUCACUU | 1449 | AAGUIAGUCAUAUUGCCAGGU | 1450 |
| INI-239 | AAUGGGCACUUUCUUGUCU | 1451 | AGACAAIAAAGUGCCCAUUUG | 1452 |
| INI-240 | CACUUUCUUGUCUGAGACU | 1453 | AGUCUCAIACAAGAAAGUGCC | 1454 |
| INI-241 | CUUUCUUGUCUGAGACUCU | 1455 | AGAIUCUCAGACAAGAAAGUG | 1456 |
| INI-242 | UGUCUGAGACUCUGGCUUA | 1457 | UAAGCCAIAGUCUCAGACAAG | 1458 |
| INI-243 | GGGUAAAGCGUUUCUUCUA | 1459 | UAGAAIAAACGCUUUACCCAU | 1460 |
| INI-244 | GGUAAAGCGUUUCUUCUAA | 1461 | UUAGAAIAAACGCUUUACCCA | 1462 |
| INI-245 | GUAAAGCGUUUCUUCUAAA | 1463 | UUUAGAAIAAACGCUUUACCC | 1464 |
| INI-246 | CCAGAAAGCAUGAUUUCCU | 1465 | AGIAAAUCAUGCUUUCUGGGU | 1466 |
| INI-247 | AAGCAUGAUUUCCUGCCCU | 1467 | AGIGCAGGAAAUCAUGCUUUC | 1468 |
| INI-248 | AUGAUUUCCUGCCCUAAGU | 1469 | ACUUAGIGCAGGAAAUCAUGC | 1470 |
| INI-249 | AGGGCUGUUGAGGUACCUU | 1471 | AAGIUACCUCAACAGCCCUUA | 1472 |
| INI-250 | GGCUGUUGAGGUACCUUAA | 1473 | UUAAGIUACCUCAACAGCCCU | 1474 |
| INI-251 | GUCAGGAAAAUGAGGCACU | 1475 | AGUICCUCAUUUUCCUGACUC | 1476 |
| INI-252 | ACAAGCAUUUAUACUUUCU | 1477 | AGAAAIUAUAAAUGCUUGU | 1478 |
| INI-253 | CAAGCAUUUAUACUUUCUU | 1479 | AAGAAAIUAUAAAUGCUUG | 1480 |
| INI-254 | GCAUUUAUACUUUCUUUCU | 1481 | AGAAAIAAAGUAUAAAUGC | 1482 |
| INI-255 | CAUUUAUACUUUCUUUCUU | 1483 | AAGAAAIAAAGUAUAAAUG | 1484 |
| INI-256 | GGAGAAAGAAAAUCAACAA | 1485 | UUGUUIAUUUUCUUUCUCC | 1486 |
| INI-257 | AGAAAGAAAAUCAACAAAU | 1487 | AUUUGUUIAUUUUCUUUCU | 1488 |
| INI-258 | AUGGUCCAGAGCAACAGUU | 1489 | AACUGUUICUCUGGACCAU | 1490 |
| INI-259 | GUCCAGAGCAACAGUUCUU | 1491 | AAGAACUIUUGCUCUGGAC | 1492 |
| INI-260 | CAGAGCAACAGUUCUUCAA | 1493 | UUGAAIAACUGUUGCUCUG | 1494 |
| INI-261 | GAGCAACAGUUCUUCAAGU | 1495 | ACUUGAAIAACUGUUGCUC | 1496 |
| INI-262 | GUGUACUCUGUAGGCUUCU | 1497 | AGAAICCUACAGAGUACAC | 1498 |
| INI-263 | GCUUCUGGGAGGUCCCUUU | 1499 | AAAGIGACCUCCCAGAAGC | 1500 |
| INI-264 | UCAGGGGUGUCCACAAAGU | 1501 | ACUUUGUIGACACCCCUGA | 1502 |
| INI-265 | UGUCCACAAAGUCAAAGCU | 1503 | AGCUUUIACUUUGUGGACA | 1504 |
| INI-266 | ACUAACAUGUUAUUUGCCU | 1505 | AGICAAAUAACAUGUUAGU | 1506 |
| INI-267 | UAACAUGUUAUUUGCCUUU | 1507 | AAAGICAAAUAACAUGUUA | 1508 |
| INI-268 | CAUGUUAUUUGCCUUUUGA | 1509 | UCAAAAGICAAAUAACAUG | 1510 |
| INI-269 | UGCCUUUUGAAUUCUCAUU | 1511 | AAUGAIAAUUCAAAAGGCA | 1512 |
| INI-270 | GCCUUUUGAAUUCUCAUUA | 1513 | UAAUGAIAAUUCAAAAGGC | 1514 |
| INI-271 | CCUUUUGAAUUCUCAUUAU | 1515 | AUAAUGAIAAUUCAAAAGG | 1516 |
| INI-272 | UGACAUGUGAUUACAUCAU | 1517 | AUGAUIUAAUCACAUGUCA | 1518 |
| INI-273 | ACAUGUGAUUACAUCAUCU | 1519 | AGAUIAUGUAAUCACAUGU | 1520 |
| INI-274 | CAUGUGAUUACAUCAUCUU | 1521 | AAGAUIAUGUAAUCACAUG | 1522 |
| INI-275 | GUGAUUACAUCAUCUUUCU | 1523 | AGAAAIAUGAUGUAAUCAC | 1524 |
| INI-276 | GAUUACAUCAUCUUUCUGA | 1525 | UCAGAAAIAUGAUGUAAUC | 1526 |
| INI-277 | CAUCAUCUUUCUGACAUCA | 1527 | UGAUIUCAGAAAGAUGAUG | 1528 |
| INI-278 | UCAUCUUUCUGACAUCAUU | 1529 | AAUGAUIUCAGAAAGAUGA | 1530 |
| INI-279 | GUGAGGGUCAAGCACAGCU | 1531 | AGCUGUICUUGACCCUCACAG | 1532 |
| INI-280 | GGUCAAGCACAGCUAUCCA | 1533 | UGGAUAICUGUGCUUGACCCU | 1534 |
| INI-281 | GUCAAGCACAGCUAUCCAU | 1535 | AUGGAUAICUGUGCUUGACCC | 1536 |
| INI-282 | CAAGCACAGCUAUCCAUCA | 1537 | UGAUGIAUAGCUGUGCUUGAC | 1538 |
| INI-283 | AAGCACAGCUAUCCAUCAG | 1539 | CUGAUGIAUAGCUGUGCUUGA | 1540 |
| INI-284 | AGCACAGCUAUCCAUCAGA | 1541 | UCUGAUGIAUAGCUGUGCUUG | 1542 |
| INI-285 | GAUGAUCUACUUUCAGCCU | 1543 | AGGCUIAAAGUAGAUCAUCUG | 1544 |
| INI-286 | AUGAUCUACUUUCAGCCUU | 1545 | AAGGCUIAAAGUAGAUCAUCU | 1546 |
| INI-287 | UAGCCAAGCAGCAAAUCCU | 1547 | AGGAUUUICUGCUUGGCUAGC | 1548 |
| INI-288 | UUUGCUACUGUCACAGACU | 1549 | AGUCUGUIACAGUAGCAAAGC | 1550 |
| INI-289 | ACUGUCACAGACUCCACUU | 1551 | AAGUGIAGUCUGUGACAGUAG | 1552 |
| INI-290 | ACCCUUCCUGGCACUCUUU | 1553 | AAAGAGUICCAGGAAGGGUGG | 1554 |
| INI-291 | GUGUCCUGAAACUGCAACU | 1555 | AGUUGCAIUUUCAGGACACCA | 1556 |
| INI-292 | AGAGCUUAAGAUCCGAGCC | 1557 | GGCUCGIAUCUUAAGCUCUAG | 1558 |
| INI-293 | GAGCUUAAGAUCCGAGCCA | 1559 | UGGCUCIGAUCUUAAGCUCUA | 1560 |
| INI-294 | CUUUCCAUUCUGCCGUCUU | 1561 | AAGACGICAGAAUGGAAAGAG | 1562 |
| INI-295 | CCUCCUCAAAGCCAACAAU | 1563 | AUUGUUGICUUUGAGGAGGCU | 1564 |
| INI-296 | CCUCAAAGCCAACAAUCCU | 1565 | AGGAUUIUUGGCUUUGAGGAG | 1566 |
| INI-297 | CUCAAAGCCAACAAUCCUU | 1567 | AAGGAUUIUUGGCUUUGAGGA | 1568 |
| INI-298 | CCUGCCAGUACCUCCUGUU | 1569 | AACAGGAIGUACUGGCAGGCC | 1570 |
| INI-299 | CCUGGCAAUAUGACUCACU | 1571 | AGUGAIUCAUAUUGCCAGGUG | 1572 |
| INI-300 | CUGGCAAUAUGACUCACUU | 1573 | AAGUGAIUCAUAUUGCCAGGU | 1574 |
| INI-301 | AAGCAUGAUUUCCUGCCCU | 1575 | AGGICAGGAAAUCAUGCUUUC | 1576 |
| INI-302 | AUGAUUUCCUGCCCUAAGU | 1577 | ACUUAGGICAGGAAAUCAUGC | 1578 |
| INI-303 | GCUUCUGGGAGGUCCCUUU | 1579 | AAAGGIACCUCCCAGAAGC | 1580 |
| INI-304 | ACAUGUGAUUACAUCAUCU | 1581 | AGAUGAUIUAAUCACAUGU | 1582 |
| INI-305 | ACUGUCACAGACUCCACUU | 1583 | AAGUGGAIUCUGUGACAGUAG | 1584 |
| INI-306 | GAGCUUAAGAUCCGAGCCA | 1585 | UGGCUCGIAUCUUAAGCUCUA | 1586 |
| INI-307 | AAGCAUGAUUUCCUGCCCU | 1587 | AGGGCAIGAAAUCAUGCUUUC | 1588 |
| INI-308 | AAGCAUGAUUUCCUGCCCU | 1589 | AGGGCAGIAAAUCAUGCUUUC | 1590 |
| IN-489 | mC*mA*mUmGmUmUfAfUfUm | 1759 | mU*dC*fAmAmAmAmGmGmCmA | 1760 |
| | UmGmCmCmUmUmUmU*mG*m | | mAmAmUfAmAmCmAmUmG*mU | |
| | A | | *mU | |
| IN-490 | mC*mA*mUmGmUmUfAfUfUm | 1761 | mU*dC*fAfAmAmAmGmGmCmA | 1762 |
| | UmGmCmCmUmUmUmU*mG*m | | mAmAmUfAmAmCmAmUmG*mU | |
| | A | | *mU | |
| IN-491 | mC*mA*mUmGmUmUfAfUfUm | 1763 | mU*dC*fAfAfAmAmGmGmCmAm | 1764 |
| | UmGmCmCmUmUmUmU*mG*m | | AmAmUfAmAmCmAmUmG*mU* | |
| | A | | mU | |
| IN-492 | mC*mA*mUmGmUmUfAfUfUm | 1765 | mU*dC*mAmAdAmAdGmGmCmA | 1766 |
| | UmGmCmCmUmUmUmU*mG*m | | mAdAmUfAmAmCmAmUmG*mU | |
| | A | | *mU | |
| IN-493 | mC*mA*fUmGmUmUfAfUfUmU | 1767 | mU*dC*mAmAdAmAdGmGmCmA | 1768 |
| | mGmCmCmUmUmUmU*mG*mA | | mAdAmUfAmAmCmAmUmG*mU | |
| | | | *mU | |
| IN-494 | mA*mA*mCmAmUmGmUmUfAf | 1769 | mU*dC*mAmAdAmAdGmGmCmA | 1770 |
| | UfUmUmGmCmCmUmUmUmU* | | mAdAmUfAmAmCmAmUmG*mU | |
| | mG*mA | | *mU | |
| IN-495 | irmA*mA*mCmAmUmGmUmUf | 1771 | mU*dC*mAmAdAmAdGmGmCmA | 1772 |
| | AfUfUmUmGmCmCmUmUmUm | | mAdAmUfAmAmCmAmUmG*mU | |
| | U*mG*mA | | *mU | |
| IN-496 | irmC*mA*mUmGmUmUfAfUfU | 1773 | mU*dC*mAmAdAmAdGmGmCmA | 1774 |
| | mUmGmCmCmUmUmUmU*mG* | | mAdAmUfAmAmCmAmUmG*mU | |
| | mA | | *mU | |
| IN-497 | irmA*mA*mCmAmUmGmUmUf | 1775 | mU*dC*mAmAdAmAdGmGmCmA | 1776 |
| | AfUfUmUmGmCmCmUmUmUm | | mAdAmUfAmAmCmAmUmG*mU | |
| | U*mG*mA | | *mU | |
| IN-498 | mC*mA*mUmGmUmUfAfUfUm | 1777 | mU*fC*mAmAmAmAmGmGmCm | 1778 |
| | UmGmCmCmUmUmUmU*mG*ir | | AmAmAmUfAmAmCmAmUmG*m | |
| | mA | | U*mU | |
| IN-499 | mG*mC*mUmGmAmGfAfCfUm | 1779 | mA*fA*mUmAmAmGmCmCmAm | 1780 |
| | CmUmGmGmCmUmUmAmUmU | | GmAmGmUfCmUmCmAmGmC*m | |
| | | | C*mA | |
| IN-500 | mG*mC*mUmGmAmGfAfCfUm | 1781 | mA*fA*mUmAmAmGmCmCmAfG | 1782 |
| | CmUmGmGmCmUmUmAmUmU | | mAmGmUfCmUmCmAmGmC*mC | |
| | | | *mA | |
| IN-501 | mG*mC*mUmGmAmGfAfCfUm | 1783 | mA*fA*mUmAmAmGmCmCmAm | 1784 |
| | CmUmGmGmCmUmUmAmUmU | | GmAmGmUfCmUmCmAmGmG*m | |
| | | | C*mA | |
| IN-502 | irmG*mC*mUmGmAmGfAfCfUm | 1785 | mA*fA*mUmAmAmGmCmCmAm | 1786 |
| | CmUmGmGmCmUmUmAmUmU | | GmAmGmUfCmUmCmAmGmC*m | |
| | | | C*mA | |
| IN-503 | mG*mC*mUmGmAmGfAfCfUm | 1787 | mA*fA*mUmAmAmImCmCmAmG | 1788 |
| | CmUmGmGmCmUmUmAmUmU | | mAmGmUfCmUmCmAmGmC*mC | |
| | | | *mA | |
| IN-504 | irmG*mC*mUmGmAmGfAfCfUm | 1789 | mA*fA*mUmAmAmImCmCmAmG | 1790 |
| | CmUmGmGmCmUmUmAmUmU | | mAmGmUfCmUmCmAmGmC*mC | |
| | | | *mA | |
| IN-505 | irmG*mC*mUmGmAmGfAfCfUm | 1791 | mA*fA*mUmAmAmGmCmCmAm | 1792 |
| | CmUmGmGmCmUmUmA*mU*m | | GmAmGmUfCmUmCmAmGmC*m | |
| | U | | C*mA | |
| IN-506 | mU*mG*mGmCmUmGmAmGfAf | 1793 | mA*fA*mUmAmAmGmCmCmAm | 1794 |
| | CfUmCmUmGmGmCmUmUmA* | | GmAmGmUfCmUmCmAmGmC*m | |
| | mU*mU | | C*mA | |
| IN-507 | irmU*mG*mGmCmUmGmAmGf | 1795 | mA*fA*mUmAmAmGmCmCmAm | 1796 |
| | AfCfUmCmUmGmGmCmUmUm | | GmAmGmUfCmUmCmAmGmC*m | |
| | A*mU*mU | | C*mA | |
| IN-508 | mU*mG*mUmCmUmGmAmGfAf | 1797 | mA*fA*mUmAmAmGmCmCmAm | 1798 |
| | CfUmCmUmGmGmCmUmUmA* | | GmAmGmUfCmUmCmAmGmA*m | |
| | mU*mU | | C*mA | |
| IN-509 | mG*mC*mUmGmAmGfAfCfUm | 1799 | mA*fA*mUmAmAmGmCmCmAm | 1800 |
| | CmUmGmGmCmUmUmA*mU*ir | | GmAmGmUfCmUmCmAmGmC*m | |
| | mU | | C*mA | |
| IN-510 | mC*mC*mAmUmCmAfGfCfUmU | 1801 | mA*fC*mAmGmUmAmGmCfAmA | 1802 |
| | mUmGmCmUmAmCmUmGmU | | mAfGmCfUmGmAmUmGmG*mU* | |
| | | | mU | |
| IN-511 | mC*mC*mAmUmCmAfGfCfUmU | 1803 | mA*fC*mAmGmUmAmGmCfAmA | 1804 |
| | mUmGmCmUmAmCmUmGmU | | mAfGmCmUmGmAmUmGmG*mU | |
| | | | *mU | |
| IN-512 | mC*mC*mAmUmCmAfGfCfUmU | 1805 | mA*fC*mAmGmUmAmGfCfAmA | 1806 |
| | mUmGmCmUmAmCmUmGmU | | mAfGmCfUmGmAmUmGmG*mU* | |
| | | | mU | |
| IN-513 | mC*mC*mAmUmCmAfGfCfUmU | 1807 | mA*fC*mAmGmUmAmGfCfAmA | 1808 |
| | mUmGmCmUmAmCmUmGmU | | mAfGmCmUmGmAmUmGmG*mU | |
| | | | *mU | |
| IN-514 | mC*mC*mAmUmCmAfGfCfUmU | 1809 | mA*fC*mAmGmUmAmGmCfAmA | 1810 |
| | mUmGmCmUmAmCmU*mG*mU | | mAfGmCfUmGmAmUmGmG*mU* | |
| | | | mU | |
| IN-515 | irmC*mC*mAmUmCmAfGfCfUm | 1811 | mA*fC*mAmGmUmAmGmCfAmA | 1812 |
| | UmUmGmCmUmAmCmU*mG*m | | mAfGmCfUmGmAmUmGmG*mU* | |
| | U | | mU | |
| IN-516 | mA*mA*mCmCmAmUmCmAfGf | 1813 | mA*fC*mAmGmUmAmGmCfAmA | 1814 |
| | CfUmUmUmGmCmUmAmCmU* | | mAfGmCfUmGmAmUmGmG*mU* | |
| | mG*mU | | mU | |
| IN-517 | irmA*mA*mCmCmAmUmCmAf | 1815 | mA*fC*mAmGmUmAmGmCfAmA | 1816 |
| | GfCfUmUmUmGmCmUmAmCm | | mAfGmCfUmGmAmUmGmG*mU* | |
| | U*mG*mU | | mU | |
| IN-518 | CAUGUGAUUACAUCAUCUU | 1817 | AAGAUIAUGUAAUCACAUGUC | 1818 |
| IN-519 | mC*mA*mUmGfUfGfAmUmUm | 1819 | mA*fA*mGmAmUmImAmUmGmU | 1820 |
| | AmCmAmUmCmAmUmCmUmU | | fAmAmUmCmAmCmAmUmG*mU | |
| | | | *mC | |
| IN-520 | mC*mA*mUmGfUfGfAmUmUm | 1821 | mA*fA*mGmAfUmImAmUmGmUf | 1822 |
| | AmCmAmUmCmAmUmCmUmU | | AmAmUmCmAmCmAmUmG*mU* | |
| | | | mC | |
| IN-521 | mC*mA*mUmGfUfGfAmUmUm | 1823 | mA*fA*mGmAfUmIfAmUmGmUf | 1824 |
| | AmCmAmUmCmAmUmCmUmU | | AmAmUmCmAmCmAmUmG*mU* | |
| | | | mC | |
| IN-522 | mC*mA*mUmGfUfGfAmUmUm | 1825 | mA*dA*mGmAdUmIdAmUmGmUf | 1826 |
| | AmCmAmUmCmAmUmCmUmU | | AdAmUmCmAmCmAmUmG*mU* | |
| | | | mC | |
| IN-523 | mC*mA*mUmGmUmGmAmUmU | 1827 | mA*fA*mGmAmUmImAmUmGmU | 1828 |
| | mAmCmAmUmCmAmUmCmUm | | fAmAmUmCmAmCmAmUmG*mU | |
| | U | | *mC | |
| IN-524 | mC*mA*mUmGfUfGfAmUmUm | 1829 | mA*fA*mGmAmUmGmAmUmGm | 1830 |
| | AmCmAmUmCmAmUmCmUmU | | UfAmAmUmCmAmCmAmUmG*m | |
| | | | U*mC | |
| IN-525 | mC*mA*mUmGfUfGfAmUmUm | 1831 | mA*fA*mGmAmUmImAmUmGmU | 1832 |
| | AmCmAmUmCmAmUmC*mU*m | | fAmAmUmCmAmCmAmUmG*mU | |
| | U | | *mC | |
| IN-526 | irmC*mA*mUmGfUfGfAmUmU | 1833 | mA*fA*mGmAmUmImAmUmGmU | 1834 |
| | mAmCmAmUmCmAmUmC*mU* | | fAmAmUmCmAmCmAmUmG*mU | |
| | mU | | *mC | |
| IN-527 | mG*mA*mCmAmUmGfUfGfAm | 1835 | mA*fA*mGmAmUmImAmUmGmU | 1836 |
| | UmUmAmCmAmUmCmAmUmC | | fAmAmUmCmAmCmAmUmG*mU | |
| | *mU*mU | | *mC | |
| IN-528 | irmG*mA*mCmAmUmGfUfGfA | 1837 | mA*fA*mGmAmUmImAmUmGmU | 1838 |
| | mUmUmAmCmAmUmCmAmUm | | fAmAmUmCmAmCmAmUmG*mU | |
| | C*mU*mU | | *mC | |
| IN-529 | mA*mG*mUmCmAmAmAmGmC | 1839 | mU*fA*mUmGmAfAmAfAmUmA | 1840 |
| | mUmAmUmUmUmUmCmA*mU* | | mGmCmUfUmUmGmAmCmU*mU | |
| | mA | | *mU | |
| IN-530 | mA*mG*mUmCmAmAmAmGmC | 1841 | mU*fA*mUmGfAfAmAfAmUmAm | 1842 |
| | mUmAmUmUmUmUmCmA*mU* | | GmCmUfUmUmGmAmCmU*mU* | |
| | mA | | mU | |
| IN-531 | mA*mG*mUmCmAmAmAmGmC | 1843 | mU*fA*mUmGmAfAfAfAmUmAm | 1844 |
| | mUmAmUmUmUmUmCmA*mU* | | GmCmUfUmUmGmAmCmU*mU* | |
| | mA | | mU | |
| IN-532 | mA*mG*mUmCmAmAmAmGmC | 1845 | mU*fA*mUmGfAfAfAfAmUmAm | 1846 |
| | mUmAmUmUmUmUmCmA*mU* | | GmCmUfUmUmGmAmCmU*mU* | |
| | mA | | mU | |
| IN-533 | mA*mG*mUmCmAmAmAmGmC | 1847 | mU*dA*mUfGdAfAdAfAmUmAm | 1848 |
| | mUmAmUmUmUmUmCmA*mU* | | GdCmUfUmUfGmAfCmU*mU*mU | |
| | mA | | | |
| IN-534 | mC*mG*mUmCmAmAmAmGmC | 1849 | mU*dA*mUfGdAfAdAfAmUmAm | 1850 |
| | mUmAmUmUmUmUmCmA*mU* | | GdCmUfUmUfGmAfCmG*mU*mU | |
| | mA | | | |
| IN-535 | mA*mG*mUmCmAmAfAfGfCm | 1851 | mU*fA*mUmGmAfAmAfAmUmA | 1852 |
| | UmAmUmUmUmUmCmA*mU*m | | mGmCmUfUmUmGmAmCmU*mU | |
| | A | | *mU | |
| IN-536 | mA*mG*mUmCmAmAfAfGfCm | 1853 | mU*dA*mUfGdAfAdAfAmUmAm | 1854 |
| | UmAmUmUmUmUmCmAmUmA | | GdCmUfUmUfGmAfCmU*mU*mU | |
| IN-537 | mA*mG*mUmCmAmAfAfGfCm | 1855 | mU*dA*mUfGdAfAdAfAmUmAm | 1856 |
| | UmAmUmUmUmUmCmAmUmA | | GdCmUfUmUmGmAmCmU*mU*m | |
| | | | U | |
| IN-538 | mA*mG*mUmCmAmAfAfGfCm | 1857 | mU*dA*mUfIdAfAdAfAmUmAmG | 1858 |
| | UmAmUmUmUmUmCmAmUmA | | dCmUfUmUfGmAfCmU*mU*mU | |
| IN-539 | mA*mG*mUmCmAmAfAfGfCm | 1859 | mU*dA*mUfIdAfAdAfAmUmAmG | 1860 |
| | UmAmUmUmUmUmCmAmUmA | | dCmUfUmUmGmAmCmU*mU*mU | |
| IN-540 | mA*mG*mUmCmAmAfAfGfCm | 1861 | mU*dA*mUfGdAfAdAfAmUmAm | 1862 |
| | UmAmUmUmUmUmCmA*mU*m | | GdCmUfUmUfGmAfCmU*mU*mU | |
| | A | | | |
| IN-541 | irmA*mG*mUmCmAmAfAfGfCm | 1863 | mU*dA*mUfGdAfAdAfAmUmAm | 1864 |
| | UmAmUmUmUmUmCmA*mU*m | | GdCmUfUmUfGmAfCmU*mU*mU | |
| | A | | | |
| IN-542 | mA*mA*mAmGmUmCmAmAfAf | 1865 | mU*dA*mUfGdAfAdAfAmUmAm | 1866 |
| | GfCmUmAmUmUmUmUmCmA* | | GdCmUfUmUfGmAfCmU*mU*mU | |
| | mU*mA | | | |
| IN-543 | irmA*mA*mAmGmUmCmAmAf | 1867 | mU*dA*mUfGdAfAdAfAmUmAm | 1868 |
| | AfGfCmUmAmUmUmUmUmCm | | GdCmUfUmUfGmAfCmU*mU*mU | |
| | A*mU*mA | | | |
| IN-544 | irmA*mG*mUmCmAmAfAfGfCm | 1869 | mU*dA*mUfGdAfAdAfAmUmAm | 1870 |
| | UmAmUmUmUmUmCmA*mU*m | | GdCmUfUmUmGmAmCmU*mU*m | |
| | A | | U | |
| IN-545 | irmA*mA*mAmGmUmCmAmAf | 1871 | mU*dA*mUfGdAfAdAfAmUmAm | 1872 |
| | AfGfCmUmAmUmUmUmUmCm | | GdCmUfUmUmGmAmCmU*mU*m | |
| | A*mU*mA | | U | |
| IN-546 | mA*mG*mUmCmAmAfAfGfCm | 1873 | mU*fA*mUmGfAfAmAfAmUmAm | 1874 |
| | UmAmUmUmUmUmCmA*mU*ir | | GmCmUfUmUmGmAmCmU*mU* | |
| | mA | | mU | |

The GalNAc conjugates were synthesized and conjugated to a solid support (CPG or PS). The GalNAc conjugated siRNA was synthesized with the GalNAc-CPG or GalNAc-PS by Suzhou Biosyntech Co., Ltd. The synthesis of targeting ligands in Formulae 1-33 was as described in WO2022266753A1. The dsRNAi agent of Formula 5, formed by linking the dsRNA in Table 1 and Table 2 with the targeting ligand, is named “duplex number-L5”.

### Example 2. In Vitro Activity Detection Method of siRNA

### 2.1 Real-Time Quantitative Polymerase Chain Reaction (Rt-PCR)

### 2.1.1 Cell Culture and Transfection

Hep3B cells were cultured at 37° C. using MEM medium (Gibco) supplemented with 10% Fetal Bovine Serum (FBS, Gibco), penicillin, and streptomycin (Gibco) at 5% CO₂. Hep3B cells were resuspended using trypsin digestion after cell growth had nearly covered the whole culture flask. The density of the resuspended cells was adjusted and cells were seeded in a 96-well plate at 1.5×10⁴ cells/well. siRNA transfection complex was obtained by mixing Opti-MEM (Gibco) containing 0.3 μl/well of Lipofectamine RNAiMAX (Thermo) with siRNA at a 1:1 ratio. After the cells were cultured for a certain time, mRNA was extracted using Dynabeads mRNA Isolation Kit (Thermo) according to the instructions and was eluted with 20 μl RNase-free H₂O at 80° C. for 5 minutes. 15 μl supernatant was quickly transferred to a new 96-well-plate on a magnetic stand. The heating process was completed by a BIO-RAD T100 Thermal Cycler PCR instrument.

### 2.1.2 Real-Time Quantitative Polymerase Chain Reaction (PCR)

The cDNA synthesis and real-time fluorescence quantitative PCR were performed using the One Step PCR method. Relative mRNA levels of INHBE and GAPDH (glyceraldehyde-3-phosphate dehydrogenase) were measured using the ΔΔCt method.

LightCycler 480 II (Roche) was used for the reaction. The reaction conditions were (1) reverse transcription at 50° C. for 3 minutes, (2) pre-denaturation at 95° C. for 30 seconds; (3) denaturation at 95° C. for 10 seconds, and annealing extension at 60° C. for 30 seconds. Step (3) was repeated for 40 cycles. The results were normalized to blank control to obtain the relative mRNAlevel and knockdown efficacy. The IC50 was obtained by four-parameter fitting using GraphPad Prism software. IN-Ref2m-L96 is the dsRNAi agent formed by linking the dsRNA of IN-Ref2m with L96. The structure and preparation of L96 is described in WO2014089313A1.

The sequences of NC, NCm, IN-Ref1˜IN-Ref9, IN-Ref1m, IN-Ref2m and IN-Ref8m, based on WO2023003922A1, are shown in Table 3 below.

TABLE 3
Sequences of negative control (NC) and Ref1-Ref8

| | | SEQ | | SEQ |
|---|---|---|---|---|
| | | ID | | ID |
| Duplex | Sense Strand Sequences 5′ to 3′ | NO: | Antisense Strand Sequences 5′ to 3′ | NO: |
| NC | AAUUCUCCGAACGUGUCACGU | 1591 | ACGUGACACGUUCGGAGAAUU | 1592 |
| IN-Ref1 | ACCAGUCGUCCCAGAAUAACU | 1593 | AGUUAUTCUGGGACGACUGGUCA | 1594 |
| IN-Ref2 | ACCAGUCGUCCCAGAAUAACU | 1595 | AGUUAUTCUGGGACGACUGGUCU | 1596 |
| IN-Ref3 | CCAGAAUAACUCAUCCUCCAU | 1597 | ATGGAGGAUGAGUUAUUCUGGGA | 1598 |
| IN-Ref4 | CUGUCACAGACUCCACUUCAU | 1599 | AUGAAGTGGAGUCUGUGACAGUA | 1600 |
| IN-Ref5 | UGUCACAGACUCCACUUCAGU | 1601 | ACUGAAGUGGAGUCUGUGACAGU | 1602 |
| IN-Ref6 | CUUUGCUUGAGGAUCUUCCGU | 1603 | ACGGAAGAUCCTCAAGCAAAGAG | 1604 |
| IN-Ref7 | AAUGGGCACUUUCUUGUCUGU | 1605 | ACAGACAAGAAAGUGCCCAUUUG | 1606 |
| IN-Ref8 | GACAAGCAUUUAUACUUUCUU | 1607 | AAGAAAGUAUAAAUGCUUGUCUC | 1608 |
| IN-Ref9 | ACAAGCAUUUAUACUUUCUUU | 1609 | AAAGAAAGUAUAAAUGCUUGUCU | 1610 |
| IN- | mA*mC*mCmAmGmUmCmGfUfC | 1611 | mA*dG*mUmUdAmUdTmCmUmGmG | 1612 |
| Ref1m | fCmCmAmGmAmAmUmAmAmC | | dGmAfCmGmAmCmUmGmGmU*mC* | |
| | mU | | mA | |
| IN- | mA*mC*mCmAmGmUmCmGfUfC | 1613 | mA*dG*mUmUdAmUdTmCmUmGmG | 1614 |
| Ref2m | fCmCmAmGmAmAmUmAmAmC | | dGmAfCmGmAmCmUmGmGmU*mC* | |
| | mU | | mU | |
| IN- | mG*mA*mCmAmAmGmCmAfUf | 1615 | mA*dA*mGmAdAmAdGmUmAmUmA | 1616 |
| Ref8m | UfUmAmUmAmCmUmUmUmCm | | dAmAfUmGmCmUmUmGmUmC*mU* | |
| | UmU | | mC | |
| NCm | mA*fA*mUfUmCfUmCfCmGmAm | 1617 | mA*mC*mGmUfGfAfCfAfCfGmUmU | 1618 |
| | AfCmGfUmGfUmCfAmC*mG*mU | | mCmGmGmAmGmAmA | |

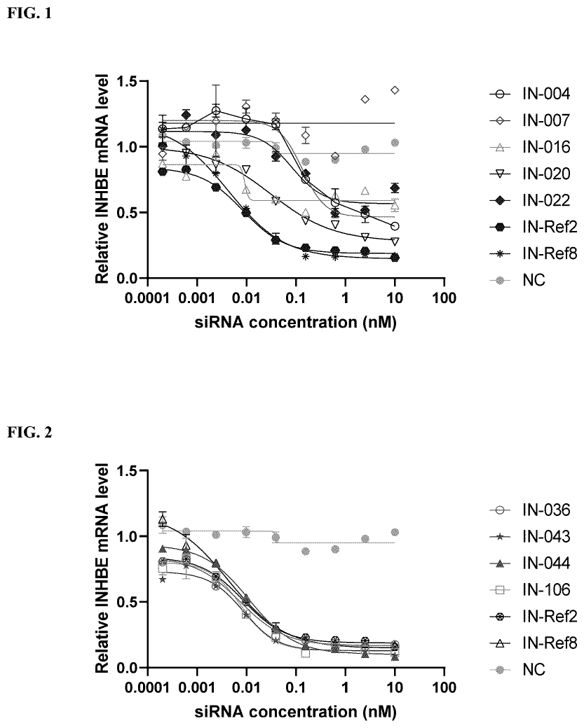

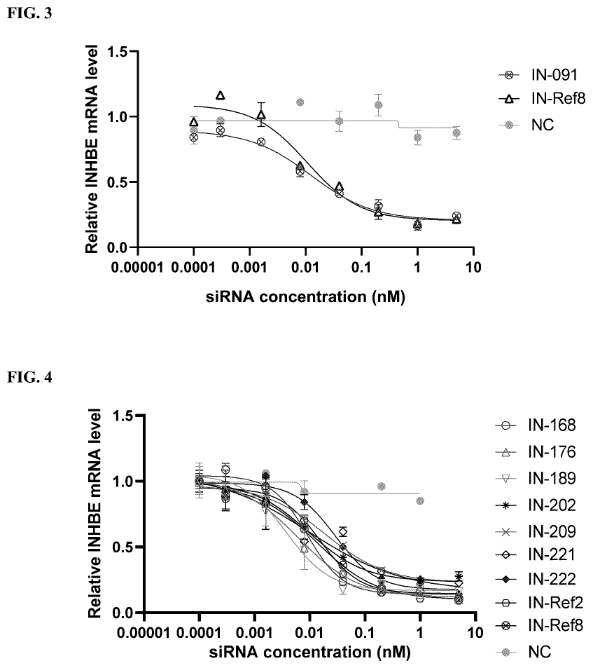

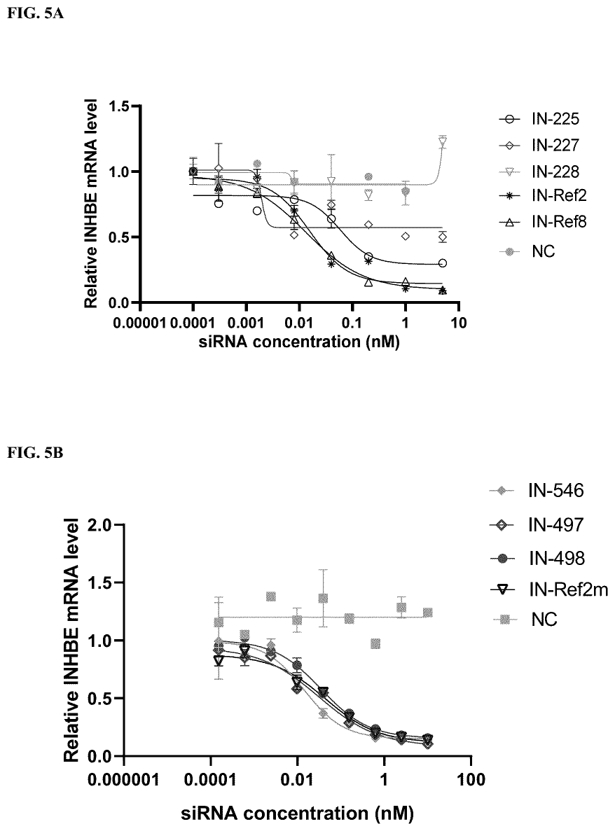

The test results are shown in FIGS. 1-4, 5A and 5B. Table 4 shows the results of IC50 determination of siRNAs and Bottom (maximum inhibition rate). “NC” means negative control. “NA” means not applicable. The numbers or letters after the decimal point in the duplex number represent different assay batches. Batch 1, batch 2 and batch 3 all involve human INHBE, and other batches also involve human INHBE. Table 5a shows the relative activities obtained after normalization by IC50 values using IN-Ref8 as the standard. Table 5b shows the inhibition rates of IN-Ref8 and IN-Ref8m at different concentrations.

TABLE 4

| | | | | Maximum |
|---|---|---|---|---|
| Duplex | IC50 (nM) | Duplex | IC50 (nM) | inhibition rate |
| IN-036.1 | 0.007 | IN-499.a | 0.04034 | 85.86% |
| IN-043.1 | 0.009 | IN-501.a | 0.06744 | 90.14% |
| IN-044.1 | 0.011 | IN-502.a | 0.0418 | 82.51% |
| IN-106.1 | 0.008 | IN-533.a | 0.03161 | 84.68% |
| IN-Ref2.1 | 0.008 | IN-529.a | 0.04679 | 79.06% |
| IN-Ref8.1 | 0.004 | IN-535.a | 0.02976 | 79.63% |
| IN-091.1 | 0.013 | IN-518.a | 0.0448 | 82.67% |
| IN-Ref8.2 | 0.011 | IN-519.a | 0.04175 | 65.44% |
| IN-168.2 | 0.009 | IN-523.a | 0.06287 | 53.20% |
| IN-176.2 | 0.006 | IN-Ref2m.a | 0.03862 | 88.78% |
| IN-189.2 | 0.004 | IN-505.b | 0.1964 | 72.75% |
| IN-202.2 | 0.009 | IN-510.b | 0.07662 | 55.36% |
| IN-209.2 | 0.019 | IN-496.b | 0.1935 | 70.50% |
| IN-221.2 | 0.01 | IN-544.b | 0.0366 | 76.76% |
| IN-222.2 | 0.029 | IN-545.b | 0.02449 | 75.52% |
| IN-Ref2.2 | 0.016 | IN-Ref2m.b | 0.04379 | 88.57% |
| IN-Ref8.3 | 0.013 | IN-546.c | 0.01433 | 85.07% |
| NC | NA | IN-497.c | 0.03111 | 91.73% |
| — | — | IN-498.c | 0.03516 | 84.71% |
| — | — | IN-Ref2m.c | 0.04379 | 88.57% |

TABLE 5a

| Duplex | Relative activity |
|---|---|
| IN-004 | 3.00% |
| IN-020 | 12.87% |
| IN-022 | 4.80% |
| IN-036 | 57.14% |
| IN-043 | 44.44% |
| IN-044 | 36.36% |
| IN-106 | 50.00% |
| IN-091 | 100.00% |
| IN-168 | 144.44% |
| IN-176 | 216.67% |
| IN-189 | 325.00% |
| IN-202 | 144.44% |
| IN-209 | 68.42% |
| IN-221 | 130.00% |
| IN-222 | 44.83% |
| IN-225 | 22.10% |
| IN-Ref2 | 81.25% |
| IN-Ref8 | 100.00% |

TABLE 5b

| | 100 nM inhibition | | 2 nM inhibition | | 0.04 nM inhibition | |
|---|---|---|---|---|---|---|
| Duplex | rate (%) | | rate (%) | | rate (%) | |
| IN-Ref8 | 88.54 | 86.83 | 90.09 | 90.82 | 73.11 | 73.67 |
| IN-Ref8m | 70.48 | 66.55 | 68.03 | 73.85 | 13.85 | 10.81 |

### 2.2 Reporter Gene Assay

SK-Hep-1-PsiCheck-INHBE cells containing the full-length INHBE mRNA sequence and luciferase gene were used for detection. Cells were cultured using MEM medium (Gibco) supplemented with 10% FBS (Gibco), 1 μg/ml puromycin, and penicillin and streptomycin (Gibco) at 5% CO₂, 37° C., and resuspended after trypsin digestion after cell growth had nearly covered the whole culture flask. The density of the resuspended cells was adjusted and cells were seeded in a 96-well plate at 1.5×10⁴ cells/well, and siRNA transfection complex was added at the same time. The siRNA transfection complex was obtained by mixing Opti-MEM (Gibco) containing 0.3 μl/well of Lipofectamine RNAiMAX (Thermo) with siRNA at a 1:1 ratio. Cells were lysed after a period of time of culture and the Renilla substrate (Vazyme) was added for fluorescence activity detection.

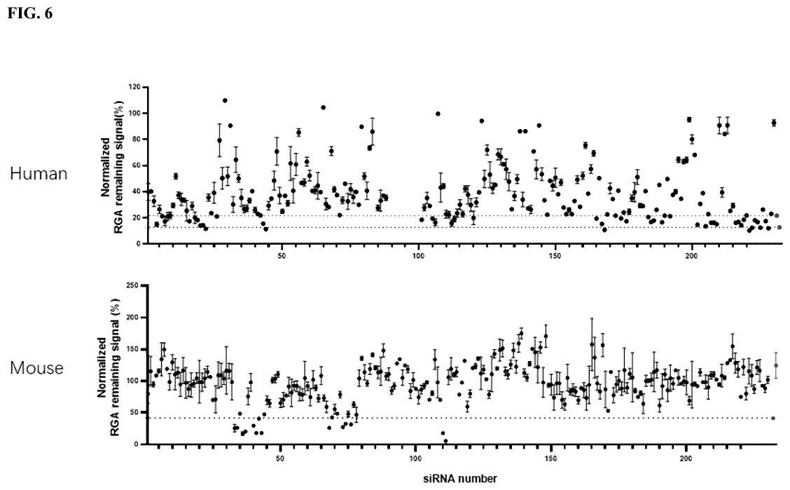

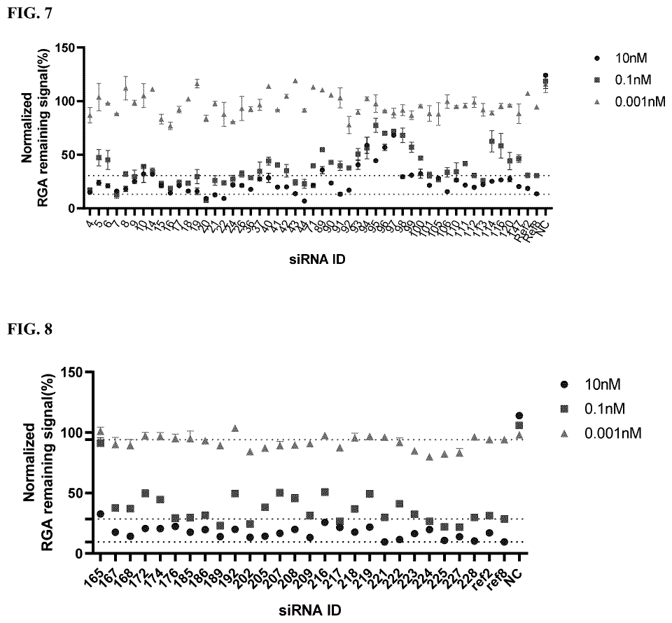

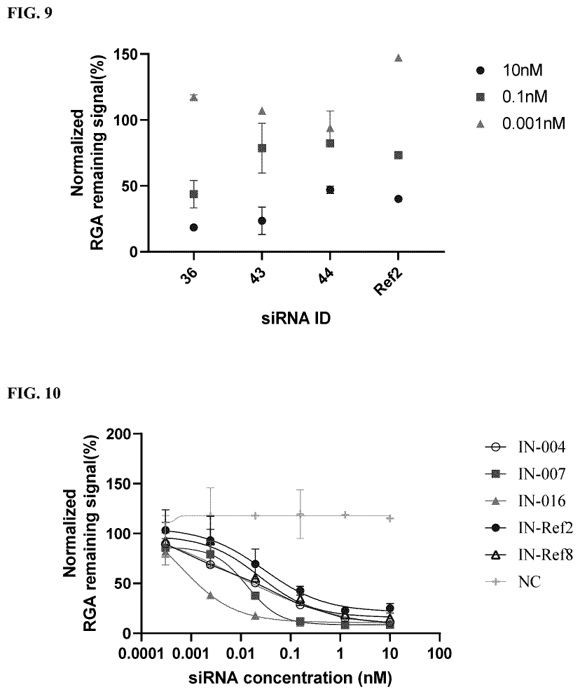

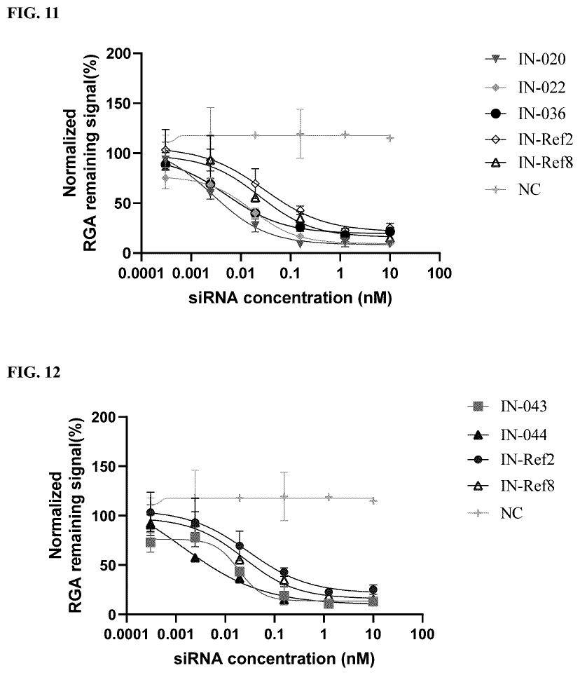

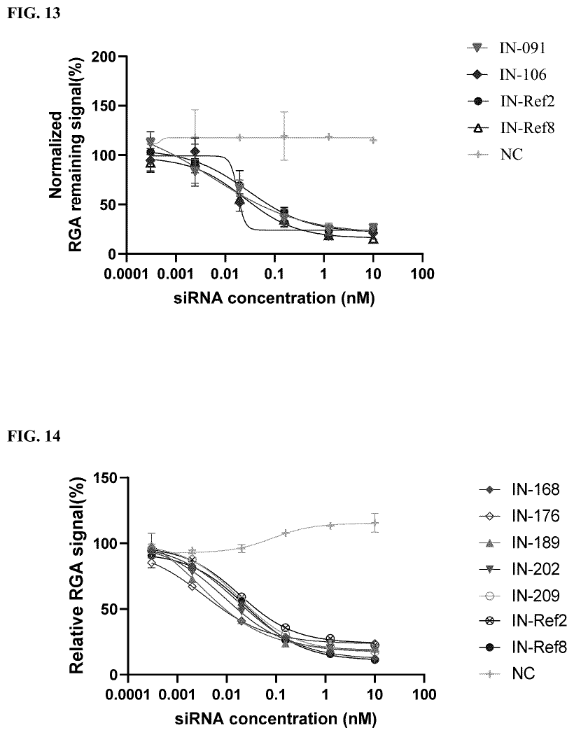

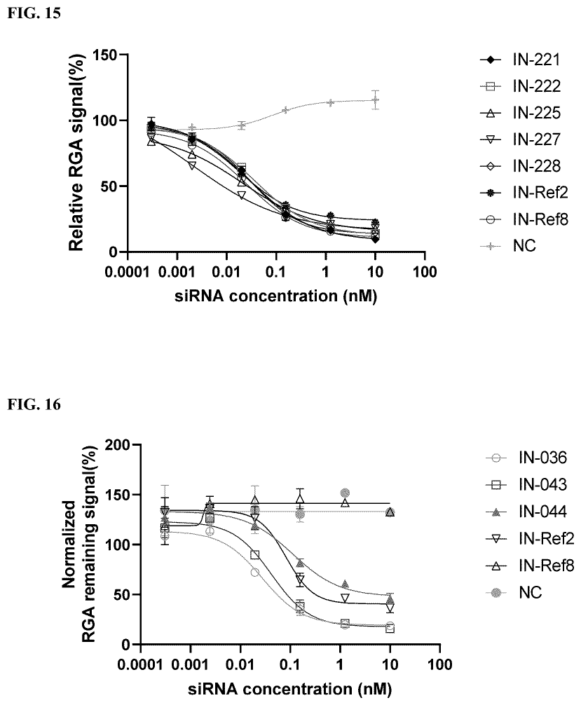

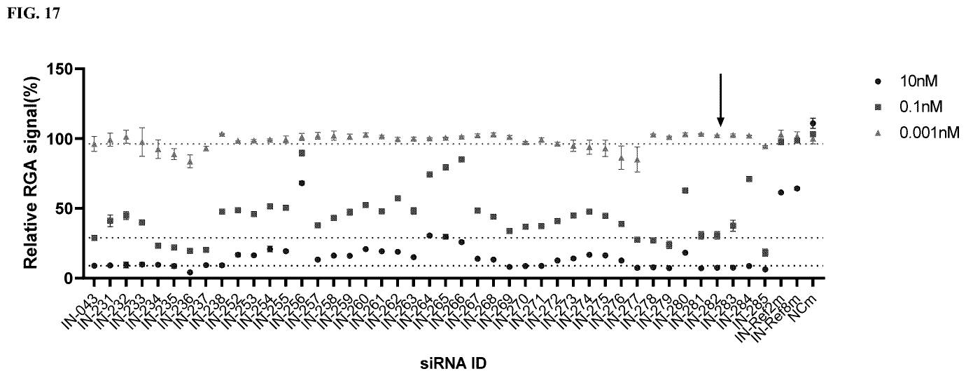

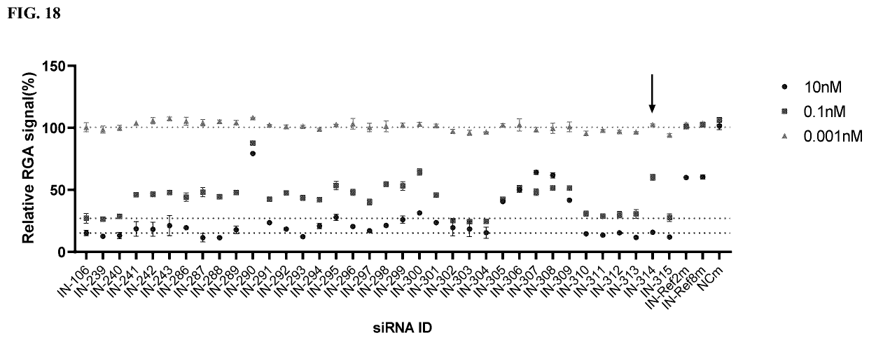

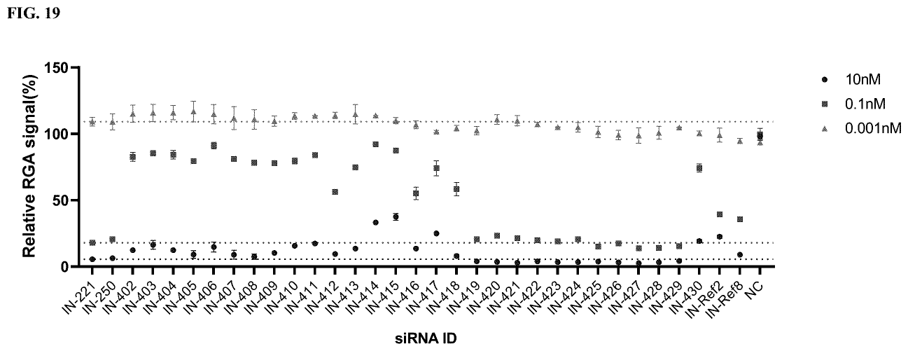

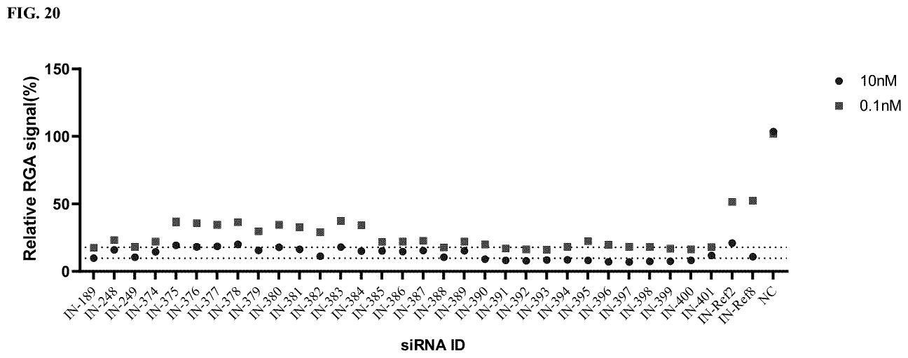

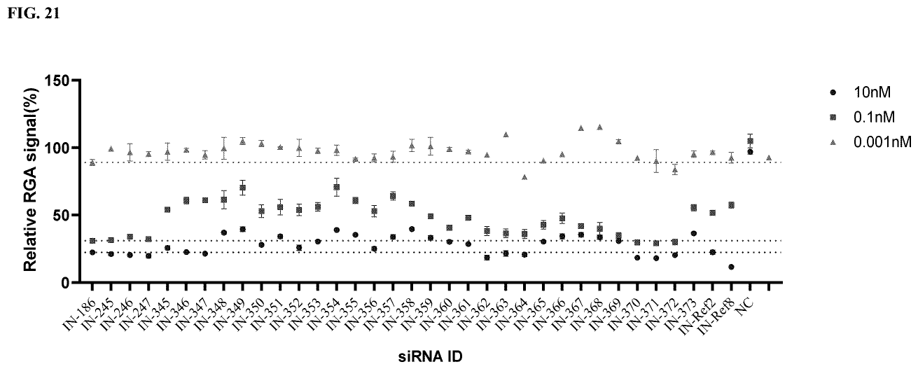

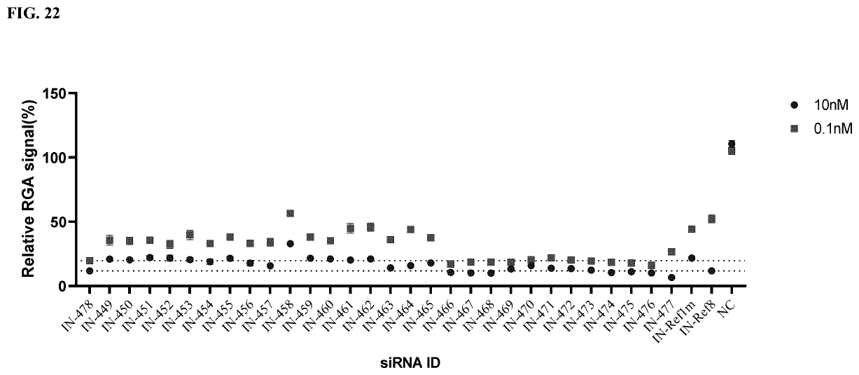

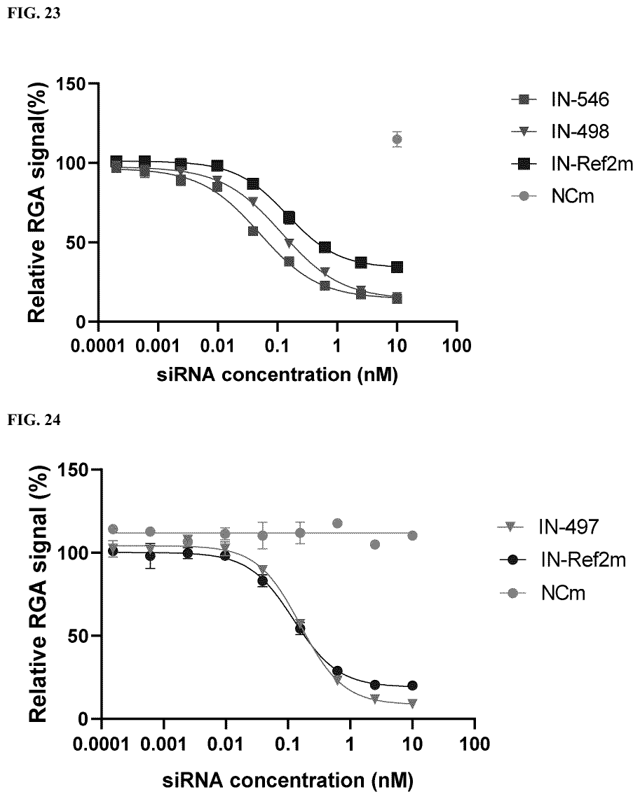

The test results are shown in FIGS. 6-24. Except for FIG. 9 and FIG. 16 which were measured in mouse INHBE, and the rest were measured in human INHBE. Table 6 and Table 8 show the IC50 assay results of siRNAs (fitted using the four-parameter method in GraphPad Prism) and the inhibition rates normalized to the blank control group. Batches 4 and 5 both involved human INHBE, batch 6 involved mouse INHBE, and other batches also involved human INHBE. Table 7 shows the IC50 values obtained after normalizing the inhibition rate at the same concentration using IN-Ref8 as the standard. Table 9 shows the inhibition rates of IN-Ref8 and IN-Ref8m at different concentrations.

TABLE 6

| Duplex | IC50 (nM) |
|---|---|
| IN-004.4 | 0.008 |
| IN-007.4 | 0.013 |
| IN-016.4 | 0.001 |
| IN-020.4 | 0.003 |
| IN-022.4 | 0.017 |
| IN-036.4 | 0.005 |
| IN-043.4 | 0.019 |
| IN-044.4 | 0.001 |
| IN-091.4 | 0.006 |
| IN-106.4 | 0.018 |
| IN-Ref2.4 | 0.03 |
| IN-Ref8.4 | 0.024 |
| IN-168.5 | 0.017 |
| IN-176.5 | 0.004 |
| IN-189.5 | 0.003 |
| IN-202.5 | 0.009 |
| IN-209.5 | 0.018 |
| IN-221.5 | 0.029 |
| IN-222.5 | 0.037 |
| IN-225.5 | 0.021 |
| IN-227.5 | 0.002 |
| IN-228.5 | 0.03 |
| IN-Ref2.5 | 0.018 |
| IN-Ref8.5 | 0.025 |
| IN-036.6 | 0.03 |
| IN-043.6 | 0.04 |
| IN-044.6 | 0.11 |
| IN-Ref2.6 | 0.08 |
| IN-Ref8.6 | NA |
| NC | NA |

TABLE 7

| Duplex | IC50 (nM) |
|---|---|
| IN-004 | 0.008 |
| IN-007 | 0.0134 |
| IN-016 | 0.0006 |
| IN-020 | 0.0027 |
| IN-022 | 0.0167 |
| IN-036 | 0.0052 |
| IN-043 | 0.0188 |
| IN-044 | 0.0013 |
| IN-091 | 0.0055 |
| IN-106 | 0.0179 |
| IN-168 | 0.0178 |
| IN-176 | 0.0113 |
| IN-189 | 0.0081 |
| IN-202 | 0.0119 |
| IN-209 | 0.0174 |
| IN-221 | 0.0296 |
| IN-222 | 0.0363 |
| IN-225 | 0.0149 |
| IN-227 | 0.0109 |
| IN-228 | 0.028 |
| IN-Ref2 | 0.0295 |
| IN-Ref8 | 0.0238 |

TABLE 8

| | | Maximum | | | Maximum |
|---|---|---|---|---|---|
| | | inhibition | | IC50 | inhibition |
| Duplex | IC50 (nM) | rate (%) | Duplex | (nM) | rate (%) |
| IN-518.d | 0.5308 | 86.45 | IN-Ref2m.i | 0.1234 | 80.96 |
| IN-519.d | 0.3315 | 85.45 | IN-510.j | 0.2742 | 74.54 |
| IN-523.d | 0.5822 | 49.41 | IN-511.j | 1.433 | 60.99 |
| IN-Ref2m.d | 0.08943 | 75.95 | IN-512.j | 0.9971 | 81.15 |
| IN-503.e | 0.2849 | 50.49 | IN-513.j | 1.431 | 60.89 |
| IN-504.e | 0.2148 | 62.89 | IN-Ref2m.j | 0.1321 | 66.81 |
| IN-505.e | 0.1685 | 73.85 | IN-514.k | 1.258 | 54.21 |
| IN-506.e | 1.064 | 64.92 | IN-515.k | 0.4826 | 68.16 |
| IN-507.e | 0.3262 | 64.08 | IN-516.k | 0.7253 | 87.68 |
| IN-508.e | ~1.460 | 54.86 | IN-517.k | 0.3504 | 77.17 |
| IN-536.e | 0.006015 | 88.40 | IN-486.k | 0.1323 | 79.33 |
| IN-537.e | 0.007107 | 89.11 | IN-487.k | 0.09262 | 83.95 |
| IN-538.e | 0.01333 | 87.68 | IN-488.k | 0.2625 | 76.84 |
| IN-539.e | 0.01698 | 89.00 | IN-492.k | 0.08549 | 88.29 |
| IN-540.e | 0.008467 | 89.36 | IN-494.k | 0.1097 | 88.80 |
| IN-541.e | 0.006741 | 89.56 | IN-495.k | 0.07071 | 87.76 |
| IN-542.e | 0.008483 | 88.14 | IN-496.k | 0.06723 | 88.05 |
| IN-543.e | 0.006184 | 86.99 | IN-524.k | 0.3675 | 52.56 |
| IN-Ref2m.e | 0.08503 | 71.42 | IN-525.k | 0.1567 | 64.17 |
| IN-239.f | 0.03846 | 87.19 | IN-526.k | 0.1135 | 67.66 |
| IN-240.f | 0.02503 | 85.94 | IN-527.k | 0.6912 | 41.73 |
| IN-499.f | 0.1218 | 72.92 | IN-528.k | 0.3194 | 39.21 |
| IN-500.f | 0.09278 | 70.30 | IN-Ref2m.k | 0.08025 | 70.00 |
| IN-501.f | 0.1427 | 75.78 | IN-189.1 | 0.01543 | 90.82 |
| IN-502.f | 0.03788 | 74.96 | IN-529.1 | 0.1083 | 78.29 |
| IN-Ref2m.f | 0.1133 | 77.94 | IN-530.1 | 0.08113 | 80.59 |
| IN-545.g | 0.07606 | 93.00 | IN-531.1 | 0.1237 | 81.02 |
| IN-Ref2m.g | 0.1045 | 73.66 | IN-532.1 | 0.09852 | 86.28 |
| IN-544.h | 0.03637 | 87.68 | IN-533.1 | 0.03468 | 93.26 |
| IN-546.h | 0.05053 | 85.69 | IN-534.1 | 0.1176 | 90.51 |
| IN-496.h | 0.1376 | 85.30 | IN-535.1 | 0.08164 | 85.04 |
| IN-498.h | 0.1177 | 85.97 | IN-221.1 | 0.0123 | 96.31 |
| IN-509.h | 0.4069 | 67.45 | IN-520.1 | 1.871 | 42.45 |
| IN-Ref2m.h | 0.1439 | 66.30 | IN-521.1 | 0.9813 | 56.78 |
| IN-Ref2m- | 0.2004 | 60.08 | IN-522.1 | 0.1135 | 66.84 |
| L96.h | | | | | |
| IN-497.i | 0.1584 | 91.48 | IN-Ref2m.1 | 0.2218 | 74.20 |

TABLE 9

| | 10 nM inhibition | | 0.1 nM inhibition | | 0.001 nM inhibition | |
|---|---|---|---|---|---|---|
| Duplex | rate | | rate | | rate | |
| IN-Ref8 | 91.39% | 91.53% | 70.81% | 71.56% | 4.6% | 1.08% |
| IN-Ref8m | 35% | 36.53% | 1.82% | 1.04% | 0.31% | −3.95% |

### Example 3. In Vitro Stability Assay for siRNAs

### 3.1. Stem-Loop PCR

The Stem-loop method has been widely used to detect the absolute concentration of siRNA and miRNA (Curr Protoc Mol Biol. 2011 July; Chapter 15:Unit 15.10). After designing the corresponding stem-loop primers for different siRNAs and using the primers for reverse transcription, the siRNA concentrations were calculated using rt-PCR with a standard curve. Reverse transcription was performed using the HiScript III 1st Strand cDNA Synthesis Kit (Vazyme), and the process was as follows: (1) Sample pretreatment: (a) 85° C., 5 min; (b) 60° C., 5 min; (2) Reverse transcription according to the manufacturer's protocol; (3) The reaction system was prepared according to the conditions described in the SYBR Green kit (TIANGEN®) and rt-PCR was performed. The reverse transcription was performed by BIO-RAD® T100 Thermal Cycler PCR instrument. The quantitative real-time-PCR was performed by Roche LC480 II.

### 3.1.1 Stability in FBS

(1) Dilute siRNA to 100 nM using FBS (Gibco, fetal bovine serum) and mark it as the zero point; (2) aliquot 10 μl from the zero point into new sample tubes then incubate at 37° C. for different times; (3) Take out the corresponding samples with different incubation time and put them into liquid nitrogen to freeze quickly, and then store the samples at −80° C. Before the experiment, the samples were thoroughly mixed, using the gradient dilution of samples from the zero point group as the standard curve, and perform the experiment using the Stem-loop PCR method described in 3.1 above.

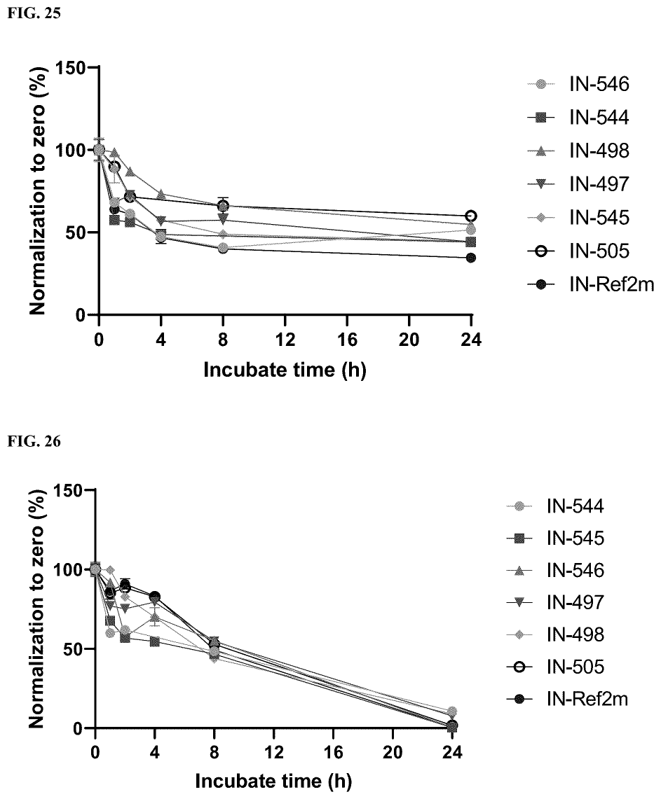

The cycle threshold (Ct) values obtained from PCR of the standards at different concentrations were linearly fitted using GraphPad Prism software to obtain a standard curve correlating the standard concentrations with Ct values. The Ct values of the samples at each incubation time point were then back-calculated using the standard curve to obtain the siRNA concentration in the samples. The concentration of the samples at each time point was normalized to the concentration of the 0-hour sample, and the percentage of residual siRNA concentration at each time point relative to the 0-hour sample was calculated. FIG. 25 shows the results of the siRNA serum stability test.

### 3.1.2 Stability in Human Liver S9

(1) Diluted siRNA with human liver S9 (BioIVT); (2) Incubated samples at 37° C. for different times. (3) Took out the corresponding samples with different incubation times and put them into liquid nitrogen to freeze quickly, and then stored the samples at −80° C. for later use. Performed the experiment using the Stem-loop PCR method described in 3.1 above.

The Ct values obtained from PCR of the standards at different concentrations were linearly fitted using GraphPad Prism software to obtain a standard curve correlating standard concentrations with Ct values. The Ct values of the samples at each incubation time point were then back-calculated using the standard curve to obtain the siRNA concentration in each sample. The concentration of the samples at each time point was normalized to the concentration of the 0-hour sample, and the percentage of residual siRNA concentration at each time point relative to the 0-hour sample was calculated. FIG. 26 shows the results of the siRNA stability test in human liver S9.

### Example 4. Pharmacokinetic Analysis of siRNA in Liver

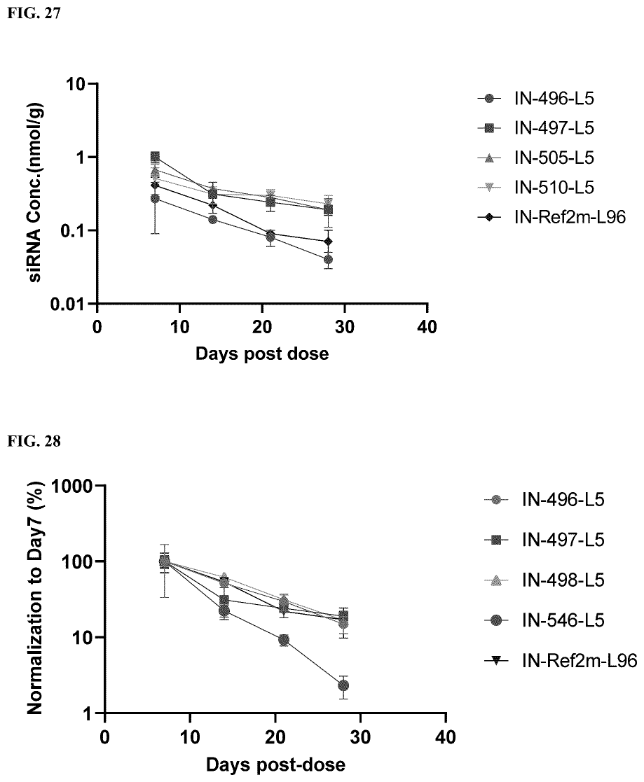

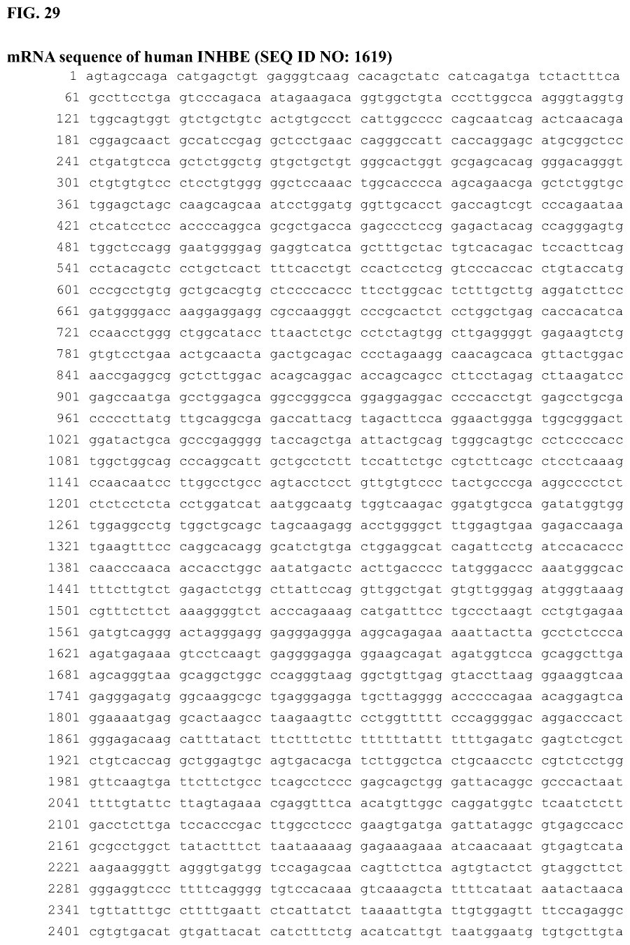

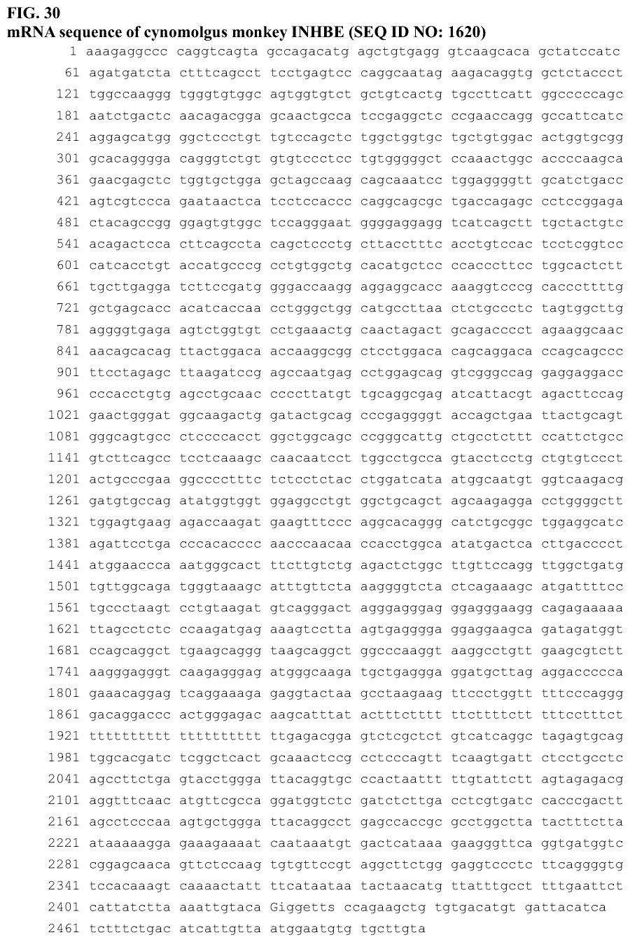

After subcutaneous administration to mice at a dose of 3 mg/kg, livers were taken from mice at specific times. The liver was weighed and homogenized in PBS at 4° C. with a homogenizer (Shanghai JXFSTPRP-64). The siRNA content was detected by Stem-loop PCR as described previously. The siRNA concentration in the liver tissues of the treated mice was determined by back-calculating based on the standard curve. FIG. 27 shows the changes in siRNA content in the liver of each group (3 mice per group) at different time points after administration. FIG. 28 shows the percentage of different siRNA content in the mouse liver, normalized to the siRNA content in the liver tissue of mice (3 mice per group) 7 days after administration.

### Example 5. In Vivo Efficacy of GalNAc-Conjugated INHBE siRNA in Healthy Cynomolgus Monkeys

To assess the in vivo activity of siRNA targeting INHBE, an in vivo activity assay was performed using healthy cynomolgus monkeys. Pre-dose liver biopsy samples were obtained 24-hour before injection. GalNAc-conjugated siRNAs were diluted using saline and injected subcutaneously on day 1 according to the experimental design. Blank saline was used as a negative control. Liver biopsy was collected on days 14, 28, 42, 56, and 70, respectively. Liver INHBE mRNA level was analyzed by quantitative real-time-PCR by HiScript II One-Step RT-PCR Kit (Vazyme) with total RNA extracted by FastPure Cell/Tissue Total RNA Isolation Kit V2 (Vazyme). The quantitative real-time PCR was performed by Roche LC480 II.

The obtained test results were standardized using the reference gene (GAPDH) to obtain the relative mRNA levels, and the INHBE mRNA levels of each animal were individually standardized. For individual standardization, at each time point, the average INHBE mRNA level of each animal was divided by the average pre-treatment expression level of that animal to determine the “standardized to pre-treatment” relative expression level. Table 10 shows the mean relative expression level of each group of animals obtained by standardizing each animal's liver tissue mRNA levels to their own pre-treatment levels. “Vehicle” indicates the blank control.

TABLE 10

| RNAi | Pre- treatment | 14 days | 28 days | 42 days | 56 days | 70 days |
|---|---|---|---|---|---|---|
| IN-546-L5 | 100.00% | 28.18% | 25.93% | 40.70% | 31.20% | 37.50% |
| IN-497-L5 | 100.00% | 18.29% | 30.10% | 50.24% | 29.93% | 36.20% |
| IN-498-L5 | 100.00% | 14.53% | 11.62% | 45.20% | 21.86% | 20.54% |
| Vehicle | 100.00% | 153.97% | 334.31% | 345.62% | 294.62% | 313.42% |

Although this invention is described in detail with reference to embodiments thereof, these embodiments are offered to illustrate but not to limit the invention. It is possible to make other embodiments that employ the principles of the invention and that fall within its spirit and scope as defined by the claims appended hereto.

The contents of all documents and references cited herein are hereby incorporated by reference in their entirety.

What is claimed is:

1. A double-stranded ribonucleic acid interference (dsRNAi) agent useful for inhibiting expression of inhibin subunit beta E (INHBE) in a cell, wherein the dsRNAi agent comprises a sense strand and an antisense strand forming a double-stranded RNA (dsRNA) region, wherein the antisense strand comprises a region of complementarity to an INHBE mRNA, wherein the region of complementarity comprises at least 15 contiguous nucleotides, and wherein the dsRNAi agent comprises at least one non-canonical base pairing nucleotide.

2.-9. (canceled)

10. The dsRNAi agent of claim 1, wherein the dsRNAi agent comprises at least one additional modified nucleotide in the sense strand and/or the antisense strand.

11.-13. (canceled)

14. The dsRNAi agent of claim 1, wherein said at least one non-canonical base pairing nucleotide(s) is independently inosine (I), xanthosine (X), 7-methylguanosine (m7G), N6-methyladenosine (m6A), dihydrouridine, 5-methylcytosine (m5C), pseudouridine (Ψ), N1-methylpseudouridine (m1Ψ), or a combination thereof.

15. The dsRNAi agent of claim 14, wherein said at least one non-canonical base pairing nucleotide is inosine (I); and/or, wherein at least one guanine (G) is replaced with hypoxanthine (I) in the sense strand and/or the antisense strand.

16.-20. (canceled)

21. The dsRNAi agent of claim 10, wherein said at least one additional modified nucleotide is a 2′-O-methyl-modified nucleotide, a 2′-fluoro-modified nucleotide, a 2′-deoxy nucleotide, a 2′-methoxyethyl-modified nucleotide, a 2′-amino-modified nucleotide, a 2′-alkyl-modified nucleotide, a 2′-alkoxy-modified nucleotide, a 2′-F-arabino nucleotide, a phosphorothioate-modified nucleotide, an abasic nucleotide, a morpholino nucleotide, a locked nucleotide, an inverted nucleotide, a hypoxanthine base-substituted nucleotide, or a combination thereof.

22. (canceled)

23. The dsRNAi agent of claim 21, wherein the inverted base nucleotide is inverted A nucleotide, inverted dA nucleotide, inverted dT nucleotide, inverted C nucleotide or inverted U nucleotide.

24. The dsRNAi agent of claim 1, wherein the region of complementarity comprises at least 15, at least 16, at least 17, at least 18, at least 19, at least 20, at least 21, at least 22, or at least 23 contiguous nucleotides, or from 15 to 23 contiguous nucleotides, or from 15 to 21 contiguous nucleotides, or wherein the region of complementarity comprises 15, 16, 17, 18, 19, 20, 21, 22 or 23 contiguous nucleotides.

25. (canceled)

26. The dsRNAi agent of claim 1, wherein the difference in calculated melting temperature (ΔTm) of the dsRNA region or a portion thereof comprising said at least one non-canonical base pairing nucleotide compared to the same dsRNA region or portion thereof having no non-canonical base pairing nucleotide is at least 2° C.

27.-31. (canceled)

32. The dsRNAi agent of claim 15, wherein said at least one guanine (G) is G₀ comprised in the following sequence, according to the direction from the 5′ end to the 3′ end:
5′- N₁N₂G₀N₃N₄ -3′
wherein N₁, N₂, N₃, and N₄ are independently adenine (A), cytosine (C), G or uracil (U); wherein, if at least three (3) bases of N₁, N₂, N₃, and N₄ are adenine (A) or uracil (U), then the guanine in G₀ is replaced with hypoxanthine (I).

33. The dsRNAi agent of claim 1, wherein the dsRNAi agent comprises the following sequence on the antisense strand and/or the sense strand, according to the direction from the 5′ end to the 3′ end:
5′- N₁N₂G₀N₃N₄ -3′
wherein N₁, N₂, N₃, and N₄ are independently adenine (A), cytosine (C), G or uracil (U); wherein, if at least three (3) bases of N₁, N₂, N₃, and N₄ are adenine (A) or uracil (U), then the guanine in G₀ is replaced with a non-canonical base,
wherein the non-canonical base is the base in inosine (I), xanthosine (X), 7-methylguanosine (m7G), N6-methyladenosine (m6A), dihydrouridine, 5-methylcytosine (m5C), pseudouridine (Ψ), or N1-methylpseudouridine (m1Ψ), or wherein the non-canonical base is hypoxanthine (I).

34.-39. (canceled)

40. The dsRNAi agent of claim 1, wherein the antisense strand comprises at least 15 contiguous nucleotides differing by no more than 3 nucleotides from any one of the nucleotide sequences shown in Table 1 and Table 2, and the sense strand comprises at least 15 nucleotides complementary to the antisense strand; or,
wherein the dsRNAi agent comprises any one of the duplex sequences selected from IN-001 to IN-488, INI-001 to INI-308, and IN-489 to IN-546; or,
wherein the sense strand is 19 nucleotides in length which comprises 15 contiguous nucleotides as set forth in any one of SEQ ID NOs. 1621 to 1689; or
wherein the antisense strand is 21 nucleotides in length which comprises 15 contiguous nucleotides as set forth in any one of SEQ ID NOs. 1690 to 1758.

41.-72. (canceled)

73. The dsRNAi agent of claim 1, wherein the antisense strand comprises the nucleotide sequence set forth in any one of SEQ ID NO: 86, 182, 212, 350, 376, 402, 440, 460, 462, 464, 466, 468, 470, 472, 474, 476, 478, 480, 482, 484, 486, 488, 490, 492, 494, 496, 498, 500, 502, 502, 504, 506, 508, 510, 512, 514, 516, 518, 520, 522, 524, 526, 528, 530, 532, 534, 536, 538, 540, 542, 544, 546, 548, 550, 552, 554, 556, 558, 560, 562, 564, 566, 568, 570, 572, 574, 576, 578, 580, 582, 584, 586, 588, 590, 592, 594, 596, 598, 600, 602, 604, 606, 608, 610, 612, 614, 616, 618, 620, 622, 624, 626, 628, 630, 632, 634, 636, 638, 640, 642, 644, 646, 648, 650, 652, 654, 656, 658, 660, 662, 664, 666, 668, 670, 672, 674, 676, 678, 680, 682, 684, 686, 688, 690, 692, 694, 696, 698, 700, 702, 704, 706, 708, 710, 712, 714, 716, 718, 720, 722, 724, 726, 728, 730, 732, 734, 736, 738, 740, 742, 744, 746, 748, 750, 752, 754, 756, 758, 760, 762, 764, 766, 768, 770, 772, 774, 776, 778, 780, 782, 784, 786, 788, 790, 792, 794, 796, 798, 800, 802, 804, 806, 808, 810, 812, 814, 816, 818, 820, 822, 824, 826, 828, 830, 832, 834, 836, 838, 840, 842, 844, 846, 848, 850, 852, 854, 856, 858, 860, 862, 864, 866, 868, 870, 872, 874, 876, 878, 880, 882, 884, 886, 888, 890, 892, 894, 896, 898, 900, 902, 904, 906, 908, 910, 912, 914, 916, 918, 920, 922, 924, 926, 928, 930, 932, 934, 936, 938, 940, 942, 944, 946, 948, 950, 952, 954, 956, 958, 960, 962, 964, 966, 968, 970, 972, 974, 1760, 1762, 1764, 1766, 1768, 1770, 1772, 1774, 1776, 1778, 1780, 1782, 1784, 1786, 1788, 1790, 1792, 1794, 1796, 1798, 1800, 1802, 1804, 1806, 1808, 1810, 1812, 1814, 1816, 1818, 1820, 1822, 1824, 1826, 1828, 1830, 1832, 1834, 1836, 1838, 1840, 1842, 1844, 1846, 1848, 1850, 1852, 1854, 1856, 1858, 1860, 1862, 1864, 1866, 1868, 1870, 1872 or 1874.

74. The dsRNAi agent of claim 1, wherein the sense strand comprises the nucleotide sequence set forth in any one of SEQ ID NO: 85, 181, 211, 349, 375, 401, 439, 459, 461, 463, 465, 467, 469, 471, 473, 475, 477, 479, 481, 483, 485, 487, 489, 491, 493, 495, 497, 499, 501, 503, 505, 507, 509, 511, 513, 515, 517, 519, 521, 523, 525, 527, 529, 531, 533, 535, 537, 539, 541, 543, 545, 547, 549, 551, 553, 555, 557, 559, 561, 563, 565, 567, 569, 571, 573, 575, 577, 579, 581, 583, 585, 587, 589, 591, 593, 595, 597, 599, 601, 603, 605, 607, 609, 611, 613, 615, 617, 619, 621, 623, 625, 627, 629, 631, 633, 635, 637, 639, 641, 643, 645, 647, 649, 651, 653, 655, 657, 659, 661, 663, 665, 667, 669, 671, 673, 675, 677, 679, 681, 683, 685, 687, 689, 691, 693, 695, 697, 699, 701, 703, 705, 707, 709, 711, 713, 715, 717, 719, 721, 723, 725, 727, 729, 731, 733, 735, 737, 739, 741, 743, 745, 747, 749, 751, 753, 755, 757, 759, 761, 763, 765, 767, 769, 771, 773, 775, 777, 779, 781, 783, 785, 787, 789, 791, 793, 795, 797, 799, 801, 803, 805, 807, 809, 811, 813, 815, 817, 819, 821, 823, 825, 827, 829, 831, 833, 835, 837, 839, 841, 843, 845, 847, 849, 851, 853, 855, 857, 859, 861, 863, 865, 867, 869, 871, 873, 875, 877, 879, 881, 883, 885, 887, 889, 891, 893, 895, 897, 899, 901, 903, 905, 907, 909, 911, 913, 915, 917, 919, 921, 923, 925, 927, 929, 931, 933, 935, 937, 939, 941, 943, 945, 947, 949, 951, 953, 955, 957, 959, 961, 963, 965, 967, 969, 971, 973, 1759, 1761, 1763, 1765, 1767, 1769, 1771, 1773, 1775, 1777, 1779, 1781, 1783, 1785, 1787, 1789, 1791, 1793, 1795, 1797, 1799, 1801, 1803, 1805, 1807, 1809, 1811, 1813, 1815, 1817, 1819, 1821, 1823, 1825, 1827, 1829, 1831, 1833, 1835, 1837, 1839, 1841, 1843, 1845, 1847, 1849, 1851, 1853, 1855, 1857, 1859, 1861, 1863, 1865, 1867, 1869, 1871 or 1873.

75. The dsRNAi agent of claim 1 wherein the dsRNAi agent comprises a sense strand and an antisense strand comprising respectively the nucleotide sequence set forth in any one of the following pairs: SEQ ID NO: 85 and 86; SEQ ID NO: 181 and 182; SEQ ID NO: 211 and 212; SEQ ID NO: 349 and 350; SEQ ID NO: 375 and 376; SEQ ID NO: 401 and 402; SEQ ID NO: 439 and 440; SEQ ID NO:459 and 460; SEQ ID NO:461 and 462; SEQ ID NO:463 and 464; SEQ ID NO:465 and 466; SEQ ID NO:467 and 468; SEQ ID NO:469 and 470; SEQ ID NO:471 and 472; SEQ ID NO:473 and 474; SEQ ID NO:475 and 476; SEQ ID NO:477 and 478; SEQ ID NO:479 and 480; SEQ ID NO:481 and 482; SEQ ID NO:483 and 484; SEQ ID NO:485 and 486; SEQ ID NO:487 and 488; SEQ ID NO:489 and 490; SEQ ID NO:491 and 492; SEQ ID NO:493 and 494; SEQ ID NO:495 and 496; SEQ ID NO:497 and 498; SEQ ID NO:499 and 500; SEQ ID NO: 501 and 502; SEQ ID NO: 503 and 504; SEQ ID NO: 505 and 506; SEQ ID NO: 507 and 508; SEQ ID NO: 509 and 510; SEQ ID NO: 511 and 512; SEQ ID NO: 513 and 514; SEQ ID NO: 515 and 516; SEQ ID NO: 517 and 518; SEQ ID NO: 519 and 520; SEQ ID NO: 521 and 522; SEQ ID NO: 523 and 524; SEQ ID NO: 525 and 526; SEQ ID NO: 527 and 528; SEQ ID NO: 529 and 530; SEQ ID NO: 531 and 532; SEQ ID NO: 533 and 534; SEQ ID NO: 535 and 536; SEQ ID NO: 537 and 538; SEQ ID NO: 539 and 540; SEQ ID NO: 541 and 542; SEQ ID NO: 543 and 544; SEQ ID NO: 545 and 546; SEQ ID NO: 547 and 548; SEQ ID NO: 549 and 550; SEQ ID NO: 551 and 552; SEQ ID NO: 553 and 554; SEQ ID NO: 555 and 556; SEQ ID NO: 557 and 558; SEQ ID NO: 559 and 560; SEQ ID NO: 561 and 562; SEQ ID NO: 563 and 564; SEQ ID NO: 565 and 566; SEQ ID NO: 567 and 568; SEQ ID NO: 569 and 570; SEQ ID NO: 571 and 572; SEQ ID NO: 573 and 574; SEQ ID NO: 575 and 576; SEQ ID NO: 577 and 578; SEQ ID NO: 579 and 580; SEQ ID NO: 581 and 582; SEQ ID NO: 583 and 584; SEQ ID NO: 585 and 586; SEQ ID NO: 587 and 588; SEQ ID NO: 589 and 590; SEQ ID NO: 591 and 592; SEQ ID NO: 593 and 594; SEQ ID NO: 595 and 596; SEQ ID NO: 597 and 598; SEQ ID NO: 599 and 600; SEQ ID NO: 601 and 602; SEQ ID NO: 603 and 604; SEQ ID NO: 605 and 606; SEQ ID NO: 607 and 608; SEQ ID NO: 609 and 610; SEQ ID NO: 611 and 612; SEQ ID NO: 613 and 614; SEQ ID NO: 615 and 616; SEQ ID NO: 617 and 618; SEQ ID NO: 619 and 620; SEQ ID NO: 621 and 622; SEQ ID NO: 623 and 624; SEQ ID NO: 625 and 626; SEQ ID NO: 627 and 628; SEQ ID NO: 629 and 630; SEQ ID NO: 631 and 632; SEQ ID NO: 633 and 634; SEQ ID NO: 635 and 636; SEQ ID NO: 637 and 638; SEQ ID NO: 639 and 640; SEQ ID NO: 641 and 642; SEQ ID NO: 643 and 644; SEQ ID NO: 645 and 646; SEQ ID NO: 647 and 648; SEQ ID NO: 649 and 650; SEQ ID NO: 651 and 652; SEQ ID NO: 653 and 654; SEQ ID NO: 655 and 656; SEQ ID NO: 657 and 658; SEQ ID NO: 659 and 660; SEQ ID NO: 661 and 662; SEQ ID NO: 663 and 664; SEQ ID NO: 665 and 666; SEQ ID NO: 667 and 668; SEQ ID NO: 669 and 670; SEQ ID NO: 671 and 672; SEQ ID NO: 673 and 674; SEQ ID NO: 675 and 676; SEQ ID NO: 677 and 678; SEQ ID NO: 679 and 680; SEQ ID NO: 681 and 682; SEQ ID NO: 683 and 684; SEQ ID NO: 685 and 686; SEQ ID NO: 687 and 688; SEQ ID NO: 689 and 690; SEQ ID NO: 691 and 692; SEQ ID NO: 693 and 694; SEQ ID NO: 695 and 696; SEQ ID NO: 697 and 698; SEQ ID NO: 699 and 700; SEQ ID NO: 701 and 702; SEQ ID NO: 703 and 704; SEQ ID NO: 705 and 706; SEQ ID NO: 707 and 708; SEQ ID NO: 709 and 710; SEQ ID NO: 711 and 712; SEQ ID NO: 713 and 714; SEQ ID NO: 715 and 716; SEQ ID NO: 717 and 718; SEQ ID NO: 719 and 720; SEQ ID NO: 721 and 722; SEQ ID NO: 723 and 724; SEQ ID NO: 725 and 726; SEQ ID NO: 727 and 728; SEQ ID NO: 729 and 730; SEQ ID NO: 731 and 732; SEQ ID NO: 733 and 734; SEQ ID NO: 735 and 736; SEQ ID NO: 737 and 738; SEQ ID NO: 739 and 740; SEQ ID NO: 741 and 742; SEQ ID NO: 743 and 744; SEQ ID NO: 745 and 746; SEQ ID NO: 747 and 748; SEQ ID NO: 749 and 750; SEQ ID NO: 751 and 752; SEQ ID NO: 753 and 754; SEQ ID NO: 755 and 756; SEQ ID NO: 757 and 758; SEQ ID NO: 759 and 760; SEQ ID NO: 761 and 762; SEQ ID NO: 763 and 764; SEQ ID NO: 765 and 766; SEQ ID NO: 767 and 768; SEQ ID NO: 769 and 770; SEQ ID NO: 771 and 772; SEQ ID NO: 773 and 774; SEQ ID NO: 775 and 776; SEQ ID NO: 777 and 778; SEQ ID NO: 779 and 780; SEQ ID NO: 781 and 782; SEQ ID NO: 783 and 784; SEQ ID NO: 785 and 786; SEQ ID NO: 787 and 788; SEQ ID NO: 789 and 790; SEQ ID NO: 791 and 792; SEQ ID NO: 793 and 794; SEQ ID NO: 795 and 796; SEQ ID NO: 797 and 798; SEQ ID NO: 799 and 800; SEQ ID NO: 801 and 802; SEQ ID NO: 803 and 804; SEQ ID NO: 805 and 806; SEQ ID NO: 807 and 808; SEQ ID NO: 809 and 810; SEQ ID NO: 811 and 812; SEQ ID NO: 813 and 814; SEQ ID NO: 815 and 816; SEQ ID NO: 817 and 818; SEQ ID NO: 819 and 820; SEQ ID NO: 821 and 822; SEQ ID NO: 823 and 824; SEQ ID NO: 825 and 826; SEQ ID NO: 827 and 828; SEQ ID NO: 829 and 830; SEQ ID NO: 831 and 832; SEQ ID NO: 833 and 834; SEQ ID NO: 835 and 836; SEQ ID NO: 837 and 838; SEQ ID NO: 839 and 840; SEQ ID NO: 841 and 842; SEQ ID NO: 843 and 844; SEQ ID NO: 845 and 846; SEQ ID NO: 847 and 848; SEQ ID NO: 849 and 850; SEQ ID NO: 851 and 852; SEQ ID NO: 853 and 854; SEQ ID NO: 855 and 856; SEQ ID NO: 857 and 858; SEQ ID NO:859 and 860; SEQ ID NO:861 and 862; SEQ ID NO:863 and 864; SEQ ID NO:865 and 866; SEQ ID NO:867 and 868; SEQ ID NO:869 and 870; SEQ ID NO:871 and 872; SEQ ID NO:873 and 874; SEQ ID NO:875 and 876; SEQ ID NO:877 and 878; SEQ ID NO:879 and 880; SEQ ID NO:881 and 882; SEQ ID NO:883 and 884; SEQ ID NO:885 and 886; SEQ ID NO:887 and 888; SEQ ID NO:889 and 890; SEQ ID NO:891 and 892; SEQ ID NO:893 and 894; SEQ ID NO:895 and 896; SEQ ID NO:897 and 898; SEQ ID NO:899 and 900; SEQ ID NO:901 and 902; SEQ ID NO:903 and 904; SEQ ID NO:905 and 906; SEQ ID NO:907 and 908; SEQ ID NO:909 and 910; SEQ ID NO:911 and 912; SEQ ID NO:913 and 914; SEQ ID NO:915 and 916; SEQ ID NO:917 and 918; SEQ ID NO:919 and 920; SEQ ID NO:921 and 922; SEQ ID NO:923 and 924; SEQ ID NO:925 and 926; SEQ ID NO:927 and 928; SEQ ID NO:929 and 930; SEQ ID NO:931 and 932; SEQ ID NO:933 and 934; SEQ ID NO:935 and 936; SEQ ID NO:937 and 938; SEQ ID NO:939 and 940; SEQ ID NO:941 and 942; SEQ ID NO:943 and 944; SEQ ID NO:945 and 946; SEQ ID NO:947 and 948; SEQ ID NO:949 and 950; SEQ ID NO:951 and 952; SEQ ID NO:953 and 954; ID NO:955 and 956; SEQ ID NO:957 and 958; SEQ ID NO:959 and 960; SEQ ID NO:961 and 962; SEQ ID NO:963 and 964; SEQ ID NO:965 and 966; SEQ ID NO:967 and 968; SEQ ID NO:969 and 970; SEQ ID NO:971 and 972; SEQ ID NO:973 and 974; SEQ ID NO:1759 and 1760; SEQ ID NO:1761 and 1762; SEQ ID NO:1763 and 1764; SEQ ID NO:1765 and 1766; SEQ ID NO:1767 and 1768; SEQ ID NO:1769 and 1770; SEQ ID NO:1771 and 1772; SEQ ID NO:1773 and 1774; SEQ ID NO:1775 and 1776; SEQ ID NO:1777 and 1778; SEQ ID NO:1779 and 1780; SEQ ID NO:1781 and 1782; SEQ ID NO:1783 and 1784; SEQ ID NO:1785 and 1786; SEQ ID NO:1787 and 1788; SEQ ID NO:1789 and 1790; SEQ ID NO:1791 and 1792; SEQ ID NO:1793 and 1794; SEQ ID NO:1795 and 1796; SEQ ID NO:1797 and 1798; SEQ ID NO:1799 and 1800; SEQ ID NO:1801 and 1802; SEQ ID NO:1803 and 1804; SEQ ID NO:1805 and 1806; SEQ ID NO:1807 and 1808; SEQ ID NO:1809 and 1810; SEQ ID NO:1811 and 1812; SEQ ID NO:1813 and 1814; SEQ ID NO:1815 and 1816; SEQ ID NO:1817 and 1818; SEQ ID NO:1819 and 1820; SEQ ID NO:1821 and 1822; SEQ ID NO:1823 and 1824; SEQ ID NO:1825 and 1826; SEQ ID NO:1827 and 1828; SEQ ID NO:1829 and 1830; SEQ ID NO:1831 and 1832; SEQ ID NO:1833 and 1834; SEQ ID NO:1835 and 1836; SEQ ID NO:1837 and 1838; SEQ ID NO:1839 and 1840; SEQ ID NO:1841 and 1842; SEQ ID NO:1843 and 1844; SEQ ID NO:1845 and 1846; SEQ ID NO:1847 and 1848; SEQ ID NO:1849 and 1850; SEQ ID NO:1851 and 1852; SEQ ID NO:1853 and 1854; SEQ ID NO:1855 and 1856; SEQ ID NO:1857 and 1858; SEQ ID NO:1859 and 1860; SEQ ID NO:1861 and 1862; SEQ ID NO:1863 and 1864; SEQ ID NO:1865 and 1866; SEQ ID NO:1867 and 1868; SEQ ID NO:1869 and 1870; SEQ ID NO:1871 and 1872; or, SEQ ID NO:1873 and 1874.

76. The dsRNAi agent of claim 1, wherein the dsRNAi agent further comprises at least one targeting ligand conjugated to the sense strand and/or the antisense strand, optionally wherein the at least one targeting ligand binds specifically to an asialoglycoprotein receptor (ASGPR) on the surface of a liver cell such as a hepatocyte or to a receptor on the surface of a fat cell such as an adipocyte.

77.-86. (canceled)

87. The dsRNAi agent of claim 76, wherein the dsRNAi agent has the structure shown in any one of the following Formulae 1 to 33, or is a pharmaceutically acceptable salt thereof:

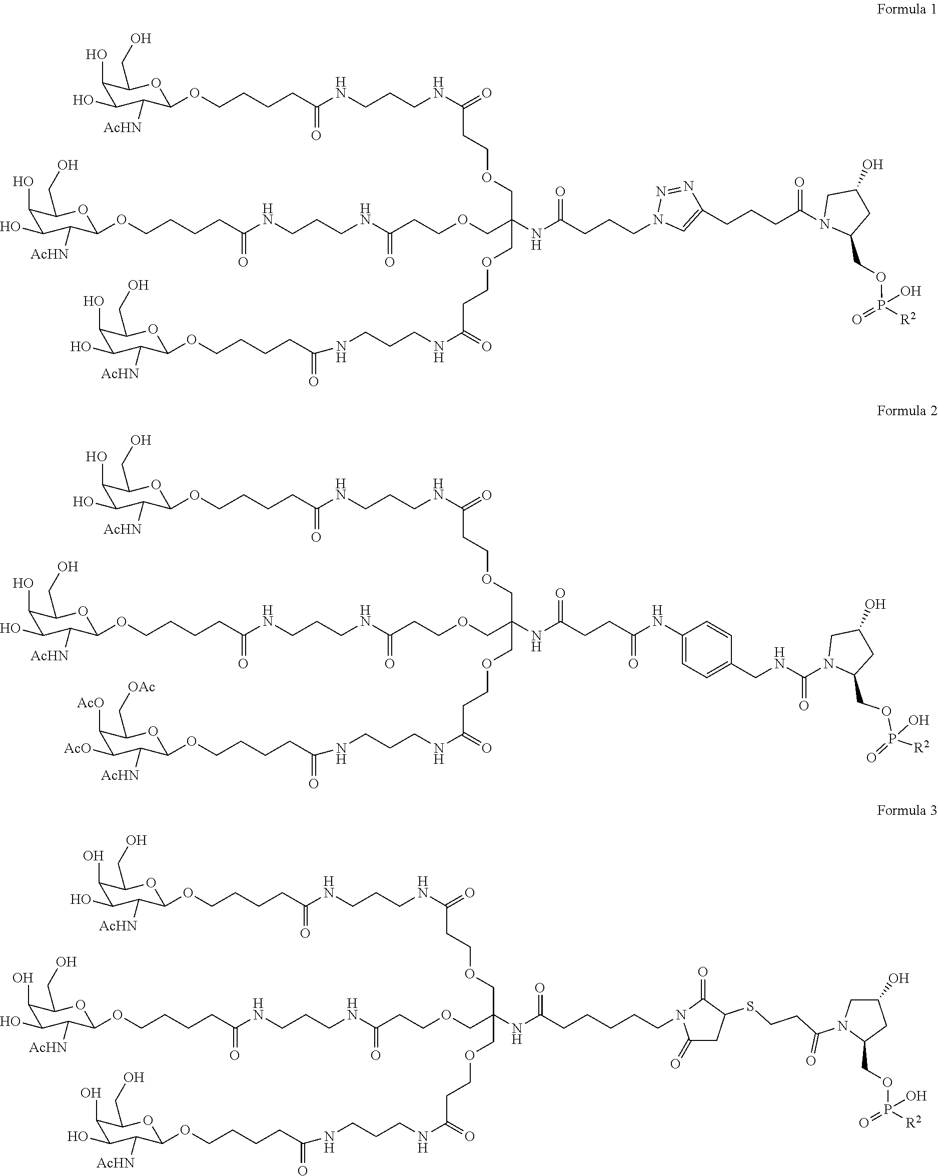

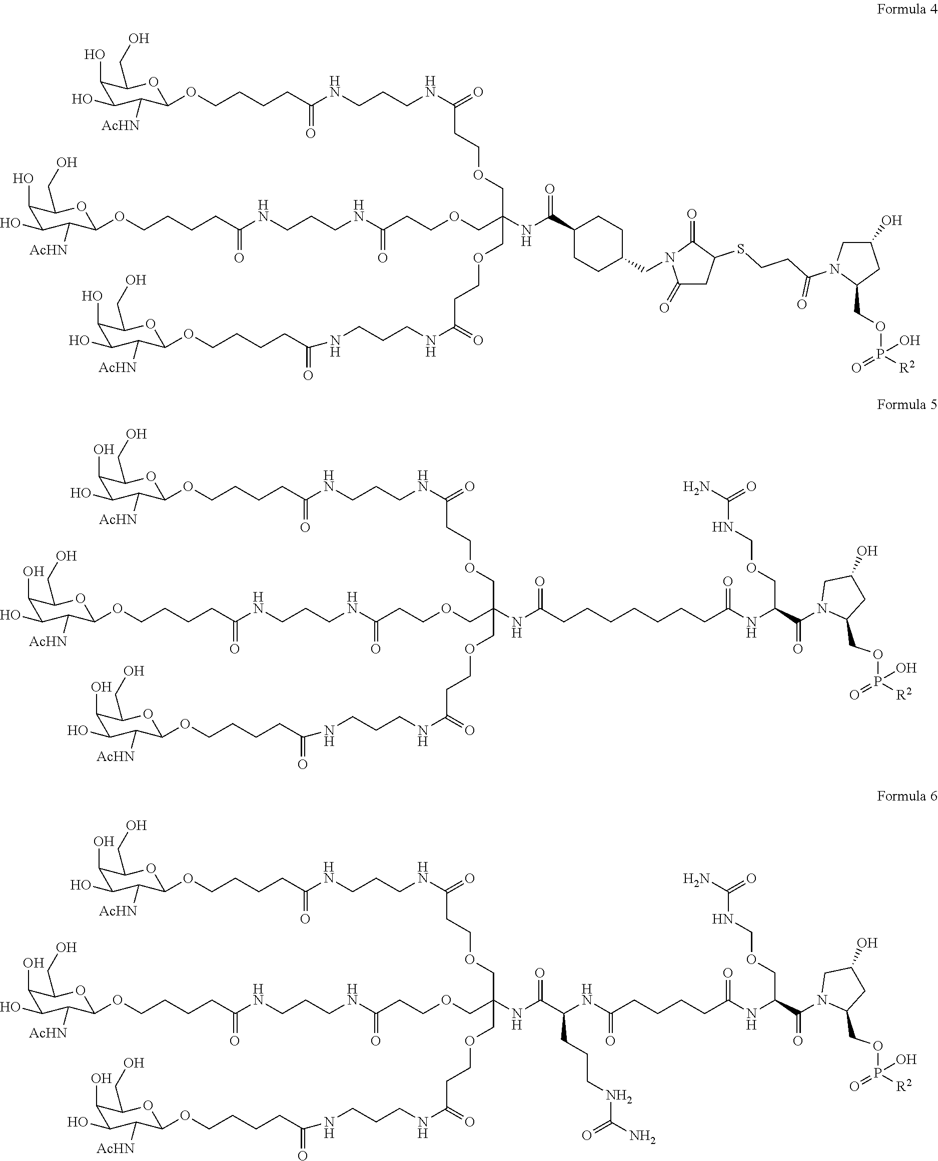

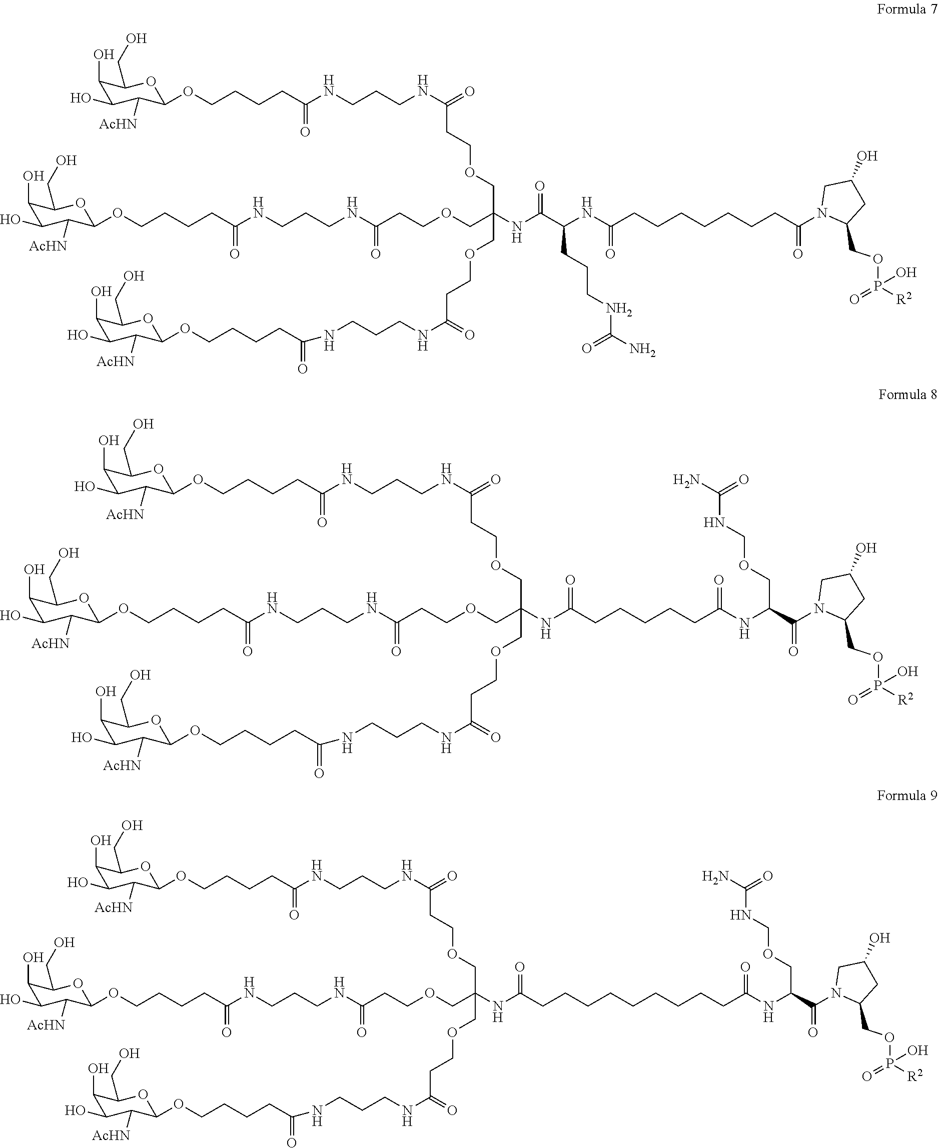

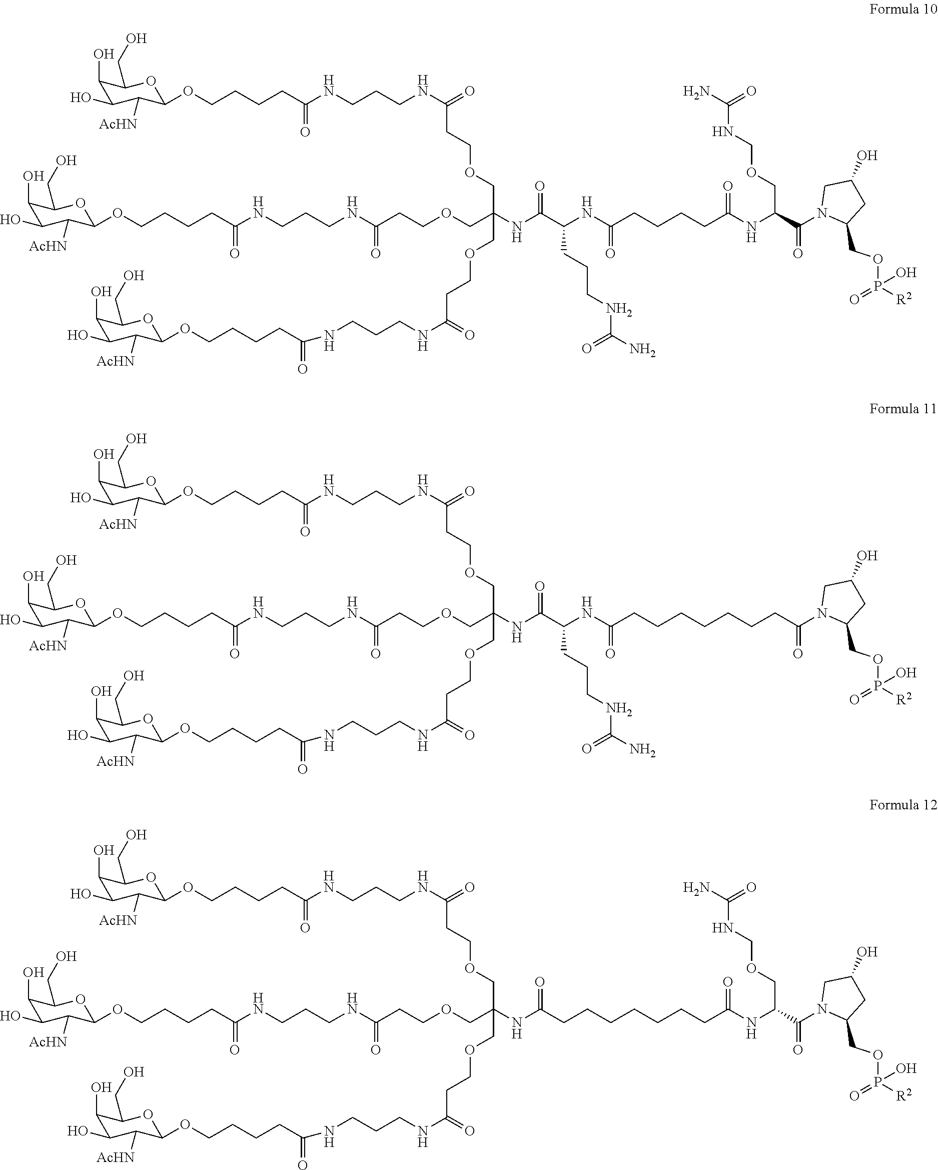

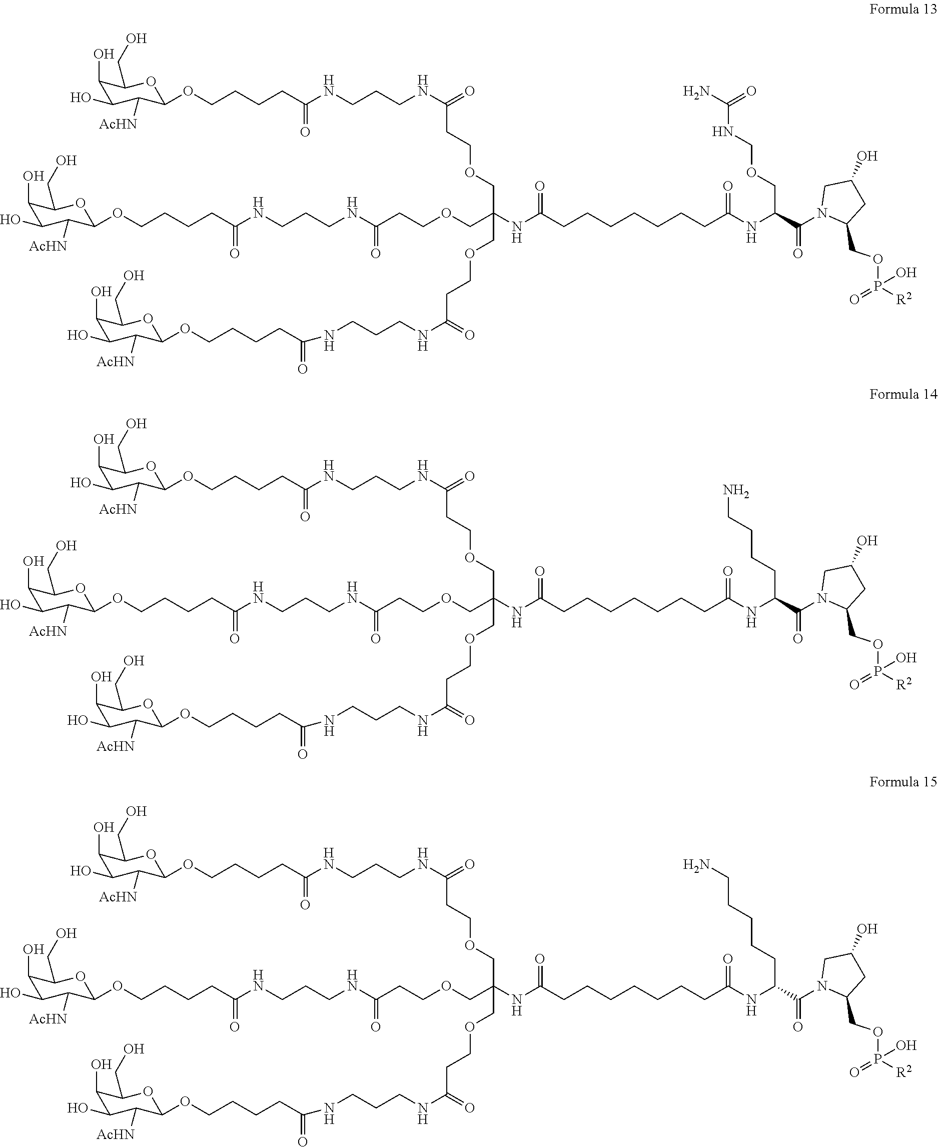

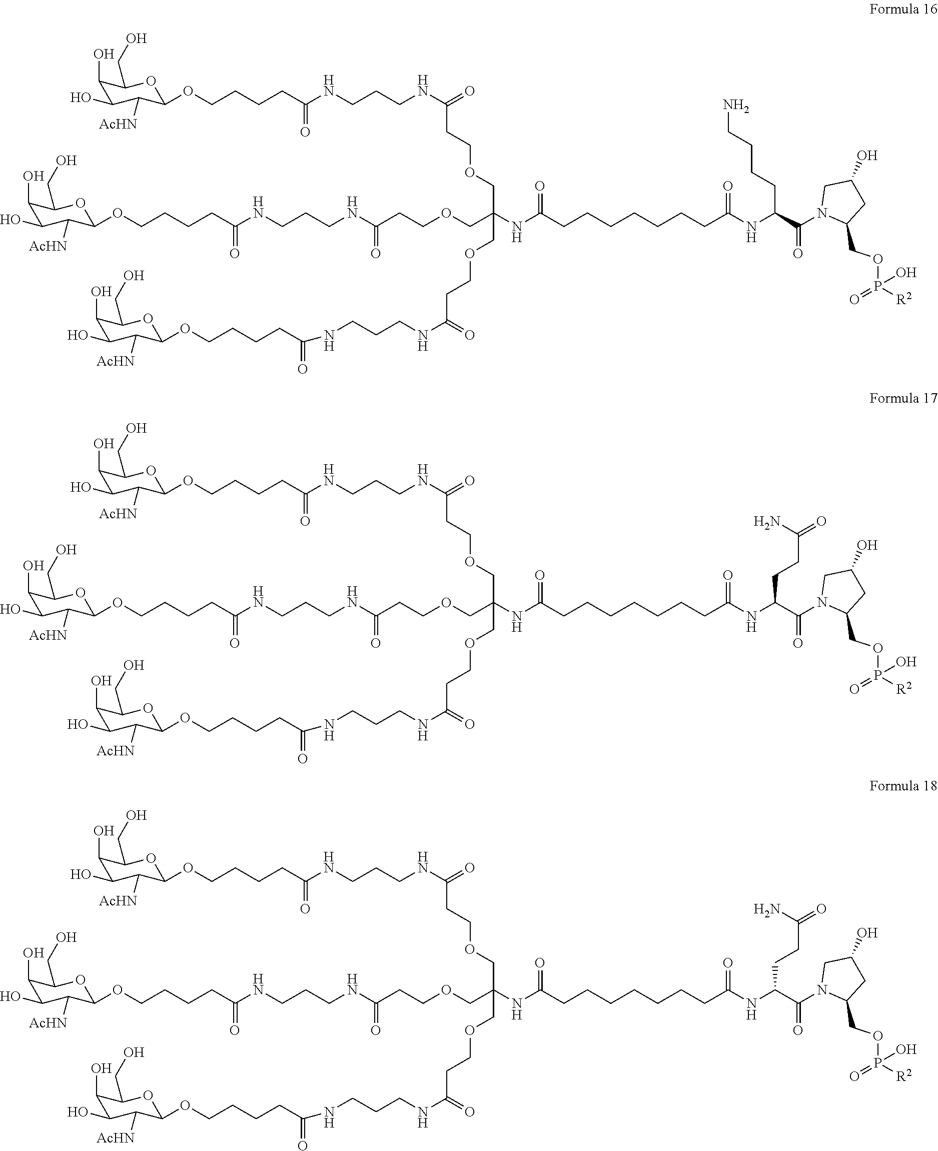

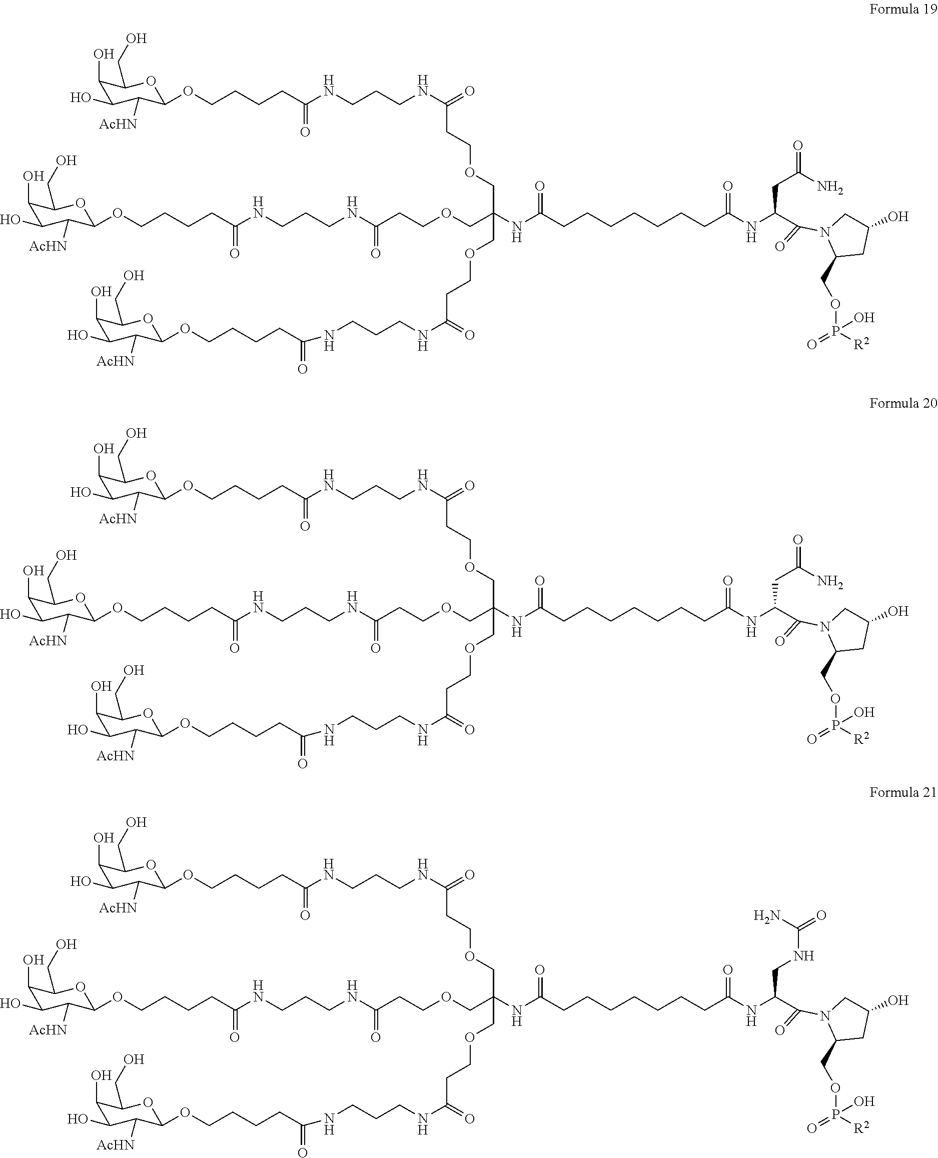

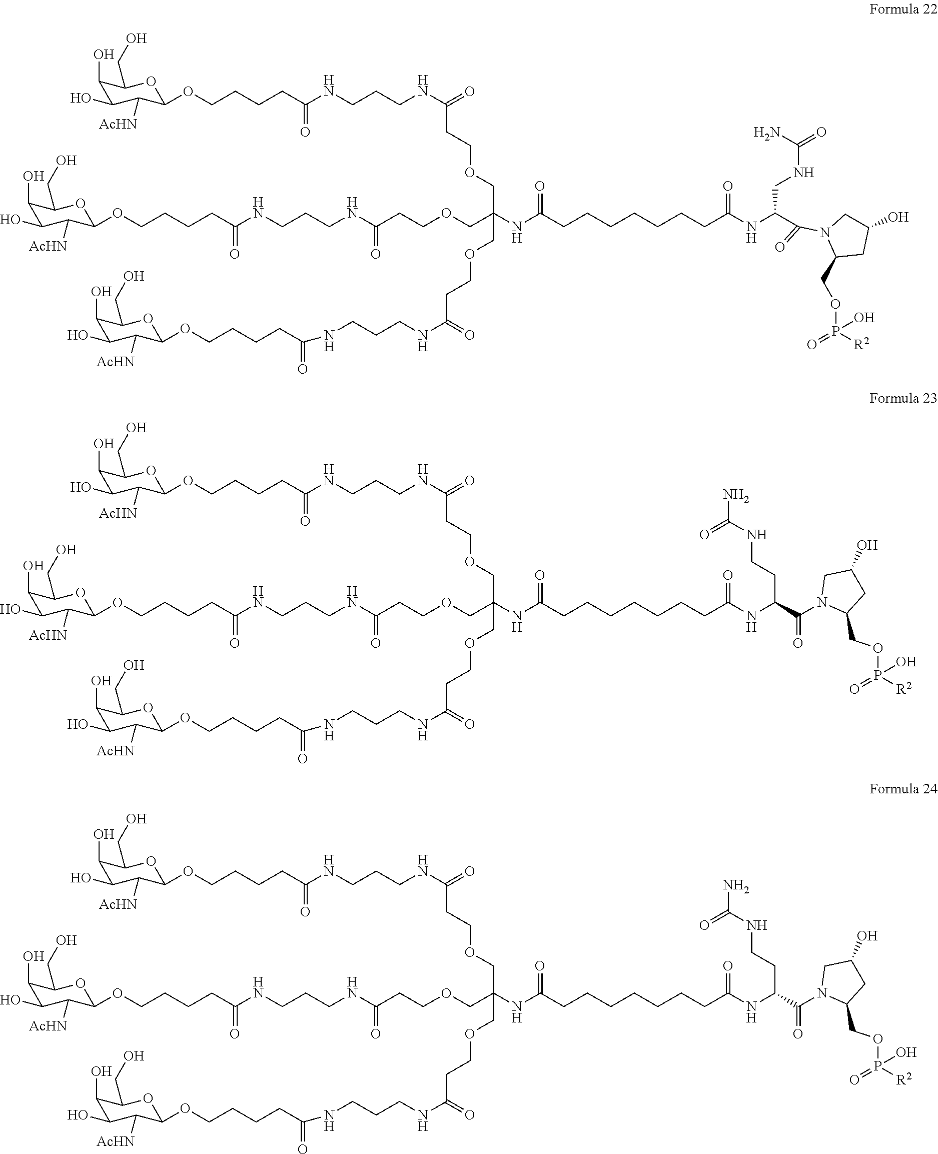

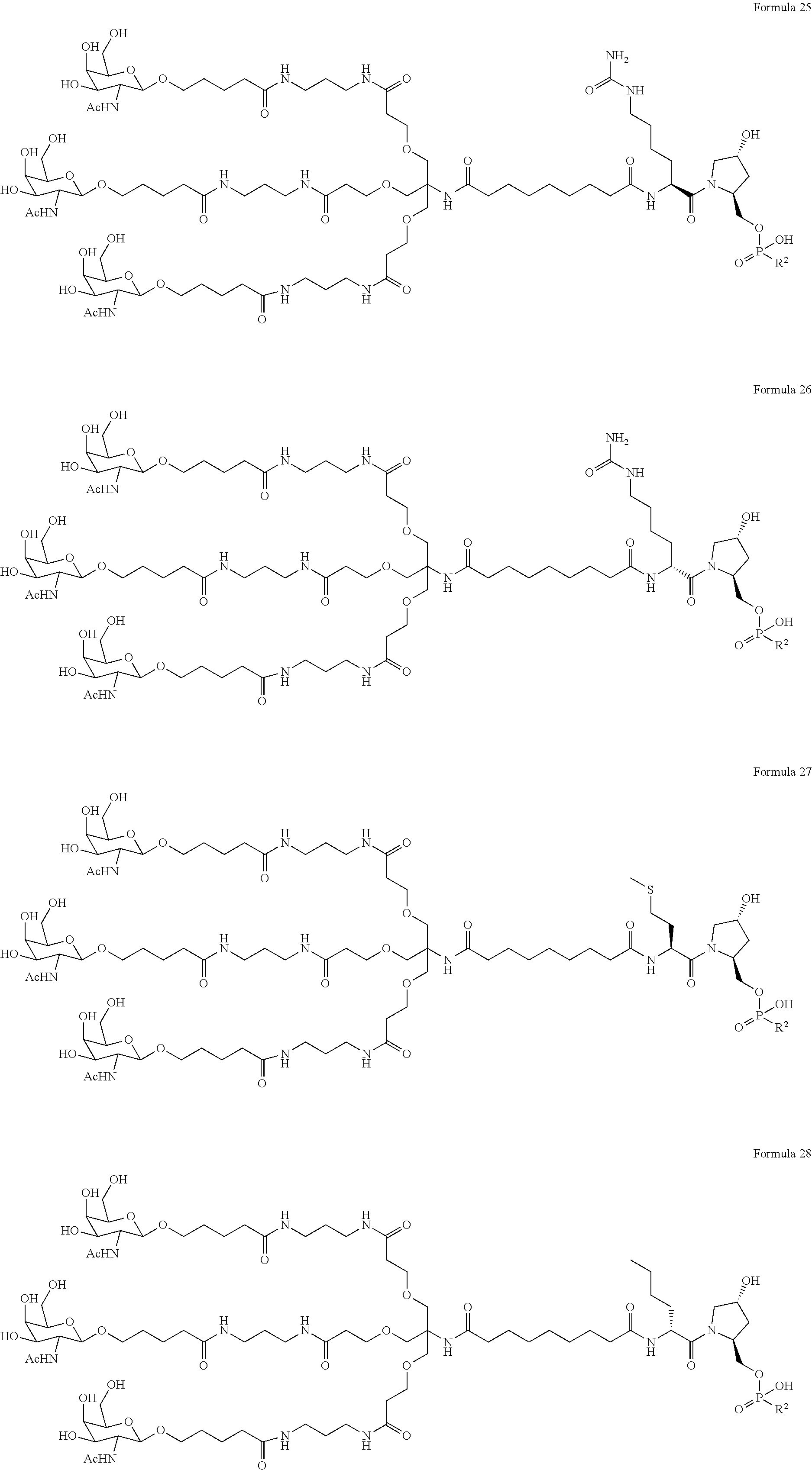

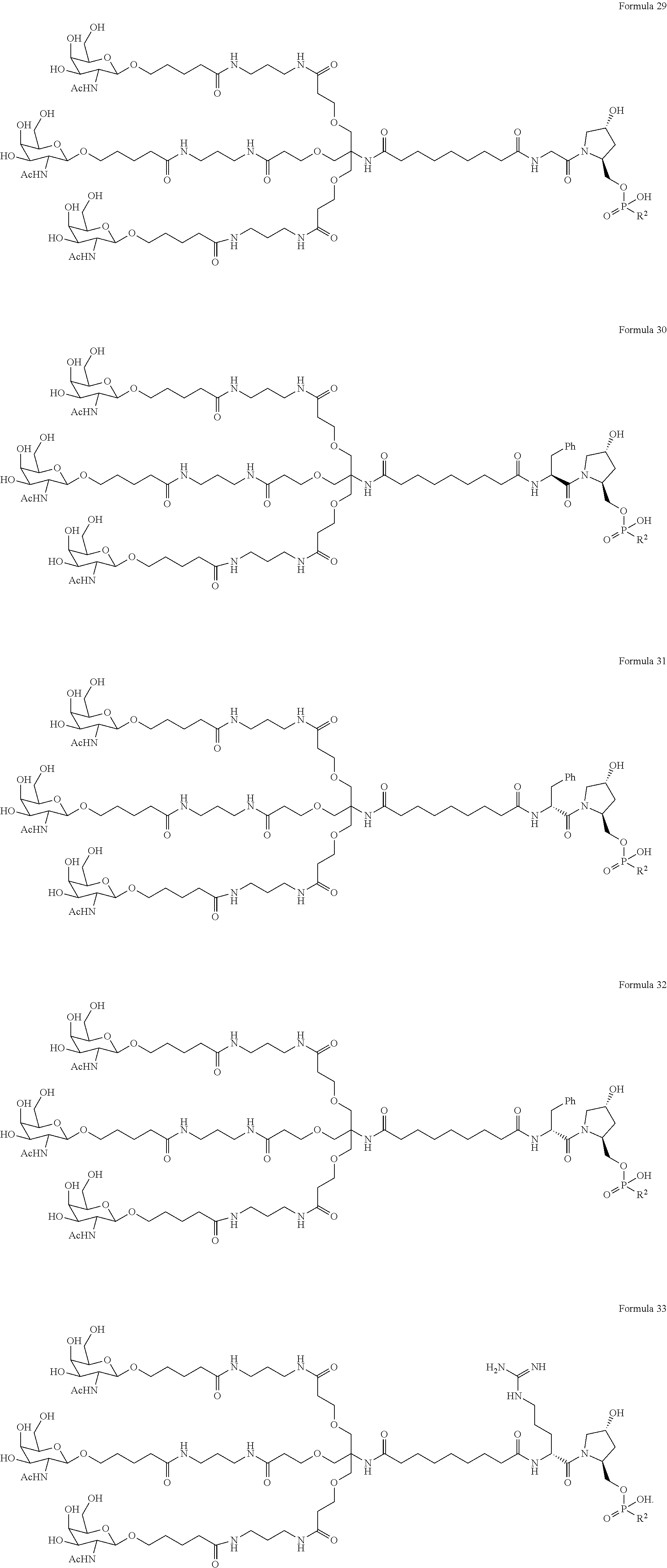

88.-90. (canceled)

91. A pharmaceutical composition comprising the dsRNAi agent of claim 1 or a pharmaceutically acceptable salt thereof, and a pharmaceutically acceptable carrier, excipient or diluent.

92.-101. (canceled)

102. A method of inhibiting the expression of inhibin subunit beta E (INHBE) in a cell, the method comprising contacting the cell with the dsRNAi agent of claim 1, such that INHBE expression in the cell is inhibited.

103.-106. (canceled)

107. A method for prevention and/or treatment of an INHBE-associated disease or disorder in a subject, the method comprising administering to the subject a therapeutically effective amount of the dsRNAi agent of claim 1, such that the INHBE-associated disease or disorder is prevented or treated.

108. The method of claim 107, wherein the subject suffers from, is at risk for, or displays at least one symptom of, an INHBE-associated disease or disorder selected from a metabolic disorder, metabolic syndrome, type 2 diabetes, obesity, pre-diabetes, elevated triglyceride levels, lipodystrophy, liver inflammation, fatty liver, hypercholesterolemia, disorders associated with elevated liver enzymes, nonalcoholic steatohepatitis, cardiovascular disease, kidney disease, a cardiometabolic disorder, abdominal obesity, insulin resistance, hypertension, hyperlipidemia, and a cancer associated with INHBE expression.

109.-126. (canceled)

127. The method of claim 107, wherein INHBE expression is inhibited by at least about 30%, at least about 35%, at least about 40%, at least about 45%, at least about 50%, at least about 55%, at least about 60%, at least about 65%, at least about 70%, at least about 75%, at least about 80%, at least about 85%, at least about 90% or at least about 95% in the cell or in the subject.

128.-145. (canceled)
